(19) United States
(12) Patent Application Publication
(10) Pub. No.: US 2009/0118283 A1
(43) Pub. Date: May 7, 2009

(54) Amide Derivatives as Kinase Inhibitors

(75) Inventors: Olivier Raynald Defert, Sebourg (FR); Gert De Wilde, Zele (BE); Petra Blom, Destelbergen (BE); Dirk Casimir Maria Leysen, Lokeren (BE); Thomas Brown, Essex (GB); Nadzeya Kaval, Gent (BE)

Correspondence Address: WOLF GREENFIELD & SACKS, P.C., 600 ATLANTIC AVENUE, BOSTON, MA, 02210-2206, US

(73) Assignee: Devgen NV, Zwijnaarde (BE)

(21) Appl. No.: 11/988,699

(22) PCT Filed: Jul. 11, 2006
(86) PCT No.: PCT/EP2006/006763

§ 371 Date: Sep. 9, 2008

Related U.S. Application Data
(60) Provisional application No. 60/698,195, filed on Jul. 11, 2005.

(30) Foreign Application Priority Data
Dec. 21, 2005 (EP) PCTEP200513771
Jun. 2, 2006 (EP) PCTEP200605305

(51) Int. Cl. A61K 31/4409; C07D 211/72; C07D 239/38; A61K 31/505; A61K 31/5377; A61P 25/08; A61P 9/00; A61P 3/10; A61K 31/501; C07D 405/12; C07D 413/12

(52) U.S. Cl. 514/235.5; 546/312; 544/316; 544/238; 544/131; 514/352; 514/274; 514/252.03

(57) ABSTRACT

The present invention relates to new AGC kinase inhibitors, in particular to compounds of Formula (I) or (II) or a stereoisomer tautomer, racemic, metabolite, pro- or predrug, salt, hydrate, or solvate thereof, wherein Ar¹, Ar², R¹, R³, p and n have the meaning defined in the claims In particular, the present invention relates to more specifically AGC kinases inhibitors, compositions, in particular pharmaceuticals, comprising such inhibitors, and to uses of such inhibitors in the treatment and prophylaxis of disease.

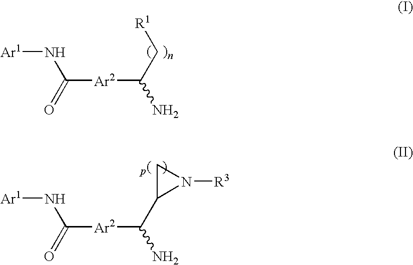

### FIELD OF THE INVENTION

The present invention relates to new kinase inhibitors, compositions, in particular pharmaceuticals, comprising such inhibitors, and to uses of such inhibitors in the treatment and prophylaxis of disease.

### BACKGROUND OF THE INVENTION

It is known in the prior art that inhibitors of certain kinases can be used in the treatment of diabetes, obesity and other metabolic diseases. Some examples of such kinases include JNK1, p38 kinase, GSK-3, IKKbeta (IKappaB kinase beta) and p70S6K.

The art also describes that several isoforms of protein kinase C (“PKC”) are associated with metabolic diseases such as diabetes and obesity. Reference is inter alia made to U.S. Pat. No. 6,376,467, U.S. Pat. No. 6,284,784, U.S. Pat. No. 6,080,784, U.S. Pat. No. 6,057,440, U.S. Pat. No. 5,962,504, WO 02/22709, WO 01/30331, WO 96/40894 and the further references cited therein.

As described in these references, there are currently 10 known isoforms of PKC, known as alpha, beta-I, beta-II, gamma, delta, epsilon, zeta, eta, iota/lambda and theta, respectively (Nishizuka, Science 258, 607-614 (1992); Selbie et al., J. Biol. Chem. 268, 24296-24302 (1993)). Based on sequence homology and biochemical properties, these PKC isozymes are generally subdivided into three groups:
(a) the group of “conventional” PKCs comprising the alpha, beta-I, beta-II and gamma isozymes, which are all regulated by calcium, diacylglycerol and/or phorbol esters;
   (b) the group of “novel” PKCs comprising the delta, epsilon, theta and eta isozymes, which are all calcium-independent, but diacylglycerol- and/or phorbol ester-sensitive; and
   (c) the group of “atypical” PKCs, the zeta and iota/lambda isozymes, which are insensitive to calcium, diacylglycerol and/or phorbol 12-myristate 13-acetate.

A further subgroup may be comprised of PKC mu and protein kinase D (see for example U.S. Pat. No. 6,376,467; Johannes et al, Biol. Chem. 269, 6140-6148 (1994); and Valverde et al, Proc. Natl. Acad. Sci. USA 91, 8572-8576 (1994)).

U.S. Pat. No. 6,057,440, U.S. Pat. No. 5,698,578 and U.S. Pat. No. 5,739,322 describe the use of bisindolylmaleimide compounds as specific inhibitors of PKC beta in the prevention and treatment of diabetes and diabetes-related complications. These aforementioned patent applications and patents also describe an assay that can be used to determine the specificity of a given inhibitor for one isoform of PKC compared to another (referred to in these patents as the “PKC Enzyme Assay”).

German patent application DE 197 40 384 A1 describes that antisense oligonucleotide sequences specific for certain PKC isoforms, and in particular against the alpha, delta, epsilon and zeta isoforms, may be used in the prevention or treatment of complications associated with diabetes.

WO 01/81633 describes the association on PKC zeta with diabetes. Similarly, WO 94/18328 describes that the “atypical” PKC isozyme iota is involved in diabetes.

The link between PKC epsilon and diabetes/obesity has been established in two model systems for diabetes and obesity, viz the sand rat Psammomys and the High Fat Fed Rat. Reference is inter alia made to Shafrir et al., Annals New York Academy of Sciences 892:223-241 (1999), Donelly and Qu, Clin. Exper. Pharmacol. And Phsyiol. 25: 79-87 (1998) and Qu et al., Journal of Endocrinology 162: 207-214 (1999). The latter two references also suggest that PKC theta may be involved in diabetes and obesity.

WO 00/01805 describes PKC-epsilon knock out mice. This animal model is used to demonstrate that PKC epsilon can be used as a target for drugs to reduce anxiety, modulate alcohol consumption and drug abuse, addiction, withdrawal syndrome, muscle spasms, convulsive seizures, epilepsy and to modulate the action of drugs that target the GABA-A receptor.

WO 00/01415 and U.S. Pat. No. 6,376,467 describe the use of inhibitors of PKC epsilon in the treatment of pain, in particular chronic hyperalgesia and/or inflammatory pain (reference is also made to WO 02/102232 and WO 03/089457). As examples of suitable inhibitors, both peptides as well as small molecules are mentioned. WO 97/15575 and WO 01/83449 describe modulators of PKC with specific binding activity with respect to PKC epsilon. Peptide inhibitors that provide isozyme-specific modulation of PKC (in particular of PKC gamma and PKC epsilon) are described in WO 03/089456 and WO 03/089457.

For the sequence of human PKC epsilon, reference is made inter alia made to Basta et al., Biochim. Biophys Acta, 1132 (1992), 154-160, as well as to SWISS-PROT entry Q02156 and EMBL entry X65293.

WO 03/04612 describes the use of inhibitors of PKC theta as an immunosuppressive agent (e.g. during organ transplant) and for treatment of systemic lupus erythematosus. Reference is also made to Castrillo et al., J. Exp. Med., 194, 9 (2001), p. 1231-1242, who describe that PKC epsilon plays a critical role as a mediator in signaling cascades of activated macrophages, and that the absence of PKC epsilon can compromise the successful initiation of an effective immune response against a range of bacterial pathogens.

US 2003/0134774 describes the use of inhibitors of PKC epsilon and PKC theta in inhibiting the onset of a cardiac disorder and the progression of heart failure.

For other potential uses of inhibitors of PKC and/or of specific isoforms of PKC, reference is for example made to US 2002/0164389, US 2003/0118529, US 2003/0176424, US 2003/0176423, US 2003/0166678, US 2003/0134774, US 2003/0166678, US 2003/0176424, US 2003/0199423, WO 03/82859, WO 02/103000 and WO 02/87417.

WO2004/056982 describes four kinases—referred to as “JIK”, “PSK”, “TAO1” and “Q9P216”, respectively)—which are potential targets in metabolic disease.

### SUMMARY OF THE INVENTION

We have surprisingly found that the compounds described herein act as inhibitors of AGC-kinases and in particular as inhibitors of the novel PKC's such as the calcium-independent but diacylglycerol- and/or phorbol ester-sensitive PKC epsilon isoform. We also surprisingly found that the compounds described herein act as inhibitors of PKC epsilon and PKC theta. We have also surprisingly found that the compounds described herein act as inhibitors of other AGC-kinases and in particular of ROCK.

Viewed from a first aspect, the invention provides a compound of Formula I or II or a stereoisomer, tautomer, racemic, metabolite, pro- or predrug, salt, hydrate, or solvate thereof,

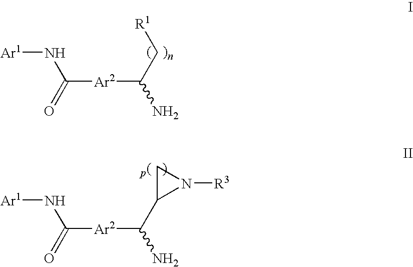

wherein:
Ar¹ is an aromatic 6-membered first ring containing carbon atoms and at least one nitrogen atom, said first ring being optionally fused to a saturated, unsaturated or aromatic 4-, 5-, 6-, or 7-membered second ring containing carbon atoms and optionally at least one nitrogen atom, said first or said second rings being independently substituted with one or more substituents independently selected from the group comprising hydrogen, halogen, alkyl, cycloalkyl, alkenyl, alkynyl, aralkyl, heteroarylalkyl, cycloalkylalkyl, acyl, aryl or heteroaryl wherein said substituents are optionally substituted by one or more further substituents selected from the group comprising halo, hydroxyl, oxo, nitro, amido, carboxy, amino, cyano, haloalkoxy, and haloalkyl;
Ar² is an aromatic 5- or 6-membered third ring containing carbon atoms and optionally one or two heteroatoms, said third ring optionally fused to an aromatic 6-membered fourth ring containing carbon atoms and optionally at least one heteroatom atom, wherein said third ring is optionally substituted with one or more substituents selected from the group comprising halogen, alkenyl, alkyl, alkynyl, acylamino, alkoxy, arylamino, nitro, haloalkoxy, aryl or heteroaryl, wherein said substituents are optionally substituted by one or more further substituents selected from the group comprising halo, hydroxyl, oxo, nitro, amido, carboxy, amino, cyano, haloalkoxy, and haloalkyl;
n is an integer selected from 1, 2 or 3; and
p is an integer selected from 2, 3, 4 or 5; preferably 3 or 4, more preferably 3, and
R¹ is selected from the Formula:

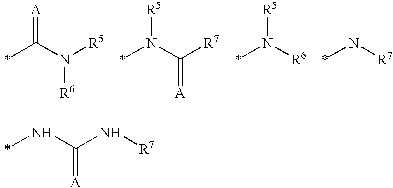

R³ is selected from the Formula:

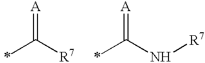

A is an oxygen or sulfur atom;
R⁵, R⁶ and R⁷ are each independently selected from hydrogen, or a group selected from alkoxy, alkyl, alkylamino, alkylaminoalkyl, alkylcarbonyl, alkylcarbonylamino, amino, aralkyl, aryl, carbonylamino, cycloalkyl, formylamino, heteroaryl, heteroarylalkyl, heterocyclyl, or fused to the cycloalkyl, aryl, heterocyclyl or heteroaryl group may be one or more cycloalkyl, aryl, heterocyclyl or heteroaryl, preferably R⁵ is selected from hydrogen, alkyl, cycloalkyl, cycloalkylalkyl, aryl or aralkyl,
each group being optionally substituted by one or more substituent selected from halo, alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylalkenylaminooxy, heteroarylalkyl, heteroarylcarbonylamino, heterocyclyl, hydroxyalkyl, nitro, oxo, sulfonyl, or fused to the cycloalkyl, aryl, heterocyclyl substituent or heteroaryl may be one or more cycloalkyl, aryl, heterocyclyl or heteroaryl,
each of said substituent being optionally substituted by one or more further substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl.

Viewed from a further aspect, the invention provides a method for synthesizing a compound having the Formula XXIII comprising the steps of reacting a compound of Formula XX:

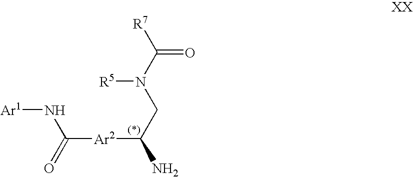

with Noyori's catalyst thereby obtaining a compound of Formula XXI:

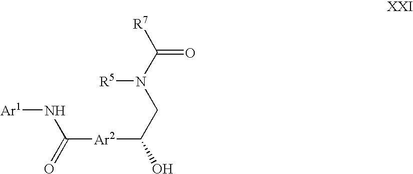

reacting compound of Formula XXI with diphenylphosphoryl azide (DPPA) and 1,8-diazabicyclo[5.4.0]undec-7-ene (DBU) thereby obtaining azide of Formula XXII, and

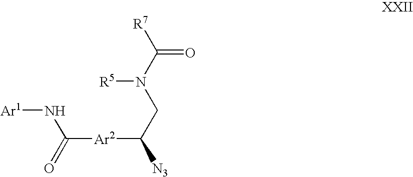

reacting compound of Formula XXII with Pd/C thereby obtaining the compound of Formula XXIII,

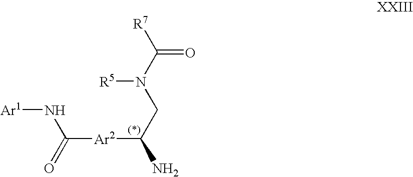

wherein Ar² is phenylene and Ar¹, R⁵ and R⁷ have the same meaning as that defined hereinabove. Viewed from a further aspect, the invention provides a pharmaceutical and/or veterinary composition comprising a compound of the invention.

Viewed from a still further aspect, the invention provides a compound of the invention for use in human or veterinary medicine.

Viewed from a still further aspect, the invention provides the use of a compound of the invention, or a composition comprising such a compound, for inhibiting the activity of PKC epsilon, in vitro or in vivo.

Viewed from a still further aspect, the invention provides the use of a compound of the invention in the preparation of a medicament for the prevention and/or treatment of at least one disease and/or disorder selected from the group comprising metabolic diseases, such as Type I and Type II diabetes; anxiety; addiction; withdrawal symptoms; muscle spasms; convulsive seizures; epilepsy; pain; cardiovascular disease, including heart disease; inflammatory diseases; and/or for regulating the immune system and/or an immune response and/or inflammatory response in a mammal.

Viewed from a still further aspect, the invention provides the use of a compound of the invention in the preparation of a medicament for the prevention and/or treatment of type II diabetes, and/or for preventing, treating and/or alleviating complications and/or symptoms associated therewith.

Viewed from a still further aspect, the invention provides the use of a compound of the invention in the preparation of a medicament for the prevention and/or treatment of obesity, and/or for preventing, treating and/or alleviating complications and/or symptoms associated therewith and/or alleviating complications and/or symptoms associated therewith.

Viewed from a still further aspect, the invention provides the use of a compound of the invention in the preparation of a medicament for the prevention, treatment and/or management of pain, and/or for preventing, treating and/or alleviating complications and/or symptoms associated therewith.

Viewed from a still further aspect, the invention provides the use of a compound of the invention in the preparation of a medicament for the prevention and/or treatment of cardiovascular diseases, such as acute stroke, congestive heart failure, cardiovascular ischemia, heart disease, cardiac remodeling, and/or for preventing, treating and/or alleviating complications and/or symptoms associated therewith.

Viewed from a still further aspect, the invention provides the use of a compound of the invention, or a composition comprising such a compound, for inhibiting the activity of PKC epsilon and PKC theta, in vitro or in vivo.

Viewed from a still further aspect, the invention provides the use of a compound of the invention in the preparation of a medicament for the prevention and/or treatment of inflammatory diseases (such as contact dermatitis, psoriasis, rheumatoid arthritis, inflammatory bowel disease, Crohn's disease, ulcerative colitis); kidney disease (such as renal dysfunction); cancer (such as cancer of the lung, intestine, nerve, skin, pancreas, liver, uterus, ovary, brain, thyroid gland, or leukemia or lymphoma melanoma); blood disease (such as sepsis, eosinophilia or endotoxemia); atherosclerosis; allergy and autoimmune diseases or disorders; AIDS; diabetes (hyperglycemia); obesity and pancreas disease; multiple sclerosis and/or for preventing, treating and/or alleviating complications and/or symptoms associated therewith.

Viewed from a still further aspect, the invention provides the use of a compound of the invention in the preparation of a medicament for the prevention and/or treatment of inflammatory diseases, such as contact dermatitis, psoriasis, rheumatoid arthritis, inflammatory bowel disease, Crohn's disease, ulcerative colitis, and/or for preventing, treating and/or alleviating complications and/or symptoms and/or inflammatory responses associated therewith.

Viewed from a still further aspect, the invention provides the use of a compound of the invention in the preparation of a medicament for the prevention and/or treatment of sepsis, such as septic shock, and/or for preventing, treating and/or alleviating complications and/or symptoms associated therewith.

Viewed from a still further aspect, the invention provides the use of a compound of the invention, or a composition comprising such a compound, for inhibiting the activity of at least one ROCK, for example ROCKII and/or ROCKI isoforms.

Viewed from a still further aspect, the invention provides the use of a compound of the invention in the preparation of a medicament for the prevention and/or treatment of at least one disease and/or disorder selected from the group comprising eye diseases; erectile dysfunction; cardiovascular diseases; vascular diseases; proliferative diseases; inflammatory diseases; neurological diseases and disease of the central nervous system (CNS); bronchial asthma; osteoporosis; renal diseases; and AIDS.

Viewed from a still further aspect, the invention provides the use of a compound of the invention in the preparation of a medicament for the prevention and/or treatment of eyes diseases including retinopathy, macular degeneration and glaucoma, and/or for preventing, treating and/or alleviating complications and/or symptoms associated therewith.

Viewed from a still further aspect, the invention provides the use of a compound of the invention in the preparation of a medicament for the prevention and/or treatment of cardiovascular and vascular diseases, including but not limited to angina, coronary vasospasm, cerebral vasospasm, pulmonary vasoconstriction, restenosis, hypertension, (pulmonary) hypertension, arteriosclerosis, thrombosis (including deep thrombosis), platelet related diseases, acute stroke, congestive heart failure, cardiovascular ischemia, heart disease, and cardiac remodeling and/or for preventing, treating and/or alleviating complications and/or symptoms associated therewith and/or alleviating complications and/or symptoms associated therewith.

Viewed from a still further aspect, the invention provides the use of a compound of the invention in the preparation of a medicament for the prevention, treatment and/or management of neurological and CNS disorders: including but not limited to stroke, multiple sclerosis, brain or spinal cord injury, inflammatory and demyelinating diseases such as Alzheimer's disease, MS and neuropathic pain, and/or for preventing, treating and/or alleviating complications and/or symptoms associated therewith.

Viewed from a still further aspect, the invention provides the use of a compound of the invention in the preparation of a medicament for the prevention and/or treatment of proliferative diseases such as cancer including but not limited to brain (gliomas), breast, colon, head and neck, kidney, lung, liver, melanoma, ovarian, pancreatic, prostate, sarcoma, or thyroid cancer, and/or for preventing, treating and/or alleviating complications and/or symptoms and/or inflammatory responses associated therewith.

Viewed from a still further aspect, the invention provides the use of a compound of the invention in the preparation of a medicament for the prevention and/or treatment of erectile dysfunction, bronchial asthma, osteoporosis, renal diseases and AIDS, and/or for preventing, treating and/or alleviating complications and/or symptoms associated therewith.

Viewed from a still further aspect, the invention provides the use of a compound of the invention, or a composition comprising such a compound, for inhibiting the activity of at least one kinase, in vitro or in vivo.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Unless a context dictates otherwise, asterisks are used herein to indicate the point at which a mono- or bivalent radical depicted is connected to the structure to which it relates and of which the radical forms part.

Undefined (racemic) asymmetric centers that may be present in the compounds of Formula I or If are interchangeably indicated by drawing a wavy bonds or a straight bond in order to visualize the undefined steric character of the bond, for example

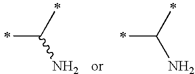

are used for the carbon bearing the amine of compounds of Formula I or II.

In an embodiment, the present invention provides a compound of Formula I or II

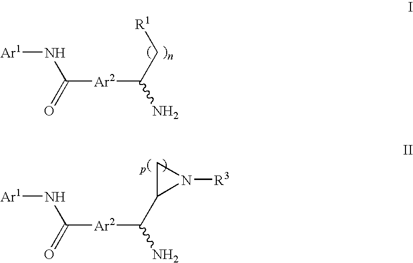

wherein:
Ar¹ is an aromatic 6-membered first ring containing carbon atoms and at least one nitrogen atom, said first ring being optionally fused to a saturated, unsaturated or aromatic 4-, 5-, 6-, or 7-membered second ring containing carbon atoms and optionally at least one nitrogen atom, said first or said second rings being independently substituted with one or more substituents (for example 1, 2, 3 or 4) independently selected from the group comprising hydrogen, halogen, alkyl, cycloalkyl, alkenyl, alkynyl, aralkyl, heteroarylalkyl, cycloalkylalkyl, acyl, aryl or heteroaryl wherein said substituents are optionally substituted by one or more further substituents (for example 1, 2, or 3) selected from the group comprising halo, hydroxyl, oxo, nitro, amido, carboxy, amino, cyano, haloalkoxy, and haloalkyl;
Ar² is an aromatic 5- or 6-membered third ring containing carbon atoms and optionally one or two heteroatoms, said third ring optionally fused to an aromatic 6-membered fourth ring containing carbon atoms and optionally one or two heteroatoms, wherein said third ring is optionally substituted with one or more substituents (for example 1, 2, 3 or 4) selected from the group comprising halogen, alkenyl, alkyl, alkynyl, acylamino, alkoxy, arylamino, nitro, haloalkoxy, aryl or heteroaryl, optionally substituted by one or more substituents;
n is an integer selected from 1 or 2; and
p is an integer selected from 2, 3, 4 or 5, preferably 3 or 4, more preferably 3,
—R¹ is selected from the Formula:

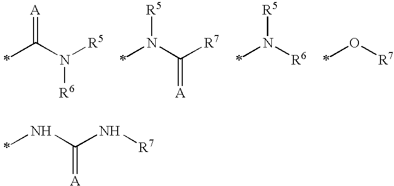

—R³ is selected from the Formula:

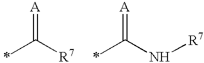

wherein
A is an oxygen or sulfur atom,
R⁵, R⁶ and R⁷ are each independently selected from the group comprising:
(A) hydrogen;
(B) alkyl, alkenyl or alkynyl, optionally substituted with:
(i) a homocyclic, heterocyclic, aryl or heteroaryl ring, to which may be a fused one or more homo or heterocyclic, aryl or heteroaryl rings, and which said ring or said one or more optional rings may be optionally substituted with one or more substituents independently selected from a first group comprising alkyl, hydroxyalkyl, haloalkyl, alkenyl, alkynyl, and homocyclic, heterocyclic, aryl or heteroaryl rings, wherein any substituents from this first group may be attached though an oxygen, sulfur or nitrogen atom or though one carbon atom; or independently selected from a second group comprising halo, oxo, nitro, amido, carboxy, hydroxyl, amino, cyano and haloalkoxy; or
(ii) a substituent selected from the second group defined in part (i); or
(iii) a substituent selected from the first group as defined in part (i) wherein said substituent is attached though an oxygen, sulfur or nitrogen atom or though one carbon atom, and wherein said a homocyclic, heterocyclic, aryl or heteroaryl rings are as defined in part (i);
(C) homocyclic and heterocyclic rings optionally substituted with:
(iv) a homocyclic, heterocyclic, aryl or heteroaryl ring as defined in part (i); or
(v) a substituent selected from the second group as defined in part (i); or
(vi) a substituent selected from the first group as defined in part (i) wherein said alkyl, hydroxyalkyl, haloalkyl, alkenyl, alkynyl, and homocyclic, heterocyclic, aryl or heteroaryl rings may, if present, be attached though an oxygen, sulfur or nitrogen atom or though one carbon atom, and wherein said homocyclic, heterocyclic, aryl or heteroaryl rings are as defined in part (i); and
(vii) where the homocyclic or heterocyclic rings comprise 4 or more ring atoms, fused to the homocyclic and heterocyclic rings may be one or more homo or heterocyclic, aryl or heteroaryl rings, and said rings, if present, may be optionally substituted with one or more substituents independently selected from the first or a second groups as defined in part (i) wherein said substituents in said second group may, if present, be attached though an oxygen, sulfur or nitrogen atom or though one carbon atom; and
(D) an aryl or heteroaryl ring optionally substituted with:
(viii) a homocyclic, heterocyclic, aryl or heteroaryl ring as defined in part (i); or
(ix) a substituent selected from the second group as defined in part (i); or
(x) a substituent selected from the first group as defined in part (i) wherein said alkyl, hydroxyalkyl, haloalkyl, alkenyl, alkynyl, and homocyclic, heterocyclic, aryl or heteroaryl rings may, if present, be attached though an oxygen, sulfur or nitrogen atom or though one carbon atom, and wherein said homocyclic, heterocyclic, aryl or heteroaryl rings are as defined in part (i); and
(xi) fused to the aryl or heteroaryl ring may be one or more homo or heterocyclic, aryl or heteroaryl rings, and said rings, if present, may be optionally substituted with one or more substituents independently selected from the first or a second groups as defined in part (i) wherein said substituents in said second group may, if present, be attached though an oxygen, sulfur or nitrogen atom or though one carbon atom;
or a stereoisomer, tautomer, racemic, metabolite, pro- or predrug, salt, hydrate, or solvate thereof.

When describing the compounds of the invention, the terms used are to be construed in accordance with the following definitions, unless a context dictates otherwise:

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 20 carbon atoms, more preferably from 1 to 10 carbon atoms, still more preferably 1 to 8 carbon atoms, in particular 1 to 6 carbon atoms, preferably 1 to 4 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl and its isomers (e.g. n-butyl, i-butyl and t-butyl); pentyl and its isomers, hexyl and its isomers, heptyl and its isomers, octyl and its isomers, nonyl and its isomers; decyl and its isomers. C₁-C₆ alkyl includes all linear, branched or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopentyl, 2-, 3- or 4-methylcyclopentyl, cyclopentylmethylene, and cyclohexyl.

The term “optionally substituted alkyl” refers to an alkyl group optionally substituted with one or more substituents (for example 1 to 4 substituents, for example 1, 2, 3 or 4 substituents) at any available point of attachment. Non-limiting examples of such substituents include halogen, hydroxy, carbonyl, nitro, amino, oximes, imines, azido, hydrazino, cyano, alkyl, aryl, heteroaryl, cycloalkyl, acyl, alkylamino, alkoxy, thiol, alkylthio, carboxylic acid, acylamino, alkyl esters, carbamates, thioamides, urea, sulphonamides and the like.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein. The term “hydroxyalkyl” refers to a —R^{a}—OH group wherein R^{a} is alkylene as defined herein. For example, “hydroxyalkyl” includes 2-hydroxyethyl, 1-(hydroxymethyl)-2-methylpropyl, 3,4-dihydroxybutyl, and so forth. “Alkoxyalkyl” refers to an alkyl group substituted with one to two of OR′, wherein R′ is alkoxy as defined below. For example, “aralkyl” or “(aryl)alkyl” refers to a substituted alkyl group as defined above wherein at least one of the alkyl substituents is an aryl as defined below, such as benzyl. For example, “heteroarylalkyl” refers to a substituted alkyl group as defined above, wherein at least one of the alkyl substituents is a heteroaryl as defined below, such as pyridinyl.

The term “cycloalkyl group” as used herein is a cyclic alkyl group, that is to say, a monovalent, saturated, or unsaturated hydrocarbyl group having 1, 2 or 3 cyclic structure. Cycloalkyl includes all saturated or partially saturated (containing 1 or 2 double bonds) hydrocarbon groups containing 1 to 3 rings, including monocyclic, bicyclic or polycyclic alkyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. The further rings of multi-ring cycloalkyls may be either fused, bridged and/or joined through one or more spiro atoms. Cycloalkyl groups may also be considered to be a subset of homocyclic rings discussed hereinafter. Examples of cycloalkyl groups, are cyclopropyl, cyclobutyl, cyclopentyl, cyclohexyl, cycloheptyl, cyclooctyl, cyclononyl and cyclodecyl with cyclopropyl being particularly preferred. An “optionally substituted cycloalkyl” refers to a cycloalkyl having optionally one or more substituents (for example 1 to 3 substituents, for example 1, 2, 3 or 4 substituents), selected from those defined above for substituted alkyl. When the suffix “ene” is used in conjunction with a cyclic group, this is intended to mean the cyclic group as defined herein having two single bonds as points of attachment to other groups.

Where alkyl groups as defined are divalent, i.e., with two single bonds for attachment to two other groups, they are termed “alkylene” groups. Non-limiting examples of alkylene groups includes methylene, ethylene, methylmethylene, trimethylene, propylene, tetramethylene, ethylethylene, 1,2-dimethylethylene, pentamethylene and hexamethylene. Similarly, where alkenyl groups as defined above and alkynyl groups as defined above, respectively, are divalent radicals having single bonds for attachment to two other groups, they are termed “alkenylene” and “alkynylene” respectively.

Generally, alkylene groups of this invention preferably comprise the same number of carbon atoms as their alkyl counterparts. “Cycloalkylene” herein refers to a saturated homocyclic hydrocarbyl biradical of Formula CₙH₂ₙ₋₂. Cycloalkylene groups of this invention preferably comprise the same number of carbon atoms as their cycloalkyl radical counterparts. Where an alkylene or cycloalkylene biradical is present, connectivity to the molecular structure of which it forms part may be through a common carbon atom or different carbon atom, preferably a common carbon atom. To illustrate this applying the asterisk nomenclature of this invention, a C₃ alkylene group may be for example *-CH₂CH₂CH₂-*, *-CH(—CH₂CH₃)-* or *-CH₂CH(—CH₃)-*. Likewise a C₃ cycloalkylene group may be

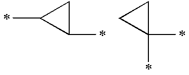

Where a cycloalkylene group is present, this is preferably a C₃-C₆ cycloalkylene group, more preferably a C₃ cycloalkylene (i.e. cyclopropylene group) wherein its connectivity to the structure of which it forms part is through a common carbon atom. Cycloalkylene and alkylene biradicals in compounds of the invention may be, but preferably are not, substituted.

The term “alkenyl” as used herein refers to an unsaturated hydrocarbyl group, which may be linear, branched or cyclic, comprising one or more carbon-carbon double bonds. Alkenyl groups thus comprise two or more carbon atoms, preferably between 2 and 20 carbon atoms, more preferably between 2 and 10 carbon atoms, still more preferably between 2 and 8 carbon atoms, for example, between 2 and 6 carbon atoms. Examples of alkenyl groups are ethenyl, 2-propenyl, 2-butenyl, 3-butenyl, 2-pentenyl and its isomers, 2-hexenyl and its isomers, 2-heptenyl and its isomers, 2-octenyl and its isomers, 2,4-pentadienyl and the like. An optionally substituted alkenyl refers to an alkenyl having optionally one or more substituents (for example 1, 2 or 3 substituents, or 1 to 2 substituents), selected from those defined above for substituted alkyl. Similarly to cycloalkyl groups, cycloalkenyl groups may be considered to be a subset of homocyclic rings discussed hereinafter.

The term “alkynyl” as used herein, similarly to alkenyl, refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Examples alkynyl groups are ethynyl, 2-propynyl, 2-butynyl, 3-butynyl, 2-pentynyl and its isomers, 2-hexynyl and its isomers, 2-heptynyl and its isomers, 2-octynyl and its isomers and the like. An optionally substituted alkynyl refers to an alkynyl having optionally one or more substituents (for example 1 to 4 substituents, or 1 to 2 substituents), selected from those defined above for substituted alkyl. Similarly to cycloalkyl groups, cycloalkynyl groups may be considered to be a subset of homocyclic rings discussed hereinafter.

The term “homocyclic ring” as used herein is a ring wherein the ring atoms comprise only carbon atoms. Examples of homocyclic rings thus include cycloalkyl, cycloalkenyl and cycloalkynyl, with cycloalkyl and cycloalkenyl being preferred. Where a ring carbon atom is replaced with a heteroatom, preferably nitrogen, oxygen of sulfur, the heteroatom-containing ring resultant from such a replacement is referred to herein as a heterocyclic ring. More than one carbon atom in a ring may be replaced so forming heterocyclic ring having a plurality of heteroatoms.

The terms “heterocyclyl” or “heterocyclo” as used herein by itself or as part of another group refer to non-aromatic, fully saturated or partially unsaturated cyclic groups (for example, 3 to 13 member monocyclic, 7 to 17 member bicyclic, or 10 to 20 member tricyclic ring systems, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. An optionally substituted heterocyclic refers to a heterocyclic having optionally one or more substituents (for example 1 to 4 substituents, or for example 1, 2, 3 or 4), selected from those defined above for substituted aryl.

Exemplary heterocyclic groups include piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, indolinyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, triazinyl, cinnolinyl, phthalazinyl, oxetanyl, thietanyl, 3-dioxolanyl, 1,4-dioxanyl, 2,5-dioximidazolidinyl, 2,2,4-piperidonyl, 2-oxopiperidinyl, 2-oxopyrrolodinyl, 2-oxoazepinyl, indolinyl, tetrahydropyranyl, tetrahydrofuranyl, tetrehydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl, 6H-1,2,5-thiadiazinyl, 2H-1,5,2-dithiazinyl, 2H-oxocinyl, 1H-pyrrolizinyl, tetrahydro-1,1-dioxothienyl, N-formylpiperazinyl, and morpholinyl.

The term “aryl” as used herein refers to a polyunsaturated, aromatic hydrocarbyl group having a single ring (i.e. phenyl) or multiple aromatic rings fused together (e.g. naphthalene or anthracene). or linked covalently, typically containing 5 to 8 atoms; wherein at least one ring is aromatic. The aromatic ring may optionally include one to three additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7- or 8-azulenyl, 1- or 2-naphthyl, 1-, 2- or 3-indenyl, 1-, 2- or 9-anthryl, 1-2-, 3-, 4- or 5-acenaphtylenyl, 3-, 4- or 5-acenaphtenyl, 1-, 2-, 3-, 4- or 10-phenanthryl, 1- or 2-pentalenyl, 1,2-, 3- or 4-fluorenyl, 4- or 5-indanyl, 5-, 6-, 7- or 8-tetrahydronaphthyl, 1,2,3,4-tetrahydronaphthyl, 1,4-dihydronaphthyl, dibenzo[a,d]cylcoheptenyl, 1-, 2-, 3-, 4- or 5-pyrenyl.

The aryl ring can optionally be substituted by one or more substituents. An “optionally substituted aryl” refers to an aryl having optionally one or more substituents (for example 1 to 5 substituents, for example 1, 2, 3 or 4) at any available point of attachment. Non-limiting examples of such substituents are selected from halogen, hydroxy, oxo, nitro, amino, hydrazine, aminocarbonyl, azido, cyano, alkyl, cycloalkyl, alkenyl, alkynyl, cycloalkylalkyl, alkylamino, alkoxy, —SO₂—NH₂, aryl, heteroaryl, aralkyl, haloalkyl, haloalkoxy, alkyloxycarbonyl, alkylaminocarbonyl, heteroarylalkyl, alkylsulfonamide, heterocyclyl, alkylcarbonylaminoalkyl, aryloxy, alkylcarbonyl, acyl, arylcarbonyl, aminocarbonyl, alkylsulfoxide, —SO₂R¹¹⁵, alkylthio, carboxy, and the like, wherein R¹¹⁵ is alkyl or cycloalkyl.

The term “arylene” as used herein is intended to include divalent carbocyclic aromatic ring systems such as phenylene, biphenylylene, naphthylene, anthracenylene, phenanthrenylene, fluorenylene, indenylene, pentalenylene, azulenylene and the like. Arylene is also intended to include the partially hydrogenated derivatives of the carbocyclic systems enumerated above. Non-limiting examples of such partially hydrogenated derivatives are 1,2,3,4-tetrahydronaphthylene, 1,4-dihydronaphthylene and the like.

Where a carbon atom in an aryl group is replaced with a heteroatom, the resultant ring is referred to herein as a heteroaryl ring.

The term “heteroaryl” as used herein by itself or as part of another group refers but is not limited to 5 to 12 carbon-atom aromatic rings or ring systems containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of heteroaryl can be 2- or 3-furyl, 2- or 3-thienyl, 1-, 2- or 3-pyrrolyl, 1-, 2-, 4- or 5-imidazolyl, 1-, 3-, 4- or 5-pyrazolyl, 3-, 4- or 5-isoxazolyl, 2-, 4- or 5-oxazolyl, 3-, 4- or 5-isothiazolyl, 2-, 4- or 5-thiazolyl, 1,2,3-triazol-1-, -2-, -4- or -5-yl, 1,2,4-triazol-1-, -3-, -4- or -5-yl, 1,2,3-oxadiazol-4- or -5-yl, 1,2,4-oxadiazol-3- or -5-yl, 1,2,5-oxadiazolyl, 1,3,4-oxadiazolyl, 1,2,3-thiadiazol-4- or -5-yl, 1,2,4-thiadiazol-3- or -5-yl, 1,2,5-thiadiazol-3- or -4-yl, 1,3,4-thiadiazolyl, 1- or 5-tetrazolyl, 2-, 3- or 4-pyridyl, 3- or 4-pyridazinyl, 2-, 4-, 5- or 6-pyrimidinyl, 2-, 3-, 4-, 5-6-2H-thiopyranyl, 2-, 3- or 4-4H-thiopyranyl, 2-, 3-, 4-, 5-, 6- or 7-benzofuryl, 1-, 3-, 4- or 5-isobenzofuryl, 2-, 3-, 4-, 5-, 6- or 7-benzothienyl, 1-, 3-, 4- or 5-isobenzothienyl, 1-, 2-, 3-, 4-, 5-, 6- or 7-indolyl, 2- or 3-pyrazinyl, 1,4-oxazin-2- or -3-yl, 1,4-dioxin-2- or -3-yl, 1,4-thiazin-2- or -3-yl, 1,2,3-triazinyl, 1,2,4-triazinyl, 1,3,5-triazin-2-, -4- or -6-yl, thieno[2,3-b]furan-2-, -3-, -4-, or -5-yl, 1-, 2-, 4- or 5-benzimidazolyl, 1-, 3-, 4-, 5-, 6- or 7-benzopyrazolyl, 3-, 4-, 5-, 6- or 7-benzisoxazolyl, 2-, 4-, 5-, 6- or 7-benzoxazolyl, 3-, 4-, 5-, 6- or 7-benzisothiazolyl, 2-, 4-, 5-, 6- or 7-benzothiazolyl, 1-, 2-thianthrenyl, 3-, 4- or 5-isobenzofuranyl, 1-, 2-, 3-, 4- or 9-xanthenyl, 1-, 2-, 3- or 4-phenoxathiinyl, 2-, 3-pyrazinyl, 1-, 2-, 3-, 4-, 5-, 6-, 7- or 8-indolizinyl, 2-, 3-, 4- or 5-isoindolyl, 1-, 2-, 3-, 4-, 5-, 6- or 7-indazolyl, 2-, 6-, 7- or 8-purinyl, 4-, 5- or 6-phthalazinyl, 2-, 3- or 4-naphthyridinyl, 2-, 5- or 6-quinoxalinyl, 2-, 4-, 5-, 6-, 7- or 8-quinazolinyl, 1-, 2-, 3- or 4-quinolizinyl, 2-, 3-, 4-, 5-, 6-, 7-, or 8-quinolinyl(quinolyl), 2-, 4-, 5-, 6-, 7- or 8-quinazolyl, 1-, 3-, 4-, 5-, 6-, 7- or 8-isoquinolinyl(isoquinolyl), 3-, 4-, 5-, 6-, 7- or 8-cinnolinyl, 2-, 4-, 6- or 7-pteridinyl, 1-, 2-, 3-, 4- or 9-carbazolyl, 1-, 2-, 3-, 4-, 5-, 6-, 7-, 8- or 9-carbolinyl, 1-, 2-, 3-, 4-, 5-, 6-, 7-, 8-, 9- or 10-phenanthridinyl, 1-, 2-, 3- or 4-acridinyl, 1-, 2-, 3-, 4-, 5-, 6-, 7-, 8- or 9-perimidinyl, 2-, 3-, 4-, 5-, 6-, 7-, 8-, 9- or 10-(1,7)phenanthrolinyl, 1- or 2-phenazinyl, 1-, 2-, 3-, 4-, or 10-phenothiazinyl, 3- or 4-furazanyl, 1-, 2-, 3-, 4-, or 10-phenoxazinyl, or additionally substituted derivatives thereof.

An “optionally substituted heteroaryl” refers to a heteroaryl having optionally one or more substituents (for example 1 to 4 substituents, for example 1, 2, 3 or 4), selected from those defined above for substituted aryl.

The term “oxo” as used herein refers to the group ═O.

The term “alkoxy” as used herein refers to a radical having the Formula —OR wherein R is alkyl. Preferably, alkoxy is C₁-C₁₀ alkoxy or C₁-C₆ alkoxy. Where the oxygen atom in an alkoxy group is substituted with sulfur, the resultant radical is referred to as thioalkoxy. Haloalkoxy is an alkoxy group wherein 1 or more hydrogen atoms in the alkyl group is substituted with halo.

The term “aryloxy” as used herein denotes a group —O-aryl, wherein aryl is as defined above.

The term “aroyl” as used herein denotes a group —C(O)-aryl, wherein aryl is as defined above.

The term “cycloalkylalkyl” by itself or as part of another substituent refers to a group having one of the aforementioned cycloalkyl groups attached to one of the aforementioned alkyl chains. Examples of such cycloalkylalkyl radicals include cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, 3-cyclopentylbutyl, cyclohexylbutyl and the like.

The term “heterocyclyl-alkyl” by itself or as part of another substituents refers to a group having one of the aforementioned heterocyclyl group attached to one of the aforementioned alkyl group, i.e., to a group —R^{b}—R^{c} wherein R^{b} is alkylene or alkylene substituted by alkyl group and R^{c} is a heterocyclyl group.

The term “acyl” by itself or as part of another substituent refers to an alkanoyl group having 2 to 6 carbon atoms or a phenylalkanoyl group whose alkanoyl moiety has 1 to 4 carbon atoms, i.e. a carbonyl group linked to a radical such as, but not limited to, alkyl, aryl, more particularly, the group —COR¹⁰, wherein R¹⁰ can be selected from alkyl, aryl, substituted alkyl, or substituted aryl, as defined herein. The term acyl therefore encompasses the group alkylcarbonyl (—COR¹⁰), wherein R¹⁰ is alkyl. Preferably, acyl is C₂-C₁₁ acyl or C₂-C₇ acyl. Where the oxygen atom is an acyl group is substituted with sulfur, the resultant radical is referred to as thioacyl. Said acyl can be exemplified by acetyl, propionyl, butyryl, valeryl and pivaloyl, benzoyl, phenylacetyl, phenylpropionyl and phenylbutylyl.

The term “amino” refers to the group —NH₂.

The term “alkylamino” by itself or as part of another substituent refers to a group consisting of an amino groups attached to one or two independently selected and optionally substituted alkyl groups, cycloalkyl groups, aralkyl or cycloalkylalkyl groups i.e., alkyl amino refers to —N(R⁸)(R⁹) wherein R⁸ and R⁹ are each independently selected from hydrogen, cycloalkyl, arylalkyl, cycloalkylalky or alkyl. Non-limiting examples of alkylamino groups include methylamino (NHCH₃), ethylamino (NHCH₂CH₃), n-propylamino, isopropylamino, n-butylamino, isobutylamino, sec-butylamino, tert-butylamino, n-hexylamino, and the like.

The term “aminoalkyl” refers to the group —R^{b}—NR^{d}R^{e} wherein R^{b} is alkylene or substituted alkylene, R^{d} is hydrogen or alkyl or substituted alkyl as defined herein, and R^{e} is hydrogen or alkyl as defined herein.

The term “aminocarbonyl” refers to the group —(C═O)—NH₂.

The term “alkylaminocarbonyl” refers to a group —(C═O)—NR^{d}R^{e} wherein R^{d} is hydrogen or alkyl or substituted alkyl as defined herein, and R^{e} is alkyl or substituted alkyl as defined herein.

The term “alkylaminocarbonylamino” refers to a group —NH(C═O)—NR^{d}R^{e} or —NR′(C═O)—NR^{d}R^{e} wherein R^{d} is hydrogen or alkyl or substituted alkyl as defined herein, and R^{e} is alkyl or substituted alkyl as defined herein, wherein R′ is alkyl or substituted alkyl.

The term “carboxy” or “carboxyl” refers to the group —CO₂H. Thus, a carboxyalkyl is an alkyl group as defined above having at least one substituent that is —CO₂H.

The term “alkoxycarbonyl” refers to a carboxy group linked to an alkyl radical i.e. to form —C(═O)OR¹⁰, wherein R¹⁰ is as defined above for acyl.

The term “alkylcarbonyloxy” refers to a —O—C(═O)R¹¹ wherein R¹¹ is as defined above for acyl.

The term “alkylcarbonylamino” refers to an group of Formula —NH(C═O)R or —NR′(C═O)R, wherein R and R′ are each independently alkyl or substituted alkyl.

The term “alkylcarbonylaminoalkyl” refers to a group —R^{b}—NR^{d}—C(═O)—R^{e} wherein R^{b} is alkylene or substituted alkylene, R^{d} is hydrogen or alkyl as defined herein, and R^{e} is alkyl as defined herein.

The term “alkoxy” by itself or as part of another substituent refers to a group consisting of an oxygen atom attached to one optionally substituted straight or branched alkyl group, cycloalkyl group, aralkyl or cycloalkylalkyl group. Non-limiting examples of suitable alkoxy group include methoxy, ethoxy, n-propoxy, isopropoxy, n-butoxy, iso-butoxy, sec-butoxy, tert-butoxy, hexanoxy and the like.

The term “alkylthio” by itself or as part of another substituent refers to a group consisting of a sulfur atom attached to one optionally substituted alkyl group, cycloalkyl group, aralkyl or cycloalkylalkyl group. Non-limiting examples of alkylthio groups include methylthio (SCH₃), ethylthio (SCH₂CH₃), n-propylthio, isopropylthio, n-butylthio, isobutylthio, sec-butylthio, tert-butylthio, n-hexylthio, and the like.

The term “acylamino” by itself or as part of another substituent refers to a group consisting of an amino group attached to one or two independently selected acyl groups as described before. In case the two acyl groups of a dicarboxylic acid are attached to the amino group these represent imides such as phtalimides, maleimides and the like, and are encompassed in the meaning of the term acylamino.

The term “halo” or “halogen” as a group or part of a group is generic for fluoro, chloro, bromo or iodo.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

The term “haloalkoxy” alone or in combination refers to a group of Formula —O-alkyl wherein the alkyl group is substituted by 1, 2 or 3 halogen atoms. For example, “haloalkoxy” includes —OCF₃ and —OCHF₂.

The term “sulfonamide” alone or in combination refers to a group of Formula —SO₂—NRR wherein each R independently is hydrogen or alkyl as defined herein.

The term “alkylsulfonylamino” alone or in combination refers to a group of Formula —NR^{d}—SO₂—R wherein R^{d} is hydrogen or alkyl as defined herein, and R independently is alkyl as defined herein.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

Where groups may be optionally substituted, such groups may be substituted with once or more, and preferably once, twice or thrice. Substituents may be selected from, for example, the group comprising halo, hydroxy, oxo, nitro, amido, carboxy, amino, cyano haloalkoxy, and haloalkyl.

As used herein the terms such as “alkyl, aryl, or cycloalkyl, each being optionally substituted with” or “alkyl, aryl, or cycloalkyl, optionally substituted with” refers to optionally substituted alkyl, optionally substituted aryl and optionally substituted cycloalkyl.

Whenever used in the present invention the term “compounds of the invention” or a similar term is meant to include the compounds of general Formula I or II and any subgroup thereof. This term also refers to the compounds as depicted in Tables 1 to 8 and their derivatives, N-oxides, salts, solvates, hydrates, stereoisomeric forms, racemic mixtures, tautomeric forms, optical isomers, analogues, pro-drugs, esters and metabolites, as well as their quaternized nitrogen analogues. The N-oxide forms of said compounds are meant to comprise compounds wherein one or several nitrogen atoms are oxidized to the so-called N-oxide.

As used in the specification and the appended claims, the singular forms “a”, “an,” and “the” include plural referents unless the context clearly dictates otherwise. By way of example, “a compound” means one compound or more than one compound.

The terms described above and others used in the specification are well understood to those in the art.

Preferred features of the compounds of this invention are now set forth.

Ar¹ is, preferably, a 4-pyridyl ring which, may be optionally substituted, or comprises a 4-pyridyl ring as part of a bicyclic structure wherein such bicyclic structure is attached to the nitrogen atom of the amide moiety shown in Formula I or II through the (1) carbon atom in the 4-pyridyl ring.

Preferred structures for Ar¹ are of the Formula:

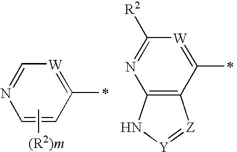

wherein
m is an integer selected from 0, 1, 2 or 3; preferably 0,
W is C(R²) or N; preferably C(R²), more preferably CH,
Y and Z are independently selected from the group comprising N and CR²;
R² is selected from hydrogen, halogen, or a group selected from alkyl, cycloalkyl, alkenyl, alkynyl, aryl or heteroaryl wherein each of said group is optionally substituted by one or more further substituents (for example 1, 2, or 3 substituents) selected from the group comprising halo, hydroxyl, amido, carboxy, amino, cyano, haloalkoxy, and haloalkyl.

In these preferred structures for Ar¹, the following features are preferred:
m is either 0 or 1, preferably 0; and
W is N or C(R²); particularly wherein the R² present in W is hydrogen.

In a particular embodiment, in these structures for Ar¹, the following features are preferred wherein Y is CH and Z is CH or wherein Y is CH and Z is N, or wherein Y is N and Z is CH.

Ar² is preferably of the Formula:

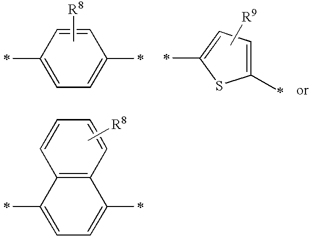

wherein R⁸ is selected from the group comprising hydrogen and halogen, alkenyl, alkyl, alkynyl, acylamino, alkoxy, arylamino, nitro, haloalkoxy, aryl or heteroaryl, each group being optionally substituted by one or more substituents; and R⁹ is selected from the group comprising hydrogen, halogen and alkyl.

Especially preferably, —Ar²— is either

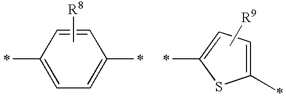

preferably wherein R⁸ and R⁹ are hydrogen.

Preferably, where A is present in R¹, A is oxygen or sulfur. In some embodiments, A is preferably sulfur. In other embodiments, A is preferably oxygen.

Generally, in the compounds of Formula I or II, in particular those in which n is 1, R¹ is preferably selected from the Formula:

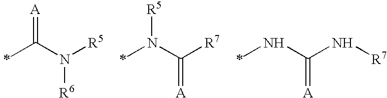

Particularly preferably, where R¹ is attached though one carbon atom, said one carbon atom is a methylene or cycloalkylene biradical, which is preferably unsubstituted. Where R¹ is attached though one carbon atom, and said one carbon atom is a cycloalkylene radical, this is preferably cyclopropylene.

In one embodiment of the invention R¹ is of the Formula *-N(H)—C(═O)—C¹—Ar³

wherein C¹ is a methylene or cycloalkylene biradical; and Ar³ is an aromatic 5- or 6-membered ring containing carbon atoms and optionally one or two heteroatoms optionally substituted with one or more substituents (for example 1, 2, 3 or 4) selected from the group comprising halogen, alkenyl, alkyl, alkynyl, acylamino, alkoxy, arylamino, nitro and haloalkoxy.

Examples of such embodiments of the invention are the compounds 4-{1-Amino-2-[2-(3,4-difluorophenyl)-acetylamino]-ethyl}-N-pyridin-4-yl-benzamide dihydrochloride and 4-(1-Amino-2-{[1-(4-chloro-phenyl)-cyclopropanecarbonyl]-amino}-ethyl)-N-pyridin-4-yl-benzamide dihydrochloride.

In a particular embodiment, the present invention provides compounds of Formula I or II having one of the structural Formula

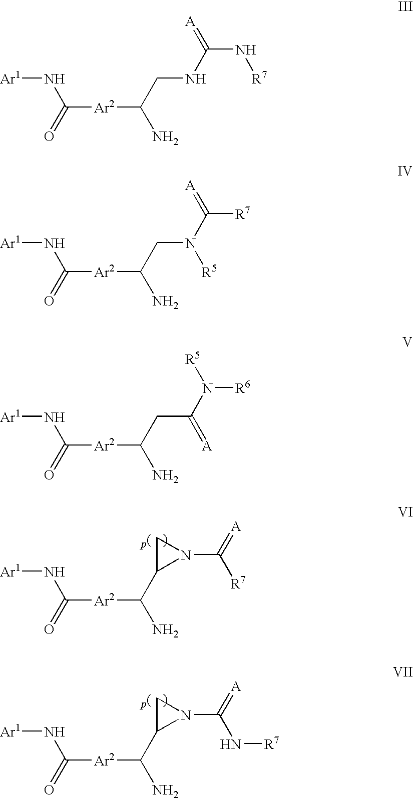

wherein Ar¹, Ar², A, R⁵, R⁶, p and R⁷ have the same meaning as described above. Preferably, wherein Ar¹, Ar², A, R⁵, R⁶ and R⁷ have the same meaning as described above and p is 2, 3 or 4, preferably p is 3 or 4, more preferably p is 3.

Preferably, R⁵ is hydrogen, alkyl or cycloalkyl and A is an oxygen or a sulfur atom.

Preferably, —Ar²— is

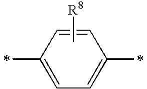

wherein R⁸ has the same meaning as that defined above. Preferably, R⁸ is hydrogen.

In a preferred embodiment, the present invention relates to compounds of Formula I, II or V wherein R⁶ and R⁷ are each independently selected from

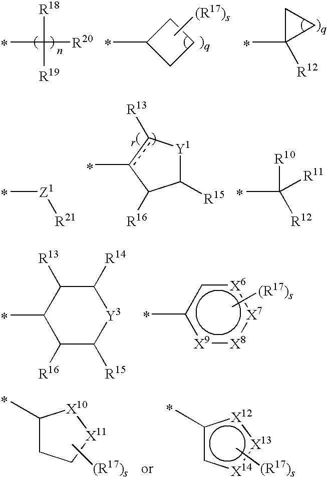

wherein
Y¹ is selected from —CH₂—, —CH(R¹⁴)—, —NH—, —O—, —S—, or —C(═O)—,
Y³ is selected from —CH₂—, —CH₂—CH₂—, —O—, —S—, or —NH—,
X⁶ is selected from N or CH,
X⁷ is selected from N, C(═O), or CH,
X⁸ is selected from N, NH or CH,
X⁹ is selected from N or CH,
X¹⁰ is selected from S, O or NH,
X¹¹ is selected from O, CH₂, C(═O), S or NH,
X¹² is selected from N. NH, O, S or CH,
X¹³ is selected from NH, O, S or CH,
X¹⁴ is selected from S, N, NH or CH,
Z¹ is selected from O or NH,
q is an integer selected from 1, 2, 3 or 4,
n is an integer selected from 1, 2, 3, 4, 5, 6 or 7,
wherein R¹⁰ and R¹¹ are each independently a selected from hydrogen, alkyl, cycloalkyl, aryl, or aralkyl,
wherein R¹² is selected from aryl, cycloalkyl, heteroaryl or heterocyclyl, each being optionally substituted by one or more substituent (for example 1, 2, 3 or 4) selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
r is an integer selected from 0, 1, 2 or 3,
wherein R¹³ and R¹⁴ are each independently selected from hydrogen or alkyl,
or R¹³ and R¹⁴ form together with the carbon atoms to which they are attached form an aryl, an heteroaryl, a cycloalkyl or a heterocyclyl,
or r is 2 and two R¹³ form together with the carbon atoms to which they are attached form an aryl, an heteroaryl, a cycloalkyl or a heterocyclyl,
wherein R¹⁵ and R¹⁶ together with the carbon atom to which they are attached form an aryl, a cycloalkyl, a heteroaryl a heterocyclyl, each being optionally substituted with one or more substituent (for example 1, 2, 3 or 4) selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
s is an integer selected from 0, 1, 2, 3 or 4,
wherein R¹⁷ is selected from halo, or a group selected from alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylalkenylaminooxy, heteroarylalkyl, heteroarylcarbonylamino, heterocyclyl, hydroxyalkyl, nitro, oxo, sulfonyl, or two R¹⁷ together with the atoms to which they are attached form an aryl, heteroaryl, cycloalkyl, or heterocyclyl, each group being optionally substituted with one or more substituents (for example 1, 2, 3 or 4) selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
wherein R¹⁸ and R¹⁹ are each independently selected from hydrogen, alkyl, cycloalkyl, cycloalkylalkyl, aryl, aralkyl, heteroaryl or heteroarylalkyl,
wherein R²⁰ is selected from hydrogen, or a group selected from alkyl, cycloalkyl, alkylaminoalkyl, alkylamino, alkylcarbonylamino, alkylcarbonylaminoalkyl, alkylsulfonyl, alkylsulfonylamino, alkylsulfonylaminoalkyl, amino, aminoalkyl, heterocyclyl, heterocyclylalkyl, cyano, cyanoalkyl, hydroxyl, hydroxyalkyl, alkoxy, alkoxyalkyl, alkoxycarbonyl, carboxy, alkoxycarbonylalkyl, aryl, aralkyl, heteroaryl, heteroarylalkyl, each group being optionally substituted by one or more substituent (for example 1, 2, 3 or 4) selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
wherein R²¹ is selected from alkyl, aryl, alkylcarbonyl, heteroaryl or heteroarylcarbonyl.

In a particular embodiment, the present invention provides compounds of Formula I or II having one of the structural Formula

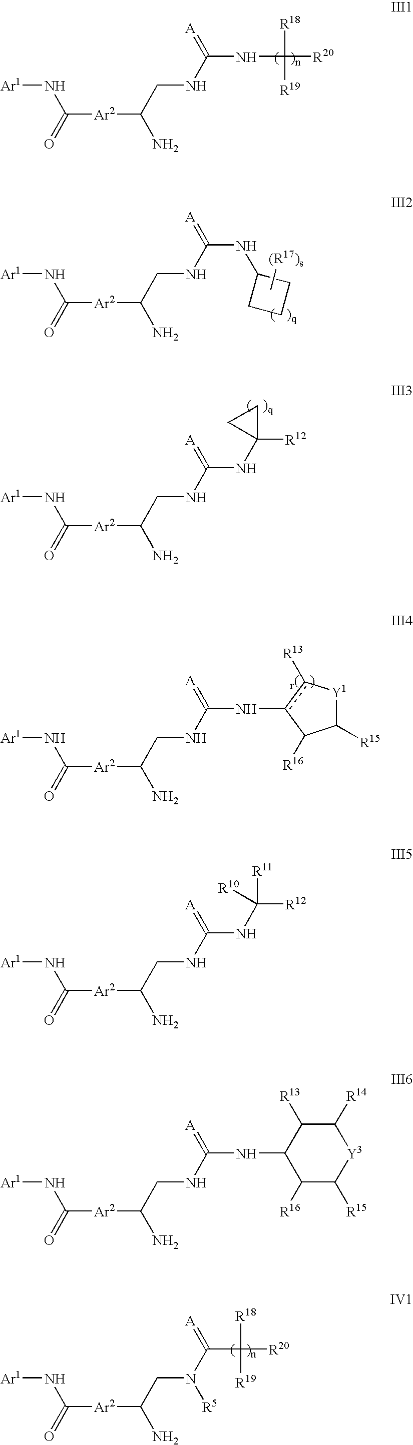

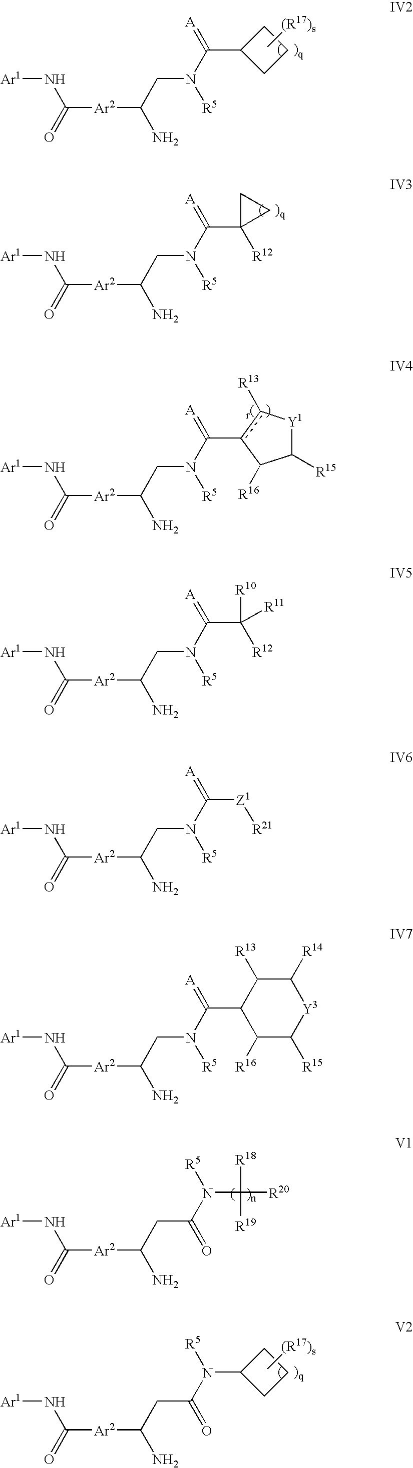

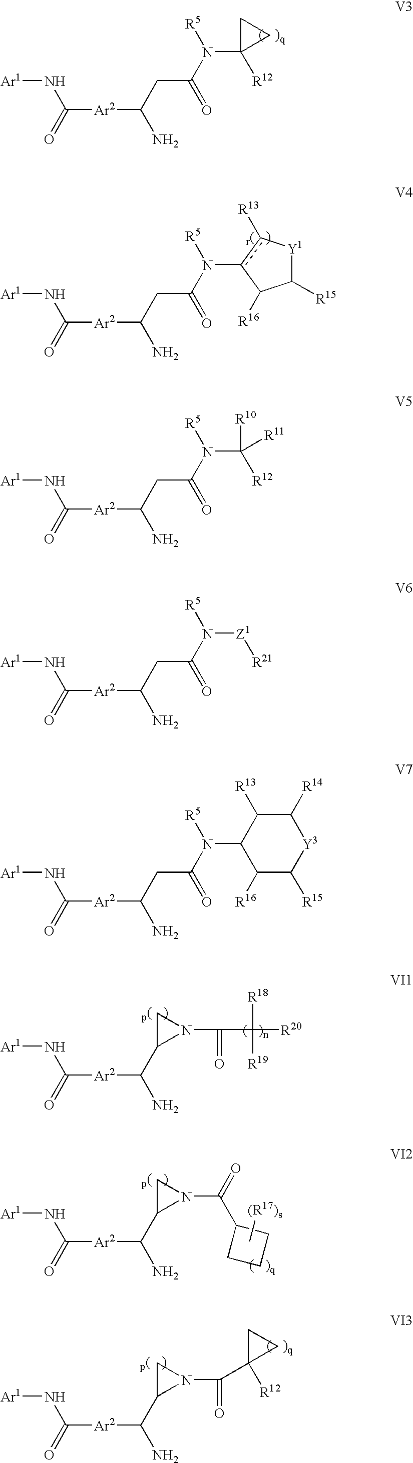

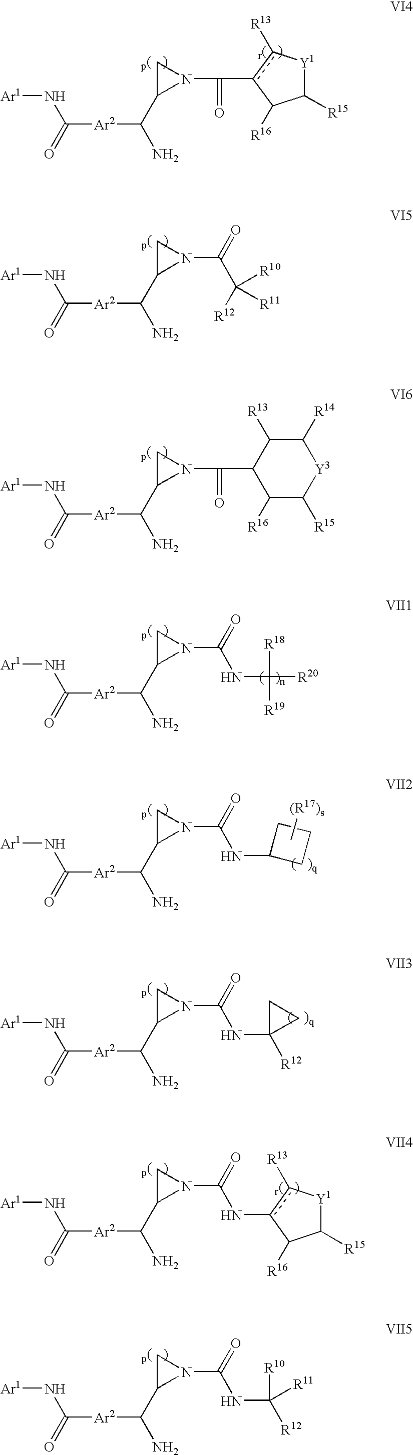

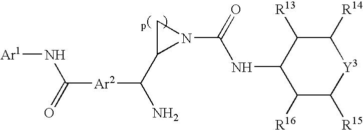

wherein Ar¹, Ar², A, s, p, q, r, n, Y¹, R⁵, R¹⁰, R¹¹, R¹², R¹³, R¹⁵, R¹⁶, and R¹⁷ have the same meaning as that define above, preferably wherein Ar¹, Ar², A, s, q, r, n, Y¹, R⁵, R¹⁰, R¹¹, R¹², R¹³, R¹⁵, R¹⁶ and R¹⁷ have the same meaning as that define above and p is selected from 3 or 4, preferably 3.

In another particular embodiment, the present invention provides compounds of Formula I or II having one of the structural Formula

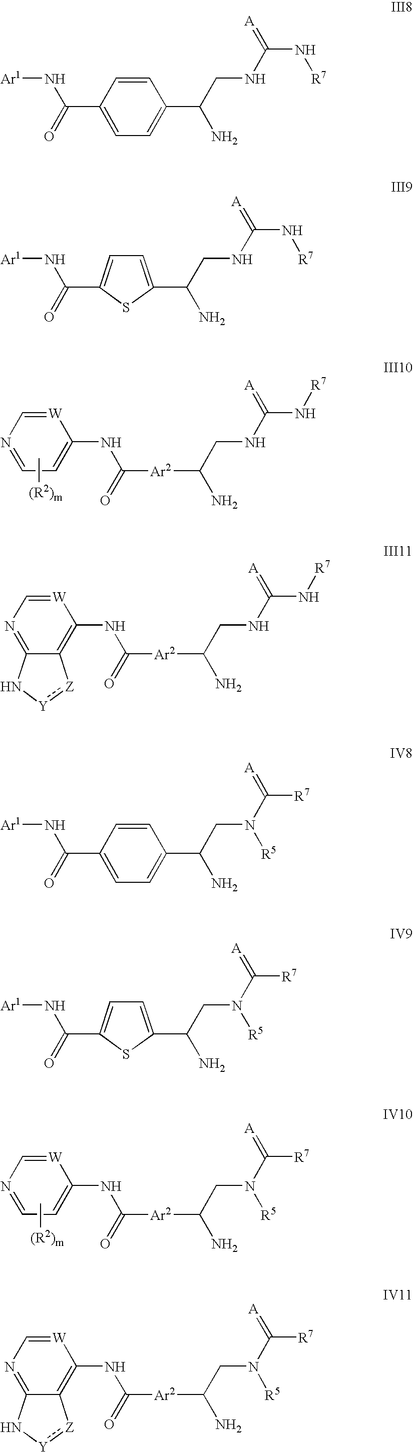

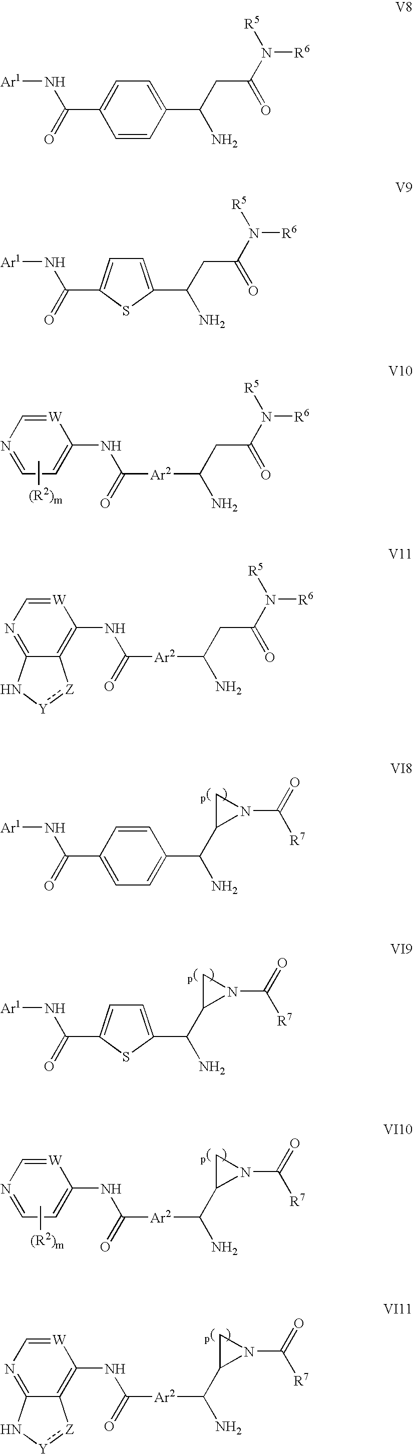

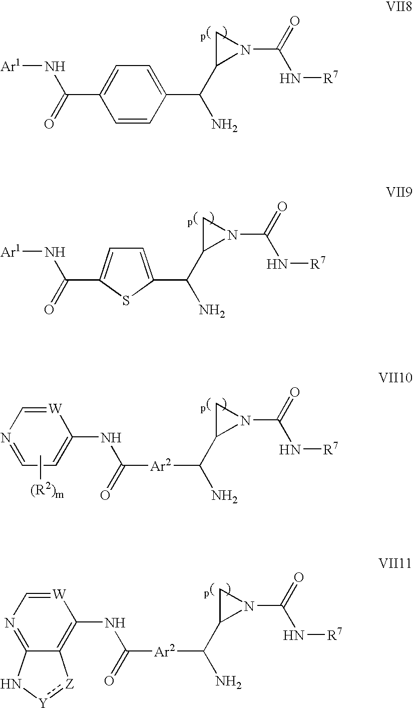

wherein Ar¹, Ar², A, s, p, q, r, n, W, Y, Y¹, Z, R², R⁵, R⁶, R⁷, R¹⁰, R¹¹, R¹², R¹³, R¹⁵, R¹⁶, and R¹⁷ have the same meaning as that defined above.

In another particular embodiment, the present invention provides compounds of Formula I or II having one of the structural Formula

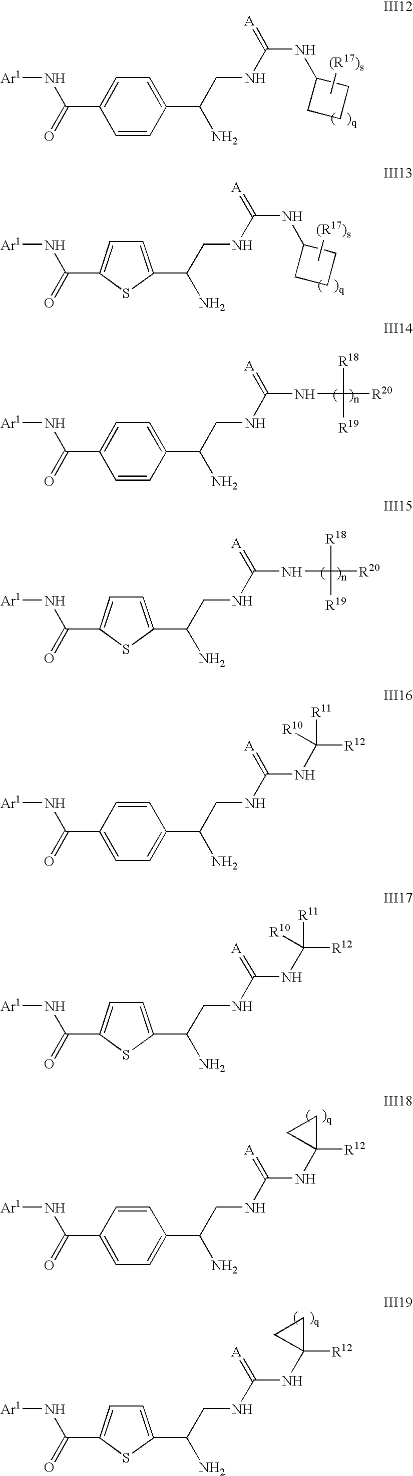

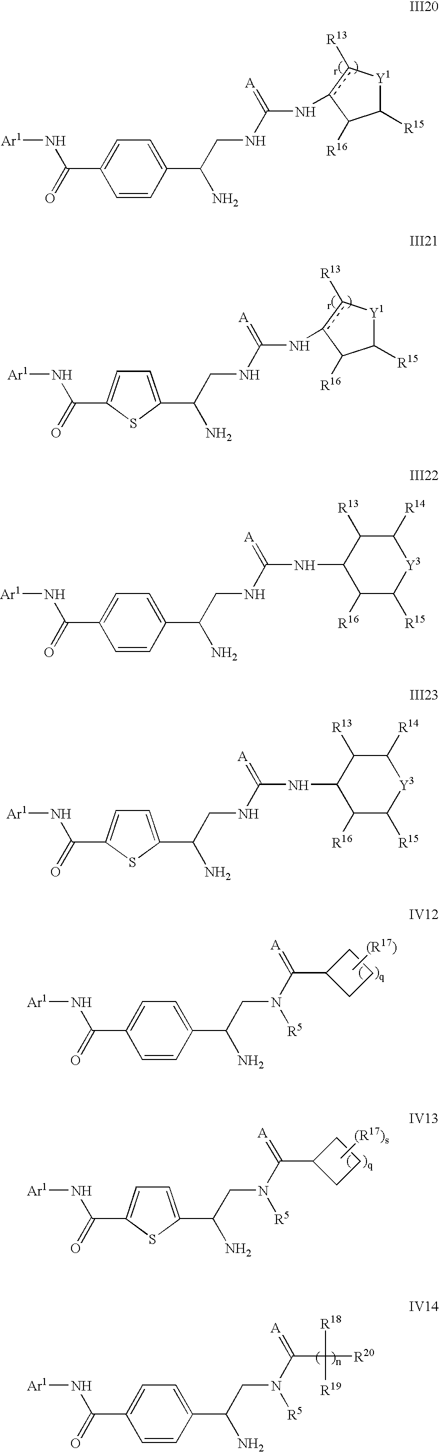

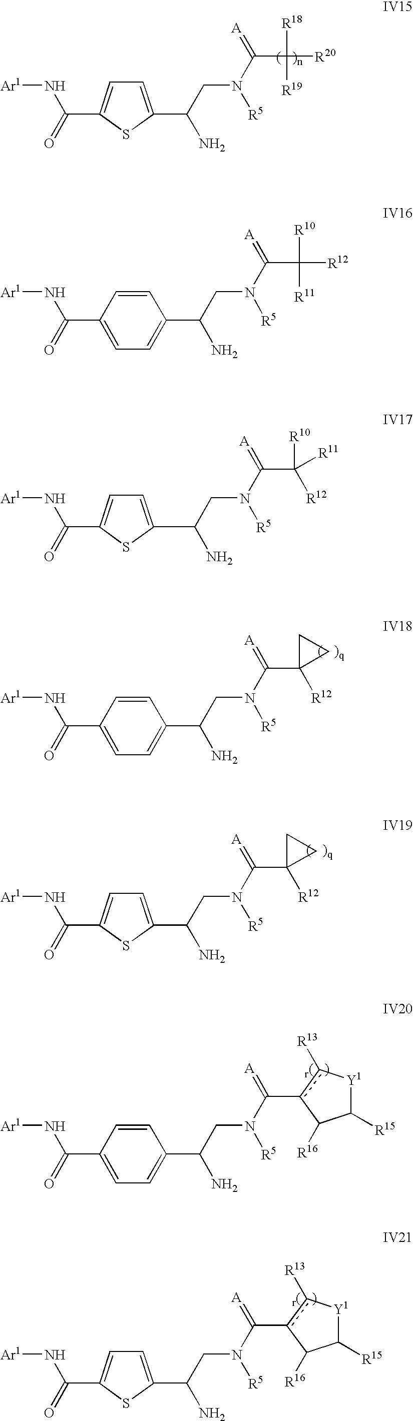

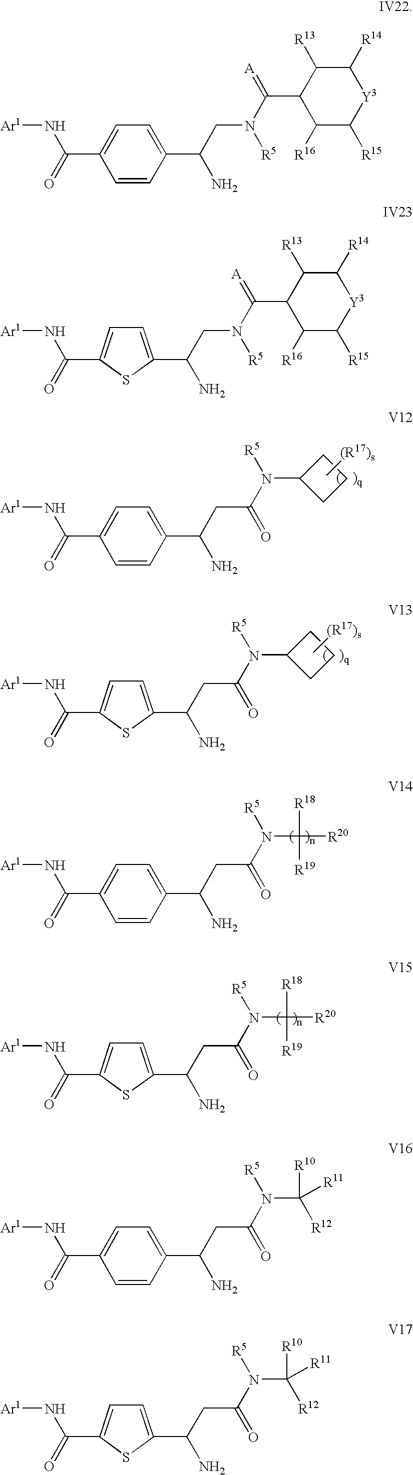

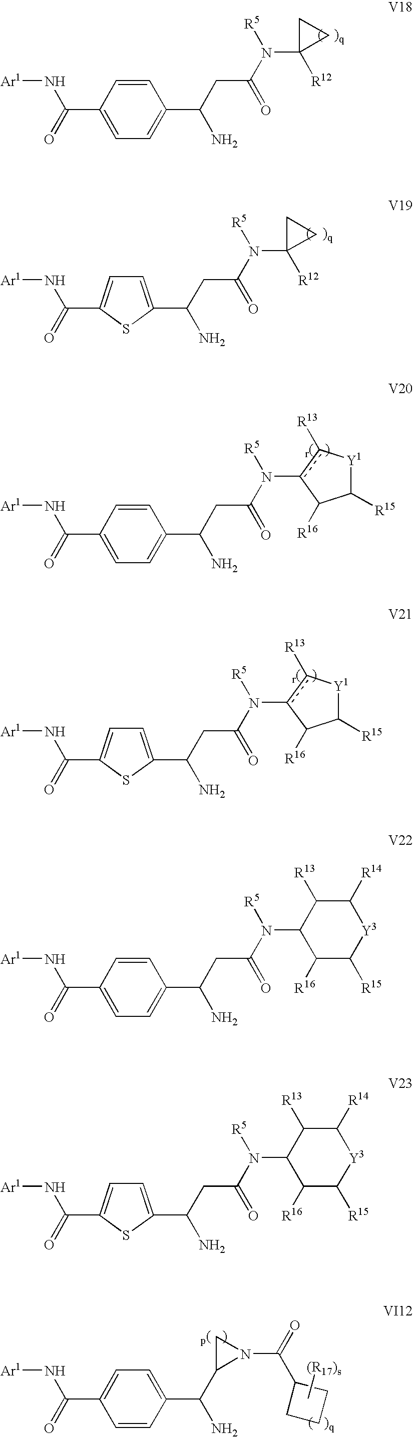

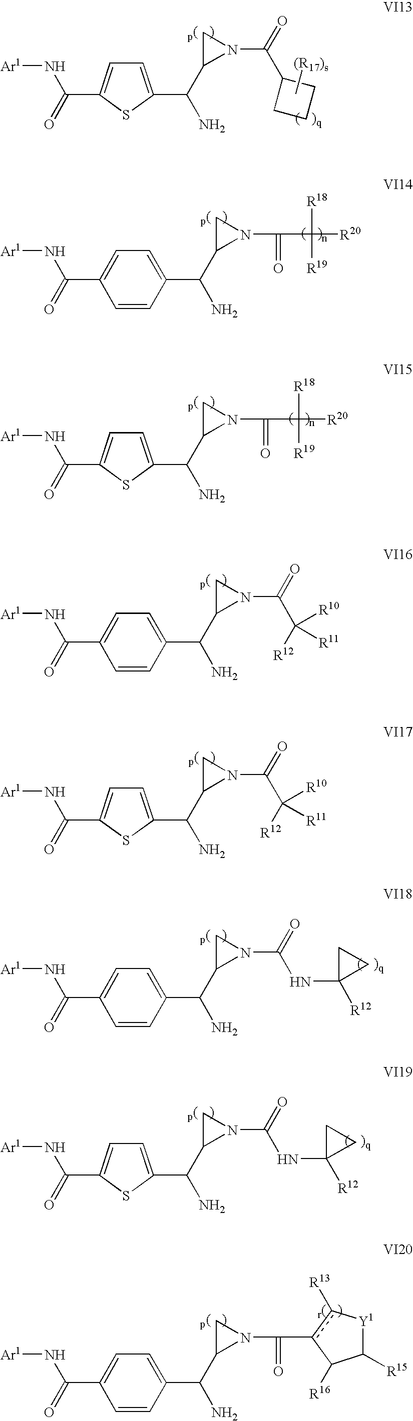

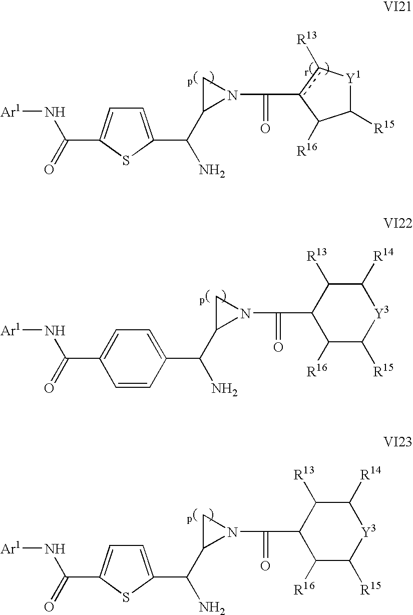

wherein Ar¹, A, s, p, q, r, n, Y¹, R⁵, R¹⁰, R¹¹, R¹², R¹³, R¹⁵, R¹⁶, R¹⁷, R¹⁸, R¹⁹ and R²⁰ have the same meaning that defined above.

In a further particular embodiment, the present invention provides compounds of Formula I or II having one of the structural Formula

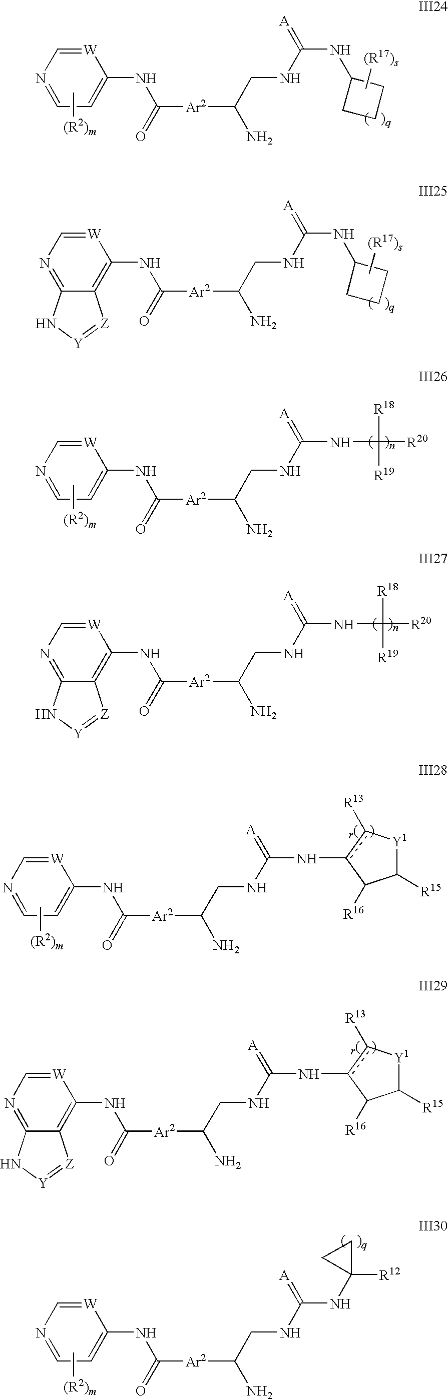

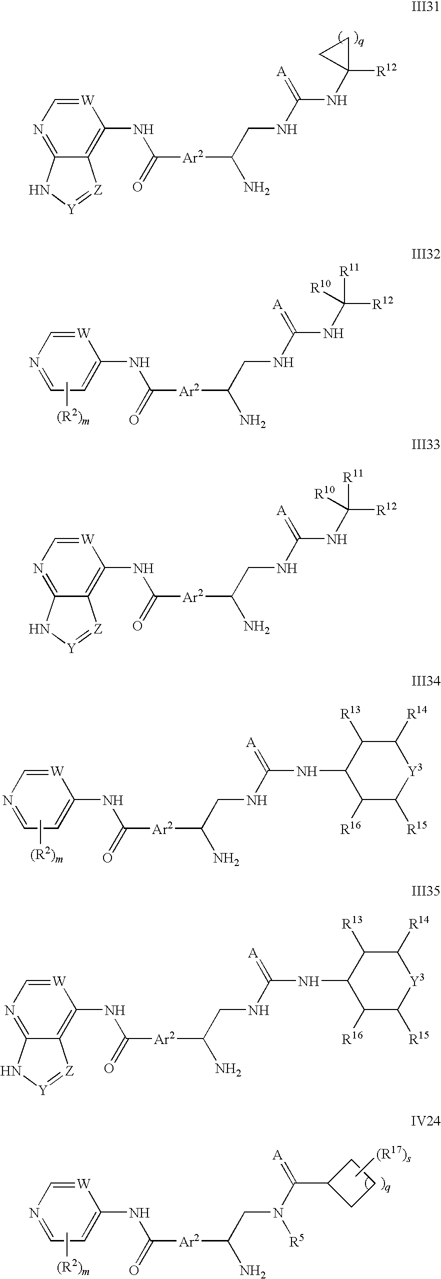

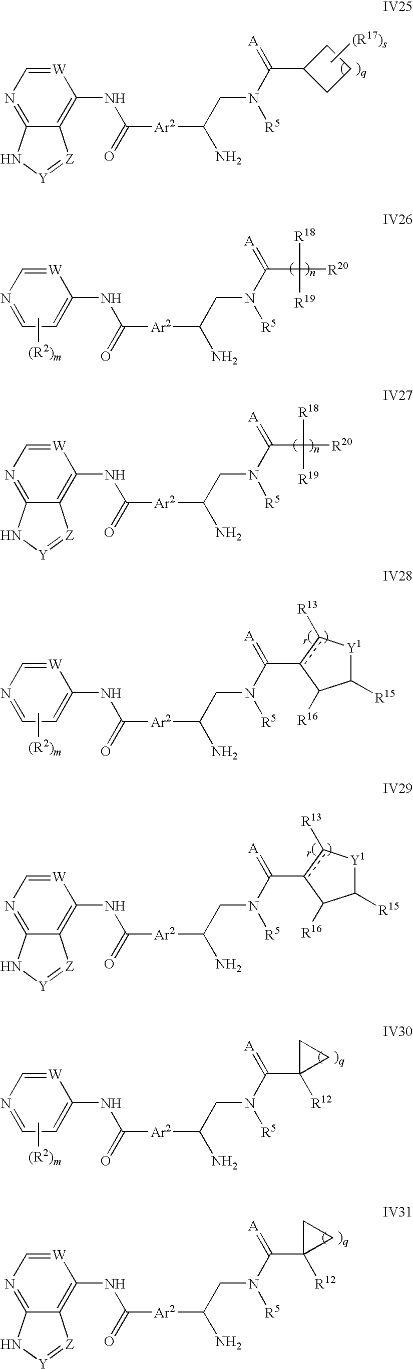

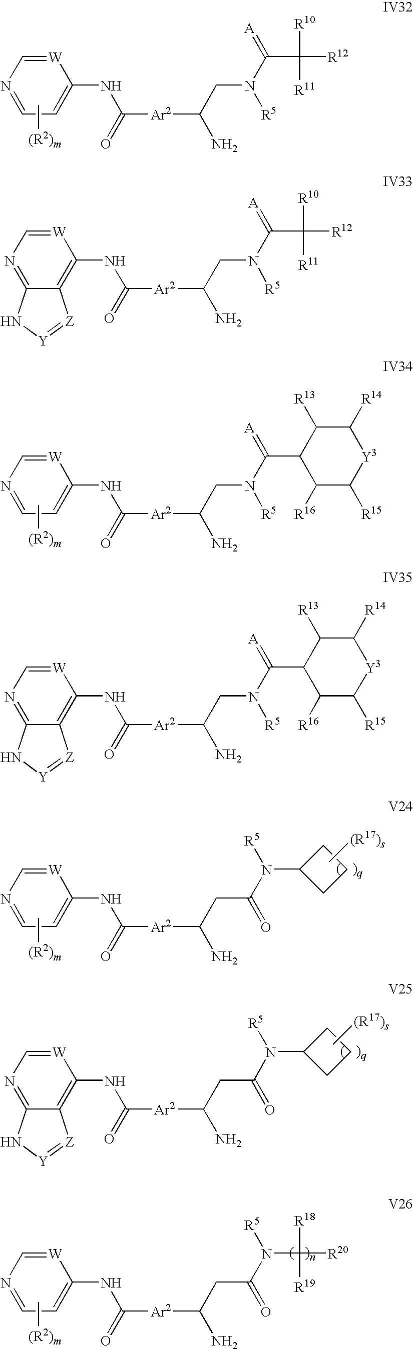

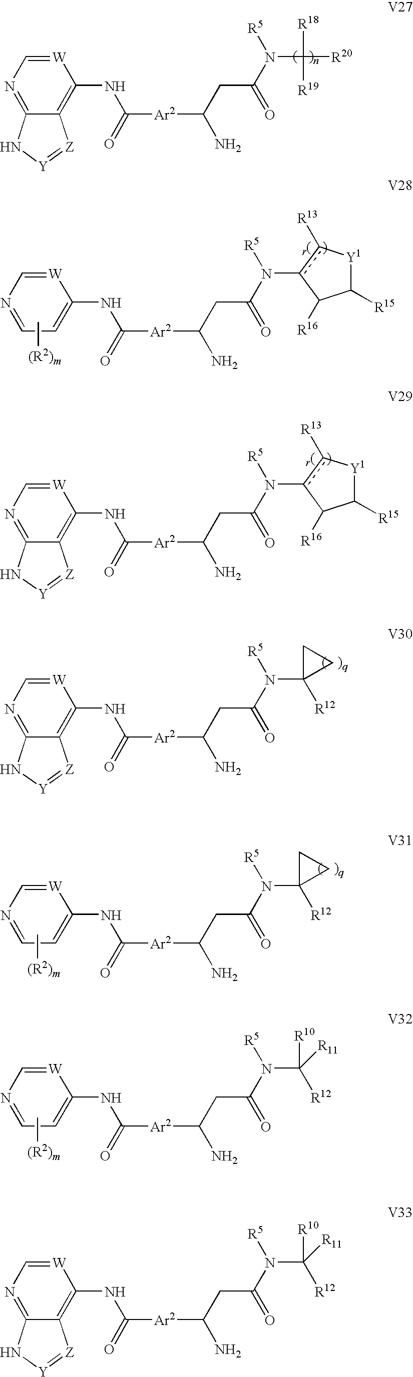

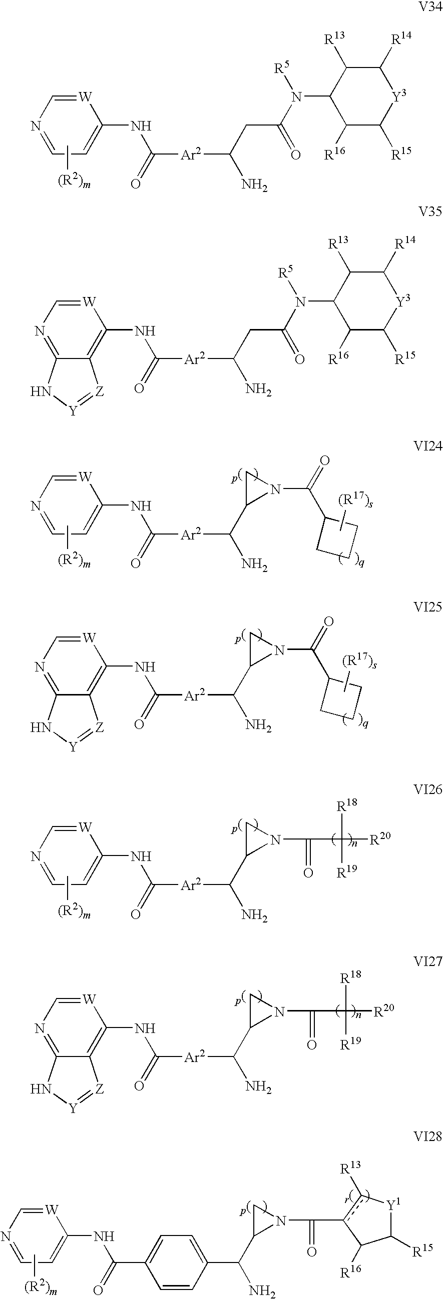

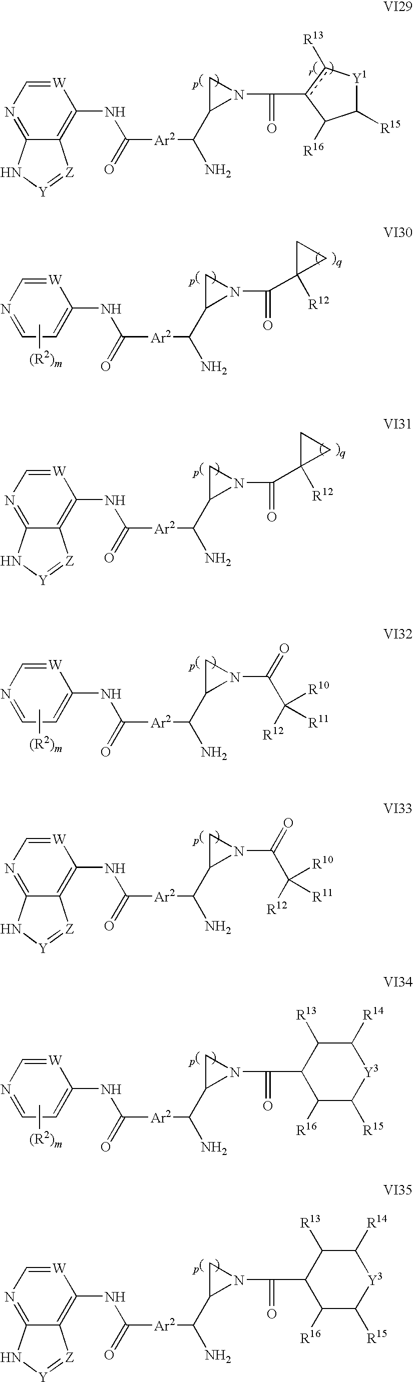

wherein Ar², A, s, p, q, r, m, n, W, Y, Y¹, Z, R², R⁵, R¹⁰, R¹¹, R¹², R¹³, R¹⁵, R¹⁶, R¹⁷, R¹⁸, R¹⁹ and R²⁰ have the same meaning as that defined above.

In a particular embodiment, the present invention provides compounds of Formula I or II having one of the structural Formula

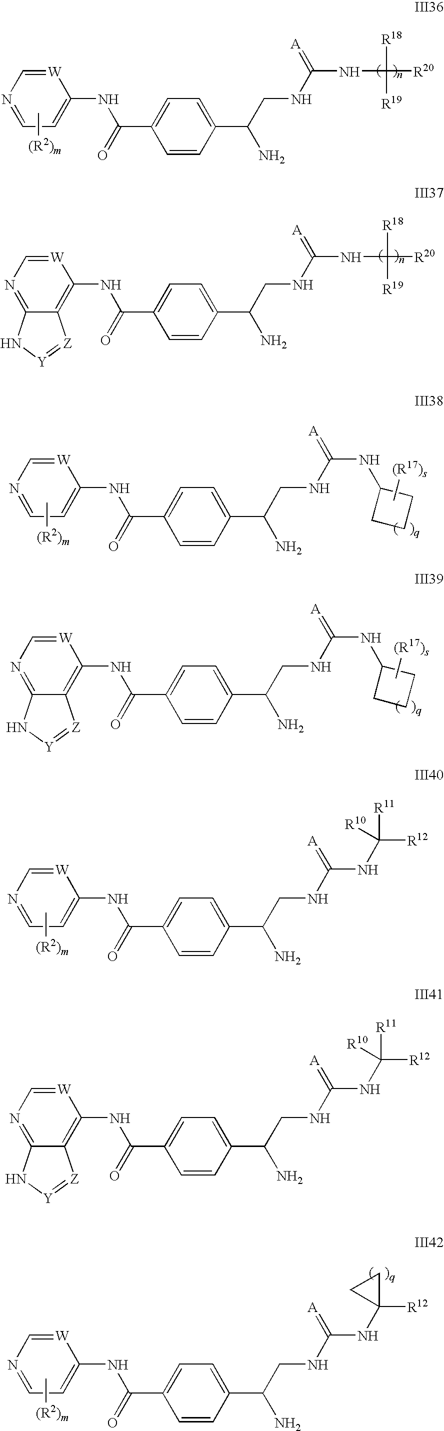

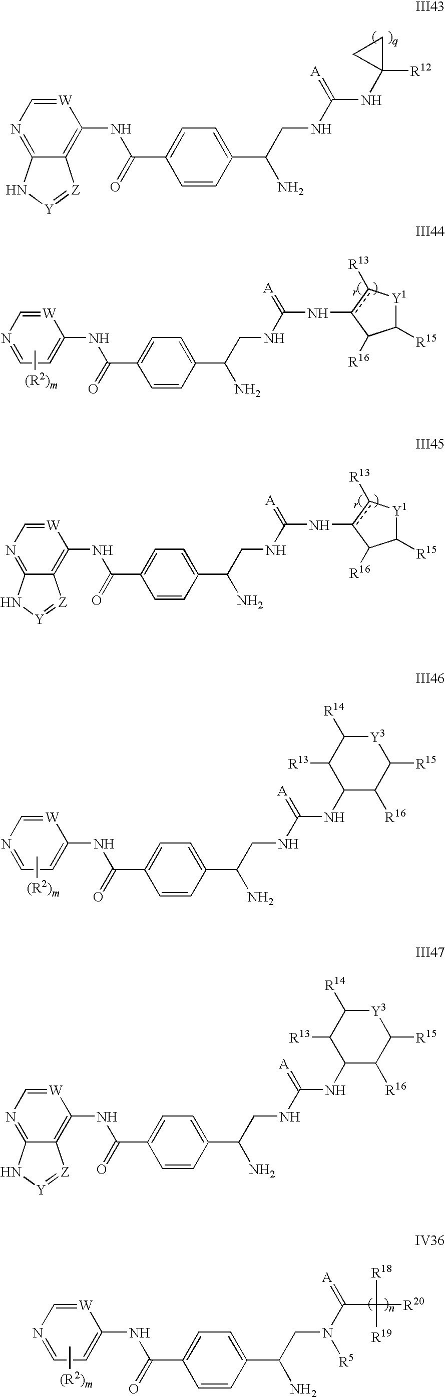

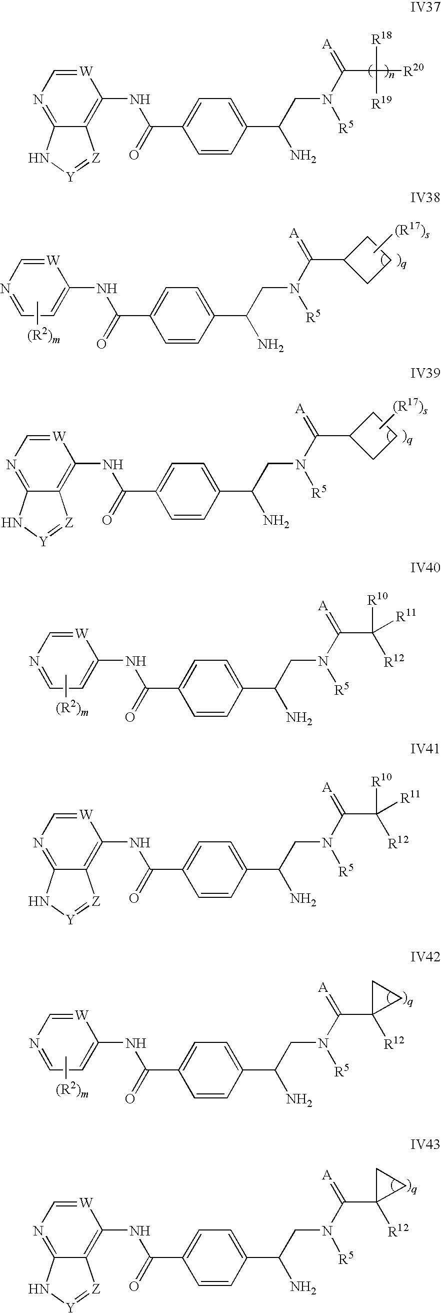

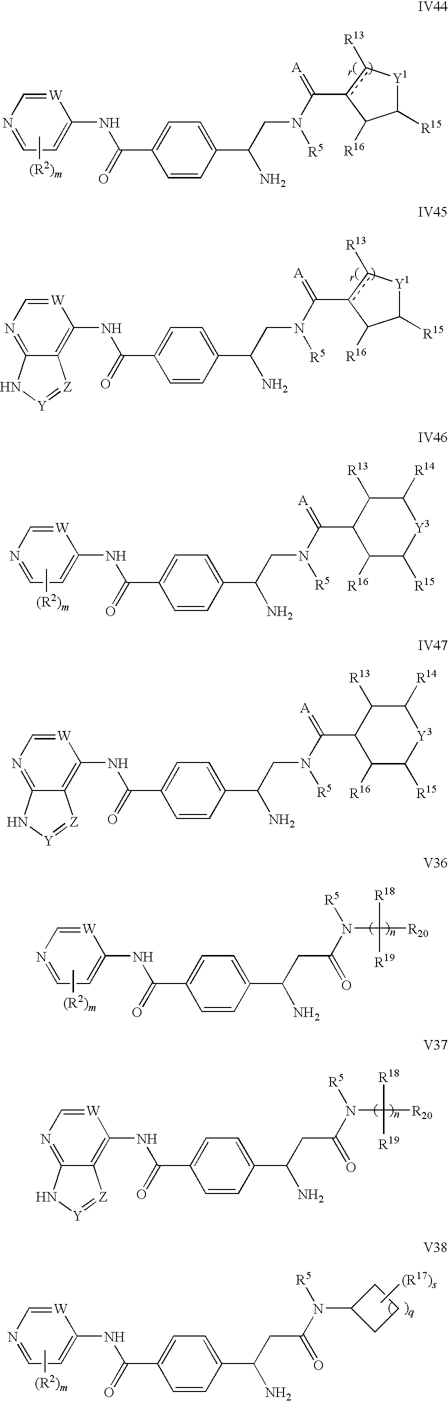

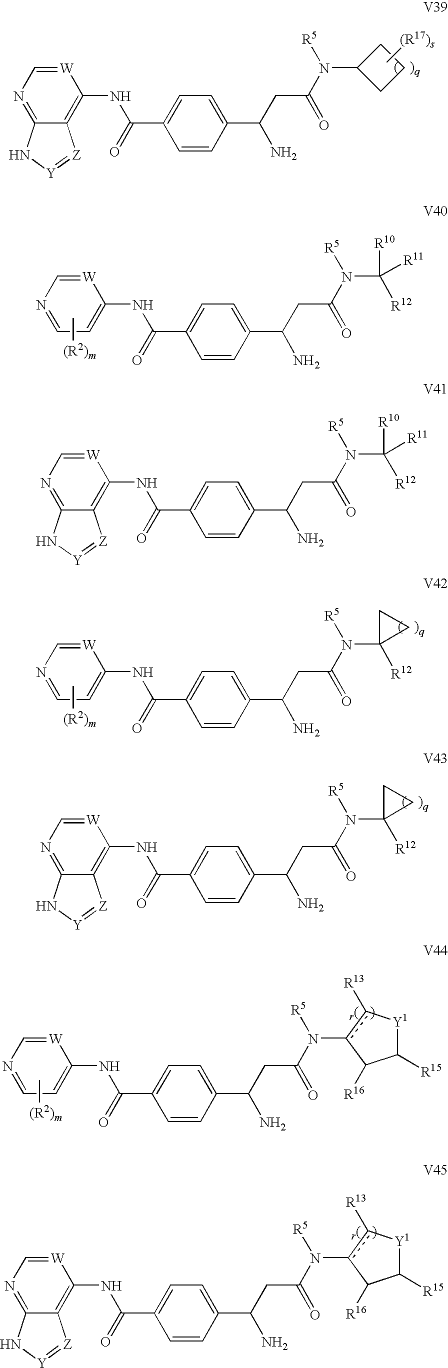

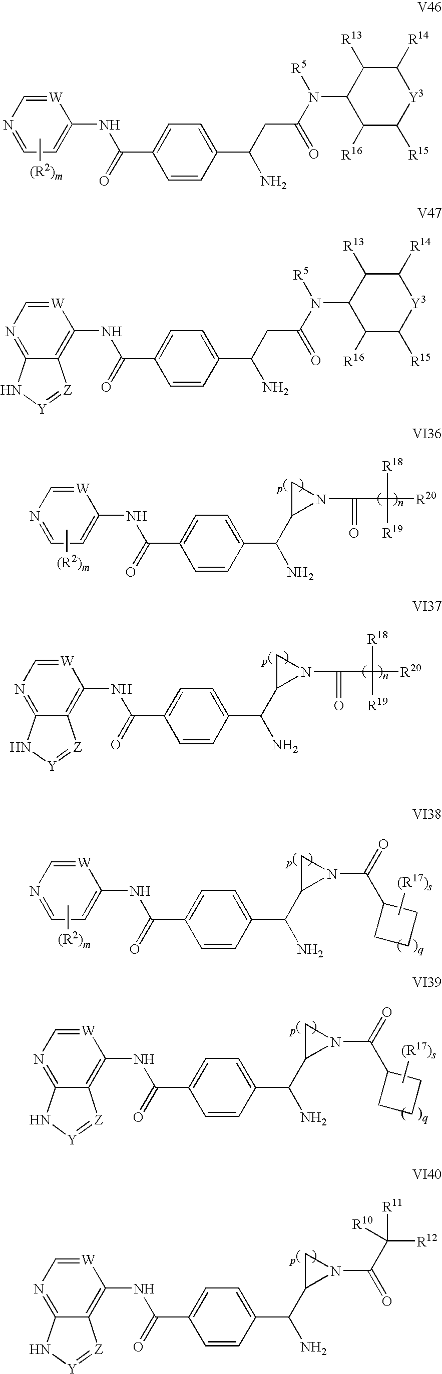

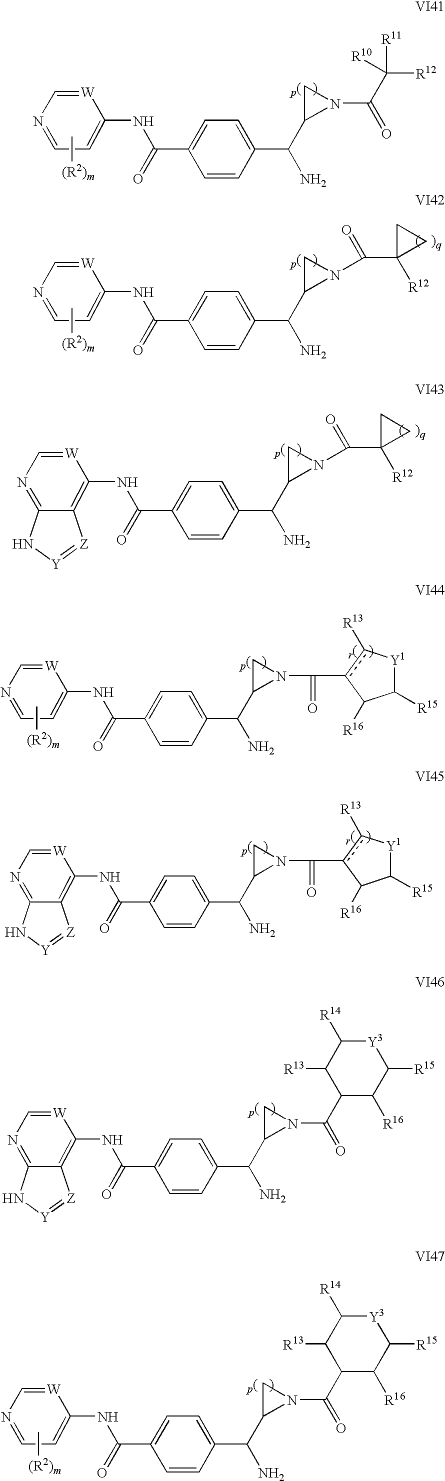

wherein A, s, p, q, r, n, m, W, Y, Y¹, Z, R², R⁵, R¹⁰R¹¹, R¹², R¹³, R¹⁵, R¹⁶, R¹⁷, R¹⁸, R¹⁹ and R²⁰ have the same meaning as that defined above.

In an embodiment, the present invention relates to any of the compounds described above wherein, Y¹ is selected from —CH₂—, —CH(R¹⁴)—, —NH—, —O—, —S— or —C(═O)—,

Y³ is selected from —CH₂—, —CH₂—CH₂—, —O—, —S— or —NH—,
A is O or S, W is N or CR², Y is N or CR², Z is N or CR², wherein R² is hydrogen or alkyl,
R⁵ is hydrogen, alkyl or cycloalkyl,
p is 3 or 4,
q is an integer selected from 1, 2, 3 or 4,
n is an integer selected from 1, 2, 3, 4, 5, 6 or 7,
wherein R¹⁰ and R¹¹ are each independently a selected from hydrogen, alkyl, cycloalkyl, aryl, or aralkyl,
wherein R¹² is selected from aryl, cycloalkyl, heteroaryl or heterocyclyl, each optionally substituted by one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
r is an integer selected from 0, 1, 2 or 3,
wherein R¹³ and R¹⁴ are each independently selected from hydrogen or alkyl,
or R¹³ and R¹⁴ form together with the carbon atoms to which they are attached form an aryl, an heteroaryl, a cycloalkyl or a heterocyclyl,
or r is 2 and two R¹³ form together with the carbon atoms to which they are attached form an aryl, an heteroaryl, a cycloalkyl or a heterocyclyl,
wherein R¹⁵ and R¹⁶ together with the carbon atom to which they are attached form an aryl, a cycloalkyl, a heteroaryl a heterocyclyl, each optionally substituted with one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
s is an integer selected from 0, 1, 2, 3 or 4,
wherein R¹⁷ is selected from halo, or a group selected from alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylalkenylaminooxy, heteroarylalkyl, heteroarylcarbonylamino, heterocyclyl, hydroxyalkyl, nitro, oxo, sulfonyl, or two R¹⁷ together with the atoms to which they are attached form an aryl, heteroaryl, cycloalkyl, or heterocyclyl, each group being optionally substituted with one or more substituents selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
wherein R¹⁸ and R¹⁹ are each independently selected from hydrogen, alkyl, cycloalkyl, cycloalkylalkyl, aryl, aralkyl, heteroaryl or heteroarylalkyl,
wherein R²⁰ is selected from hydrogen, or a group selected from alkyl, cycloalkyl, alkylaminoalkyl, alkylamino, alkylcarbonylamino, alkylcarbonylaminoalkyl, alkylsulfonyl, alkylsulfonylamino, alkylsulfonylaminoalkyl, amino, aminoalkyl, heterocyclyl, heterocyclylalkyl, cyano, cyanoalkyl, hydroxyl, hydroxyalkyl, alkoxy, alkoxyalkyl, alkoxycarbonyl, carboxy, alkoxycarbonylalkyl, aryl, aralkyl, heteroaryl, heteroarylalkyl, each group being optionally substituted by one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
wherein R²¹ is selected from alkyl, aryl, alkylcarbonyl, heteroaryl or heteroarylcarbonyl

In a preferred embodiment, the present invention provides compounds of Formula I or II having one of the structural Formula

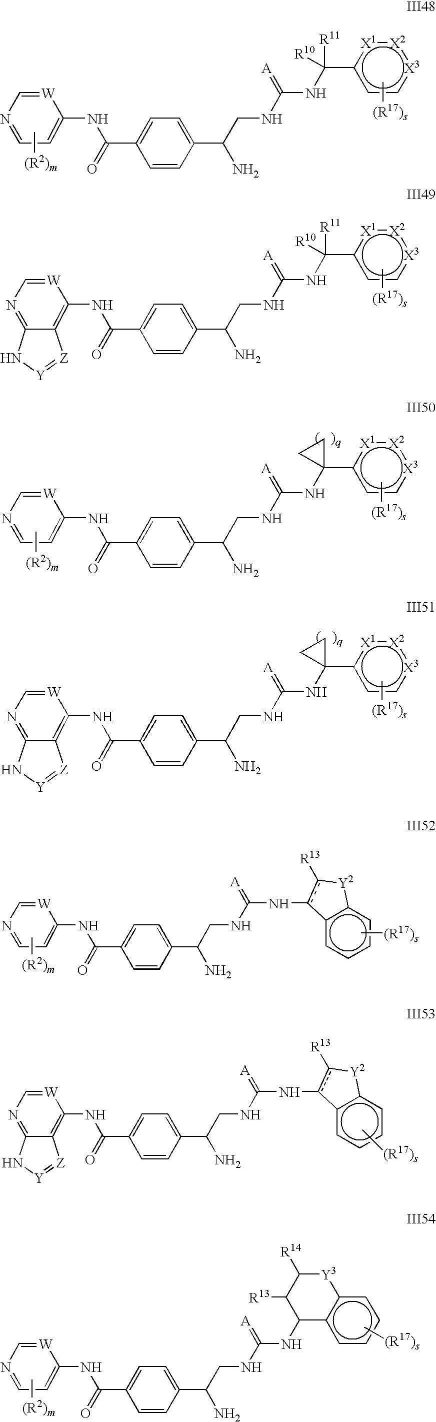

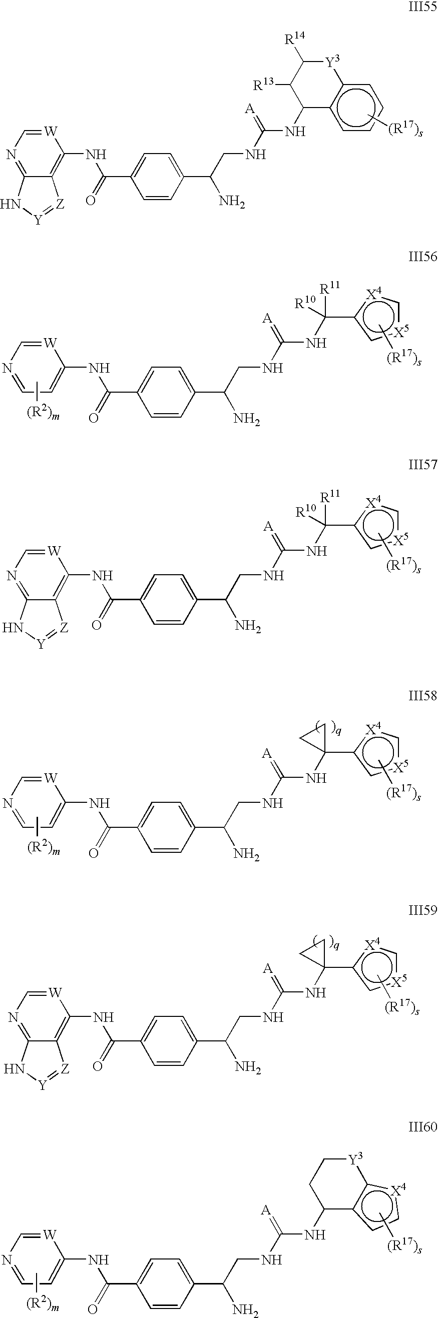

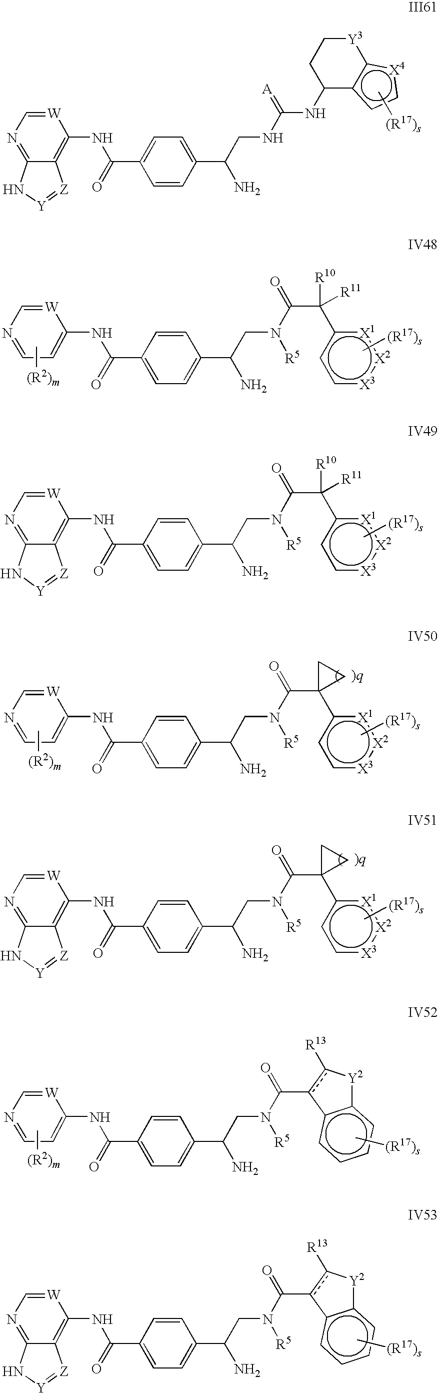

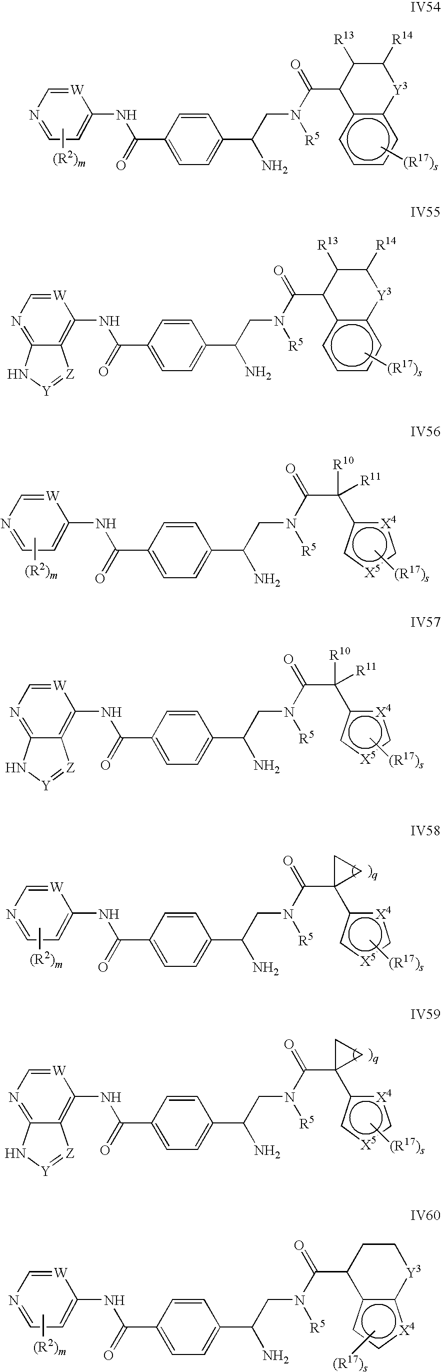

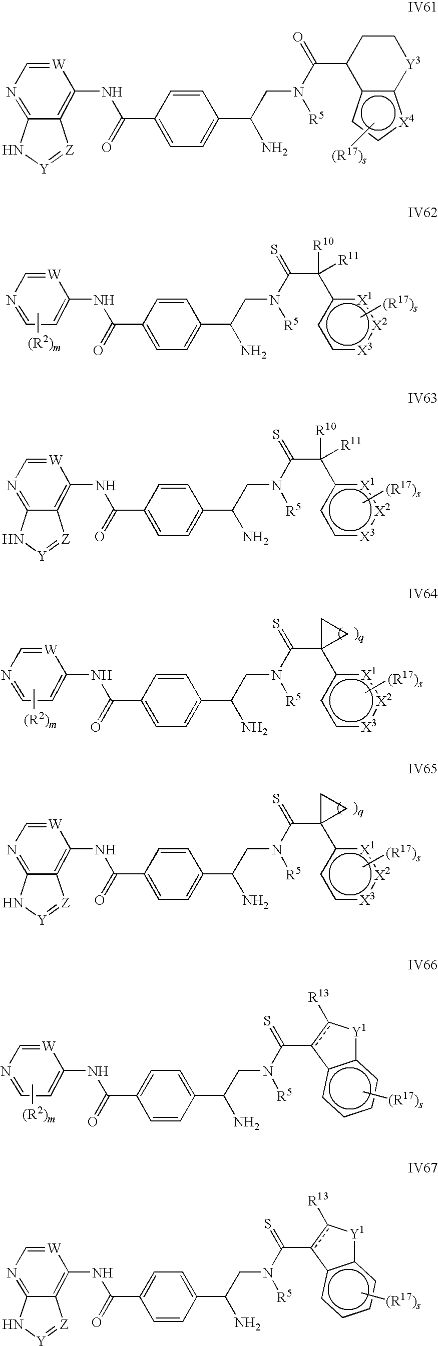

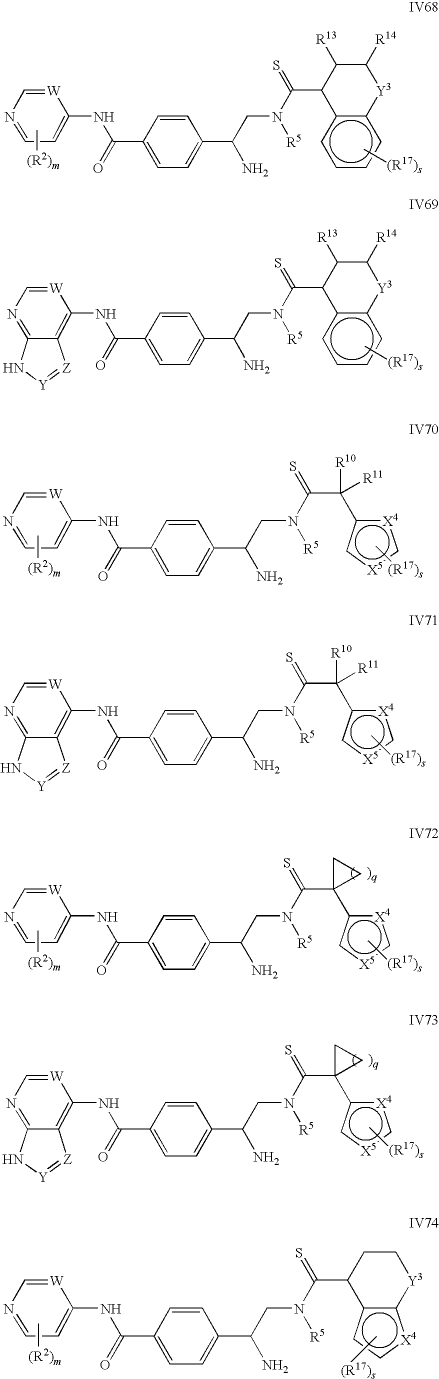

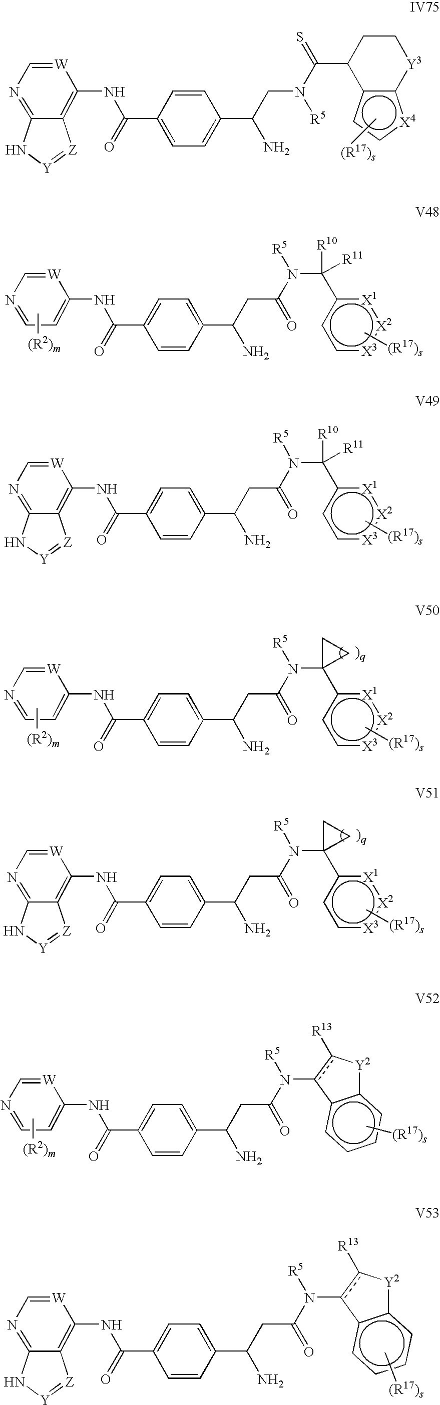

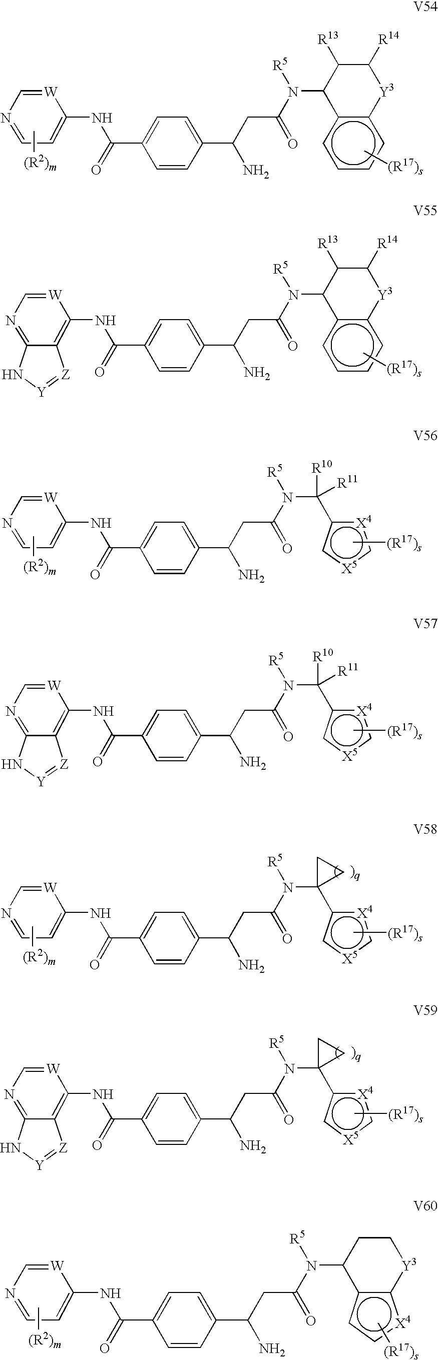

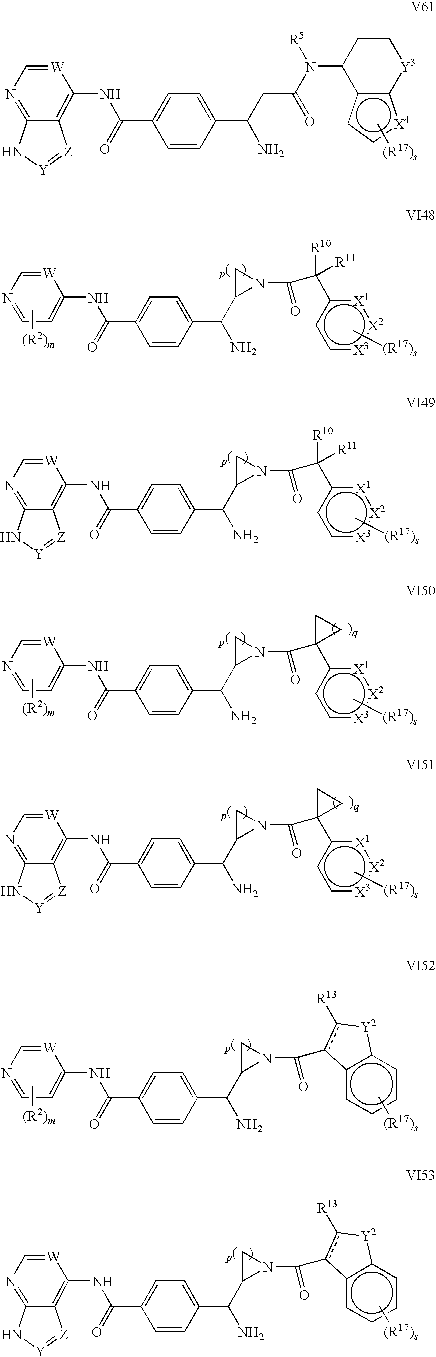

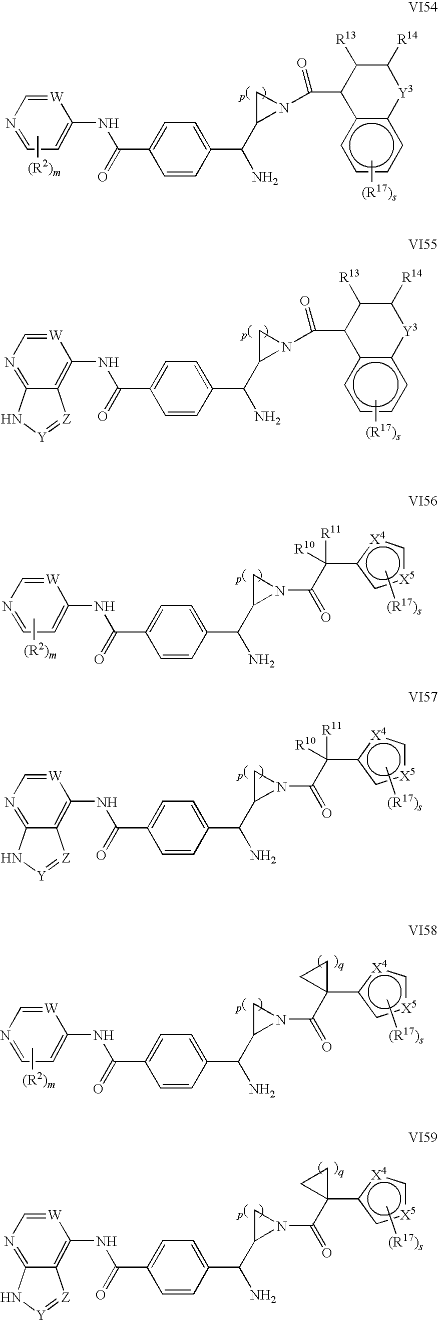

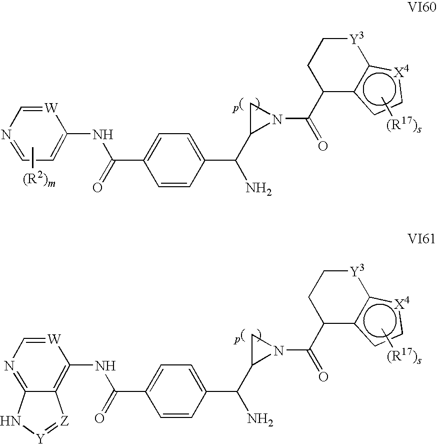

wherein A, s, p, q, r, n, m, W, Y, Y¹, Z, R², R⁵, R¹⁰, R¹¹, R¹², R¹³, R¹⁵, R¹⁶, R¹⁷, R¹⁸, R¹⁹ and R²⁰ have the same meaning as that described above.

In an embodiment, the present invention relates to any of the compounds described above wherein, A is O or S, W is N or CR², Y is N or CR², Z is CH or N, wherein R² is hydrogen or methyl, p is 3 or 4, m is 0, s is selected from 0, 1, 2 or 3, r is 1 or 2, wherein R⁵ is selected from hydrogen, alkyl or cycloalkyl, q is an integer selected from 1, 2, 3 or 4, preferably 3 or 4, n is selected from 0, 1, 2, 3, 4, 5, 6, 7 or 8,

R¹⁰ and R¹¹ are each independently a selected from hydrogen, alkyl, cycloalkyl, aryl, or aralkyl,
R¹⁷ is selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
Y² is selected from —CH(R¹⁴)—, —S—, —NH—, —O—, —C(═O)—,
R¹³ and R¹⁴ are each independently selected from hydrogen or alkyl or together with the carbon atoms to which they are attached form an aryl ring,
Y³ is selected from —CH₂—, —S—, —CH₂—CH₂—, —NH—, —O—, —C(═O)—,
wherein when X¹, X² or X³ are each independently selected from CH or N,
X⁴ is selected from N, S or CH, and
X⁵ is selected from CH or S.

It is clear to a person skilled in the art that the compounds of Formula I or II contain at least one asymmetric center and thus may exist as different stereoisomeric forms. This asymmetric center is indicated with an asterisk (*) in the figure below.

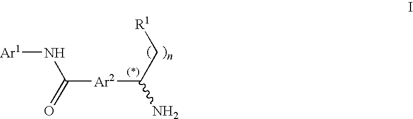

The absolute configuration of each asymmetric center that may be present in the compounds of Formula I or II may be indicated by the stereochemical descriptors R and S. When two chiral centers are present in the compound, in the configuration R,R for example the first letter refers to the configuration of the carbon bearing the amine group (*).

In a particular embodiment of the present invention, as illustrated hereunder at least one enantiomer is preferred for PKC and the other ones are preferred for ROCK.

In a particular embodiment, for the compounds of Formula IIa, when Ar² is phenylene or napthylene and R⁵ and/or R⁶ do not contain any chiral centers, the carbon atom marked with the asterisk (*) preferably has the R configuration (Formula IIaa) for PKC inhibition, and the S configuration (Formula IIba) for ROCK inhibition. It is the inverse when Ar² is thienylene.

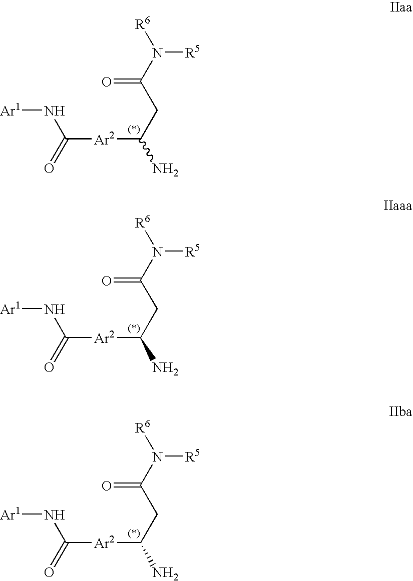

When R⁵ and/or R⁶ contain a chiral center, for example with a cyclized benzylamine as R⁵ or R⁶, as shown by Formula Vc, then 4 diastereoisomers are possible:

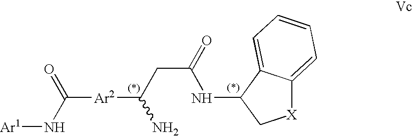

(i) If X is O or S, compounds with configuration R,S are preferred for PKC inhibition and compounds with configuration R,R; S,R or S,S are preferred for ROCK inhibition.

For example, compound 405 (4-{(R)-1-Amino-2-[(S)-(2,3-dihydro-benzofuran-3-yl)carbamoyl]-ethyl}-N-pyridin-4-yl-benzamide) is preferred as PKC inhibitor and compounds 406 (4-{(R)-1-Amino-2-[(R)-(2,3-dihydro-benzofuran-3-yl)carbamoyl]-ethyl}-N-pyridin-4-yl-benzamide), 408 (4-{(S)-1-Amino-2-[(R)-(2,3-dihydro-benzofuran-3-yl)carbamoyl]-ethyl}-N-pyridin-4-yl-benzamide) or 407 (4-{(S)-1-Amino-2-[(S)-(2,3-dihydro-benzofuran-3-yl)carbamoyl]-ethyl}-N-pyridin-4-yl-benzamide) are preferred as ROCK inhibitors.

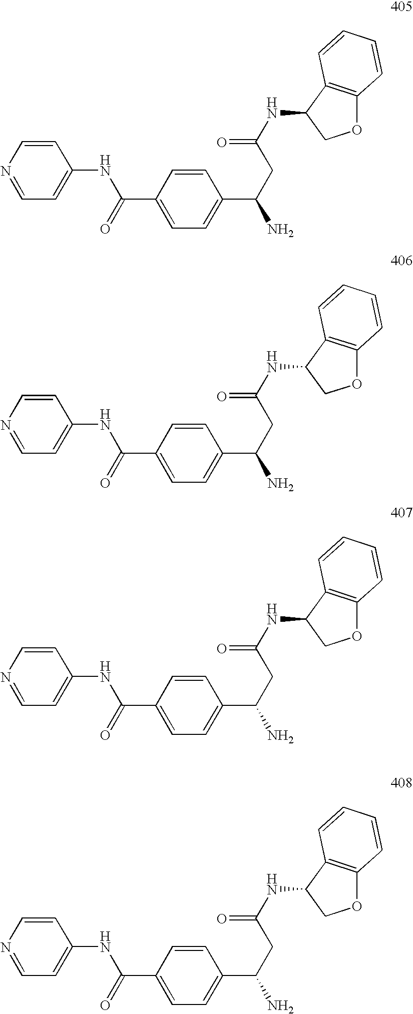

(ii) If X is CH₂, compounds with configuration R,R are preferred for PKC inhibition and compounds with configuration R,S; S,R or S,S are preferred for ROCK inhibition.

For example, compound 228 has four stereoisomers wherein compound 228a (4-[(R)-1-Amino-2-((R)-indan-1-ylcarbamoyl)-ethyl]-N-pyridin-4-yl-benzamide) is preferred as PKC inhibitor and compounds 228b (4-[(R)-1-Amino-2-((S)-indan-1-ylcarbamoyl)-ethyl]-N-pyridin-4-yl-benzamide), 228c (4-[(S)-1-Amino-2-((S)-indan-1-ylcarbamoyl)-ethyl]-N-pyridin-4-yl-benzamide) or 228d (4-[(S)-1-Amino-2-((R)-indan-1-ylcarbamoyl)-ethyl]-N-pyridin-4-yl-benzamide) are preferred as ROCK inhibitors.

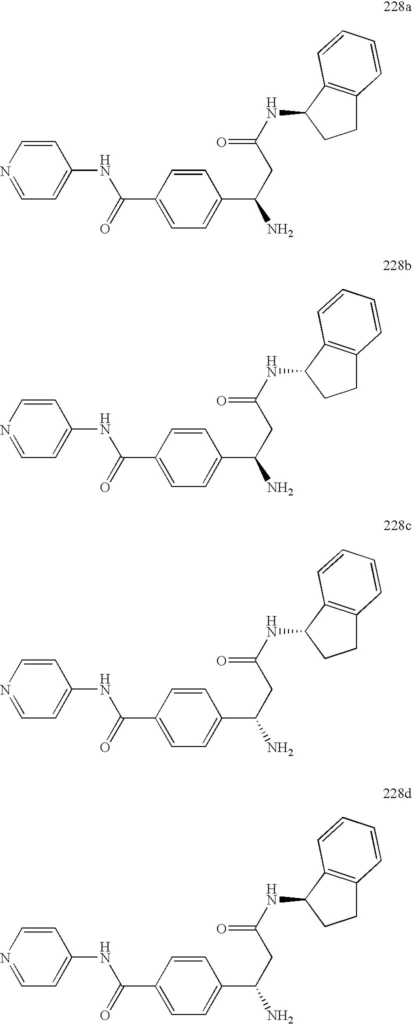

When R⁵ and/or R⁶ contain a chiral center, with for example a cyclized benzylamine as R⁵ or R⁶, as shown by Formula VIII,

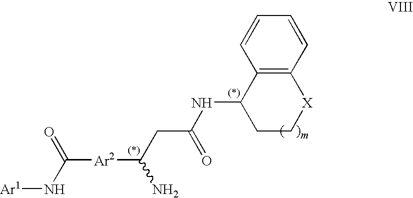

wherein X is O, S or CH₂ and m is 1 or 2, then 4 diastereoisomers are possible: compounds with configuration R,R are preferred for PKC inhibition and compounds with configuration R,S; S,R or S,S are preferred for ROCK inhibition, more preferably R,S or S,R.

In a particular embodiment, for the compounds of Formula IIIa, when Ar² is phenylene or napthylene and R⁷ does not contain any chiral centers, the carbon atom marked with the asterisk (*) preferably has the S configuration (Formula IIIaa) for PKC inhibition, and the R configuration (Formula IIIba) for ROCK inhibition. It is the inverse when Ar² is thienylene.

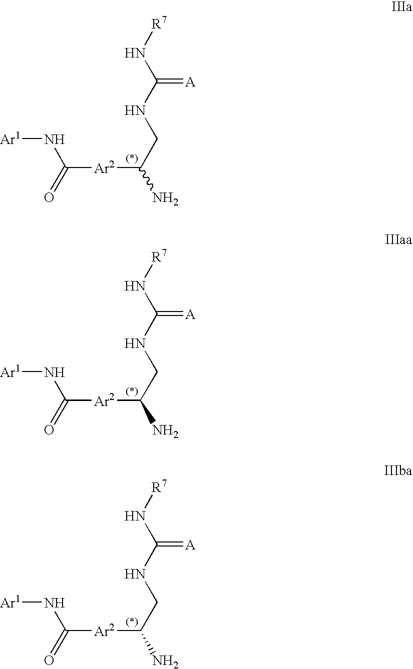

In a particular embodiment, for the compounds of Formula IVa, when Ar² is phenylene or napthylene and when R⁷ does not contain any chiral centers, the carbon atom marked with the asterisk (*) preferably has the S configuration (Formula IVaa) for PKC inhibition, and the R configuration (Formula IVaa) for ROCK inhibition. It is the inverse when Ar² is thienylene.

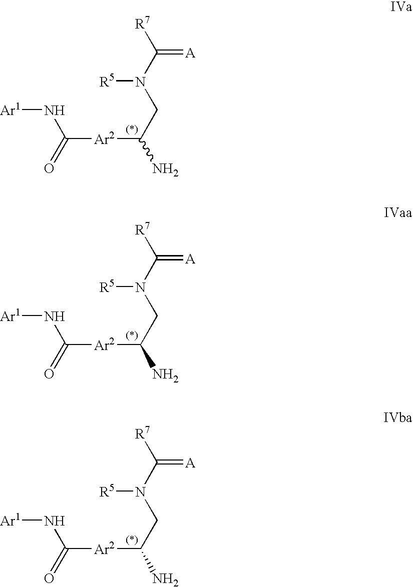

When R⁷ contains a chiral center, for example with a cyclized benzylamine, as shown by Formula IX, then 4 diastereoisomers are possible:

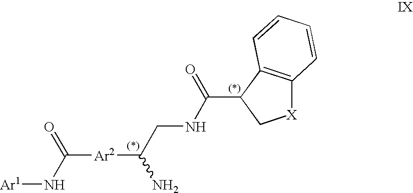

(i) If X is O or S, compounds with configuration S,S are preferred for PKC inhibition and compounds with configuration R,R; S,R or R,S are preferred for ROCK inhibition.

For example, compound 646 has four stereoisomers wherein compound 646a ((S)-2,3-Dihydro-benzofuran-3-carboxylic acid {(S)-2-amino-2-[4-(pyridin-4-ylcarbamoyl)-phenyl]-ethyl}-amide) is preferred as PKC inhibitor and compounds 646b ((R)-2,3-Dihydro-benzofuran-3-carboxylic acid {(S)-2-amino-2-[4-(pyridin-4-ylcarbamoyl)-phenyl]-ethyl}-amide), 646c ((S)-2,3-Dihydro-benzofuran-3-carboxylic acid {(R)-2-amino-2-[4-(pyridin-4-ylcarbamoyl)-phenyl]-ethyl}-amide) or 646d ((R)-2,3-Dihydro-benzofuran-3-carboxylic acid {(R)-2-amino-2-[4-(pyridin-4-ylcarbamoyl)-phenyl]-ethyl}-amide) are preferred as ROCK inhibitors.

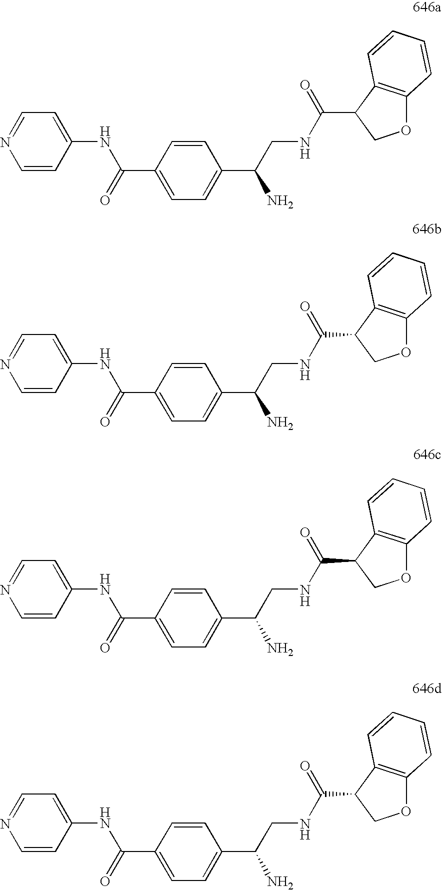

(ii) If X is CH₂, compounds with configuration S,R are preferred for PKC inhibition and compounds with configuration R,S; R,R or S,S are preferred for ROCK inhibition.

For example, compound 413 ((R)-Indan-1-carboxylic acid {(S)-2-amino-2-[4-(pyridin-4-ylcarbamoyl)-phenyl]-ethyl}-amide) is preferred as PKC inhibitor and compounds 414 ((S)-Indan-1-carboxylic acid {(S)-2-amino-2-[4-(pyridin-4-ylcarbamoyl)-phenyl]-ethyl}-amide), 416 (S)-Indan-1-carboxylic acid {(R)-2-amino-2-[4-(pyridin-4-ylcarbamoyl)-phenyl]-ethyl}-amide) or 415 ((R)-Indan-1-carboxylic acid {(R)-2-amino-2-[4-(pyridin-4-ylcarbamoyl)-phenyl]-ethyl}-amide) are preferred as ROCK inhibitors.

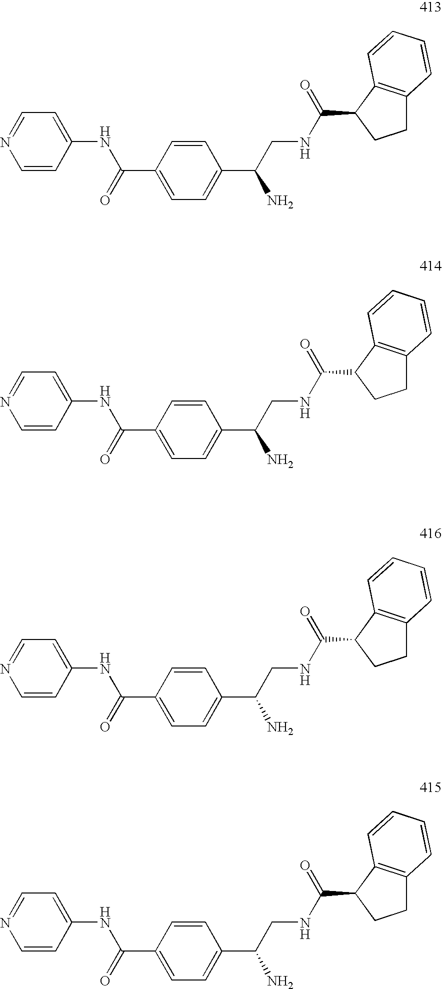

When R⁷ contains a chiral center, with for example a cyclized benzylamine, as shown by Formula XI,

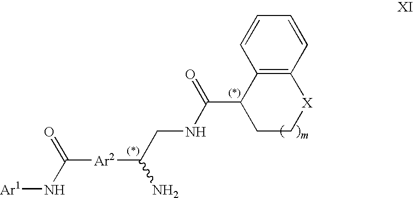

wherein X is O, S, NH, NMe or CH₂ and m is 1 or 2, then 4 diastereoisomers are possible: compounds with configuration S,R are preferred for PKC inhibition and compounds with configuration R,S; R,R or S,S are preferred for ROCK inhibition.

For example, compound 409 ((R)-1,2,3,4-Tetrahydro-naphthalene-1-carboxylic acid {(S)-2-amino-2-[4-(pyridin-4-ylcarbamoyl)-phenyl]-ethyl}-amide) is preferred as PKC inhibitor and compounds 410 ((S)-1,2,3,4-Tetrahydro-naphthalene-1-carboxylic acid {(S)-2-amino-2-[4-(pyridin-4-ylcarbamoyl)-phenyl]-ethyl}-amide), 412 ((S)-1,2,3,4-Tetrahydro-naphthalene-1-carboxylic acid {(R)-2-amino-2-[4-(pyridin-4-ylcarbamoyl)-phenyl]-ethyl}-amide) or 411 ((R)-1,2,3,4-Tetrahydro-naphthalene-1-carboxylic acid {(R)-2-amino-2-[4-(pyridin-4-ylcarbamoyl)-phenyl]-ethyl}-amide) are preferred as ROCK inhibitors.

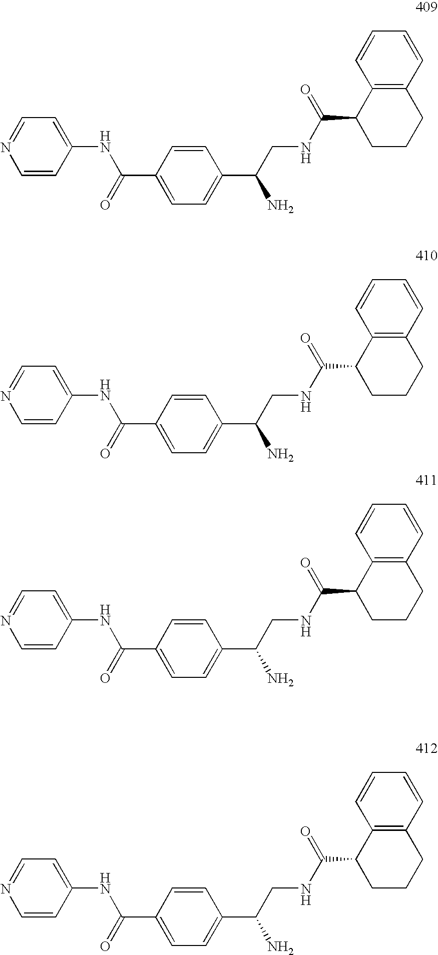

In a particular embodiment, for the compounds of Formula XX, when Ar² is phenylene or napthylene and when R⁷ does not contain any chiral centers,

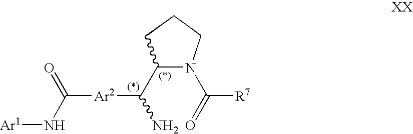

The preferred configuration (for PKC) of the carbon bearing the NH₂ group is S.

The preferred configuration (for PKC) of the carbon of the proline moiety group is R.

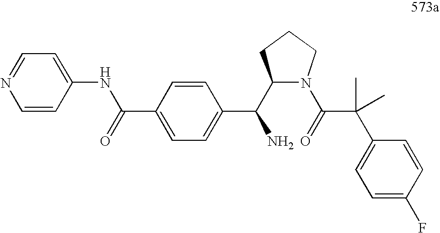

### Compound 573a 4-((S)-Amino-{(R)-1-[2-(4-fluoro-phenyl)-2-methyl-propionyl]-pyrrolidin-2-yl}-methyl)-N-pyridin-4-yl-benzamide

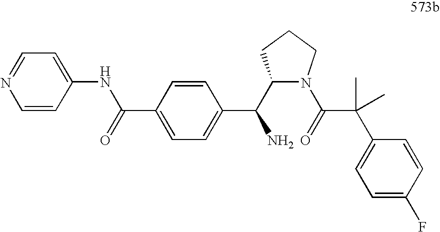

### Compound 573b 4-((S)-Amino-{(S)-1-[2-(4-fluoro-phenyl)-2-methyl-propionyl]-pyrrolidin-2-yl}-methyl)-N-pyridin-4-yl-benzamide

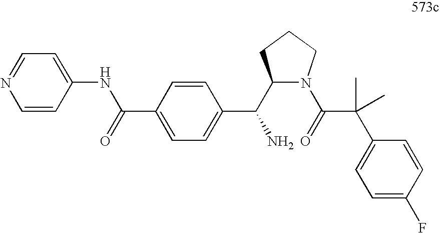

### Compound 573c 4-((R)-Amino-{(R)-1-[2-(4-fluoro-phenyl)-2-methyl-propionyl]-pyrrolidin-2-yl}-methyl)-N-pyridin-4-yl-benzamide

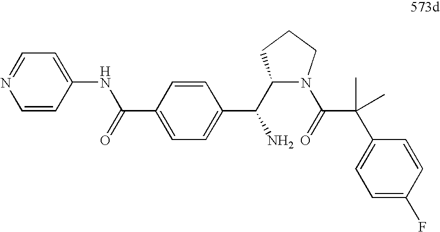

### Compound 573d 4-((R)-Amino-{(S)-1-[2-(4-fluoro-phenyl)-2-methyl-propionyl]-pyrrolidin-2-yl}-methyl)-N-pyridin-4-yl-benzamide

When R⁷ contains a chiral center, as shown by Formula XXa or XXb, then 6 diastereoisomers are possible:

For Formula XXa:

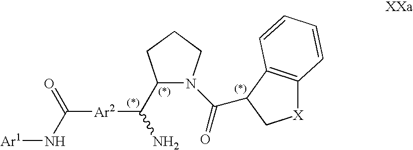

If X═O, S

The preferred configuration (for PKC) of the carbon bearing the NH₂ group is S.

The preferred configuration (for PKC) of the carbon of the proline moiety group is R.

The preferred configuration (for PKC) of the last asymmetric carbon is S.

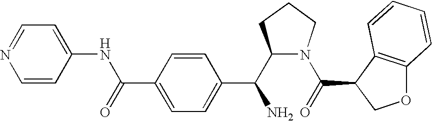

### 4-{(S)-Amino-[(R)-1-((S)-2,3-dihydro-benzofuran-3-carbonyl)-pyrrolidin-2-yl]-methyl}-N-pyridin-4-benzamide

If X═CH₂

The preferred configuration (for PKC) of the carbon bearing the NH₂ group is S.

The preferred configuration (for PKC) of the carbon of the proline moiety group is R.

The preferred configuration (for PKC) of the last asymmetric carbon is R.

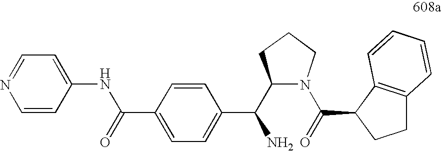

### Compound 608a 4-{(S)-Amino-[(R)-1-((R)-indane-1-carbonyl)-pyrrolidin-2-yl]-methyl}-N-pyridin-4-yl-benzamide

For Formula XXb:

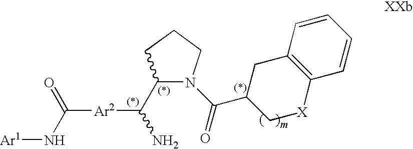

X can be CH₂, O, S, NH or NMe:

The preferred configuration (for PKC) of the carbon bearing the NH₂ group is S.

The preferred configuration (for PKC) of the carbon of the proline moiety group is R.

The preferred configuration (for PKC) of the last asymmetric carbon is R.

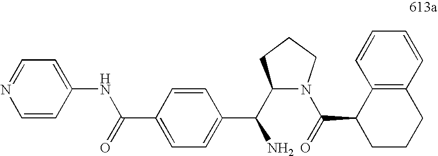

### Compound 613a 4-{(S)-Amino-[(R)-1-((R)-1,2,3,4-tetrahydro-naphthalene-1-carbonyl)-pyrrolidin-2-yl]-methyl}-N-pyridin-4-yl-benzamide

The compounds of the invention may be in the form of pharmaceutically and/or veterinary acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I or II above.

The invention also generally covers all pharmaceutically acceptable predrugs and prodrugs of the compounds of Formula I or II, for which general reference is made to the prior art cited hereinbelow.

The term “pro-drug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793 and WO 99/33792 all incorporated herein by reference. Pro-drugs are characterized by increased bio-availability and are readily metabolized into the active inhibitors in vivo. The term “pre-drug”, as used herein, means any compound that will be modified to form a drug species, wherein the modification may take place either inside or outside of the body, and either before or after the pre-drug reaches the area of the body where administration of the drug is indicated.

As described above, some of the compounds of the invention may contain one or more asymmetric carbon atoms that serve as a chiral center, which may lead to different optical forms (e.g. enantiomers or diastereoisomers). The invention comprises all such optical forms in all possible configurations, as well as mixtures thereof.

More generally, from the above, it will be clear to the skilled person that the compounds of the invention may exist in the form of different isomers and/or tautomers, including but not limited to geometrical isomers, conformational isomers, E/Z-isomers, stereochemical isomers (i.e. enantiomers and diastereoisomers) and isomers that correspond to the presence of the same substituents on different positions of the rings present in the compounds of the invention. All such possible isomers, tautomers and mixtures thereof are included within the scope of the invention.

The compounds of Formula I or II may be prepared as described in the experimental section below using methods and chemistries with which those skilled in the art shall be familiar.

It will also be clear that when the desired compounds of the invention, and/or the starting materials, precursors and/or intermediates used in the preparation thereof, contain functional groups that are sensitive to the reaction conditions used in the preparation of the compounds of the invention (i.e. that would undergo undesired reactions under those conditions if they were not suitably protected) can be protected during said reaction with one or more suitable protective group, which protective group can then be suitably removed after either completion of said reaction and/or as a later or final step in the preparation of the compounds of the invention. Protected forms of the inventive compounds are included within the scope of the present invention. Suitable protective groups, as well as methods and conditions for inserting them and removing them, will be clear to the skilled person and are generally described in the standard handbooks of organic chemistry, such as Greene and Wuts, “Protective groups in organic synthesis”, 3rd Edition, Wiley and Sons, 1999, which is incorporated herein by reference in its entirety. It will also be clear to the skilled person that compounds of the invention in which one or more functional groups have been protected with suitable functional groups can find use as intermediates in the production and/or synthesis of the compounds of the invention, and as such form a further aspect of the invention.

Generally, the compounds of the invention are prepared from amine- or carboxylic acid-containing intermediates described hereinafter which may be reacted with complementary reactive molecules so as to form the desired compound. The intermediates and complementary reactive molecules are either commercially available or may be easily prepared by the skilled person.

According to a particular embodiment, the present invention encompasses the method for the preparation of enantiomers of Formula IVaa, and compounds obtainable therewith wherein Ar² is phenylene, A is O, and wherein Ar¹, R⁵ and R⁷ have the same meaning as that defined above. Enantiomers of Formula IVaa with Ar² being phenylene and A being O (compound of Formula XXIII) can be obtained: by reacting a compound of Formula XX with Noyori's catalyst (JACS, 1996, 118, 2521; JACS, 2005, 127, 4596), thereby obtaining compound of Formula XXI.

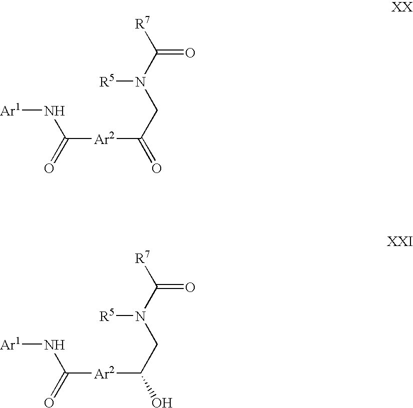

Noyori's catalyst can be obtained by reacting dichloro(p-cymene)ruthenium (II) dimer (0.05 eq.) with (1S,2S)-(+)-N-p-tosyl-1,2-diphenylethylenediamine.

Compound of Formula XXI is then reacted with diphenylphosphoryl azide (DPPA) and with 1,8-diazabicyclo[5.4.0]undec-7-ene (DBU) to give azide of Formula XXII.

The azide of Formula XXII is then reacted with Pd/C to give the amine of Formula XXIII.

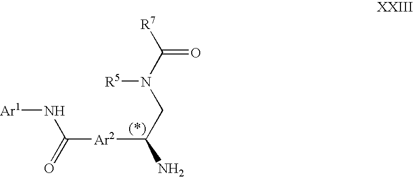

In a preferred embodiment, enantiomers IVaa with Ar² being phenylene, R⁵ being H and A being O can be obtained according to the protocol illustrated in scheme 1. Using this protocol, the carbon atom bearing the amine has always the S configuration for the enantiomer obtained.

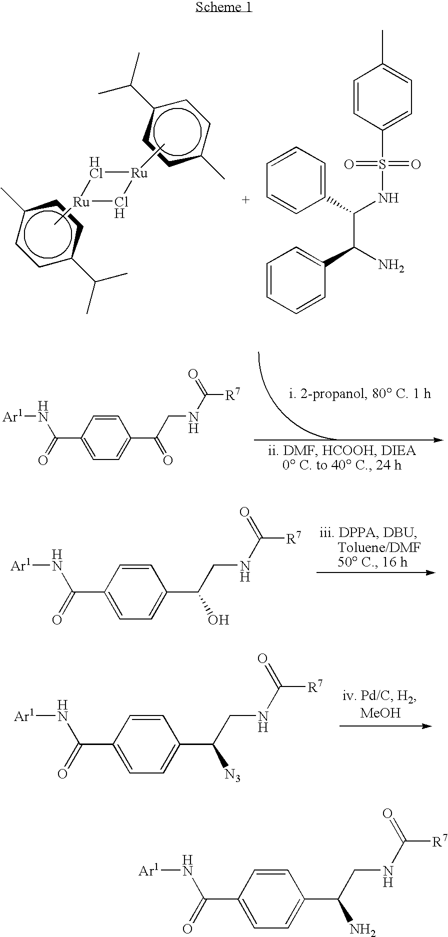

The compounds of the invention may be used for the inhibition of kinases in vitro or in vivo, preferably in vitro, for modulating biological pathways and/or processes in which such kinases are involved; and/or to prevent and/or treat diseases or disorders in which such kinases, pathways and/or processes are involved.

In a particular embodiment, the compounds of the invention may be used for the inhibition of PKC epsilon in vitro or in vivo, preferably in vitro, for modulating biological pathways and/or processes in which PKC epsilon is involved; and/or to prevent and/or treat diseases or disorders in which PKC epsilon, pathways and/or processes are involved.

According to one preferred, but non-limiting embodiment, the compounds of the invention may be used for any purposes known per se for inhibitors of PKC epsilon.

PKC epsilon are described in the prior art mentioned above and/or are commercially available, such as the Protein Kinase C Assay Kits available from Invitrogen.

In the invention, particular preference is given to compounds of Formula I or II above that in the inhibition assay for PKC epsilon described below inhibit PKC epsilon with an IC₅₀ value of less than 100 μM, preferably less than 50 μM, more preferably less than 10 μM, preferably less than 5 μM, even more preferably less than 1 μM, preferably less than 0.1 μM, and in particular less than 10 nM, for example less than or 1 nM, as determined by a suitable assay, such as the assay used in the Examples below.

The present invention also relates to the use of the compounds of Formula I or II above in (the preparation of a composition for) inhibiting PKC epsilon. Said inhibition may be effected in vitro and/or in vivo, and when effected in vivo, is preferably effected in a selective manner, as defined above. In another embodiment, the present invention also relates to the use of the compounds of Formula I or II above in (the preparation of a composition for) inhibiting PKC theta. Said inhibition may be effected in vitro and/or in vivo, and when effected in vivo, is preferably effected in a selective manner, as defined above.

According to particularly preferred embodiments, the compounds of the invention are preferably used in the prevention and/or treatment of at least one disease or disorder, preferably in which PKC epsilon is involved. According to an even more particularly preferred embodiment, the compounds of the invention may be used in the prevention and/or treatment of at least one disease or disorder in which the epsilon isoform of PKC is involved.

For example, the compounds of the invention may be used in the prevention and/or treatment of diseases and disorders such as:
metabolic diseases, such as:
(1) hyperglycemic conditions and/or other conditions and/or diseases that are (primarily) associated with (the response or sensitivity to) insulin, including but not limited to all forms of diabetes and disorders resulting from insulin resistance, such as Type I and Type II diabetes, as well as severe insulin resistance, hyperinsulinemia, and hyperlipidemia, e.g., obese subjects, and insulin-resistant diabetes, such as Mendenhall's Syndrome, Werner Syndrome, leprechaunism, lipoatrophic diabetes, and other lipoatrophies;
(2) conditions caused or usually associated with hyperglycemic conditions and/or obesity, such as hypertension, osteoporosis and/or lipodystrophy;
(3) so-called “metabolic syndrome” (also known as “Syndrome X”) which is a condition where several of the following conditions coexist: hypertension; insulin resistance; diabetes; dyslipidemia; and/or obesity;
   as well as various inherited metabolic diseases known per se; and may also be used also for preventing, treating and/or alleviating complications and/or symptoms associated with these metabolic diseases;
anxiety, addiction such as alcohol abuse or drug abuse, withdrawal syndrome, muscle spasms, convulsive seizures, epilepsy and other prophylactic and/or therapeutic uses mentioned in WO 00/01895 (for example, to modulate the action of drugs that target the GABA-A receptor);
pain, such as chronic hyperalgesia, inflammatory pain and the other diseases and disorders mentioned in WO 00/01415, U.S. Pat. No. 6,376,467, WO 02/102232, WO 03/089456 and WO 03/089457 and the further prior art listed above;
Cardiovascular disease or heart disease, as mentioned in US 2003/0134774;
Proliferative disease such as cancer,
and also for regulating the immune system and/or regulating an immune response in a mammal, as mentioned in WO 03/04612 and/or regulating an inflammatory response in a mammal.

The compounds of the invention may also be used as an alternative for the peptide inhibitors described in WO 03/089456 and WO 03/089457, e.g. for the same disease indications mentioned in these references for the peptide inhibitors, such as the management of pain. In doing so, the compounds of the invention will have all the usual advantages of small molecules compared to small peptides, for example that they can conveniently be formulated for oral administration, that they are usually easier to manufacture, and that they often are more stable under storage.

Preferably, the compounds and compositions of the invention may be used for preventing and/or treating diabetes, especially Type I and Type II diabetes, obesity and pain, especially preferably diabetes, as well as the complications and/or symptoms associated therewith. “Diabetes” itself refers to a progressive disease of carbohydrate metabolism involving inadequate production or utilization of insulin and is characterized by hyperglycemia and glycosuria.

According to a specific, very preferred, embodiment, the compounds and compositions of the invention are particularly suited for preventing and/or treating Type II diabetes.

In one specific non-limiting embodiment, the present invention relates to the use of the compounds of Formula I or II above in (the preparation of a composition for) the prevention and/or treatment of metabolic diseases such as diabetes and obesity.

In another specific non-limiting embodiment, the present invention relates to the use of the compounds of Formula I or II above in (the preparation of a composition for) the prevention, treatment and/or management of pain, including but not limited to chronic hyperalgesia and inflammatory pain.

In another specific non-limiting embodiment, the present invention relates to the use of the compounds of Formula I or II above in (the preparation of a composition for) the prevention, treatment and/or management of coronary heart disease, heart attack, cerebral vasospasm, stroke, kidney failure, kidney diseases or disorders, peripheral vasospasm, diabetic nephropathy, diabetic complications.

In another specific non-limiting embodiment, the present invention relates to the use of the compounds of Formula I or II above in (the preparation of a composition for) the prevention, treatment and/or management of diseases or disorders due to oxygen deprivation such as heart attack, stroke, kidney failure and the like.

In another specific non-limiting embodiment, the present invention relates to the use of the compounds of Formula I or II above in (the preparation of a composition for) the prevention, treatment and/or management of cardiovascular complications due to diabetes, high blood pressure, hypercholesterolemia, kidney failure and the like.

In another specific non-limiting embodiment, the present invention relates to the use of the compounds of Formula I or II above in (the preparation of a composition for) the prevention, treatment and/or management of transplant rejection (acute and chronic) as well as transplant dysfunction.

In another particular embodiment, the compounds of the invention may be used for the inhibition of PKC epsilon and PKC theta in vitro or in vivo, preferably in vitro, and also for modulating biological pathways and/or processes in which such kinases are involved; and/or to prevent and/or treat diseases or disorders in which such kinases, pathways and/or processes are involved.

In another specific non-limiting embodiment, the present invention relates to the use of the compounds of Formula I or II above in (the preparation of a composition for) the prevention, treatment and/or management of inflammatory diseases and auto-immune diseases such as contact dermatitis, psoriasis, rheumatoid arthritis, inflammatory bowel disease, Crohn's disease, ulcerative colitis, allergy and autoimmune diseases or disorders, AIDS and/or multiple sclerosis.

In another particular embodiment, the compounds of the invention may be used for the inhibition of ROCK in vitro or in vivo, preferably in vitro, and also for modulating biological pathways and/or processes in which such kinases are involved; and/or to prevent and/or treat diseases or disorders in which such kinases, pathways and/or processes are involved.

According to one preferred, but non-limiting embodiment, the compounds of the invention may be used to inhibit (at least one isoform of) ROCK; and as such may be used for any purposes known per se for inhibitors of ROCK.

In the invention, particular preference is given to compounds of Formula I or II above that in the inhibition assay for ROCK described below inhibit ROCK with an IC₅₀ value of less than 100 μM, preferably less than 50 μM, more preferably less than 10 μM, preferably less than 5 μM, even more preferably less than 1 μM, preferably less than 0.1 μM, and in particular less than 10 nM, for example less than or 1 nM, as determined by a suitable assay, such as the assay used in the Examples below.

The present invention also relates to the use of the compounds of Formula I or II above in (the preparation of a composition for) inhibiting at least one kinase, in particular for inhibiting at least one isoform of ROCK, more in particular for inhibiting ROCK I and/or ROCK II isoforms. As used herein, the term “ROCKI” can also be referred as ROK-β, p160ROCK, or Rho-kinase β and the term “ROCKII” can also be referred as ROK-α or Rho-kinase α. Said inhibition may be effected in vitro and/or in vivo, and when effected in vivo, is preferably effected in a selective manner, as defined above.

According to an embodiment, the invention provides a method for treating or lessening the severity of a ROCK-mediated disease or condition in a patient comprising the step of administering to said patient a compound according to the present invention.

The term “ROCK-mediated condition” or “disease”, as used herein, means any disease or other deleterious condition in which is known to play a role. The term “ROCK-mediated condition” or “disease” also means those diseases or conditions that are alleviated by treatment with a ROCK inhibitor. Accordingly, another embodiment of the present invention relates to treating or lessening the severity of one or more diseases in which ROCK is known to play a role.

According to particularly preferred embodiments, the compounds of the invention are preferably used in the prevention and/or treatment of at least one disease or disorder, preferably in which at least one isoform of ROCK is involved. According to an even more particularly preferred embodiment, the compounds of the invention may be used in the prevention and/or treatment of at least one disease or disorder in which the ROCK I or ROCK II is involved, such as inflammatory diseases, chronic obstructive bladder disease (COBD) and the related erectile dysfunction as well as in diabetes related ED

Specifically, the present invention relates to the use of a compound according to the invention for the preparation of a medicament for treating or lessening the severity of a disease or condition selected from eye disease or disorder (such as but not limited to retinopathy, glaucoma and degenerative retinal diseases such as macular degeneration and retinitis pigmentosa), kidney disease (such as but not limited to renal dysfunction), erectile and bladder dysfunction, neurological and CNS (brain) disease or disorder (such as but not limited to Alzheimer, meningitis and convulsions), hypertension, lung disease (such as but not limited to asthma, fibrosis, pneumonia, cystic fibrosis and respiratory distress syndrome), premature birth, cancer (such as but not limited to cancer of the lung, intestine, nerve, skin, pancreas, liver, uterus, ovary, brain, thyroid gland, and leukemia, lymphoma and melanoma), cardiovascular and vascular (blood vessel artery) disease or disorder (such as but not limited to cerebrovascular contraction, ischemia, reperfusion, hypoxia peripheral circulation disorder, atherosclerosis, thrombosis, aneurism and hemorrhage), blood disease (such as but not limited to sepsis, eosinophilia and endotoxemia), musculoskeletal disease (such as but not limited to spasm), inflammatory disease, infection, allergy and autoimmune diseases or disorders, AIDS, bone disease (such as but not limited to osteoporosis), inflammatory diseases, diabetes (such as but not limited to hyperglycemia), obesity and pancreas disease.

For example, the compounds of the invention may be used in the prevention and/or treatment of diseases and disorders such as:

Cardiovascular and vascular diseases: including but not limited to acute stroke, congestive heart failure, cardiovascular ischemia, heart disease, cardiac remodeling, angina, coronary vasospasm, cerebral vasospasm, pulmonary vasoconstriction, restenosis, hypertension, (pulmonary) hypertension, arteriosclerosis, thrombosis (including deep thrombosis) and platelet related diseases.

Neurological and CNS disorders: including but not limited to stroke, multiple sclerosis, brain or spinal cord injury, inflammatory and demyelinating diseases such as Alzheimer's disease, MS and neuropathic pain. The present compounds are therefore suitable for preventing neurodegeneration and stimulating neurogeneration in various neurological disorders.

Proliferative diseases: such as cancer including but not limited to cancer of the brain (gliomas), breast, colon, intestine, skin, head and neck, kidney, lung, liver, ovarian, pancreatic, prostate, or thyroid; leukemia; lymphoma; sarcoma; and melanoma.

Inflammatory diseases: including but not limited to contact dermatitis, psoriasis, rheumatoid arthritis, inflammatory bowel disease, Crohn's disease or ulcerative colitis.

In addition, the compounds of the invention may be used in the prevention and/or treatment of diseases and disorders such as erectile dysfunction, bronchial asthma, osteoporosis, eye diseases such as glaucoma, macular degeneration and retinopathy, renal diseases and AIDS.

The present invention therefore relates to a method of treating or lessening the severity of a disease or condition selected from cardiovascular and vascular diseases including but not limited to angina, coronary vasospasm, cerebral vasospasm, pulmonary vasoconstriction, restenosis, hypertension, (pulmonary), arteriosclerosis, thrombosis (including deep thrombosis), platelet related diseases, acute stroke, congestive heart failure, cardiovascular ischemia, heart disease, and cardiac remodeling; neurological and CNS disorders including but not limited to stroke, multiple sclerosis, brain or spinal cord injury, inflammatory and demyelinating diseases such as Alzheimer s disease, MS and neuropathic pain; proliferative diseases such as cancer including but not limited to brain (gliomas), breast, colon, head and neck, kidney, lung, liver, melanoma, ovarian, pancreatic, prostate, sarcoma, or thyroid cancer; erectile dysfunction; bronchial asthma; osteoporosis; eye diseases such as glaucoma, macular degeneration and retinopathy; renal diseases; AIDS; hypertension; preterm labor; vascular smooth muscle cell proliferation; myocardial hypertrophy; malignoma; ischemia/reperfusion-induced injury; endothelial dysfunction; Crohn's Disease and colitis: neurite outgrowth; Raynaud's Disease; benign prostatic hyperplasia; and atherosclerosis; wherein said method comprises administering to a patient in need thereof a compound or a composition according to the present invention.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087 and U.S. Pat. No. 6,372,733.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

Generally, for pharmaceutical use, the compounds of the inventions may be formulated as a pharmaceutical preparation comprising at least one compound of the invention and at least one pharmaceutically acceptable carrier, diluent or excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is again made to for instance U.S. Pat. No. 6,372,778, U.S. Pat. No. 6,369,086, U.S. Pat. No. 6,369,087 and U.S. Pat. No. 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical composition according to the invention, it can be advantageous to employ α-, β- or γ-cyclodextrins or their derivatives. In addition, co-solvents such as alcohols may improve the solubility and/or the stability of the compounds. In the preparation of aqueous compositions, addition of salts of the compounds of the invention can be more suitable due to their increased water solubility.

Appropriate cyclodextrins are α-, β- or γ-cyclodextrins (CDs) or ethers and mixed ethers thereof wherein one or more of the hydroxy groups of the anhydroglucose units of the cyclodextrin are substituted with alkyl, particularly methyl, ethyl or isopropyl, e.g. randomly methylated β-CD; hydroxyalkyl, particularly hydroxyethyl, hydroxypropyl or hydroxybutyl; carboxyalkyl, particularly carboxymethyl or carboxyethyl; alkylcarbonyl, particularly acetyl; alkoxycarbonylalkyl or carboxyalkoxyalkyl, particularly carboxymethoxypropyl or carboxyethoxypropyl; alkylcarbonyloxyalkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-β-CD (2-HP-β-CD). The term mixed ether denotes cyclodextrin derivatives wherein at least two cyclodextrin hydroxy groups are etherified with different groups such as, for example, hydroxypropyl and hydroxyethyl. An interesting way of formulating the compounds in combination with a cyclodextrin or a derivative thereof has been described in EP-A-721,331. Although the formulations described therein are with antifungal active ingredients, they are equally interesting for formulating the compounds. Said formulations may also be rendered more palatable by adding pharmaceutically acceptable sweeteners and/or flavors. In particular, the present invention encompasses a pharmaceutical composition comprising an effective amount of a compound according to the invention with a pharmaceutically acceptable cyclodextrin. The present invention also encompasses cyclodextrin complexes consisting of a compound according to the invention and a cyclodextrin.

Particular reference is made to the compositions, formulations (and carriers, excipients, diluents, etc. for use therein), routes of administration etc., which are known per se for analogous pyridinocarboxamides, such as those described in U.S. Pat. No. 4,997,834 and EP-A-0 370 498.

For the treatment of pain, the compounds of the invention may be used locally or systemically, e.g. as described for the peptide inhibitors of PKC in WO 03/089456 and 03/089457. For local administration, the compounds may advantageously be used in the form of a spray, ointment or transdermal patch or another suitable form for topical, transdermal and/or intradermal administration; and for systemic administration, the compounds of the invention may advantageously be administered orally.

For ophthalmic application, solutions, gels, tablets and the like are often prepared using a physiological saline solution, gel or excipient as a major vehicle. Ophthalmic formulations should preferably be prepared at a comfortable pH with an appropriate buffer system.

More in particular, the compositions may be formulated in a pharmaceutical formulation comprising a therapeutically effective amount of particles consisting of a solid dispersion of the compounds of the invention and one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered. The term “a solid dispersion” also comprises dispersions that are less homogenous throughout than solid solutions. Such dispersions are not chemically and physically uniform throughout or comprise more than one phase.

The water-soluble polymer is conveniently a polymer that has an apparent viscosity of 1 to 100 mPa·s when dissolved in a 2% aqueous solution at 20° C. solution. Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule.

It may further be convenient to formulate the compounds in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bio-availability which can conveniently be manufactured and which is suitable for preparing pharmaceutical dosage forms for oral administration. Said beads comprise (a) a central, rounded or spherical core, (b) a coating film of a hydrophilic polymer and an antiretroviral agent and (c) a seal-coating polymer layer. Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

The preparations may be prepared in a manner known per se, which usually involves mixing the at least one compound according to the invention with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of the Formula I or II above that, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram, more often between 0.1 and 500 mg, such as between 1 and 250 mg, for example about 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778,U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

Thus, in a further aspect, the invention relates to a composition, and in particular a composition for pharmaceutical use, that contains at least one compound of the invention and at least one suitable carrier (i.e. a carrier suitable for pharmaceutical use). The invention also relates to the use of a compound of the invention in the preparation of such a composition.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

For an oral administration form, the compositions of the present invention can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug.

The compositions are of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a composition for veterinary use that contains at least one compound of the invention (e.g. a compound that has been identified, discovered and/or developed using a nematode or method as described herein) and at least one suitable carrier (i.e. a carrier suitable for veterinary use). The invention also relates to the use of a compound of the invention in the preparation of such a composition.

The invention will now be illustrated by means of the following synthetic and biological examples, which do not limited the scope of the invention in any way.

### EXAMPLES

Unless indicated otherwise, the purity of the compounds was confirmed by liquid chromatography/mass spectrometry (LC/MS), as follows:
HPLC system: Waters 2690 with photodiode array detector Waters 996; Column: C18; Gradient: solvent A (H₂O/formic acid 26.5 nM) 0%, to solvent B (CH₃CN/formic acid 17 nM) 80% in 3 min. Flow: 2.75 ml/min.
Mass spectrometer: Micromass Platform LC. Ionization: electrospray (polarity: negative and positive).

NMR spectra were determined on a Varian Mercury 300 MHz NMR using the indicated solvent as an internal reference. Melting points were determined on a Büchi B-540 and are non-corrected. All reagents used were either obtained commercially or were prepared in a manner known per se.

### Extra Analytical (or Preparative) Techniques:

Unless indicated otherwise, purification by preparative HPLC, was performed on a Shimadzu SCL-10A (UV detection at 215 and 254 nm, detector SPD-10A) using C-18 column (Nucleosil, 100 Å, 100 μm, 20×200 mm) and different gradients (water, acetonitrile, formic acid).

Chiral HPLC (analytical and preparative) was performed on a Shimadzu SCL-10A (UV detection at 215 and 254 nm, detector SPD-10A) using different column such as Chiralcel OD-H (tris-3,5-dimethylphenylcarbamate, 46×250 or 100×250 mm, 5 μm), Chiralcel OJ (tris-methylbenzoate, 46×250 or 100×250 mm, 5 μm), Chiralpak AD (tris-3,5-dimethylphenylcarbamate, 46×250 mm, 10 μm) and Chiralpak AS (tris-(S)-1-phenylethylcarbamate, 46×250 mm, 10 μm) from Chiral Technologies Europe (Illkirch, France):
Eluent: mixture of solvent such as ethanol, 1-propanol, 2-propanol, methanol, butanol, pentane, hexane, heptane, cyclohexane, diisopropylethylamine, triethylamine.
Flow: between 1 and 50 ml/min.

The following intermediates and general procedures were used to prepare the compounds described herein.

### INTERMEDIATES

### Intermediate 1

### 3-tert-butoxycarbonylamino-3-[4-(pyridin-4-ylcarbamoyl)-phenyl]-propionic acid

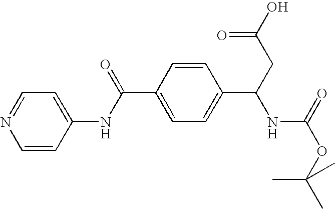

4-(1-Amino-2-carboxy-ethyl)-benzoic acid methyl ester (2.465 g) was suspended in 100 ml of a mixture acetone/1M Na₂CO₃ (9/1). BOC₂O (1.1 eq) was added and the reaction mixture was stirred at RT for 3 hours. Further 2 equivalents of BOC₂O were added and the reaction mixture was stirred for 2 hours. Acetone was removed under reduced pressure. The residue was acidified (pH=2) with 1M HCl. The precipitate was filtered off and washed with water to give the 4-(1-tert-butoxycarbonylamino-2-carboxy-ethyl)-benzoic acid methyl ester as a white powder (88% yield).

4-(1-tert-Butoxycarbonylamino-2-carboxy-ethyl)-benzoic acid methyl ester (2.845 g) was suspended in DMF (60 ml). K₂CO₃ (26 eq), Benzyl-triethyl ammonium chloride (BTEAC; 1 eq) and tert-butyl bromide (48 eq) were added. The reaction mixture was stirred at 55° C. for 5 hours before addition of 10 equivalents tert-butyl bromide. The reaction mixture was stirred for 2 hours further more and then concentrated under reduced pressure. The solution was concentrated and water was added. The solution was extracted with ethyl acetate. The organic layer was washed with 0.05 M NaHCO₃, dried over MgSO₄ and evaporated under reduced pressure. The residue was purified by flash chromatography (cyclohexane/EtOAc: 100/0 to 80/20) to give the 4-(2-tert-butoxycarbonyl-1-tert-butoxycarbonylamino-ethyl)-benzoic acid methyl ester as a yellow powder (67% yield).

To a solution of 4-(2-tert-butoxycarbonyl-1-tert-butoxycarbonylamino-ethyl)-benzoic acid methyl ester (2.13 g) in methanol (0.25 M) was 1M LiOH (5.6 ml). The reaction mixture was stirred at 35° C. for 6 hours. Further 0.5 eq of LiOH was added and the reaction mixture was stirred at RT overnight. The reaction mixture was evaporated. The residue was taken in water. The solution was acidified (pH=2) with 1 M HCl and extracted with ethyl acetate. The organic layers were dried over MgSO₄, and evaporated. The residue was purified by flash chromatography (cyclohexane /ethyl acetate 80/20 to 60/40) to give the 4-(2-tert-butoxycarbonyl-1-tert-butoxycarbonylamino-ethyl)benzoic acid as a white powder (44% yield).

To a solution of 4-(2-tert-butoxycarbonyl-1-tert-butoxycarbonylamino-ethyl)-benzoic acid in DMF (0.25 M) were added DIEA (5 eq) and a solution of TBTU/HOBt (1 eq/0.2 eq) 0.4M in DMF. After 4 min of stirring, the 4-aminopyridine (1 eq) was added and the reaction mixture was stirred for 1 h before addition of 1 extra equivalent of DIEA and TBTU. After a total of 3 h of stirring, the reaction was completed. The reaction mixture was concentrated under reduced pressure. The residue was taken in ethyl acetate, and washed with 1 M NaHCO₃, and then with brine. The organic layer was dried over MgSO₄, filtered and evaporated. The residue was purified by flash chromatography (AcOEt/cyclohexane 1/4 to 4/1) to give 3-tert-butoxycarbonylamino-3-[4-(pyridin-4-ylcarbamoyl)phenyl]-propionic acid tert-butyl ester as a pale orange powder (95% yield).

To a solution of 3-tert-butoxycarbonylamino-3-[4-(pyridin-4-ylcarbamoyl)-phenyl]-propionic acid tert-butyl ester (2.25 g) in THF (0.25 M) was added 1M LiOH (3 eq). The reaction mixture was stirred at 30° C. for 20 The solution was concentrated under reduced pressure and then acidified with 1 M HCl (pH=5). The solution was extracted with ethyl acetate (3×100 ml). The combined organic layers were dried over MgSO₄, filtered and evaporated. The residue was purified by flash chromatography (AcOEt/MeOH: 1/0 to 0/1) to give 3-tert-butoxycarbonylamino-3-[4-(pyridin-4-ylcarbamoyl)-phenyl]-propionic acid as a white powder (58% yield). ¹H NMR (300 MHz, DMSO-d6): 1.33 ppm (s, 9H); 2.57-2.75 ppm (m, 2H); 4.93 ppm (m, 1H); 7.46 ppm (d, 2H, J=8.3 Hz); 7.52 ppm (d, 1H, J=8.5 Hz); 7.80 ppm (d, 2H, J=6.5 Hz); 7.89 ppm (d, 2H, J=8.2 Hz); 8.47 ppm (d, 2H, J=6.5 Hz); 10.62 ppm (s, 1H).

### Intermediate 2

### {2-Amino-1-[4-(pyridin-4-ylcarbamoyl)-phenyl]-ethyl}-carbamic acid tert-butyl ester

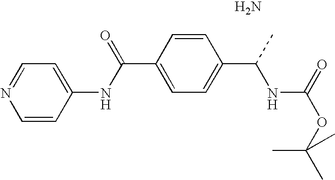

To a solution of 2-amino-1-(4-bromo-phenyl)ethan-1-one hydrochloride (10 g) in dry THF (200 ml), were added DIEA (1 eq) and benzylchloroformate (1.1 eq). The reaction mixture was stirred overnight at RT. The solution was concentrated under reduced pressure. The resulting white solid was separated between DCM (400 ml) and water (175 ml). The organic phase was dried over MgSO₄, filtered and evaporated. The residue was dried to give the [2-(4-Bromo-phenyl)-2-oxo-ethyl]-carbamic acid benzyl ester as a white powder (84% yield).

The [2-(4-Bromo-phenyl)-2-oxo-ethyl]-carbamic acid benzyl ester (6.8 g) was dissolved in THF (52 ml) and water (8 ml). Potassium acetate (1 eq), 1,3-bis-diphenylphosphinopropane (0.02 eq) and Pd(OAc)₂ (0.04 eq) were added. The mixture was stirred under 50 atm of carbon monoxyde at 150° C. for 3 hours. The reaction mixture was cooled down at RT and then filtered. The solvent was evaporated under reduced pressure. The residue was dissolved in EtOAc and extracted with 0.1N HCl. The organic layer was dried over MgSO₄, filtered and the solvent was removed under reduced pressure. The residue was dried to give the 4-(2-benzyloxycarbonylamino-acetyl)-benzoic acid as an orange powder (94% yield).

To a solution of 4-(2-benzyloxycarbonylamino-acetyl)-benzoic acid (2.6 g) in DCM (0.25 M) were added oxalyle chloride (2.5 eq) and a few drops of DMF. The solution was stirred at RT for 2 hours and then evaporated to give the 4-(2-benzyloxycarbonylamino-acetyl)-benzoyl chloride. The 4-aminopyridine (0.78 g, 1 eq) was dissolved in acetonitrile (0.25 M) and DIEA (3 eq) was added. The solution was cooled at 0° C. (in an ice bath). The 4-(2-benzyloxycarbonylamino-acetyl)-benzoyl chloride in the minimum of acetonitrile was then added dropwise (under nitrogen). After addition, the ice bath was removed and the reaction mixture was stirred at RT for 3 hours. The solvent was evaporated and the residue was dissolved in DCM and extracted with 1N NaOH. The organic layer was dried over MgSO₄, filtered and the solvent was removed under reduced pressure. The residue was purified by flash chromatography (DCM/MeOH 97/3 to 95/5) to give the {2-oxo-2-[4-(pyridin 4-ylcarbamoyl)-phenyl]-ethyl}-carbamic acid benzyl ester as a white powder (37% yield).

The {2-oxo-2-[4-(pyridin-4-ylcarbamoyl)-phenyl]-ethyl}-carbamic acid benzyl ester (1.3 g) was dissolved in EtOH (0.25 M). DIEA (5 eq) and hydroxylamine hydrochloride (5 eq) were added. The reaction mixture was stirred at 60° C. for 12 hours and then cooled down at RT. The solvent was concentrated under reduced pressure, and then water was added to the residue. The {2-hydroxyimino-2-[4-(pyridin-4-ylcarbamoyl)-phenyl]-ethyl}-carbamic acid benzyl ester was collected by filtration and dried (yellowish powder, 59% yield).

The oxime was dissolved in acetic acid (0.25 M), and then zinc powder was added (10 eq). The reaction was stirred at RT for 3 hours. Zinc was filtered off and washed with water. The filtrate was evaporated, and the resulting white solid was dissolved in water. The pH was brought to 14 (with NaOH) and the aqueous phase was extracted with EtOAc. The organic layer was dried over MgSO₄, filtered and the solvent was removed under reduced pressure. The residue was dried to give the {2-amino-2-[4-(pyridin-4-ylcarbamoy)-phenyl]-ethyl}-carbamic acid benzyl ester as a white powder. The amine (1.2 g) was dissolved in acetonitrile (0.25 M) and then, DIEA (3 eq) and (BOC)₂O (1.1 eq) were added. The reaction mixture was stirred at RT for 2 hours and then was evaporated. The residue was dissolved in EtOAc and extracted with 1N NaHCO₃. The organic layer was dried over MgSO₄, filtered and the solvent was removed under reduced pressure. The residue was purified by flash chromatography (cyclohexane/EtOAc, 20/80 10/90 and 0/100) to give the {2-benzyloxycarbonylamino-1-[4-(pyridin-4-ylcarbamoyl)-phenyl]-ethyl}-carbamic acid tert-butyl ester (60% yield).

To a solution of the {2-benzyloxycarbonylamino-1-[4-(pyridin-4-ylcarbamoyl)-phenyl]-ethyl}-carbamic acid tert-butyl ester (0.5 g) in EtOH/water (1/1) were added acetic acid (2 eq) and Pd (10%, 500 mg). The reaction mixture was stirred at RT under hydrogen (2 atm) for 1 hour. The palladium was filtered off. The filtrate was neutralize with 1N NaOH, and then was evaporated. The residue was dissolved in water. The pH was brought to 14 (with NaOH) and the aqueous phase was extracted with EtOAc. The organic layer was dried over MgSO₄, filtered and the solvent was removed under reduced pressure. The residue was dried to give the title compound as a white powder (47% yield).

### Intermediate 3

### (R)-3-tert-butoxycarbonylamino-3-[4-(pyridin ylcarbamoyl)-phenyl]-propionic acid

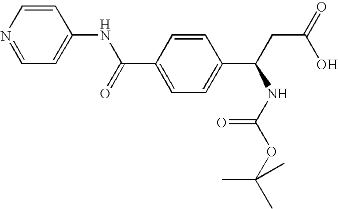

To a solution of (R)-3-(4-bromo-phenyl)-3-tert-butoxycarbonylamino-propionic acid (5.3 g) in DCM (100 ml), were added TBTU (1 eq.) and HOBt (1 eq.). The mixture was cooled at 0° C. and then, DIEA (1.2 eq.) was added dropwise. The reaction mixture was stirred at 0° C. for 15 min. Methanol (20 ml) was then added, and the solution was stirred at RT for 12 hours. Solvent was evaporated, DCM (100 ml) was added and the solution was washed with 1M NaHCO₃ (2×100 ml), 20% KHSO₄ (2×100 ml) and brine (2×100 ml). The organic layer was dried over Na₂SO₄ and evaporated, yielding the (R)-3-(4-bromo-phenyl)-3-tert-butoxycarbonylamino-propionic acid methyl ester (99% yield). ¹H NMR (300 MHz, DMSO-d6): 1.34 ppm (s, 9H); 2.70 ppm (m, 2H); 3.53 ppm (s, 3H); 4.85 ppm (m, 1H); 7.24 ppm (d, 2H, J=8.4 Hz); 7.49 ppm (m, 3H).

To a solution of (R)-3-(4-bromo-phenyl)-3-tert-butoxycarbonylamino-propionic acid methyl ester (5.2 g) in a mixture of THF (65 ml) and water (10 ml) were successively added potassium acetate (1 eq.), palladium acetate (0.04 eq.) and DPPP (0.02 eq.). The reaction mixture was stirred at 150° C. under 50 atm of carbon monoxide, for 3 hours. The reaction mixture was cooled down at RT, and then was filtered off. The filtrate was evaporated. The residue was purified by flash chromatography on silica gel (DCM/MeOH, 100/0 to 95/5). The 4-((R)-1-tert-butoxycarbonylamino-2-methoxycarbonyl-ethyl)-benzoic acid was obtained as a white powder (53% yield).

To a solution of 4-((R)-1-tert-butoxycarbonylamino-2-methoxycarbonyl-ethyl)-benzoic acid (2.4 g) in DMF (25 ml) were added TBTU (1.3 eq.), HOBt (0.2 eq.) and DIEA (3 eq.). The reaction mixture was stirred at RT for 5 min, and the 4-aminopyridine (1 eq.) was added. The solution was stirred at RT for 3 hours. DMF was evaporated, and water was added to the residue. The product was extracted with EtOAc (150 ml). The organic layer was washed with brine (100 ml), dried over MgSO₄ and evaporated. The residue was purified by flash chromatography on silica gel (EtOAc), yielding the (R)-3-tert-butoxycarbonylamino-3-[4-(pyridin-4-ylcarbamoyl)-phenyl]-propionic acid methyl ester as a white powder (67% yield). ¹H NMR (300 MHz, DMSO-d6): 1.34 ppm (s, 9H); 2.75 ppm (m, 2H); 3.55 ppm (s, 3H); 4.97 ppm (m, 1H); 7.46 ppm (d, 2H, J=8.3 Hz); 7.58 ppm (d, 1H, J=8.7 Hz); 7.76 ppm (d, 2H, J=5.1 Hz); 7.88 ppm (d, 2H, J=8.2 Hz); 8.46 ppm (d, 2H, J=5.1 Hz); 10.55 ppm (s, 1H).

To a suspension of (R)-3-tert-butoxycarbonylamino-3-[4-(pyridin-4-ylcarbamoyl)-phenyl]-propionic acid methyl ester (1.9 g) in 1,4-dioxane (35 ml) was added 1N LiOH (1.1 eq. of LiOH). The reaction mixture was stirred at RT for 2 hours and at 4° C. overnight. The pH was adjusted to 7 by addition of 1N HCl (4.5 ml). The solution was lyophilized without further workup (the title product was in mixture with salts which could be removed in the next step). The title compound was obtained as a white powder (yield not determined). ¹H NMR (300 MHz, DMSO-d6): 1.34 ppm (s, 9H); 2.62 ppm (m, 2H); 4.91 ppm (m, 1H); 7.45 ppm (d, 2H, J=8.3 Hz); 7.58 ppm (d, 1H, J=9.7 Hz); 7.76 ppm (d, 2H, J=6.4 Hz); 7.88 ppm (d, 2H, J=8.2 Hz), 8.45 ppm (d, 2H, J=6.4 Hz); 10.55 ppm (s, 1H).

### Intermediate 4

### (S)-3-tert-butoxycarbonylamino-3-[4-(pyridin-4-ylcarbamoyl)-phenyl]-propionic acid

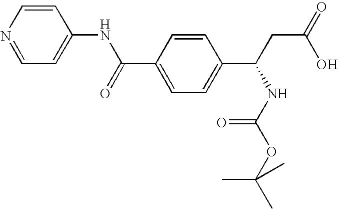

The title compound was prepared according to the protocol described for Intermediate 3 ¹H NMR (300 MHz, DMSO-d6): 1.34 ppm (s, 9H); 2.62 ppm (m, 2H); 4.91 ppm (m, 1H); 7.45 ppm (d, 2H, J=8.3 Hz); 7.58 ppm (d, 1H, J=9.7 Hz); 7.76 ppm (d, 2H, J=6.4 Hz); 7.88 ppm (d, 2H, J=8.2 Hz); 8.45 ppm (d, 2H, J=6.4 Hz); 10.55 ppm (s, 1H).

### Intermediate 7

### {2-Methylamino-1-[4-(pyridin-4-ylcarbamoyl)-phenyl]-ethyl}-carbamic acid tert-butyl ester

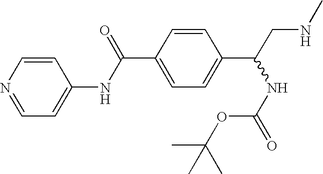

To a solution of 4-acetyl-benzoic acid methyl ester (345.7 g, 1.94 mmol, 1 eq.) in chloroform (1700 ml) was added dropwise bromine (100 ml, 310 g, 1.94 mmol, 1 eq.) in chloroform (3100 ml) with stirring at RT. During addition of bromine the reaction displayed an exotherm of 10° C. After 2 h at room temperature, the mixture was diluted with ice water (1000 ml) and aqueous Na₂S₂O₃ (700 ml) and extracted with DCM (3×1200 ml). The organic layer was washed with water (4500 ml), dried over MgSO₄ and concentrated in vacuo to give the 4-(2-Bromo-acetyl)-benzoic acid methyl ester (527.2 g). The crude residue was recrystallized from methanol (2500 ml) to give 334 g (67% yield).

To a stirred solution of bromoketone (590.5 g) in MeOH (5900 ml) at 0° C. was added NaBH₄ (91.2 g) portionwise. The reaction was allowed to warm to room temperature and stirred for 1 h after which time TLC analysis indicated the formation of the bromo alcohol. K₂CO₃ (318 g) was added to the same flask and the reaction mixture stirred over the weekend. TLC analysis indicated the reaction was complete. Water (3000 ml) was added and the mixture extracted with Et₂O (3×5000 ml). The organic extracts were washed with brine (2×5000 ml), dried over MgSO₄ and concentrated in vacuum to give the 4-oxiranyl-benzoic acid methyl ester as an orange solid, 405.8 g (99% yield).

The 4-oxiranyl-benzoic acid methyl ester (405 g) was dissolved in methylamine 33 wt % in EtOH and stirred overnight. TLC analysis indicated the reaction was complete. Water was added and the mixture extracted with EtOAc (4×500 ml). The organic extracts were washed with water (3×500 ml), dried over MgSO₄ and concentrated in vacuum to give 495 g of 4-(1-hydroxy-2-methylamino ethyl)-benzoic acid methyl ester.

The amino alcohol (412.3 g) was dissolved in THF (6000 ml) and NaHCO₃ (336 g, 2 eq.) was added with stirring. The solution was cooled to 0-5° C. and benzyl chloroformate (416 ml, 1.5 equiv.) in THF (6000 ml) was added dropwise. The mixture was stirred at 0-5° C. for 1 h and allowed to warm to room temperature overnight. The analysis indicated the reaction was complete. Water (9000 ml) was added and the aqueous layer extracted with EtOAc (2×5000 ml). The organic layer was back extracted with saturated aqueous NaHCO₃ solution (2×2500 ml). The combined organic layers were dried over MgSO₄ and concentrated in vacuo to give a crude product, 760.7 g. The crude product was purified by column chromatography to give 4-[2-(Benzyloxycarbonyl-methyl amino)-1-hydroxy-ethyl]-benzoic acid methyl ester (137 g, 20% yield from the bromoketone).

To a solution of the previous alcohol (137 g, 0.4 mol) in DCM (1400 ml) was added triethylamine (123 ml, 0.88 mol, 2.2 eq.) and the reaction cooled to <5° C. Mesylate chloride (48 ml, 0.6 mol, 1.5 equiv) was added dropwise and after complete addition the reaction mixture was allowed to warm to room temperature. After 1 h LC analysis indicated the reaction was complete. The DCM layer was washed with H₂O (1400 ml), 1M HCl (1400 ml) and H₂O (1400 ml). The DCM layer was dried over MgSO₄ and concentrated in vacuo to give the 4-[2-(benzyloxycarbonyl-methyl-amino)-1-methanesulfonyloxy-ethyl]-benzoic acid methyl ester (166.7 g, 99% yield).

To a 2000 ml flask was added the previous mesylated product (166.7 g, 0.4 mol) and DMF (1700 ml). NaN₃ (25.7 g, 0.4 mol, 1 eq.) was added portionwise. The reaction mixture was heated to 50° C. and stirred for 14 h. LC analysis indicated the reaction was complete. The reaction was cooled to room temperature and Ph₃P (105 g, 0.4 mol, 1 eq.) and H₂O (105 ml) were added. The reaction was stirred for 2 h and LC analysis indicated the reaction was complete. The reaction mixture was concentrated in vacuo to give the 4-[1-amino-2-(benzyloxycarbonyl-methyl-amino)-ethyl]-benzoic acid methyl ester as a sticky solid (351.8 g), which was used without further purification.

The amine (351.8 g, active charge 135 g, 0.39 mol) was dissolved in a mixture of 1:1 acetone/1M Na₂CO₃ solution (5000 ml). Boc anhydride (197 ml, 0.86 mol, 2.2 equiv) was added and the reaction mixture stirred overnight at room temperature. LC analysis indicated the reaction was complete. The acetone was removed in vacuo and the aqueous layer was extracted with EtOAc (3×2000 ml). The combined organic extracts were washed with brine (3000 ml), dried over MgSO₄ and concentrated in vacuo. The 4-[2-(benzyloxycarbonyl-methyl-amino)-1-tert-butoxycarbonylamino-ethyl]-benzoic acid methyl ester was purified by flash chromatography on silica gel (21% yield from the CBz protected amino alcohol)

To a solution of the previous ester (35.9 g) in MeOH (1500 ml) was added 1M NaOH solution (700 ml) slowly. The reaction mixture was stirred for 4 h after which time the reaction was complete. The methanol was removed in vacuo, the aqueous layer acidified to pH 5-6 using 0.5M HCl (1400 ml) and the product extracted with EtOAc (3×1500 ml). The organic layer was dried over MgSO₄, filtered and concentrated in vacuo to give the corresponding benzoic acid (100% yield).

To a solution of the acid (34.76 g, 0.081 mol) in DMF (1000 ml) were added DIEA (42 ml, 0.243 mol, 3 equiv.), HBTU (40 g, 0.1053 mol, 1.3 equiv.), HOBt (3.2 g, 0.0243 mol, 0.3 equiv.) and 4-aminopyridine (9.15 g, 0.0972 mol, 1.2 equiv.). The reaction mixture was stirred overnight at room temperature. TLC analysis indicated the reaction was complete. DMF was evaporated and the residue taken up in EtOAc (2500 ml) and 1M Na₂CO₃ (2500 ml). The layers were separated and the aqueous extracted with EtOAc (2500 ml). The organic layer was washed with brine (5000 ml), dried over MgSO₄ and concentrated in vacuo. The {2-(benzyloxycarbonyl-methyl-amino)-1-[4-(pyridin-4-ylcarbamoyl)-phenyl]-ethyl}-carbamic acid tert-butyl ester was purified by flash chromatography on silica gel (85% yield).

The previous compound (34.7 g) was dissolved in MeOH (250 ml) and transferred to a 300 ml Parr hydrogenator vessel. The vessel was purged with N₂ and added 10% Pd/C (wet catalyst) (20 g). The reaction was purged with hydrogen and stirred for 5 h at 5 bar pressure of hydrogen. The analysis indicated the reaction was complete. The reaction mixture was filtered through Celite (100 g) and the filter cake washed with MeOH (750 ml). The reaction mixture was concentrated in vacuo. The crude residue was purified by flash chromatography on silica gel to give the title compound (67% yield).

Intermediates 8 to 19 shown under Table A were synthesized using known procedures as hydrochloric acid salt. Reference is made to WO 03/045924.

TABLE A

| Name | Intermediate | Structure |
|---|---|---|
| Chroman-4-ylamine | 8 | 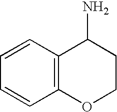 |
| Thiochroman-4-ylamine | 9 | 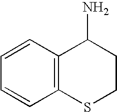 |
| 5-Fluoro-indan-1-ylamine | 10 | 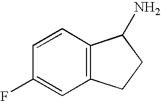 |
| 6-Fluoro-indan-1-ylamine | 11 | 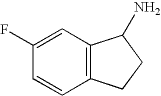 |
| 5-Chloro-indan-1-ylamine | 12 | 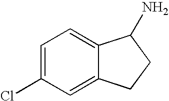 |
| 6,7,8,9-Tetrahydro-5H- benzocyclohepten- 5-ylamine | 13 | 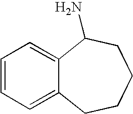 |
| 3-Fluoro-6,7,8,9- tetrahydro-5H-benzocyclo- hepten-5-ylamine | 14 | 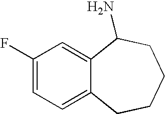 |
| 4,5,6,7-Tetrahydro- benzofuran-4-ylamine | 15 | 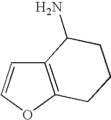 |
| 4,5,6,7-Tetrahydro- benzo[b]thiophen-4- ylamine | 16 | 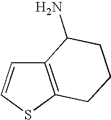 |
| 2,3-Dihydro-benzofuran-3- ylamine | 17 | 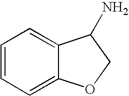 |
| C-Cyclobutyl-C-(4-fluoro- phenyl)-methylamine | 18 | 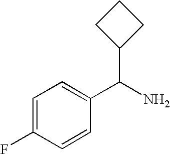 |
| C-(4-Chloro-phenyl)-C- cyclopropyl-methylamine | 19 | 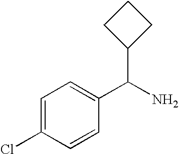 |

Intermediates 20 to 41 shown in Table B were synthesized using known procedures. Reference is made to Peretto et al., J. Med. Chem., 2005, 48, p 5705; Butler and Pollatz, J. Org. Chem., 1971, 36, p 1308, and Yamashita et al., Tetrahedron, 2004, 60, p 2843.

TABLE B

| Name | Intermediate | Structure | MP |
|---|---|---|---|
| 2-(4-Fluoro-phenyl)-2-methyl- propionic acid | 20 | 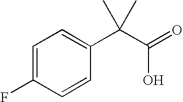 | 90.0-92.1° C. |
| 1-(4-Fluoro-phenyl)- cyclopropanecarboxylic acid | 21 | 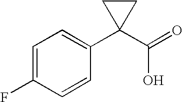 | 129.9-132.2° C. |
| 1-(4-Fluoro-phenyl)- cyclobutanecarboxylic acid | 22 | 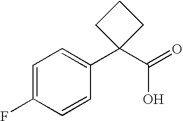 | 82.8-83.6° C. |
| 2-(3,4-Dichloro-phenyl)-2-methyl- propionic acid | 23 | 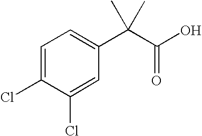 | 91.6-94.2° C. |
| 1-(3,4-Dichloro-phenyl)- cyclopropanecarboxylic acid | 24 | 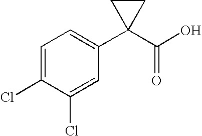 | 147.0-147.2° C. |
| 1-(3,4-Dichloro-phenyl)- cyclobutanecarboxylic acid | 25 | 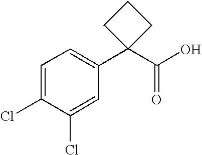 | 117.2-119.0° C. |
| 2-(3,4-Difluoro-phenyl)-2-methyl- propionic acid | 26 | 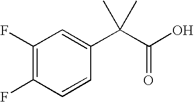 | nd |
| 1-(3,4-Difluoro-phenyl)- cyclopropanecarboxylic acid | 27 | 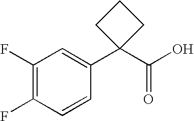 | 94.0-98.6° C. |
| 1-(3,4-Difluoro-phenyl)- cyclobutanecarboxylic acid | 28 | 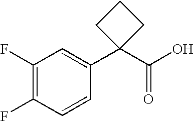 | 71.0-72.5° C. |
| 2-(4-Chloro-2-fluoro-phenyl)-2- methyl-propionic acid | 29 | 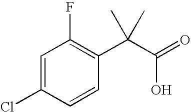 | 136.4-138.0° C. |
| 1-(4-Chloro-2-fluoro-phenyl)- cyclopropanecarboxylic acid | 30 | 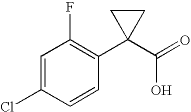 | 151.1-153.3° C. |
| 2-Methyl-2-thiophen-2-yl- propionic acid | 31 | 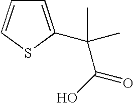 | 79.8-80.7° C. |
| 1-Thiophen-2-yl- cyclopropanecarboxylic acid | 32 | 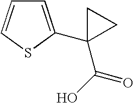 | 130.9-132.6° C. |
| 2-Methyl-2-thiophen-3-yl- propionic acid | 33 | 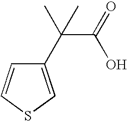 | 72.5-73.9° C. |
| 1-Thiophen-3-yl- cyclopropanecarboxylic acid | 34 | 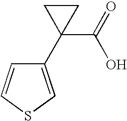 | 134.7-136.9° C. |
| 1-(3,4-Difluoro-phenyl)- cyclopentanecarboxylic acid | 35 | 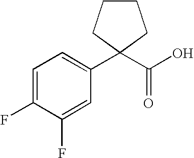 | 130.5-134.3° C. |
| 2-(2-Chloro-4-fluoro-phenyl)-2- methyl-propionic acid | 36 | 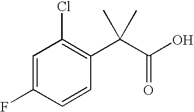 | nd |
| 1-(2-Chloro-4-fluoro-phenyl)- cyclopropanecarboxylic acid | 37 | 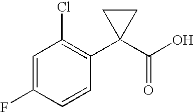 | 91.7-98.9° C. |
| 2-(3-Chloro-4-fluoro-phenyl)-2- methyl-propionic acid | 38 | 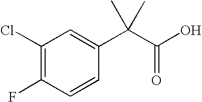 | nd |
| 1-(3-Chloro-4-fluoro-phenyl)- cyclopropanecarboxylic acid | 39 | 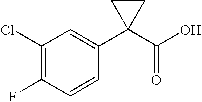 | nd |
| 2-(2,4-Difluoro-phenyl)-2-methyl- propionic acid | 40 | 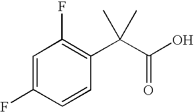 | nd |
| 1-(2,4-Difluoro-phenyl)- cyclopropanecarboxylic acid | 41 | 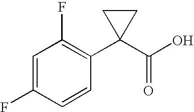 | nd |

nd: not determined.

Intermediates 42 to 47 shown in Table C were synthesized using known procedures. Reference is made to Aono et al., Chem. Pharm. Bull., 1978, 25, p 1153; Oldenziel et al., J. Org. Chem., 1977, 42(19), p 3114; Yoneda et al., J. Chem. Soc. Perkin Trans 1, 1990, p 607; U.S. Pat. No. 430,919 and U.S. Pat. No. 5,294,635 patents.

TABLE C

| Name | Intermediate | Structure | Melting Point |
|---|---|---|---|
| 6-Methoxy-1,2,3,4-tetrahydro- naphthalene-1-carboxylic acid | 42 | 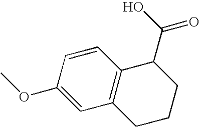 | nd |
| 5-Fluoro-indan-1-carboxylic acid | 43 | 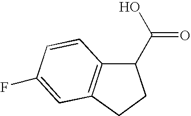 | 86.6-88.0° C. |
| 5-Chloro-indan-1-carboxylic acid | 44 | 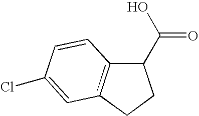 | 162.8-165.8° C. |
| 6-Fluoro-indan-1-carboxylic acid | 45 | 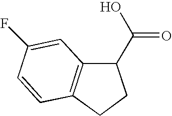 | 211.1-212.6° C. |
| 6,7,8,9-Tetrahydro-5H- benzocycloheptene-5-carboxylic acid | 46 | 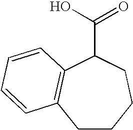 | nd |
| 3-Fluoro-6,7,8,9-tetrahydro-5H- benzocycloheptene-5-carboxylic acid | 47 | 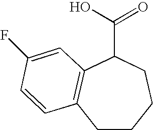 | 98.9-100.2° C. |

nd: not determined.

### Intermediate 48

### 4-(1-tert-butoxycarbonylamino-2-{([1-(chloro-phenyl)-cyclopropanecarbonyl]-amino}-ethyl)-benzoic acid

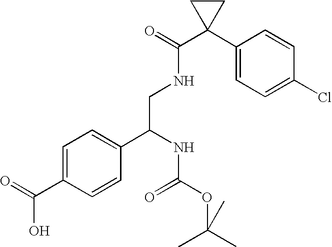

To a solution of 1-(4-chloro-phenyl)-cyclopropanecarboxylic acid (3.9 g) in DMF (0.25 M), were successively added HOBt (0.3 eq), TBTU (1.3 eq) and DIEA (4 eq). The solution was stirred 10 minutes at RT. The 2-amino-1-(4-bromo-phenyl)-ethanone hydrochloric acid salt (1 eq) was then added. The reaction mixture was stirred at RT for 4 hours. The solvent was evaporated and the residue was taken in EtOAc and extracted with 1M NaHCO₃. The organic layer was washed with 0.1 N HCl, was dried over MgSO₄ and finally was evaporated to give the 1-(4-chloro-phenyl)cyclopropanecarboxylic acid [2-(4-bromo-phenyl)-2-oxo-ethyl]-amide as a yellow powder (90% yield).

The 1-(4-chloro-phenyl)-cyclopropanecarboxylic acid [2-(4-bromo-phenyl)-2-oxo-ethyl]-amide (7 g) was dissolved in THF (52 ml) and water (8 ml). Potassium acetate (1 eq), 1,3-bis-diphenylphosphinopropane (0.02 eq) and Pd(OAc)₂ (0.04 eq) were added. The mixture was stirred under 50 atm of carbon monoxide at 150° C. for 3 hours. The reaction mixture was cooled down at RT and then filtered. The solvent was evaporated under reduced pressure. The residue was dissolved in EtOAc and extracted with 0.1N HCl. The organic layer was dried over MgSO₄, filtered and the solvent was removed under reduced pressure. The residue was dried to give the 4-(2-{[1-(4-chloro-phenyl)-cyclopropanecarbonyl]-amino}-acetyl)-benzoic acid as an orange powder (100% yield).

To a solution of the previous intermediate (6.4 g) in MeOH (0.25 M), at 0° C., was added dropwise oxalyle chloride (10 eq). After addition, the reaction mixture was stirred at RT overnight and then was evaporated to give 4-(2-{[1-(4-chloro-phenyl)-cyclopropanecarbonyl)-amino}-acetyl)-benzoic acid methyl ester (100% yield).

The 4-(2-{(1-(4-chloro-phenyl)-cyclopropanecarbonyl]-amino}-1-hydroxyimino-ethyl)-benzoic acid methyl ester was prepared using the procedure described for Intermediate 2 (50% yield, white powder). The oxime was then reduced using the procedure described for Intermediate 2, yielding the 4-(1-amino-2-{[1-(4-chloro-phenyl)-cyclopropanecarbonyl]-amino}-ethyl)-benzoic acid methyl ester (93% yield). The 4-(1-tert-butoxycarbonylamino-2-{[1-(4-chloro-phenyl)cyclopropanecarbonyl]-amino}-ethyl)-benzoic acid methyl ester was prepared using the procedure described for Intermediate 2 (100% yield, white powder).

The 4-(1-tert-butoxycarbonylamino-2-{[1-(4-chloro-phenyl)-cyclopropanecarbonyl]-amino}-ethyl)benzoic acid methyl ester (3.7 g) was suspended in MeOH (1 V) and 1N NaOH (1 V) was added, The reaction mixture was stirred at 55° C. for 1 hour. The solution was concentrated and was then acidified with 1 N HCl. The product was extracted with EtOAc. The organic layer was dried over MgSO₄, filtered and the solvent was removed under reduced pressure. The residue was dried to give the 4-(1-tert-butoxycarbonylamino-2-{[1-(4-chloro-phenyl)-cyclopropanecarbonyl]-amino}-ethyl)-benzoic acid as a white powder (83% yield).

### Intermediate 49

### {[4-(Pyridin-4-ylcarbamoyl)-phenyl]-pyrrolidin-2-yl-methyl}-carbamic acid tert-butyl ester

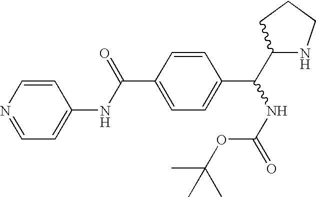

To a solution of Z-L-proline (10 g, 1.2 eq.) in DCM/DMF (15/1 ml), was added oxalyl chloride (1.8 eq.). The reaction mixture was stirred at RT for 3 hours, then the solvent was evaporated to give the Z-L-proline acyl chloride, which was used without further purification.

A solution of methyl 4-iodobenzoate (1 eq.) in dry THF (40 ml) was cooled down to −78° C. Isopropyl magnesium chloride was added dropwise followed by addition of THF (15 ml) that the whole precipitate was dissolved, and the reaction mixture was stirred at −78° C. for 30 min. A solution of CuCN.2LiCl in dry THF (1 molar, 33.6 ml) was added and the reaction mixture was stirred for 15 min more. A solution of freshly prepared Z-L-proline acyl chloride in THF (40 ml) was added dropwise, the reaction mixture stirred at −78° C. for 5 min and then was stirred for 55 min, while allowing to reach R.T. Saturated solution of NH4Cl was added. The reaction mixture was then extracted with ethyl acetate (×3). The combined organic layers were washed with ammonia solution and than with water, dried over MgSO₄, filtered, and evaporated. The residue was purified by flash chromatography on silica gel (DCM/pentane 1/1 to 2/3) to give the 2-(4-methoxycarbonyl-benzoyl)-pyrrolidine-1-carboxylic acid benzyl ester as an oil (57% yield).

To a solution of the ester in MeOH (0.25 M) was added 1M LiOH (1.1 eq.). The reaction mixture was stirred at R.T. for 3.5 hours. The solvent was evaporated and the reaction mixture was acidified with 1M HCl and then extracted with ethyl acetate (×3). The combined organic layers were washed with water, dried over MgSO₄, filtered, and evaporated to afford the 2-(4-carboxybenzoyl)-pyrrolidine-1-carboxylic acid benzyl ester as a white powder (80% yield).

To a solution of the acid in DMF (0.25), were successively added TBTU (1.3 eq), HOBt (0.3 eq.) and DIEA (3 eq.). The reaction mixture stirred for 5 min and then, 4-aminopyridine (1 eq.) was added and the reaction mixture stirred at R.T. for 1 h. The solvent was evaporated and the residue was poured into of water and extracted with ethyl acetate (×3). The combined organic layers were washed with NaHCO₃ solution and then with water, dried over MgSO₄, filtered and evaporated to give the 2-[4-(Pyridin-4-ylcarbamoyl)-benzoyl]-pyrrolidine-1-carboxylic acid benzyl ester as a yellow oil (88% yield).

To a solution of the ketone in EtOH (0.25 M) was added hydroxylamine hydrochloride (2 eq.) followed by addition of DIEA (3 eq.). The reaction mixture was heated at 80° C. for 24 hours. The reaction mixture was cooled down to RT and the solvent was evaporated. The residue was purified by flash chromatography on silica gel (DCM/MeOH 99/1 to 95/5) to afford the 2-{hydroxyimino-[4-(pyridin-4-ylcarbamoyl)-phenyl]-methyl}-pyrrolidine-1-carboxylic acid benzyl ester as pale yellow foam (82% yield).

To a solution of the oxime (1 eq.) in acetic acid (0.25 M), was added zinc powder (8.5 eq.) and the reaction mixture was stirred at RT for 3 hours. The reaction mixture was filtered. Zinc was washed with AcOH, and the filtrate was evaporated. The residue was acidified with 1M HCl and washed with ethyl acetate. The aqueous layer was basified with 2M NaOH and extracted with ethyl acetate (×3). The combined organic layers were washed with water, dried over MgSO₄, filtered, and evaporated to give the 2-{amino-[4-(pyridin-4-ylcarbamoyl)-phenyl]-methyl}-pyrrolidine-1-carboxylic acid benzyl ester as a white foam (29% yield), which was used without further purification.

To a solution of the amine in DCM was added to di-tert-butyl dicarbonate in DCM dropwise at RT with vigorous stirring. After the starting material was disappeared (15 min), water was added and the reaction mixture was stirred for another 10 min. The organic layer was separated, then washed with water, dried over MgSO₄ and finally evaporated. The residue was purified by flash chromatography on silica gel (DCM/MeOH 99/1 to 95/5) to afford the 2-{tert-butoxycarbonylamino-[4-(pyridin-4-ylcarbamoyl)-phenyl]-methyl}-pyrrolidine-1-carboxylic acid benzyl ester as a white powder (64% yield).

To a solution of 2-{tert-butoxycarbonylamino-[4-(pyridin-4-ylcarbamoyl)-phenyl]-methyl}-pyrrolidine-1-carboxylic acid benzyl ester in MeOH was added Pd/C and the reaction mixture was stirred at R.T. under 1 atm of hydrogen for 5 hours. The reaction mixture was filtered through a layer of Celite, thoroughly washed with MeOH. The solvent was evaporated to give the title compound as a white powder (80% yield)

Intermediates 50 to 73 shown in Table D were synthesized using known procedures. Reference is made to Lebel et al. Org. Lett., 2005, 17(9), 4107 for example.

TABLE D

| Name | Intermediate | Structure |
|---|---|---|
| 1-(4-Chloro-phenyl)-1-methyl-ethylamine | 50 | 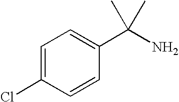 |
| 1-Phenyl-cyclopropylamine | 51 | 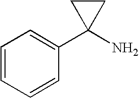 |
| 1-(4-Fluoro-phenyl)-1-methyl-ethylamine | 52 | 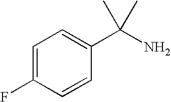 |
| 1-(4-Fluoro-phenyl-cyclopropylamine | 53 | 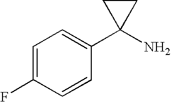 |
| 1-(4-Fluoro-phenyl-cyclobutylamine | 54 | 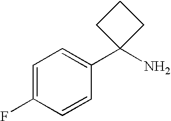 |
| 1-(4-Fluoro-phenyl-cyclopentylamine | 55 | 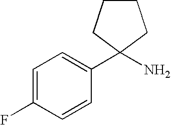 |
| 1-Methyl-1-thiophen-2-yl-ethylamine | 56 | 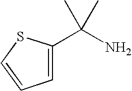 |
| 1-Thiophen-2-yl-cyclopropylamine | 57 | 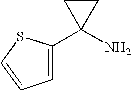 |
| 1-Methyl-1-thiophen-3-yl-ethylamine | 58 | 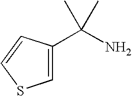 |
| 1-Thiophen-3-yl-cyclopropylamine | 59 | 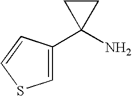 |
| 1-(3,4-Difluoro-phenyl)-1-methyl- ethylamine | 60 | 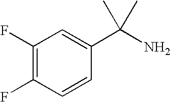 |
| 1-(3,4-Difluoro-phenyl-cyclopropylamine | 61 | 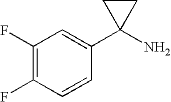 |
| 1-(3,4-Difluoro-phenyl-cyclobutylamine | 62 | 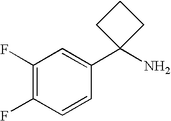 |
| 1-(3,4-Difluoro-phenyl-cyclopentylamine | 63 | 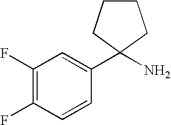 |
| 1-(3,4-Dichloro-phenyl)-1-methyl- ethylamine | 64 | 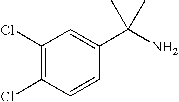 |
| 1-(3,4-Dichloro-phenyl-cyclopropylamine | 65 | 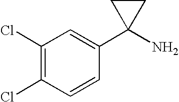 |
| 1-(3-Chloro-4-fluoro-phenyl)-1-methyl- ethylamine | 66 | 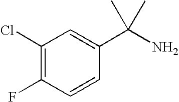 |
| 1-(3-Chloro-4-fluoro-phenyl- cyclopropylamine | 67 | 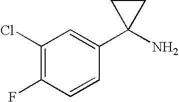 |
| 1-(2,4-Difluoro-phenyl)-1-methyl- ethylamine | 68 | 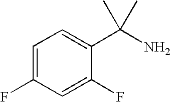 |
| 1-(2,4-Difluoro-phenyl-cyclopropylamine | 69 | 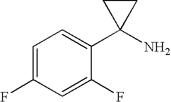 |
| 1-(2-Chloro-4-fluoro-phenyl)-1-methyl- ethylamine | 70 | 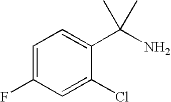 |
| 1-(2-Chloro-4-fluoro-phenyl- cyclopropylamine | 71 | 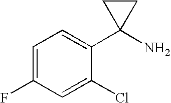 |
| 1-(4-Chloro-2-fluoro-phenyl)-1-methyl- ethylamine | 72 | 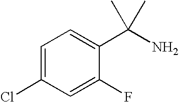 |
| 1-(4-Chloro-2-fluoro-phenyl- cyclopropylamine | 73 | 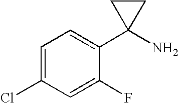 |

### General Procedures:

### Protocol A

To a solution of the corresponding amine (5.25 μmol) in DMF (0.1 M) at neutrality (with DIEA if needed) was added a solution of the corresponding isocyanate or isothiocyanate (1 eq) in dry THF (0.292 M). The reaction mixture was stirred at RT for 2 to 8 hours, and then evaporated.

Deprotection of tert-butoxycarbonylamino group: a mixture of DCM and trifluoroacetic acid (1/1; 100 μl) was added to the residue. The solution was stirred at RT for 2 hours, and then evaporated under reduced pressure. Compounds were used without further purification.

### Protocol B:

To a solution of the corresponding carboxylic acid (5.25 μmol) in DMF (0.437 M) with DIEA (3 eq) was added 1 eq of a solution of TBTU/HOBt (1/0.2) in DMF (0.4 M). The reaction mixture was stirred at RT for 3 to 10 minutes and a solution of the corresponding amine (1 eq) in DMF (0.1 M) at neutrality (with DIEA). The reaction mixture was stirred at RT for 3 hours, and then 0.7 eq of a solution of TBTU/HOBt (1/0.2) in DMF (0.4 M) was added. After 4 hours the reaction mixture was evaporated.

Deprotection of tert-butoxycarbonylamino group: a mixture of DCM and trifluoroacetic acid (1 /1; 100 μl with 5% water) was added to the residue. The solution was stirred at RT for 2 hours, and then evaporated under reduced pressure. Compounds were used without further purification.

### Protocol C:

To a solution of the corresponding carboxylic acid (1 eq.) in DMF (0.25 M) with DIEA (3 eq) was added TBTU (1.3 eq) and HOBt (0.3 eq.). The reaction mixture was stirred at RT for 3 to 10 minutes and the corresponding amine (1 eq) was added. The reaction mixture was stirred at RT for 3 hours to 3 days. The solvent was evaporated. The residue was partitioned between EtOAc and 2N Na₂CO₃ (or 1N NaOH). The product was extracted with EtOAc. The organic layer was separated, washed with brine, dried over magnesium sulfate, and evaporated.

Alternative protocol: To a solution of the corresponding carboxylic acid (1 eq.) in a mixture DMF/DCM (0.25 M) were successively added DCC (1 eq.), HOBt (1 eq.) and DIEA (3 eq.). The solution was stirred at RT for 30 minutes before the addition of the corresponding amine (1 eq.). The reaction mixture was stirred at RT for 2 hours to 3 days. The solvent was evaporated. The residue was partitioned between DCM and water. The product was extracted with DCM. The organic layer was separated, washed with 2N Na₂CO₃ (or 1N NaOH), brine, dried over magnesium sulfate, and evaporated.

Deprotection of tert-butoxycarbonylamino group: The crude product was dissolved in freshly distillated 1,4-dioxane. HCl gas was bubbled in the solution for 10 to 30 minutes. The solvent was evaporated and then, the residue was purified by preparative HPLC.

### Protocol D: Compound 648, 647, 646, 650, 651, 653, 654, 655 can be Made Following the General Scheme Hereafter (Synthesis of Compound 648 as an Example):

1H-Pyrrolo[2,3-b]pyridine (10 g) was dissolved in DME/heptane ((1:2), 200 ml). The reaction mixture was cooled down to 0° C. and mCPBA (2.1 eq.) was added slowly. The reaction mixture turned yellow and a precipitate was formed. DME-heptane (1:2) mixture (50 ml) was added and the slurry was stirred at RT for 6.5 hours. The precipitate was filtered out and washed with DME-heptane (1:2). Slurry of the salt of azaindole N-oxide in water (100 ml) was treated with 30% (in mass) K₂CO₃ to raise the pH to about 9.5-10.5. First, a dark solution was formed. The slurry was cooled to 0 to 5° C. for 16 hours and then filtered to recover the precipitate. The precipitate was washed with additional water and then dried to provide 1H-pyrrolo[2,3-b]pyridine 7-oxide as a pink powder (70% yield).

The 1H-pyrrolo[2,3-b]pyridine 7-oxide (3.67 g) and tetramethylammonium bromide (1.5 eq.) were placed in DMF (15 ml). The mixture was cooled to 0° C. and methanesulfonic anhydride (2 eq.) was added portion wise. The suspension was allowed to reach RT and stirred for 5.5 hours. The reaction mixture was then poured into water (70 ml) and neutralized with 4M NaOH. Water (60 ml) was added. The product was extracted with DCM, washed with water, dried over MgSO₄, filtered. The solvent was evaporated and the residue was purified by flash chromatography on silica gel (DCM to DCM/MeOH 9:1) to give the 4-bromo-1H-pyrrolo[2,3-b]pyridine as a yellow oil (21% yield).

A flask was charged with 4-bromo-1H-pyrrolo[2,3-b]pyridine (1.5 g) and dry THF (12 ml). The mixture was cooled to 0° C. and sodium hydride (418 mg, 60% dispersion in mineral oil) was added portion wise. After 15 min, chloro-triisopropylsilane (0.75 eq.) was added and the flask was sealed and heated at 80° C. for 3.5 hours. The reaction mixture was cooled down to RT, neutralized with saturated NH₄Cl solution and extracted with hexanes. Combined organic layers were dried over MgSO₄ and concentrated under vacuum. Filtration through a small column with silica gel (eluent: 100% hexanes) gave the 4-bromo-1-triisopropylsilanyl-1H-pyrrolo[2,3-b]pyridine as a colorless oil (15% yield).

A two-necked round-bottom flask was dried in the flow of nitrogen. The flask was charged with 4-bromo-1-triisopropylsilanyl-1H-pyrrolo[2,3-b]pyridine (140 mg) and dry THF (3 ml). The mixture was cooled to −85° C. and a solution of tert-butyl lithium (1.5M in pentane, 1.6 eq.) was added dropwise. After 15 min (yellow color), iodine (1 eq.) in THF (2 ml) was added. After 50 min, a saturated aqueous solution of ammonium chloride was added and the mixture was allowed to reach RT. The product was extracted with ethyl acetate (×3), washed with Na₂S₂O₃ solution and water, and then dried over MgSO₄. The solvent was evaporated to provide a mixture of the iodide and de-brominated compound (4:1). The 4-iodo-1-triisopropylsilanyl-1H-pyrrolo[2,3-b]pyridine was purified by flash chromatography on silica gel (hexane 100%) to give a colorless oil (46% yield).

A reaction vessel was charged with 4-iodo-1-triisopropylsilanyl-1H-pyrrolo[2,3-b]pyridine (75 mg), 4-{2-[2-(4-Chloro-phenyl)-2-methyl-propionylamino]-acetyl}-benzamide (1.2 eq.), CuI (0.5 eq.), N,N′-Dimethyl-ethane-1,2-diamine (0.25 eq.) and Cs₂CO₃ (1 eq.) Then dry dioxane (2 ml) was added and the resulting mixture was heated at 120° C. for 3.5 hours. The reaction mixture was cooled down at RT and was filtered through a silica gel plug (which caused a partial TIPS-deprotection). The mixture of 4-{1-amino-2-[2-(4-chloro-phenyl)-2-methyl-propionylamino]-ethyl}-N-(1-triisopropylsilanyl-1H-pyrrolo[2,3-b]pyridin-4-yl)-benzamide and 4-{2-[2-(4-Chloro-phenyl)-2-methyl-propionylamino]-acetyl}-N-(1H-pyrrolo[2,3-b]pyridin-4-yl)-benzamide was purified by flash chromatography (DCM to DCM/EtOAc 4:1) to give a yellow oil (50% yield, mixture 4:1).

To a solution of the previous mixture (protected/unprotected ˜4:1) in ethanol (2 ml) were added hydroxylamine (2 eq.) and DIEA (3 eq.). The reaction mixture was stirred at 80° C. for 1 day. Hydroxylamine (10 mg) and DIEA (35 μL) were added and the reaction mixture was heated at 80° C. for 1 day more. The reaction mixture was cooled down at RT and ethanol was evaporated. The residue was extracted with ethyl acetate, washed with water, dried over MgSO₄, filtered off and the filtrate was evaporated to give a mixture of protected and unprotected 4-{2-[2-(4-Chloro-phenyl)-2-methyl-propionylamino]-1-[hydroxyimino]-ethyl}-N-(1H-pyrrolo[2,3-b]pyridin-4-yl)-benzamide (3:2, 100% yield).

To a solution of the previous oxime (mixture) in AcOH (1.5 ml) was added Zn and the reaction mixture was stirred at RT for 2.5 hours. LCMS indicated a clean deprotection of TIPS group. Zn (20 mg) was added and the reaction mixture was stirred for 1.5 h more. The reaction mixture was filtered off. The solid was washed with AcOH and the filtrate evaporated. The residue was basified with 1M NaOH until pH˜10, extracted with ethyl acetate, washed with water, dried over MgSO₄, filtered off. The filtrate was evaporated to give a free amine (26 mg, 81% pure). HCl salt of amine was extracted from organic layer with 2M HCl to give the title compound as a white powder (35% yield).

### Biological Activity

PKC epsilon is shown to play a role in met metabolic diseases, such as Type I and Type II diabetes, as well as in inflammatory diseases and in the regulating of the immune system and/or an immune response and/or inflammatory response in mammals.

PKC epsilon also plays a role in Toll-like receptor (TLR 4) mediated cytokine expression in macrophages and dendritic cells. Inhibitors of PKC epsilon impair expression of inflammatory cytokines including TNFα, IL-1, and IL-6 (but not IL-10) from macrophages and IL-12 secretion from dendritic cells.

PKC theta inhibition causes impaired activation of NF-AT in CD3/CD28 activated T cells resulting in reduced secretion of IL-2.

The compounds of the invention act as inhibitors of AGC-kinases and in particular as inhibitors of PKC isoenzymes and as inhibitors of ROCK.

The present compounds are inhibitors of PKC epsilon, and PKC theta to treat a variety of inflammatory and autoimmune diseases.

The compounds of the invention show good oral bioavailability.

The compounds of the invention are very potent with a range of selectivity for these PKCs and outstanding selectivity against other kinases. These drug like molecules show good specificity and good PK-properties.

The compounds modulate secretion of inflammatory cytokines, for instance by showing in vivo efficacy in LPS induced TNFα release. These molecules are further tested in in vivo models for instance of R^{A}, inflammation and autoimmune disease.

Advantageous properties of the compounds are:
nanomolar (nM) candidates with drug-like physicochemical properties
in vitro potency: candidates showing IC₅₀ values in low nanomolar ranges, for instance below 30 nM
far more active than the most potent, but nonselective BIM derivative (for instance 10 to 50 fold more)
highly selective (for instance 10×, 20×, 30×, 50× to 100×) versus classical and atypical PKC isoenzymes
highly selective (for instance about 10×, 20×, 30×) versus kinases within the AGC and other kinase families
IC₅₀ in micromolar or nanomolar range in various cell based assays, for instance IC₅₀ of 200 nM potency in cellular assay
very favorable metabolic stabilities and in vivo clearance rates (for instance available for >2 h, >4 h, >6 h)
good in vivo oral bio-availability (for instance >10%, >20%)
clean in vitro tox profile and well tolerated via the oral route
in vivo activity on LPS induced TNFα release proven.

### Biochemical Assay for PKC Epsilon

The compounds were tested for inhibition of PKC epsilon.

The inhibition assays were performed with a fluorescence polarization (FP) assay using the commercially available Protein Kinase C Assay Kit, Red, from Invitrogen (Product ID. No. P2941), essentially in accordance with the protocol supplied by the manufacturer. The substrate used was RFARKGSLRQKNV (M_{w} 1561), also obtained from Invitrogen (Product ID No. P2760). The isozyme PKC epsilon was also obtained from Invitrogen (Product ID No. P2282).

In summary, the compound of the invention was screened in the wells of a 384 well plate for inhibition of each of the isozyme with concentrations varying from 100 μM to 2 pM using a stepwise 2 (or 3)-fold dilution. Staurosporine was used as a positive control (2 μM).

To perform the assay, 2 μl of a solution of the compound to be tested in DMSO (at each concentration) was added to 6 μl of a solution of the enzyme in 10 mM HEPES, 5 mM dithiotreitol, 0.1% Triton X-100, pH 7.4. The final concentration of the enzymes was 10 ng/ml.

After incubating for 30 minutes at room temperature, 4 μl of a mixture of ATP and the protein substrate in 60 mM HEPES (pH7.4), 15 mM MgCl₂, 0.3 mM CaCl₂, 0.06% NP40 was added. The final concentration of the ATP was 2.5 μM and final concentration of protein substrate was 1 μM.

After incubating for 80 minutes at room temperature, 3 μl of a mix solution of 500 mM EDTA (stop solution) and the Rhodamine-based PKC Red Tracer (from the Protein Kinase C Assay Kit) in BGG/phosphate buffer (pH7.4) with 0.02% NaN₃ and 0.1% Triton X-100 was added and 5 μl of a the Anti-Phosphoserine antibody (also from the Protein Kinase C Assay Kit) in BGG/phosphate buffer (pH7.4) with 0.02% NaN₃.

The mixture thus obtained (total volume: 20 μl) was incubated for 60 minutes at room temperature, upon which the fluorescence polarization was measured using an automated plate reader (Perkin Elmer, Model Envision 2100-0010 HTS) with FP filters for rhodamine: excitation filter FITC FP 531 and emission filters FITC FP P-pol 595 and FITC FP S-pol 595 (Perkin-Elmer). The results were fitted to a curve using the XL-Fit algorithm and ICs values were calculated for each fitted curve, again using the XL-Fit algorithm. The IC₅₀ value for the reference compound (Y-27632) was 1 μM for PKC epsilon.

### Biochemical Assay for PKC Theta

The compounds were tested for inhibition of PKC theta.

The inhibition assays were performed with a fluorescence polarization (FP) assay using the commercially available Protein Kinase C Assay Kit, Red, from Invitrogen (Product ID. No. P2941), essentially in accordance with the protocol supplied by the manufacturer. The substrate used was RFARKGSLRQKNV (M_{w} 1561), also obtained from Invitrogen (Product ID No. P2760). The isozyme PKC theta was also obtained from Invitrogen (Product ID No. P2996).

In summary, compound was screened in the wells of a 384 well plate for inhibition of each of the isozyme with concentrations varying from 100 μM to 2 pM using a stepwise 2 (or 3)-fold dilution. Staurosporine was used as a positive control (2 μM).

To perform the assay, 2 μl, of a solution of the compound to be tested in DMSO (at each concentration) was added to 6 μl of a solution of the enzyme in 10 mM HEPES, 5 mM dithiotreitol, 0.1% Triton X-100, pH 7.4. The final concentration of the enzymes was 60 ng/ml.

After incubating for 30 minutes at room temperature, 4 μl of a mixture of ATP and the protein substrate in 60 mM HEPES (pH7.4), 15 mM MgCl₂, 0.3 mM CaCl₂, 0.06% NP40 was added. The final concentration of the ATP was 2.5 μM and final concentration of protein substrate was 1 μM.

After incubating for 80 minutes at room temperature, 3 μl of a mix solution of 500 mM EDTA (stop solution) and the Rhodamine-based PKC Red Tracer (from the Protein Kinase C Assay Kit) in BGG/phosphate buffer (pH7.4) with 0.02% NaN₃ and 0.1% Triton X-100 was added and 5 μl of a the Anti-Phosphoserine antibody (also from the Protein Kinase C Assay Kit) in BGG/phosphate buffer (pH7.4) with 0.02% NaN₃.

The mixture thus obtained (total volume: 20 μl) was incubated for 60 minutes at room temperature, upon which the fluorescence polarization was measured using an automated plate reader (Perkin Elmer, Model Envision 2100-0010 HTS) with FP filters for rhodamine: excitation fitter FITC FP 531 and emission filters FITC FP P-pol 595 and FITC FP S-pol 595 (Perkin-Elmer). The results were fitted to a curve using the XL-Fit algorithm and IC₅₀ values were calculated for each fitted curve, again using the XL-Fit algorithm. The IC₅₀ value for the reference compound (Y-27632) was 2 μM for PKC theta.

### Biochemical Assay for ROCK

The compounds were tested for inhibition of human ROCKα/ROCKII mix.

The inhibition assays were performed with a fluorescence polarization (FP) assay using the commercially available ROCK IMAP Kit from Molecular Devices (Product ID. No. R8093), essentially in accordance with the protocol supplied by the manufacturer. The S6 ribosomal protein-derived substrate used was (FI)-AKRRRLSSLRA, also obtained from Molecular Devices (Product ID No. R7184). The enzyme mix ROCKα/ROCKII was obtained from Upstate Biotechnology (Product ID No 14-451).

In summary, the compound was screened in the wells of a 384 well plate for enzymatic inhibition with concentrations varying from 100 μM to 0.3 nM using a stepwise 3 (or 2)-fold dilution. Y compound (Y-27632 commercially available from Tocris) was used as a reference (0.4 μM).

To perform the assay, 1 μl of a solution of the compound to be tested in DMSO (at each concentration) was added to 2 μl of a solution of the enzyme in 10 mM Tris-HCl , 10 mM MgCl₂, 0.1% BSA, 0.05% NaN₃, pH 7.2. The final concentration of the enzyme was 2.6 nM.

After incubating for 30 minutes at room temperature, 2 μl of a mixture of ATP and the protein substrate in 10 mM Tris-HCl, 10 mM MgCl₂, 0.1% BSA, 0.05% NaN₃, pH 7.2 was added. The final concentration of the ATP was 10 μM and final concentration of protein substrate was 0.2 μM.

After incubating for 60 minutes at room temperature, 12 μl of the IMAP Binding Solution (mix of the IMAP Binding Buffer A (1×) and the IMAP Binding Reagent (from the ROCK IMAP kit)) was added.

The mixture thus obtained (total volume: 17 μl) was incubated for 60 minutes at room temperature, upon which the fluorescence polarization was measured using an automated plate reader (Perkin Elmer, Model Envision 2100-0010 HTS) with FP filters: excitation filter FITC FP 480 and emission filters FITC FP P-pol 535 and FITC FP S-pol 535 (Perkin-Elmer). The results were fitted to a curve using the XL-Fit algorithm and IC₅₀ values were calculated for each fitted curve, again using the XL-Fit algorithm.

The IC₅₀ value for the reference compound (Y compound Y-27632) was 0.4 μM.

### Compounds of the Invention

In the tables which are set forth below, exemplary compounds of the invention are set out in tabulated form. In these tables, the name of the compound, an arbitrarily assigned compound number and structural information are set out. In addition, the protocol by which the compounds were made is provided and the IC₅₀ value obtained (in accordance with the protocol set forth above) is given. the IC₅₀ value obtained (in accordance with the protocol set forth above) is represented as follows: “++++” means CO₅₀ below 0.05 μM; “+++” means IC₅₀ between 0.05 and 0.5 μM; “++” means IC₅₀ between 0.5 and 5 μM, “+” means IC₅₀ between 5 and 50 μM and “nd” means “not determined yet”.

### Attribution of the Configuration:

The Cahn-Ingold-Prelog system was used to attribute the absolute configuration of chiral center, in which the four groups on an asymmetric carbon are ranked to a set of sequences rules. Reference is made to Cahn; Ingold; Prelog Angew. Chem. Int Ed. Engl. 1966, 5, 385-415.

In the configuration R,R for example the first letter refers to the configuration of the carbon bearing the amine group.

### Name of the Molecule

The software MDL ISIS™/Draw 2.5 was used to assign the name of the molecules.

Table 1 shows the results for compounds of Formula XII. Table 2 shows the results for compounds of Formula XIII. Table 3 shows the results for compounds of Formula XIV. Table 4 shows the inhibition results for compounds 372 to 375. Table 5 shows the inhibition results for compounds 397 to 419. Table 6 shows the results for compounds of Formula XV Table 7 shows the results for compounds of Formula XV. Table 8 shows the results for compounds of Formula XVI. As used herein the term “nd” means “not determined yet” and “Pr” is “Protocol”.

TABLE 1

| | |
|---|---|
| | XII |
| 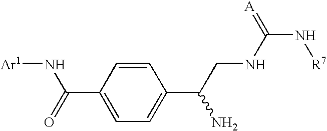 | |

| | | | | | | | |
|---|---|---|---|---|---|---|---|
| | | | | | | IC₅₀ | IC₅₀ |
| | | | | | | μM | μM |
| Name | Compound | Ar¹— | A | —R⁷ | Pr | PKCε | ROCK |

| | | | | | | | |
|---|---|---|---|---|---|---|---|
| 4-[1-amino-2-(3-ethyl-thioureido)- ethyl]-N-pyridin-4-yl-benzamide | Compound 1 | 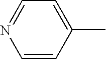 | S | 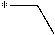 | A | ++ | ++ |
| 4-[1-amino-2-(3-butyl-ureido)-ethyl]-N- pyridin-4-yl-benzamide | Compound 2 | 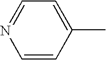 | O | 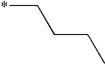 | A | ++ | ++ |
| 4-[1-amino-2-(3-tert-butyl-ureido)- ethyl]-N-pyridin-4-yl-benzamide | Compound 3 | 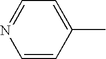 | O | 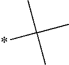 | A | ++ | ++ |
| 4-[1-amino-2-(3-cyclopentyl-ureido)- ethyl]-N-pyridin-4-yl-benzamide | Compound 4 | 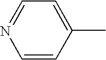 | O | 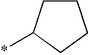 | A | ++ | ++ |
| 4-[1-amino-2-(3-cyclohexyl-ureido)- ethyl]-N-pyridin-4-yl-benzamide | Compound 5 | 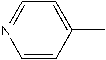 | O | 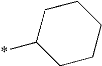 | A | + | ++ |
| 3-(3-{2-amino-2-[4-(pyridin-4- ylcarbamoyl)-phenyl]-ethyl}-ureido)- propionic acid ethyl ester | Compound 6 | 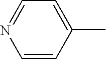 | O | 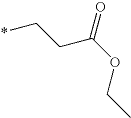 | A | + | ++ |
| 4-[1-amino-2-(3-benzyl-ureido)-ethyl]- N-pyridin-4-yl-benzamide | Compound 7 | 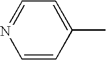 | O | 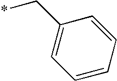 | A | ++ | +++ |
| 4-{1-amino-2-[3-(4-methyl-benzyl)- ureido]-ethyl}-N-pyridin-4-yl- benzamide | Compound 8 | 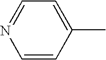 | O | 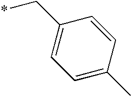 | A | ++ | +++ |
| 4-{1-amino-2-[3-(2-methyl-benzyl)- ureido]-ethyl}-N-pyridin-4-yl- benzamide | Compound 9 | 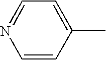 | O | 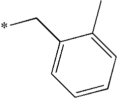 | A | nd | +++ |
| 4-{1-amino-2-[3-(4-methoxy-benzyl)- ureido]-ethyl}-N-pyridin-4-yl- benzamide | Compound 10 | 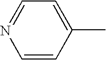 | O | 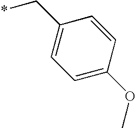 | A | nd | +++ |
| 4-[1-amino-2-(3-phenyl-thioureido)- ethyl]-N-pyridin-4-yl-benzamide | Compound 11 | 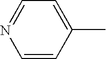 | S | 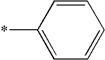 | A | ++ | +++ |
| 4-[1-amino-2-(3-o-tolyl-thioureido)- ethyl]-N-pyridin-4-yl-benzamide | Compound 12 | 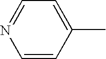 | S | 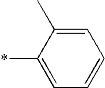 | A | +++ | ++ |
| 4-{1-amino-2-[3-(4-chloro-phenyl)- thioureido]-ethyl}-N-pyridin-4-yl- benzamide | Compound 13 | 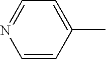 | S | 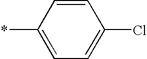 | A | +++ | +++ |
| 4-{1-amino-2-[3-(4-methoxy-phenyl)- thioureido]-ethyl}-N-pyridin-4-yl- benzamide | Compound 14 | 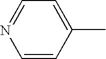 | S | 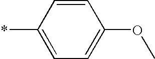 | A | ++ | ++ |
| 4-{1-amino-2-[3-(4-trifluoromethoxy- phenyl)-thioureido]-ethyl}-N-pyridin-4- yl-benzamide | Compound 15 | 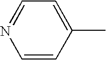 | S | 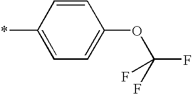 | A | + | ++ |
| 4-{1-amino-2-[3-(4-trifluoromethyl- phenyl)-thioureido]-ethyl}-N-pyridin-4- yl-benzamide | Compound 16 | 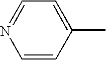 | S | 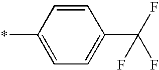 | A | ++ | ++ |
| 4-[1-amino-2-(3-naphtalen-1-yl- thioureido)-ethyl]-N-pyridin-4-yl- benzamide | Compound 17 | 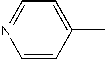 | S | 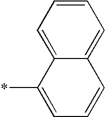 | A | +++ | +++ |
| 4-{1-amino-2-[3-(4-tert-butyl-phenyl)- thioureido]-ethyl}-N-pyridin-4-yl- benzamide | Compound 18 | 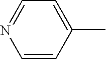 | S | 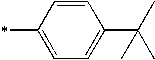 | A | + | ++ |
| 4-{1-amino-2-[3-(4-fluoro-phenyl)- ureido]-ethyl}-N-pyridin-4-yl- benzamide | Compound 19 | 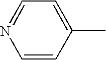 | O | 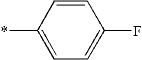 | A | nd | +++ |
| 4-{1-amino-2-[3-(2-fluoro-phenyl)- ureido]-ethyl}-N-pyridin-4-yl- benzamide | Compound 20 | 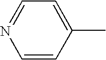 | O | 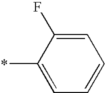 | A | ++ | +++ |
| 4-{1-amino-2-[3-(4-dimethylamino- phenyl)-ureido]-ethyl}-N-pyridin-4-yl- benzamide | Compound 21 | 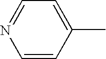 | O | 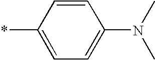 | A | ++ | +++ |
| 4-[1-amino-2-(3-biphenyl-4-yl-ureido)- ethyl]-N-pyridin-4-yl-benzamide | Compound 22 | 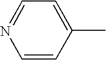 | O | 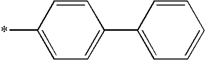 | A | ++ | ++ |
| 4-[1-amino-2-(3-naphtalen-2-yl- ureido)-ethyl]-N-pyridin-4-yl- benzamide | Compound 23 | 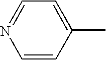 | O | 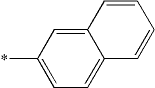 | A | ++ | +++ |
| 4-{1-amino-2-[3-(4-tert-butyl-phenyl)- ureido]-ethyl}-N-pyridin-4-yl- benzamide | Compound 24 | 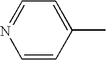 | O | 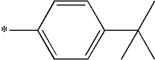 | A | + | ++ |
| 4-[1-amino-2-(3-o-tolyl-ureido)-ethyl]- N-pyridin-4-yl-benzamide | Compound 25 | 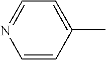 | O | 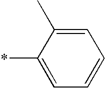 | A | ++ | +++ |
| 4-{1-amino-2-[3-(4-chloro-2-methyl- phenyl)-ureido]-ethyl}-N-pyridin-4-yl- benzamide | Compound 26 | 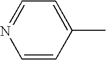 | O | 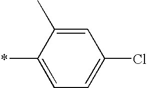 | A | +++ | +++ |
| 4-{1-amino-2-[3-(5-chloro-2-methoxy- phenyl)-ureido]-ethyl}-N-pyridin-4-yl- benzamide | Compound 27 | 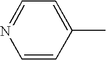 | O | 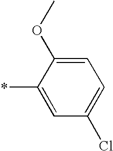 | A | nd | +++ |
| 4-[1-amino-2-(3-naphtalen-1-yl- ureido)-ethyl]-N-pyridin-4-yl- benzamide | Compound 28 | 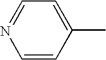 | O | 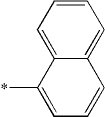 | A | ++ | +++ |
| 4-{1-amino-2-[3-(3-methoxy-phenyl)- ureido]-ethyl}-N-pyridin-4-yl- benzamide | Compound 29 | 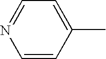 | O | 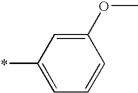 | A | ++ | +++ |
| 4-{1-amino-2-[3-(4-methoxy-phenyl)- ureido]-ethyl}-N-pyridin-4-yl- benzamide | Compound 30 | 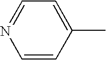 | O | 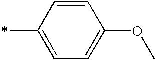 | A | ++ | +++ |
| 4-{1-Amino-2-[3-(4-chloro-phenyl)- ureido]-ethyl}-N-pyridin-4-yl- benzamide | Compound 326 | 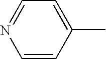 | O | 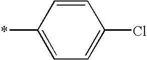 | A | +++ | +++ |

TABLE 2

| | |
|---|---|
| | XIII |
| 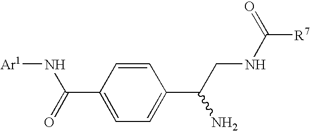 | |

| | | | | | IC₅₀ | IC₅₀ |
|---|---|---|---|---|---|---|
| | | | | | μM | μM |
| Name | Compound | Ar¹— | —R⁷ | Pr | PKCε | ROCK |
| 4-(2-Acetylamino-1-amino-ethyl)-N- pyridin-4-yl-benzamide | Compound 31 | 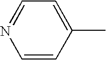 | —CH₃ | B | + | ++ |
| 4-(1-Amino-2-butyrylamino-ethyl)-N- pyridin-4-y-benzamide | Compound 32 | 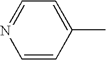 | 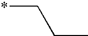 | B | ++ | ++ |
| 4-(1-Amino-2-pentanoylamino- ethyl)-N-pyridin-4-yl-benzamide | Compound 33 | 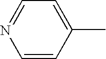 | 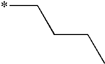 | B | ++ | ++ |
| 4-[1-Amino-2-(3-methyl- butyrylamino)-ethyl]-N-pyridin-4-yl- benzamide | Compound 34 | 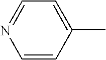 | 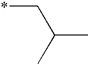 | B | ++ | ++ |
| 4-[1-Amino-2-(2-methyl- butyrylamino)-ethyl]-N-pyridin-4-yl- benzamide | Compound 35 | 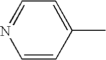 | 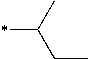 | B | ++ | ++ |
| 4-[1-Amino-2-(4-methyl- pentanoylamino)-ethyl]-N-pyridin-4- yl-benzamide | Compound 36 | 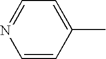 | 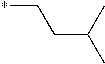 | B | ++ | ++ |
| 4-{1-amino-2-[2-(4-methy)- octanoylamino]-ethyl}-N-pyridin-4-yl- benzamide | Compound 37 | 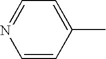 | 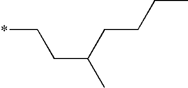 | B | ++ | +++ |
| 4-(1-Amino-2-nonanoylamino-ethyl)- N-pyridin-4-yl-benzamide | Compound 38 | 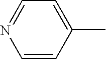 | 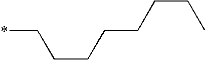 | B | ++ | +++ |
| 4-[1-Amino-2- (cyclopropanecarbonyl-amino)- ethyl]-N-pyridin-4-yl-benzamide | Compound 39 | 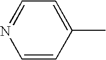 | 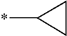 | B | ++ | ++ |
| 4-{1-Amino-2-[(2-methyl- cyclopropanecarbonyl)-amino]- ethyl}-N-pyridin-4-yl-benzamide | Compound 40 | 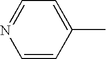 | 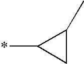 | B | ++ | ++ |
| 4-[1-Amino-2-(cyclobutanecarbonyl- amino)-ethyl]-N-pyridin-4-yl- benzamide | Compound 41 | 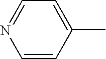 | 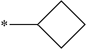 | B | ++ | ++ |
| 4-[1-Amino-2- (cyclopentanecarbonyl-amino)- ethyl]-N-pyridin-4-yl-benzamide | Compound 42 | 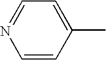 | 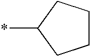 | B | ++ | ++ |
| 4-[1-Amino-2-(cyclohexanecarbonyl- amino)-ethyl]-N-pyridin-4-yl- benzamide | Compound 43 | 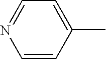 | 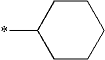 | B | + | ++ |
| 4-{1-Amino-2-[(4-methoxy- cyclohexanecarbonyl)-amino]-ethyl}- N-pyridin-4-yl-benzamide | Compound 44 | 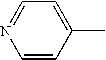 | 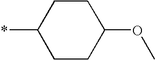 | B | + | ++ |
| 4-{1-Amino-2-[(3-methyl- cyclohexanecarbonyl)-amino]-ethyl}- N-pyridin-4-yl-benzamide | Compound 45 | 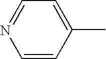 | 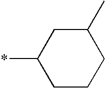 | B | ++ | ++ |
| 4-[1-Amino-2-(2-cycopropyl- acetylamino)-ethyl]-N-pyridin-4-yl- benzamide | Compound 46 | 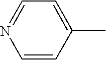 | 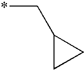 | B | ++ | ++ |
| 4-[1-Amino-2-(2-cyclopentyl- acetylamino)-ethyl]-N-pyridin-4-yl- benzamide | Compound 47 | 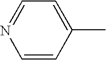 | 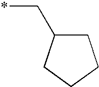 | B | ++ | ++ |
| 4-[1-Amino-2-(2-cyclopent-2-enyl- acetylamino)-ethyl]-N-pyridin-4-yl- benzamide | Compound 48 | 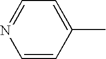 | 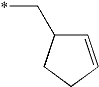 | B | ++ | +++ |
| 4-[1-Amino-2-(2-indan-2-yl- acetylamino)-ethyl]-N-pyridin-4-yl- benzamide | Compound 49 | 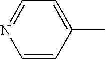 | 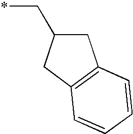 | B | ++ | +++ |
| 4-[1-Amino-2-(2-cyclohexyl- acetylamino)-ethyl]-N-pyridin-4-yl- benzamide | Compound 50 | 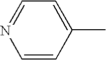 | 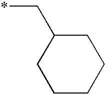 | B | ++ | ++ |
| 4-[1-Amino-2-(3-cyclopentyl- propionylamino)-ethyl]-N-pyridin-4- yl-benzamide | Compound 51 | 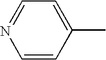 | 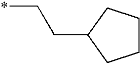 | B | ++ | +++ |
| 4-[1-Amino-2-(3-cyclohexyl- propionylamino)-ethyl]-N-pyridin-4- yl-benzamide | Compound 52 | 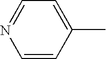 | 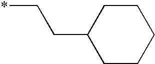 | B | ++ | +++ |
| 4-[1-Amino-2-(4-dimethylamino- butyrylamino)-ethyl]-N-pyridin-4-yl- benzamide | Compound 53 | 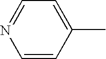 | 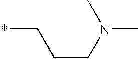 | B | + | ++ |
| 4-[1-Amino-2-(2-cyano- acetylamino)-ethyl]-N-pyridin-4-yl- benzamide | Compound 54 | 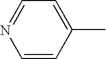 | 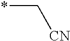 | B | ++ | +++ |
| 4-[1-Amino-2-(2-benzoylamino- acetylamino)-ethyl]-N-pyridin-4-yl- benzamide | Compound 55 | 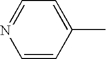 | 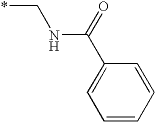 | B | + | +++ |
| Furan-2-carboxylic acid ({2-amino-2- [4-(pyridin-4-ylcarbamoyl)-phenyl]- ethylcarbamoyl}-methyl)-amide | Compound 56 | 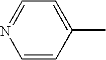 | 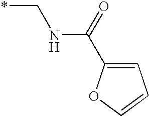 | B | + | +++ |
| 4-[1-Amino-2-(2-methoxy- acetylamino)-ethyl]-N-pyridin-4-yl- benzamide | Compound 57 | 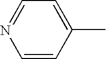 | 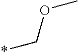 | B | + | ++ |
| 4-[1-Amino-2-(3-methoxy- propionylamino)-ethyl]-N-pyridin-4- yl-benzamide | Compound 58 | 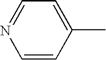 | 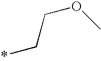 | B | + | ++ |
| 4-[1-Amino-2-(2- isopropylideneaminooxy- propionylamino)-ethyl]-N-pyridin-4- yl-benzamide | Compound 59 | 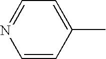 | 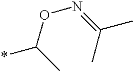 | B | ++ | ++ |
| 4-{1-Amino-2-[2-(1-phenyl- ethylideneaminooxy)-acetylamino]- ethyl}-N-pyridin-4-yl-benzamide | Compound 60 | 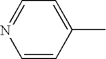 | 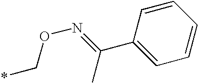 | B | ++ | +++ |
| 4-{1-Amino-2-[2-(3-methyl-thiophen- 2-ylmethyleneaminooxy)- acetylamino]-ethyl}-N-pyridin-4-yl- benzamide | Compound 61 | 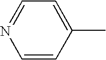 | 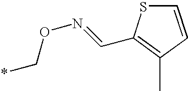 | B | + | ++ |
| 4-[1-amino-2-(2-ethoxy- acetylamino)-ethyl]-N-pyridin-4-yl- benzamide | Compound 62 | 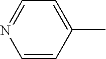 | 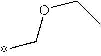 | B | + | ++ |
| 4-[1-amino-2-(2-ethylsulfanyl- acetylamino)-ethyl]-N-pyridin-4-yl- benzamide | Compound 63 | 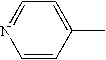 | 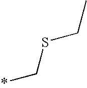 | B | ++ | +++ |
| 4-{1-amino-2-[2-(pyrimidin-2- ylsulfanyl)-acetylamino]-ethyl}-N- pyridin-4-yl-benzamide | Compound 64 | 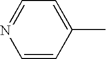 | 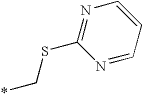 | B | + | ++ |
| Tetrahydro-furan-3-carboxylic acid {2-amino-2-[4-(pyridin-4- ylcarbamoyl)-phenyl]-ethyl}-amide | Compound 65 | 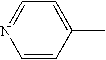 | 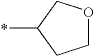 | B | ++ | ++ |
| Tetrahydro-furan-2-carboxylic acid {2-amino-2-[4-(pyridin-4- ylcarbamoyl)-phenyl]-ethyl}-amide | Compound 66 | 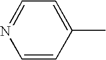 | 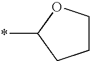 | B | + | ++ |
| 5-Oxo-pyrrolidine-2-(S)-carboxylic acid {2-amino-2-[4-(pyridin-4- ylcarbamoyl)-phenyl]-ethyl}-amide | Compound 67 | 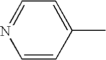 | 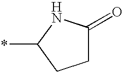 | B | + | ++ |
| 5-Oxo-pyrrolidine-2-(R)-carboxylic acid {2-amino-2-[4-(pyridin-4- ylcarbamoyl)-phenyl]-ethyl}-amide | Compound 68 | 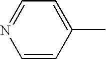 | 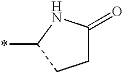 | B | + | +++ |
| 4-(1-amino-2-benzoylamino-ethyl)- N-pyridin-4-yl-benzamide | Compound 69 | 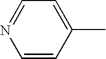 | 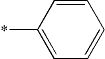 | B | ++ | +++ |
| Naphthalene-2-carboxylic acid {2- amino-2-[4-pyridin-4-ylcarbamoyl)- phenyl]-ethyl}-amide | Compound 70 | 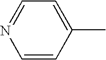 | 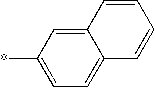 | B | + | +++ |
| Naphthalene-1-carboxylic acid {2- amino-2-[4-pyridin-4-ylcarbamoyl)- phenyl]-ethyl}-amide | Compound 71 | 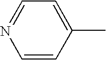 | 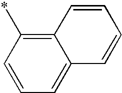 | B | ++ | ++ |
| 4-[1-amino-2-(2-methoxy- benzoylamino)-ethyl]-N-pyridin-4-yl- benzamide | Compound 72 | 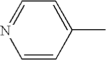 | 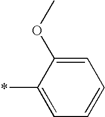 | B | ++ | ++ |
| 4-[1-amino-2-(3-methoxy- benzoylamino)-ethyl]-N-pyridin-4-yl- benzamide | Compound 73 | 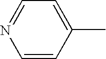 | 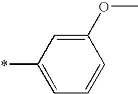 | B | ++ | ++ |
| 4-[1-amino-2-(4-methoxy- benzoylamino)-ethyl]-N-pyridin-4-yl- benzamide | Compound 74 | 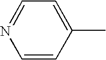 | 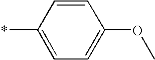 | B | + | ++ |
| 4-[1-amino-2-(4-propoxy- benzoylamino)-ethyl]-N-pyridin-4-yl- benzamide | Compound 75 | 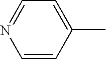 | 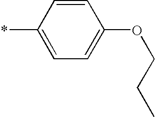 | B | + | ++ |
| 4-[1-amino-2-(2-phenoxy- benzoylamino)-ethyl]-N-pyridin-4-yl- benzamide | Compound 76 | 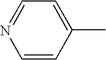 | 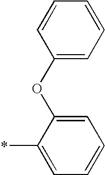 | B | ++ | ++ |
| 4-[1-amino-2-(3-phenoxy- benzoylamino)-ethyl]-N-pyridin-4-yl- benzamide | Compound 77 | 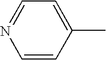 | 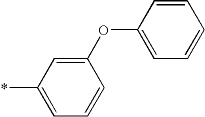 | B | + | ++ |
| 4-[1-amino-2-(4-phenoxy- benzoylamino)-ethyl]-N-pyridin-4-yl- benzamide | Compound 78 | 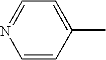 | 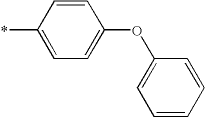 | B | + | ++ |
| 4-[1-amino-2-(3,5-dimethoxy- benzoylamino)-ethyl]-N-pyridin-4-yl- benzamide | Compound 79 | 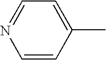 | 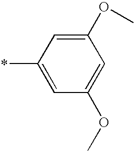 | B | ++ | ++ |
| Benzo[1,3]dioxole-5-carboxylic acid {2-amino-2-[4-(pyridin-4- ylcarbamoyl)-phenyl]-ethyl}amide | Compound 80 | 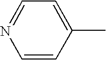 | 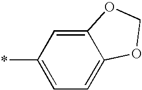 | B | + | +++ |
| 4-[1-amino-2-(4-diethylamino- benzoylamino)-ethyl]-N-pyridin-4-yl- benzamide | Compound 81 | 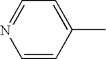 | 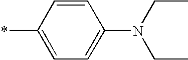 | B | + | ++ |
| 4-[1-amino-2-(2-phenylamino- benzoylamino)-ethyl]-N-pyridin-4-yl- benzamide | Compound 82 | 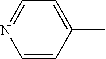 | 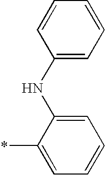 | B | ++ | ++ |
| 4-[1-amino-2-(4-acetylamino- benzoylamino)-ethyl]-N-pyridin-4-yl- benzamide | Compound 83 | 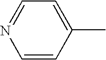 | 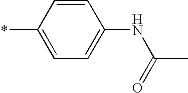 | B | + | ++ |
| 4-[1-amino-2-(2-methyl- benzoylamino)-ethyl]-N-pyridin-4-yl- benzamide | Compound 84 | 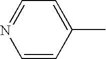 | 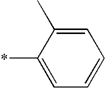 | B | + | ++ |
| 4-[1-amino-2-(3-methyl- benzoylamino)-ethyl]-N-pyridin-4-yl- benzamide | Compound 85 | 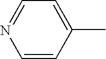 | 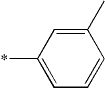 | B | ++ | ++ |
| 4-[1-amino-2-(4-methyl- benzoylamino)-ethyl]-N-pyridin-4-yl- benzamide | Compound 86 | 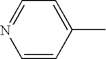 | 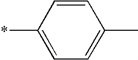 | B | + | ++ |
| Biphenyl-2-carboxylic acid {2-amino- 2-[4-(pyridin-4-ylcarbamoyl)-phenyl]- ethyl}-amide | Compound 87 | 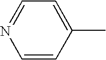 | 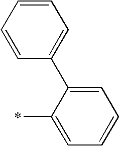 | B | ++ | ++ |
| Biphenyl-4-carboxylic acid {2-amino- 2-[4-(pyridin-4-ylcarbamoyl)-phenyl]- ethyl}-amide | Compound 88 | 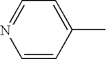 | 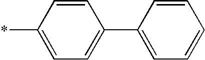 | B | + | ++ |
| 4-[1-amino-2-(3-chloro- benzoylamino)-ethyl]-N-pyridin-4-yl- benzamide | Compound 89 | 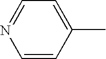 | 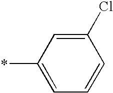 | B | ++ | ++ |
| 4-[1-amino-2-(4-chloro- benzoylamino)-ethyl]-N-pyridin-4-yl- benzamide | Compound 90 | 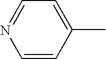 | 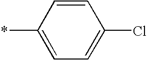 | B | + | ++ |
| 4-[1-amino-2-(3-bromo- benzoylamino)-ethyl]-N-pyridin-4-yl- benzamide | Compound 91 | 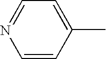 | 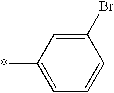 | B | ++ | ++ |
| 4-[1-amino-2-(3-fluoro- benzoylamino)-ethyl]-N-pyridin-4-yl- benzamide | Compound 92 | 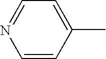 | 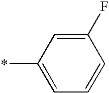 | B | ++ | ++ |
| 4-[1-amino-2-(4-fluoro- benzoylamino)-ethyl]-N-pyridin-4-yl- benzamide | Compound 93 | 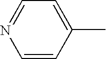 | 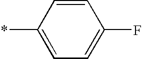 | B | ++ | ++ |
| 4-[1-amino-2-(3-cyano- benzoylamino)-ethyl]-N-pyridin-4-yl- benzamide | Compound 94 | 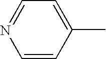 | 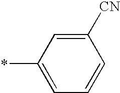 | B | ++ | ++ |
| 4-[1-amino-2-(4-cyano- benzoylamino)-ethyl]-N-pyridin-4-yl- benzamide | Compound 95 | 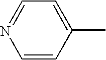 | 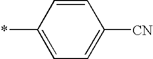 | B | + | ++ |
| 4-[1-amino-2-(3-methanesulfonyl- benzoylamino)-ethyl]-N-pyridin-4-yl- benzamide | Compound 96 | 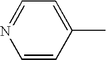 | 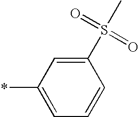 | B | ++ | ++ |
| 4-[1-amino-2-(3-ethylsulfamoyl- benzoylamino)-ethyl]-N-pyridin-4-yl- benzamide | Compound 97 | 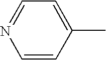 | 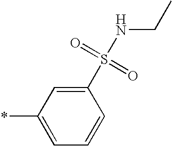 | B | ++ | ++ |
| 4-[1-amino-2-(4-methylsulfamoyl- benzoylamino)-ethyl]-N-pyridin-4-yl- benzamide | Compound 98 | 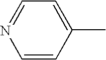 | 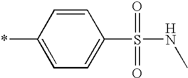 | B | + | ++ |
| 4-[1-amino-2-(3-phenylsulfamoyl- benzoylamino)-ethyl]-N-pyridin-4-yl- benzamide | Compound 99 | 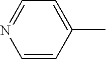 | 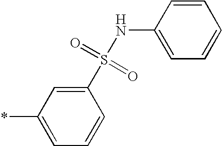 | B | ++ | ++ |
| 4-[1-amino-2-(4-benzoyl- benzoylamino)-ethyl]-N-pyridin-4-yl- benzamide | Compound 100 | 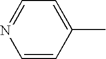 | 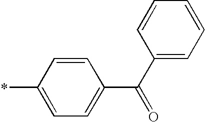 | B | + | ++ |
| 4-{1-amino-2-[(3-tert-butoxyimino- methyl)-benzoylamino]-ethyl}-N- pyridin-4-yl-benzamide | Compound 101 | 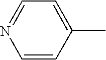 | 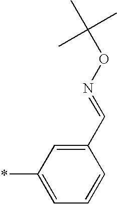 | B | ++ | ++ |
| 4-{1-amino-2-[(4-tert-butoxyimino- methyl)-benzoylamino]-ethyl}-N- pyridin-4-yl-benzamide | Compound 102 | 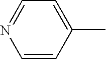 | 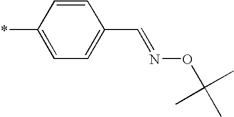 | B | + | ++ |
| 4-{1-amino-2-[(3-pyrazol-1ylmethyl)- benzoylamino]-ethyl}-N-pyridin-4-yl- benzamide | Compound 103 | 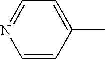 | 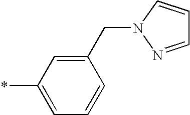 | B | ++ | ++ |
| N-{2-Amino-2-[4-(pyridin-4- ylcarbamoyl)-phenyl]-ethyl}- isonicotinamide | Compound 104 | 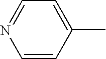 | 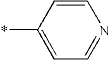 | B | + | ++ |
| Pyridine-2-carboxylic acid {2-amino- 2-[4-(pyridin-4-ylcarbamoyl)-phenyl]- ethyl}-amide | Compound 105 | 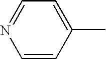 | 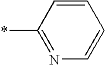 | B | + | ++ |
| N-{2-Amino-2-[4-(pyridin-4- ylcarbamoyl)-phenyl]-ethyl}- nicotinamide | Compound 106 | 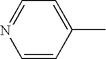 | 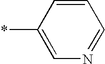 | B | ++ | ++ |
| N-{2-Amino-2-[4-(pyridin-4- ylcarbamoyl)-phenyl]-ethyl}- nicotinamide | Compound 107 | 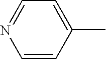 | 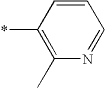 | B | ++ | ++ |
| Pyrazine-2-carboxylic acid {2- amino-2-[4-(pyridin-4-ylcarbamoyl)- phenyl]-ethyl}-amide | Compound 108 | 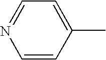 | 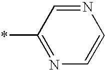 | B | + | ++ |
| 6-Oxo-1,6-dihydro-pyridazine-3- carboxylic acid {2-amino-2-[4- (pyridin-4-ylcarbamoyl)-phenyl]- ethyl}-amide | Compound 109 | 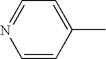 | 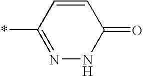 | B | + | ++ |
| Isoquinoline-1-carboxylic acid {2- amino-2-[4-(pyridin-4-ylcarbamoyl)- phenyl]-ethyl}-amide | Compound 110 | 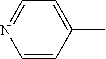 | 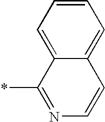 | B | ++ | ++ |
| Quinoline-4-carboxylic acid {2- amino-2-[4-(pyridin-4-ylcarbamoyl)- phenyl]-ethyl}-amide | Compound 111 | 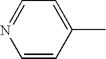 | 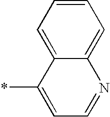 | B | ++ | ++ |
| Quinoline-2-carboxylic acid {2- amino-2-[4-(pyridin-4-ylcarbamoyl)- phenyl]-ethyl}-amide | Compound 112 | 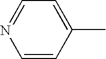 | 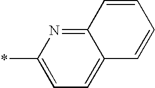 | B | nd | +++ |
| Cinnoline-4-carboxylic acid {2- amino-2-[4-(pyridin-4-ylcarbamoyl)- phenyl]-ethyl}-amide | Compound 113 | 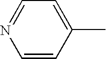 | 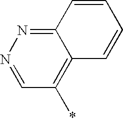 | B | ++ | ++ |
| Quinoline-6-carboxylic acid {2- amino-2-[4-(pyridin-4-ylcarbamoyl)- phenyl]-ethyl}-amide | Compound 114 | 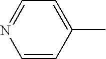 | 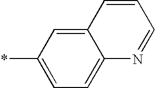 | B | + | ++ |
| Thiophene-3-carboxylic acid {2- amino-2-[4-(pyridin-4-ylcarbamoyl)- phenyl]-ethyl}-amide | Compound 115 | 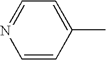 | 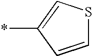 | B | ++ | +++ |
| Thiophene-2-carboxylic acid {2- amino-2-[4-(pyridin-4-ylcarbamoyl)- phenyl]-ethyl}-amide | Compound 116 | 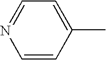 | 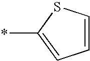 | B | +++ | +++ |
| 5-Methyl-thiophene-2-carboxylic acid {2-amino-2-[4-(pyridin-4- ylcarbamoyl)-phenyl]-ethyl}-amide | Compound 117 | 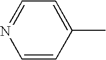 | 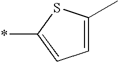 | B | ++ | ++ |
| 5-(1-tert-Butoxyimino-ethyl)- thiophene-2-carboxylic acid {2- amino-2-[4-(pyridin-4-ylcarbamoyl)- phenyl]-ethyl}-amide | Compound 118 | 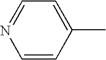 | 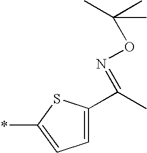 | B | + | ++ |
| Furan-3-carboxylic acid {2-amino-2- [4-(pyridin-4-ylcarbamoyl)-phenyl]- ethyl}-amide | Compound 119 | 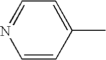 | 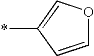 | B | ++ | +++ |
| 5-Nitro-furan-2-carboxylic acid {2- amino-2-[4-(pyridin-4-ylcarbamoyl)- phenyl]-ethyl}-amide | Compound 120 | 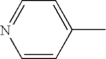 | 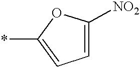 | B | ++ | ++ |
| 1-Methyl-1H-pyrrole-2-carboxylic acid {2-amino-2-[4-(pyridin-4- ylcarbamoyl)-phenyl]-ethyl}-amide | Compound 121 | 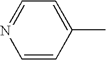 | 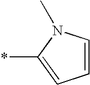 | B | ++ | ++ |
| 1H-Imidazole-4-carboxylic acid {2- amino-2-[4-(pyridin-4-ylcarbamoyl)- phenyl]-ethyl}-amide | Compound 122 | 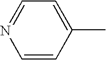 | 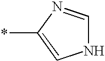 | B | + | ++ |
| 5-Amino-1-(4-fluorophenyl)-1H- pyrazole-4-carboxylic acid {2-amino- 2-[4-(pyridin-4-ylcarbamoyl)-phenyl]- ethyl}-amide | Compound 123 | 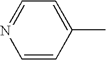 | 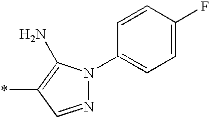 | B | + | ++ |
| 2-Phenyl-thiazole-4-carboxylic acid {2-amino-2-[4-(pyridin-4- ylcarbamoyl)-phenyl]-ethyl}-amide | Compound 124 | 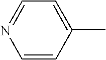 | 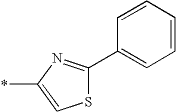 | B | + | ++ |
| 2-(4-Fluoro-phenylamino)-thiazole- 4-carboxylic acid {2-amino-2-[4- (pyridin-4-ylcarbamoyl)phenyl]- ethyl}-amide | Compound 125 | 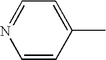 | 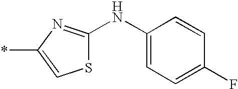 | B | ++ | ++ |
| 4-Methyl-2-Phenyl-thiazole-5- carboxylic acid {2-amino-2-[4- (pyridin-4-ylcarbamoyl)-phenyl]- ethyl}-amide | Compound 126 | 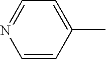 | 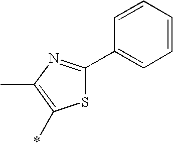 | B | + | ++ |
| 1H-Benzotriazole-5-carboxylic acid {2-amino-2-[4-(pyridin-4- ylcarbamoyl)-phenyl]-ethyl}-amide | Compound 127 | 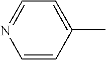 | 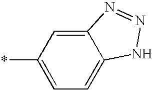 | B | + | ++ |
| 5-Methoxy-1H-indole-2-carboxylic acid {2-amino-2-[4-(pyridin-4- ylcarbamoyl)-phenyl]-ethyl}-amide | Compound 128 | 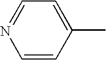 | 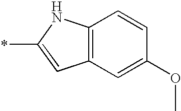 | B | + | ++ |
| 5-Fluoro-1H-indole-2-carboxylic acid {2-amino-2-[4-(pyridin-4- ylcarbamoyl)-phenyl]-ethyl}-amide | Compound 129 | 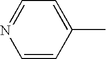 | 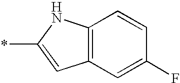 | B | + | ++ |
| 1H-Indole-2-carboxylic acid {2- amino-2-[4-(pyridin-4-ylcarbamoyl)- phenyl]-ethyl}-amide | Compound 130 | 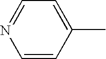 | 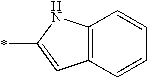 | B | + | ++ |
| 1H-Indole-6-carboxylic acid {2- amino-2-[4-(pyridin-4-ylcarbamoyl)- phenyl]-ethyl}-amide | Compound 131 | 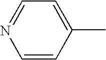 | 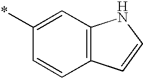 | B | + | ++ |
| 1H-Indole-5-carboxylic acid {2- amino-2-[4-(pyridin-4-ylcarbamoyl)- phenyl]-ethyl}-amide | Compound 132 | 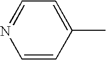 | 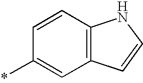 | B | ++ | +++ |
| 7-Methoxy-benzofuran-2-carboxylic acid {2-amino-2-[4-(pyridin-4- ylcarbamoyl)-phenyl]-ethyl}-amide | Compound 133 | 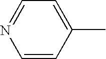 | 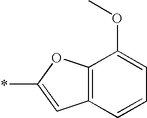 | B | + | +++ |
| 5-Chloro-benzofuran-2-carboxylic acid {2-amino-2-[4-(pyridin-4- ylcarbamoyl)-phenyl]-ethyl}-amide | Compound 134 | 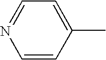 | 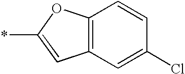 | B | + | ++ |
| 4-[1-Amino-2-(2-naphtalen-2-yl- acetylamino)-ethyl]-N-pyridin-4-yl- bennzamide | Compound 135 | 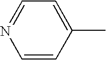 | 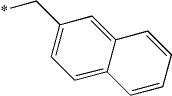 | B | + | ++ |
| 4-[1-Amino-2-(2-cyclopentyl-2- phenyl-acetylamino)-ethyl]-N- pyridin-4-yl-bennzamide | Compound 136 | 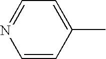 | 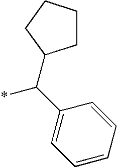 | B | + | ++ |
| 4-{1-Amino-2-[(1-phenyl- cyclopropanecarbonyl)-amino]- ethyl}-N-pyridin-4-yl-benzamide | Compound 137 | 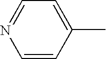 | 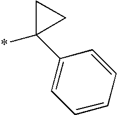 | B | +++ | ++ |
| 4-{1-amino-2-[2-(2-methoxy-phenyl)- acetylamino]-ethyl}-N-pyridin-4-yl- benzamide | Compound 138 | 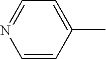 | 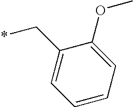 | B | + | ++ |
| 4-{1-amino-2-[2-(3-methoxy-phenyl)- acetylamino]-ethyl}-N-pyridin-4-yl- benzamide | Compound 139 | 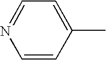 | 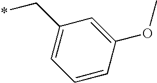 | B | ++ | +++ |
| 4-{1-amino-2-[2-(4-methoxy-phenyl)- acetylamino]-ethyl}-N-pyridin-4-yl- benzamide | Compound 140 | 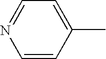 | 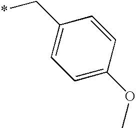 | B | + | +++ |
| 4-{1-amino-2-(2-benzo[1,3]dioxol-5- yl-acetylamino)-ethyl]-N-pyridin-4-yl- benzamide | Compound 141 | 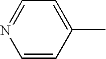 | 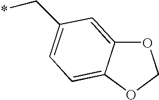 | B | + | +++ |
| 4-{1-Amino-2-[2-(2-methyl-phenyl)- acetylamino]-ethyl}-N-pyridin-4-yl- benzamide | Compound 142 | 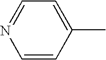 | 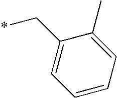 | B | ++ | +++ |
| 4-{1-Amino-2-[2-(3-methyl-phenyl)- acetylamino]-ethyl}-N-pyridin-4-yl- benzamide | Compound 143 | 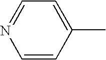 | 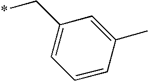 | B | ++ | +++ |
| 4-{1-Amino-2-[2-(2,5-dimethyl- phenyl)-aceylamino]-ethyl}-N- pyridin-4-yl-benzamide | Compound 144 | 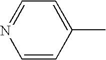 | 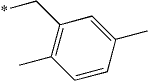 | B | ++ | ++ |
| 4-{1-Amino-2-[2-(4-methyl-phenyl)- acetylamino]-ethyl}-N-pyridin-4-yl- benzamide | Compound 145 | 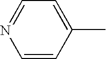 | 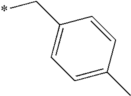 | B | ++ | +++ |
| 4-[1-Amino-2-(2-biphenyl-4-yl- acetylamino)-ethyl]-N-pyridin-4-yl- benzamide | Compound 146 | 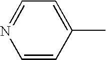 | 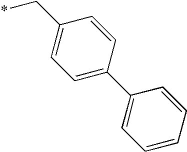 | B | + | ++ |
| 4-{1-Amino-2-[2-(3-chloro-phenyl)- acetylamino]-ethyl}-N-pyridin-4-yl- benzamide | Compound 147 | 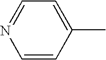 | 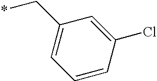 | B | ++ | +++ |
| 4-{1-Amino-2-[2-(4-fluoro-phenyl)- acetylamino]-ethyl}-N-pyridin-4-yl- benzamide | Compound 148 | 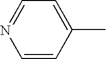 | 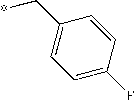 | B | +++ | +++ |
| 4-{1-Amino-2-[2-(2,6-difluororo- phenyl)-acetylamino]-ethyl}-N- pyridin-4-yl-benzamide | Compound 149 | 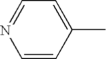 | 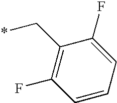 | B | ++ | ++ |
| 4-[1-Amino-2-(2-pyridin-2-yl- acetylamino)-ethyl]-N-pyridin-4-yl- benzamide | Compound 150 | 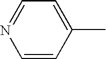 | 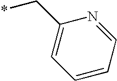 | B | + | ++ |
| 4-[1-Amino-2-(2-pyridin-4-yl- acetylamino)-ethyl]-N-pyridin-4-yl- benzamide | Compound 151 | 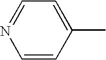 | 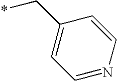 | B | + | ++ |
| 4-[1-Amino-2-(2-1H-indol-3-yl- acetylamino)-ethyl]-N-pyridin-4-yl- benzamide | Compound 152 | 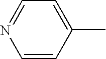 | 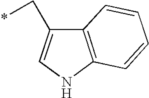 | B | ++ | +++ |
| 4-[1-amino-2-(2-thiophen-2-yl- acetylamino)-ethyl]-N-pyridin-4-yl- benzamide | Compound 153 | 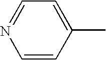 | 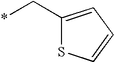 | B | ++ | +++ |
| 4-{1-Amino-2-[2-(2-phenyl-thiazol-4- yl)-acetylamino]-ethyl}-N-pyridin-4- yl-benzamide | Compound 154 | 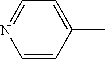 | 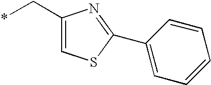 | B | + | +++ |
| 4-{1-Amino-2-[2-(2-phenylamino- thiazol-4-yl)-acetylamino]-ethyl}-N- pyridin-4-yl-benzamide | Compound 155 | 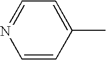 | 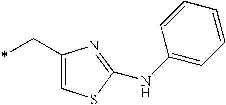 | B | ++ | ++ |
| 4-[1-Amino-2-(2-phenoxy- acetylamino)-ethyl]-N-pyridin-4-yl- benzamide | Compound 156 | 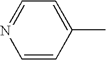 | 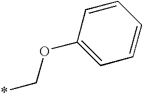 | B | +++ | +++ |
| 4-{1-Amino-2-[2-(4-fluoro-phenoxy)- acetylamino]-ethyl}-N-pyridin-4-yl- benzamide | Compound 157 | 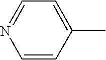 | 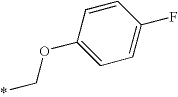 | B | +++ | +++ |
| 4-[1-Amino-2-(2-phenoxy- butyrylamino)-ethyl]-N-pyridin-4-yl- benzamide | Compound 158 | 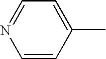 | 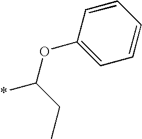 | B | ++ | +++ |
| 4-{1-Amino-2-[2-(4-chloro-2-methyl- phenoxy)-propionylamino]-ethyl}-N- pyridin-4-yl-benzamide | Compound 159 | 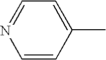 | 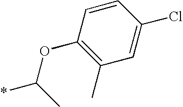 | B | ++ | +++ |
| 4-[1-Amino-2-(3,3-diphenyl- propionylamino)-ethyl]-N-pyridin-4- yl-benzamide | Compound 160 | 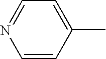 | 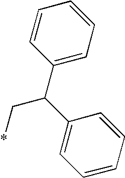 | B | + | +++ |
| Bicyclo[4.2.0]octa-1,3,5-triene-7- carboxylic acid {2-amino-2-[4- (pyridin-4-ylcarbamoyl)-phenyl]- ethyl}-amide | Compound 161 | 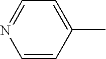 | 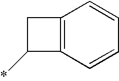 | B | ++ | +++ |
| 4-[1-amino-2-(3-phenyl)- propionylamino)-ethyl]-N-pyridin-4- yl-benzamide | Compound 162 | 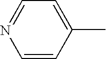 | 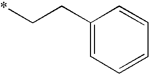 | B | ++ | +++ |
| 4-{1-amino-2-[3-(4-fluoro-phenyl)- propionylamino]-ethyl}-N-pyridin-4- yl-benzamide | Compound 163 | 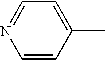 | 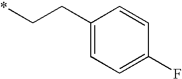 | B | +++ | +++ |
| 1,2,3,4-Tetrahydro-naphtalene-2- carboxylic acid {2-amino-2-[4- (pyridin-4-ylcarbamoyl)-phenyl]- ethyl}-amide | Compound 164 | 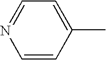 | 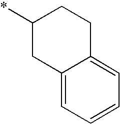 | B | ++ | ++ |
| 3-Methyl-1H-indene-2-carboxylic acid {2-amino-2-[4-(pyridin-4- ylcarbamoyl)-phenyl]-ethyl}-amide | Compound 165 | 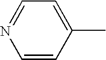 | 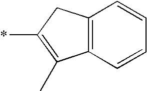 | B | ++ | ++ |
| 4-[1-Amino-2-(3-pyridin-3-yl- propionylamino)-ethyl]-N-pyridin-4- yl-benzamide | Compound 166 | 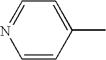 | 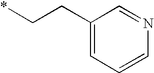 | B | + | ++ |
| 4-{1-Amino-2-[2-(3,4-difluoro- phenyl)-acetylamino]-ethyl}-N- pyridin-4-yl-benzamide dihydrochloride | Compound 167 | 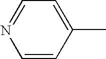 | 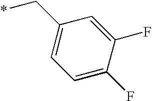 | B or C | +++ | +++ |
| 4-(1-Amino-2-{[1-(4-chloro-phenyl)- cyclopropanecarbonyl]-amino}- ethyl)-N-pyridin-4-yl-benzamide dihydrochloride | Compound 168 | 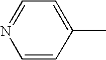 | 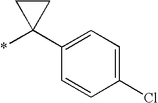 | B or C | ++++ | +++ |
| 4-{1-Amino-2-[2-(3-fluoro-4- methoxy-phenyl)-acetylamino]- ethyl}-N-pyridin-4-yl-benzamide | Compound 327 | 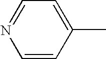 | 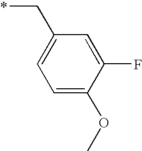 | C | ++ | +++ |
| 4-(1-Amino-2-phenylacetylamino- ethyl)-N-pyridin-4-yl-benzamide | Compound 328 | 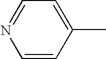 | 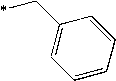 | C | +++ | nd |
| 4-{1-Amino-2-[2-(3,4-dichloro- phenyl)-acetylamino]-ethyl}-N- pyridin-4-yl-benzamide | Compound 329 | 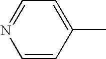 | 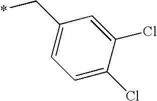 | C | +++ | nd |
| 4-{1-Amino-2-[2-(3-chloro-4-fluoro- phenyl)-acetylamino]-ethyl}-N- pyridin-4-yl-benzamide | Compound 330 | 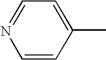 | 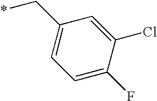 | C | +++ | nd |
| 4-{1-Amino-2-[2-(2,5-difluoro- phenyl)-acetylamino]-ethyl}-N- pyridin-4-yl-benzamide | Compound 331 | 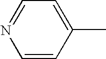 | 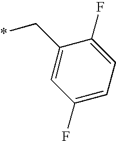 | C | ++ | nd |
| 4-{1-Amino-2-[2-(2-chloro-4-fluoro- phenyl)-acetylamino]-ethyl}-N- pyridin-4-yl-benzamide | Compound 332 | 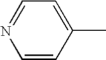 | 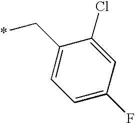 | C | +++ | nd |
| 4-{1-Amino-2-[2-(4-chloro-2-fluoro- phenyl)-acetylamino]-ethyl}-N- pyridin-4-yl-benzamide | Compound 333 | 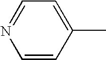 | 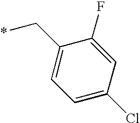 | C | +++ | nd |
| 4-[1-Amino-2-(2-ethyl-2-phenyl- butyrylamino)-ethyl]-N-pyridin-4-yl- benzamide | Compound 334 | 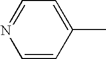 | 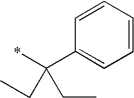 | C | ++++ | nd |
| 4-{1-Amino-2-[2-(4-chloro-phenyl)-2- methyl-propionylamino]-ethyl}-N- pyridin-4-yl-benzamide | Compound 335 | 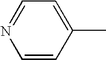 | 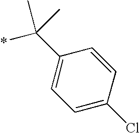 | C | ++++ | nd |
| 4-(1-Amino-2-{[1-(4-fluoro-phenyl)- cyclopentanecarbonyl]-amino}- ethyl)-N-pyridin-4-yl-benzamide | Compound 336 | 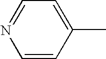 | 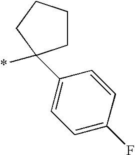 | C | ++++ | nd |
| 4-(1-Amino-2-{[1-(4-chloro-phenyl)- cyclobutanecarbonyl]-amino}-ethyl)- N-pyridin-4-yl-benzamide | Compound 337 | 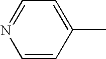 | 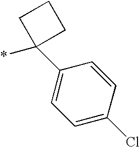 | C | +++ | nd |
| 4-(1-Amino-2-{[1-(4-fluoro-phenyl)- cyclobutanecarbonyl]-amino}-ethyl)- N-pyridin-4-yl-benzamide | Compound 338 | 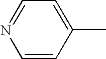 | 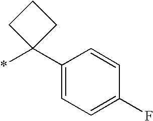 | C | ++++ | nd |
| 4-{1-Amino-2-[2-(4-fluoro-phenyl)-2- methyl-propionylamino]-ethyl}-N- pyridin-4-yl-benzamide | Compound 339 | 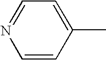 | 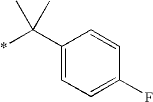 | C | ++++ | +++ |
| 4-(1-Amino-2-{[1-(4-fluoro-phenyl)- cyclopropanecarbonyl]-amino}- ethyl)-N-pyridin-4-yl-benzamide | Compound 340 | 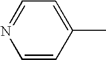 | 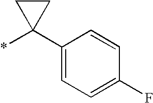 | C | ++++ | <0.2 |
| 4-(1-Amino-2-{[1-(3-fluoro-phenyl)- cyclopentanecarbonyl]-amino}- ethyl)-N-pyridin-4-yl-benzamide | Compound 341 | 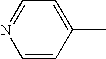 | 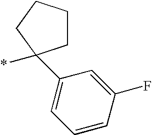 | C | +++ | nd |
| 4-(1-Amino-2-{[1-(2-chloro-4-fluoro- phenyl)-cyclopentanecarbonyl]- amino}-ethyl)-N-pyridin-4-yl- benzamide | Compound 342 | 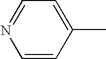 | 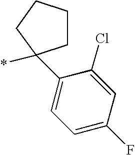 | C | ++++ | nd |
| 4-(1-Amino-2-{[1-(4-fluoro-phenyl)- cyclohexanecarbonyl]-amino}-ethyl)- N-pyridin-4-yl-benzamide | Compound 343 | 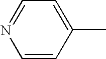 | 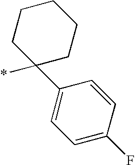 | C | ++++ | nd |
| 4-(1-Amino-2-{[1-(4-methoxy- phenyl)-cyclopentanecarbonyl]- amino}-ethyl)-N-pyridin-4-yl- benzamide | Compound 344 | 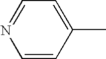 | 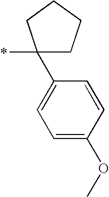 | C | +++ | +++ |
| 4-(1-Amino-2-{[1-(3,4-dichloro- phenyl)-cyclopropanecarbonyl]- amino}-ethyl)-N-pyridin-4-yl- benzamide | Compound 345 | 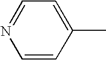 | 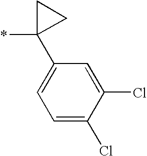 | C | +++ | +++ |
| 4-[1-Amino-2-(2-methyl-2-thiophen- 3-yl-propionylamino)-ethyl]-N- pyridin-4-yl-benzamide | Compound 346 | 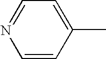 | 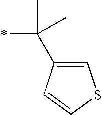 | C | +++ | +++ |
| 4-{1-Amino-2-[(1-thiophen-3-yl- cyclopropanecarbonyl)-amino]- ethyl}-N-pyridin-4-yl-benzamide | Compound 347 | 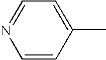 | 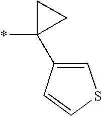 | C | +++ | +++ |
| 4-[1-Amino-2-(2-methyl-2-thiophen- 2-yl-propionylamino)-ethyl]-N- pyridin-4-yl-benzamide | Compound 348 | 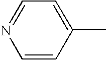 | 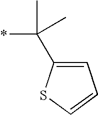 | C | ++++ | +++ |
| 4-{1-Amino-2-[(1-thiophen-2-yl- cyclopropanecarbonyl)-amino]- ethyl}-N-pyridin-4-yl-benzamide | Compound 349 | 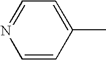 | 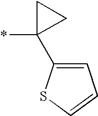 | C | +++ | +++ |
| 1,2,3,4-Tetrahydro-naphthalene-1- carboxylic acid {2-amino-2-[4- (pyridin-4-ylcarbamoyl)-phenyl]- ethyl}-amide | Compound 350 | 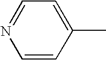 | 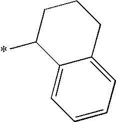 | C | +++ | nd |
| Indan-1-carboxylic acid {2-amino-2- [4-(pyridin-4-ylcarbamoyl)-phenyl]- ethyl}-amide | Compound 351 | 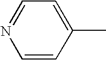 | 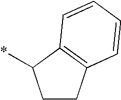 | C | +++ | nd |
| 5-Chloro-indan-1-carboxylic acid {2- amino-2-[4-(pyridin-4-ylcarbamoyl)- phenyl]-ethyl}-amide | Compound 352 | 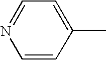 | 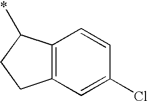 | C | ++++ | nd |
| 7-Fluoro-indan-1-carboxylic acid {2- amino-2-[4-(pyridin-4-ylcarbamoyl)- phenyl]-ethyl}-amide | Compound 353 | 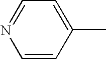 | 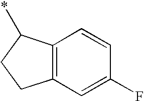 | C | +++ | nd |
| 6-Fluoro-indan-1-carboxylic acid {2- amino-2-[4-(pyridin-4-ylcarbamoyl)- phenyl]-ethyl}-amide | Compound 354 | 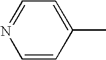 | 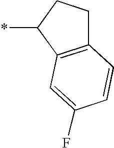 | C | +++ | nd |
| 6-Methoxy-1,2,3,4-tetrahydro- naphthalene-1-carboxylic acid {2- amino-2-[4-(pyridin-4-ylcarbamoyl)- phenyl]-ethyl}-amide | Compound 355 | 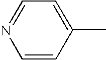 | 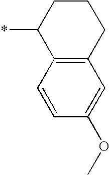 | C | +++ | +++ |
| 3-Fluoro-6,7,8,9-tetrahydro-5H- benzocycloheptene-5-carboxylic acid {2- amino-2-[4-(pyridin-4-ylcarbamoyl)- phenyl]-ethyl}-amide | Compound 356 | 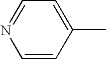 | 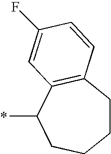 | C | ++++ | +++ |
| 4-(1-Amino-2-{[1-(4-chloro-phenyl)- cyclopropanecarbonyl]-amino}- ethyl)-N-(1H-pyrazolo[3,4-b]pyridin- 4-yl)-benzamide | Compound 652 | 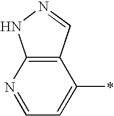 | 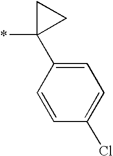 | | nd | nd |
| 4-{1-Amino-2-[2-(3,4-difluoro- phenyl)-2-methyl-propionylamino]- ethyl}-N-pyridin-4-yl-benzamide | Compound 376 | 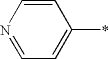 | 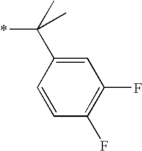 | C | ++++ | nd |
| 4-(1-Amino-2-{[1-(3,4-difluoro- phenyl)-cyclopropanecarbonyl]- amino}-ethyl)-N-pyridin-4-yl- benzamide | Compound 377 | 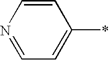 | 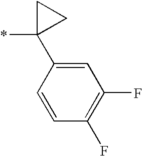 | C | ++++ | +++ |
| 4-(1-Amino-2-{[1-(3,4-difluoro- phenyl)-cyclobutanecarbonyl]- amino}-ethyl)-N-pyridin-4-yl- benzamide | Compound 378 | 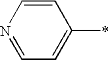 | 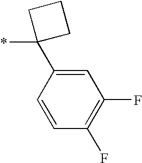 | C | ++++ | +++ |
| 4-(1-Amino-2-{[1-(3,4-difluoro- phenyl)-cyclopentanecarbonyl]- amino}-ethyl)-N-pyridin-4-yl- benzamide | Compound 379 | 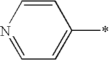 | 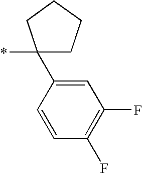 | C | ++++ | +++ |
| 4-{1-Amino-2-[2-(3,4-dichloro- phenyl)-2-methyl-propionylamino]- ethyl}-N-pyridin-4-yl-benzamide | Compound 380 | 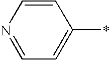 | 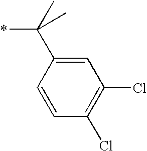 | C | ++++ | +++ |
| 4-{1-Amino-2-[2-(4-fluoro-phenyl)-2- methyl-propionylamino]-ethyl}-N-(1H- pyrrolo[2,3-b]pyridin-4-yl)-benzamide | Compound 649 | 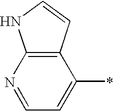 | 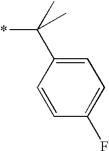 | | nd | nd |
| 4-(1-Amino-2-{[1-(3,4-dichloro- phenyl)-cyclobutanecarbonyl]- amino}-ethyl)-N-pyridin-4-yl- benzamide | Compound 381 | 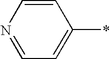 | 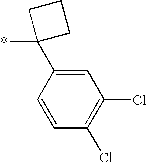 | C | +++ | +++ |
| 4-{1-Amino-2-[2-(3-chloro-4-fluoro- phenyl)-2-methyl-propionylamino]- ethyl}-N-pyridin-4-yl-benzamide | Compound 382 | 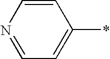 | 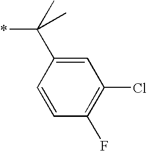 | C | ++++ | +++ |
| 4-(1-Amino-2-{[1-(3-chloro-4-fluoro- phenyl)-cyclopropanecarbonyl]- amino}-ethyl)-N-pyridin-4-yl- benzamide | Compound 383 | 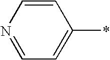 | 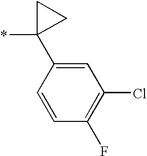 | C | nd | nd |
| 4-{1-Amino-2-[2-(2,4-difluoro- phenyl)-2-methyl-propionylamino]- ethyl}-N-pyridin-4-yl-benzamide | Compound 384 | 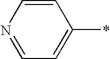 | 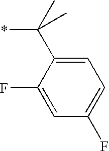 | C | ++++ | +++ |
| 4-{1-Amino-2-[2-(4-chloro-phenyl)-2- methyl-propionylamino]-ethyl}-N-(1H- pyrrolo[2,3-b]pyridin-4-yl)-benzamide | Compound 648 | 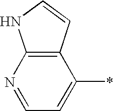 | 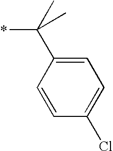 | | nd | nd |
| 4-(1-Amino-2-{[1-(2,4-difluoro- phenyl)-cyclopropanecarbonyl]- amino}-ethyl)-N-pyridin-4-yl- benzamide | Compound 385 | 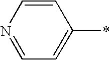 | 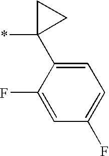 | C | ++++ | +++ |
| 4-(1-Amino-2-{[1-(4-chloro-phenyl)- cyclopropanecarbonyl]-amino}- ethyl)-N-(1H-pyrrolo[2,3-b]pyridin-4- yl)-benzamide | Compound 647 | 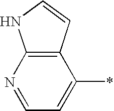 | 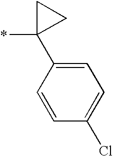 | | nd | nd |
| 4-{1-Amino-2-[2-(2-chloro-4-fluoro- phenyl)-2-methyl-propionylamino]- ethyl}-N-pyridin-4-yl-benzamide | Compound 386 | 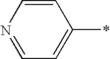 | 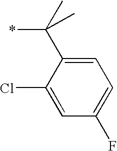 | C | ++++ | +++ |
| 4-(1-Amino-2-{[1-(2-chloro-4-fluoro- phenyl)-cyclopropanecarbonyl]- amino}-ethyl)-N-pyridin-4-yl- benzamide | Compound 387 | 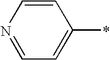 | 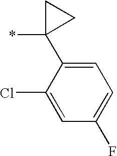 | C | ++++ | +++ |
| 4-{1-Amino-2-[(1-phenyl- cyclopropanecarbonyl)-amino]- ethyl}-N-(1H-pyrrolo[2-3-b]pyridin-4- yl)-benzamide | Compound 646 | 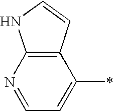 | 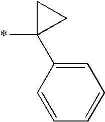 | | nd | nd |
| 4-{1-Amino-2-[2-(4-chloro-2-fluoro- phenyl)-2-methyl-propionylamino]- ethyl}-N-pyridin-4-yl-benzamide | Compound 388 | 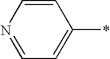 | 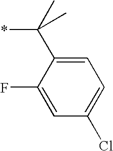 | C | ++++ | +++ |
| 4-(1-Amino-2-{[1-(4-chloro-2-fluoro- phenyl)-cyclopropanecarbonyl]- amino}-ethyl)-N-pyridin-4-yl- benzamide | Compound 389 | 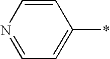 | 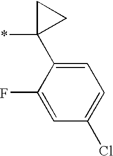 | C | ++++ | +++ |
| Chroman-4-carboxylic acid {2- amino-2-[4-(pyridin-4-ylcarbamoyl)- phenyl]-ethyl}-amide | Compound 390 | 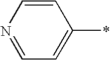 | 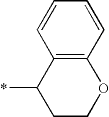 | C | +++ | +++ |
| Thiochroman-4-carboxylic acid {2- amino-2-[4-(pyridin-4-ylcarbamoyl)- phenyl]-ethyl}-amide | Compound 391 | 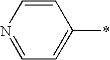 | 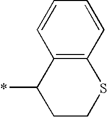 | C | +++ | +++ |
| 6-Fluoro-thiochroman-4-carboxylic acid {2-amino-2-[4-(pyridin-4- ylcarbamoyl)-phenyl]-ethyl}-amide | Compound 392 | 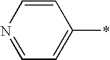 | 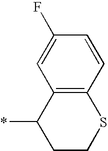 | C | nd | nd |
| 6-Fluoro-chroman-4-carboxylic acid {2-amino-2-[4-(pyridin-4- ylcarbamoyl)-phenyl]-ethyl}-amide | Compound 393 | 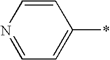 | 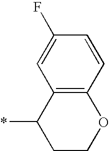 | C | +++ | +++ |
| 6-Chloro-chroman-4-carboxylic acid {2-amino-2-[4-(pyridin-4- ylcarbamoyl)-phenyl]-ethyl}-amide | Compound 394 | 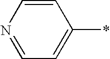 | 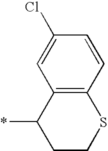 | C | nd | nd |
| 1,2,3,4-Tetrahydro-phenanthrene-4- carboxylic acid {2-amino-2-[4- (pyridin-4-ylcarbamoyl)-phenyl]- ethyl}-amide | Compound 395 | 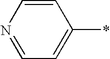 | 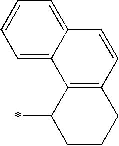 | C | nd | nd |
| 4-(1-Amino-2-[2-(5-chloro-thiophen- 2-yl)-acetylamino]-ethyl}-N-pyridin- 4-yl-benzamide | Compound 396 | 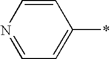 | 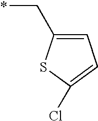 | C | nd | nd |
| 4-{1-Amino-2-[2-(3,4-dichloro- phenyl)-2-methyl-propionylamino]- ethyl}-N-(1H-pyrrolo[2,3-b]pyridin-4- yl)-benzamide | Compound 650 | 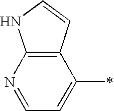 | 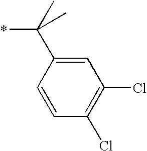 | | nd | nd |
| 4-{1-Amino-2-[(1-phenyl- cyclopropanecarbonyl)-amino]- ethyl}-N-(1H-pyrazolo[3,4-b]pyridin- 4-yl)-benzamide | Compound 651 | 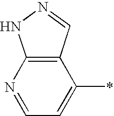 | 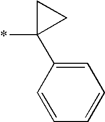 | | nd | nd |
| 4-{1-Amino-2-[2-(4-chloro-phenyl)-2- methyl-propionylamino]-ethyl}-N-(1H- pyrazolo[3,4-b]pyridin-4-yl)- benzamide | Compound 653 | 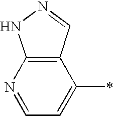 | 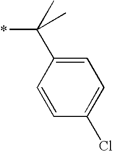 | | nd | nd |
| 4-{1-Amino-2-[2-(4-fluoro-phenyl)-2- methyl-propionylamino]-ethyl}-N-(1H- pyrazolo[3,4-b]pyridin-4-yl)- benzamide | Compound 654 | 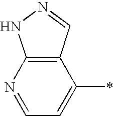 | 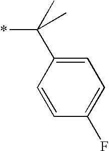 | | nd | nd |
| 4-{1-Amino-2-[2-(3,4-dichloro- phenyl)-2-methyl-propionylamino]- ethyl}-N-(1H-pyrazolo[3,4-b]pyridin- 4-yl)-benzamide | Compound 655 | 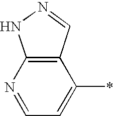 | 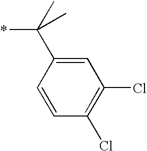 | | nd | nd |
| 4-{1-Amino-2-[2-(4-chloro-phenyl)-2- methyl-propionylamino]-ethyl}-N-(3H- imidazo[4,5-b]pyridin-7-yl)- benzamide | Compound 661 | 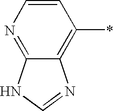 | 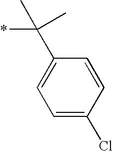 | D | nd | nd |
| 4-{1-Amino-2-[2-(4-chloro-phenyl)-2- methyl-propionylamino]-ethyl}-N-(9H- purin-6-yl)-benzamide | Compound 662 | 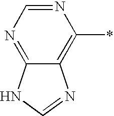 | 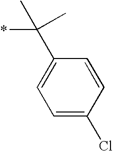 | D | nd | nd |

Table 3 shows the results for compounds of Formula XIV. As used herein the term “nd” means “not determined yet” and “Pr” is “Protocol”.

TABLE 3

| | |
|---|---|
| | XIV |
| 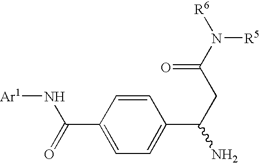 | |

| | | | | | | IC₅₀ | IC₅₀ |
|---|---|---|---|---|---|---|---|
| | | | | | | μM | μM |
| Name | Compound | Ar¹— | —R⁵ | —R⁶ | Pr | PKCε | ROCK |
| 4-(1-amino-2- ethylcarbamoyl-ethyl-N- pyridin-4-yl-benzamide | Compound 169 | 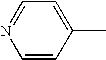 | —H | 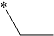 | B | ++ | +++ |
| 4-[1-amino-2-(1,2-dimethyl- propylcarbamoyl)-ethyl]-N- pyridin-4-yl-benzamide | Compound 170 | 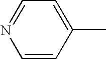 | —H | 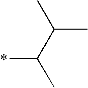 | B | ++ | +++ |
| 4-[1-amino-2-(cyclopropyl- methylcarbamoyl)-ethyl]-N- pyridin-4-yl-benzamide | Compound 171 | 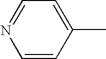 | —H | 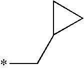 | B | ++ | +++ |
| 4-(1-amino-2- cyclohexylcarbamoyl-ethyl)- N-pyridin-4-yl-benzamide | Compound 172 | 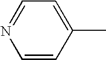 | —H | 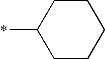 | B | ++ | +++ |
| 4-[1-amino-2-(4-methyl- cyclohexylcarbamoyl)-ethyl]- N-pyridin-4-yl-benzamide | Compound 173 | 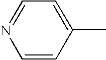 | —H | 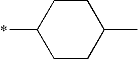 | B | ++ | +++ |
| 4-[1-amino-2-(2- dimethylamino- ethylcarbamoyl)-ethyl]-N- pyridin-4-yl-benzamide | Compound 174 | 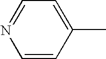 | —H | 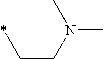 | B | ++ | +++ |
| 4-[2-(2-acetylamino- ethylcaramoyl)-1-amino- ethyl]-N-pyridin-4-yl- benzamide | Compound 175 | 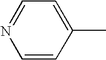 | —H | 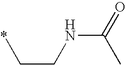 | B | ++ | +++ |
| 4-[1-amino-2-(2- methanesulfonylamino- ethylcarbamoyl)-ethyl]-N- pyridin-4-yl-benzamide | Compound 176 | 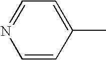 | —H | 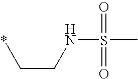 | B | ++ | +++ |
| 4-[1-amino-2-(2-morpholin- 4-yl-ethylcarbamoyl)-ethyl]- N-pyridin-4-yl-benzamide | Compound 177 | 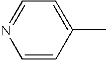 | —H | 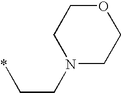 | B | + | +++ |
| 4-{1-amino-2-[(2-cyano- ethyl)-cyclopropyl- carbamoyl]-ethyl}-N-pyridin- 4-yl-benzamide | Compound 178 | 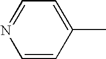 | 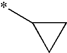 | 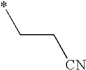 | B | + | ++ |
| 4-{1-amino-2-[(2-hydroxy- ethyl)-methyl-carbamoyl]- ethyl}-N-pyridin-4-yl- benzamide | Compound 179 | 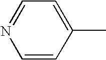 | —CH₃ | 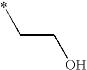 | B | ++ | +++ |
| 4-[1-amino-2-(2-methoxy- ethylcarbamoyl)-ethyl]-N- pyridin-4-yl-benzamide | Compound 180 | 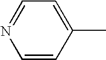 | —H | 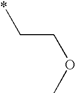 | B | ++ | +++ |
| 4-[1-amino-2-(2-methoxy-1- methyl-ethylcarbamoyl)- ethyl]-N-pyridin-4-yl- benzamide | Compound 181 | 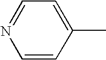 | —H | 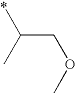 | B | ++ | +++ |
| 2-{3-amino-3-[4-pyridin-4- ylcarbamoyl)-phenyl]- propionylamino}-propionic acid methyl ester | Compound 182 | 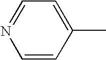 | —H | 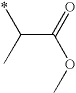 | B | ++ | +++ |
| 4-(1-amino-2- methoxycarbamoyl-ethyl)-N- pyridin-4-yl-benzamide | Compound 183 | 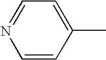 | —H | 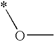 | B | ++ | +++ |
| 4-{1-amino-2-[(tetrahydro- furan-2-ylmethyl)- carbamoyl]-ethyl}-N-pyridin- 4-yl-benzamide | Compound 184 | 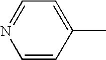 | —H | 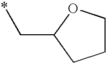 | B | ++ | +++ |
| 4-[1-amino-2-(1-benzyl- piperidin-4-ylcarbamoyl)- ethyl]-N-pyridin-4-yl- benzamide | Compound 200 | 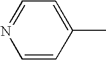 | —H | 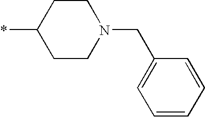 | B | ++ | +++ |
| 4-{1-amino-2-[1-(3-methyl- benzyl)-piperidin-4- ylcarbamoyl]-ethyl}-N- pyridin-4-yl-benzamide | Compound 201 | 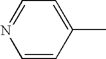 | —H | 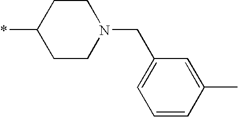 | B | ++ | +++ |
| 4-{1-amino-2-[(1-(5-methyl- thiophen-2-ylmethyl)- piperidin-4-ylcarbamoyl]- ethyl}-N-pyridin-4-yl- benzamide | Compound 202 | 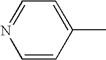 | —H | 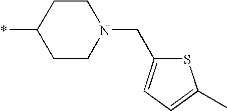 | B | ++ | +++ |
| 4-(1-Amino-2- benzylcarbamoyl-ethyl)-N- pyridin-4-yl-benzamide | Compound 213 | 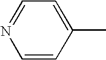 | —H | 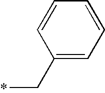 | B | ++ | +++ |
| 4-[1-Amino-2-(benzyl- methyl-carbamoyl)-ethyl]-N- pyridin-4-yl-benzamide | Compound 214 | 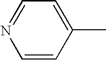 | —CH₃ | 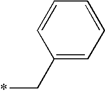 | B | nd | +++ |
| 4-[1-Amino-2-(1-phenyl- ethylcarbamoyl)-ethyl]-N- pyridin-4-yl-benzamide | Compound 215 | 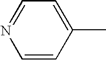 | —H | 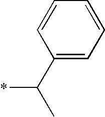 | B | ++ | +++ |
| 4-[1-Amino-2-(1-(R)-phenyl- propylcarbamoyl)-ethyl]-N- pyridin-4-yl-benzamide | Compound 216 | 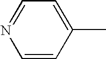 | —H | 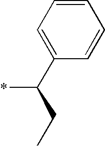 | B | ++ | +++ |
| 4-[1-Amino-2-(1-(S)-phenyl- propylcarbamoyl)-ethyl]-N- pyridin-4-yl-benzamide | Compound 217 | 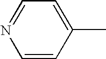 | —H | 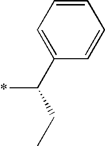 | B | ++ | +++ |
| 4-[1-Amino-2-(1-methyl-1- phenyl-ethylcarbamoyl)- ethyl]-N-pyridin-4-yl- benzamide | Compound 218 | 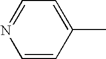 | —H | 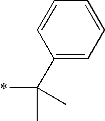 | B | ++ | +++ |
| 4-[1-Amino-2-(1-(S)-p-tolyl- propylcarbamoyl)-ethyl]-N- pyridin-4-yl-benzamide | Compound 219 | 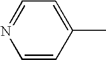 | —H | 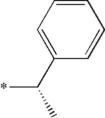 | B | ++ | +++ |
| 4-[1-Amino-2-(1-(R)-p-tolyl- propylcarbamoyl)-ethyl]-N- pyridin-4-yl-benzamide | Compound 220 | 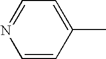 | —H | 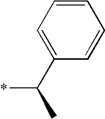 | B | ++ | +++ |
| 4-[1-Amino-2-(1,2,3,4- tetrahydro-naphtalen-1-(R)- ylcarbamoyl)-ethyl]-N- pyridin-4-yl-benzamide | Compound 221 | 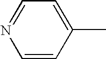 | —H | 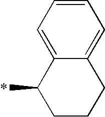 | B | +++ | +++ |
| 4-[1-Amino-2-(1,2,3,4- tetrahydro-naphtalen-1-(S)- ylcarbamoyl)-ethyl]-N- pyridin-4-yl-benzamide | Compound 222 | 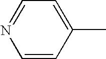 | —H | 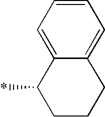 | B | + | +++ |
| 4-[1-Amino-2-(1,2,3,4- tetrahydro-naphtalen-1- ylcarbamoyl)-ethyl]-N- pyridin-4-yl-benzamide | Compound 223 | 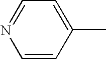 | —H | 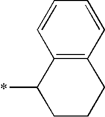 | B | +++ | +++ |
| 4-[1-Amino-2-(benzhydryl- carbamoyl)-ethyl]-N-pyridin- 4-yl-benzamide | Compound 225 | 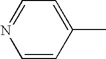 | —H | 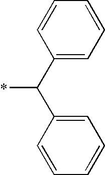 | B | ++ | +++ |
| 4-[1-Amino-2-(benzyl- phenethyl-carbamoyl)-ethyl]- N-pyridin-4-yl-benzamide | Compound 226 | 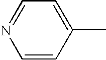 | 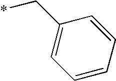 | 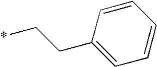 | B | ++ | +++ |
| 4-[1-Amino-2-(9H-fluoren-9- ylcarbamoyl)-ethyl]-N- pyridin-4-yl-benzamide | Compound 227 | 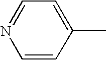 | —H | 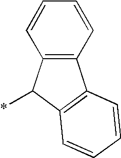 | B | ++ | +++ |
| 4-[1-Amino-2-(indan-1- ylcarbamoyl)-ethyl]-N- pyridin-4-yl-benzamide | Compound 228 | 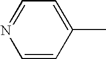 | —H | 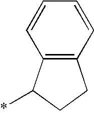 | B | +++ | +++ |
| 4-{1-Amino-2-(1-naphtalen- 1-yl-ethylcarbamoyl)-ethyl]- N-pyridin-4-yl-benzamide | Compound 229 | 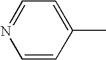 | —H | 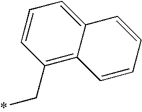 | B | ++ | +++ |
| 4-[1-Amino-2-(1-naphtalen- 1-yl-ethylcarbamoyl)-ethyl]- N-pyridin-4-yl-benzamide | Compound 230 | 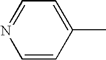 | —H | 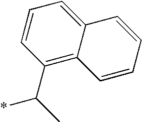 | B | ++ | +++ |
| 4-[1-Amino-2-(2-methyl- benzylcarbamoyl)-ethyl]-N- pyridin-4-yl-benzamide | Compound 231 | 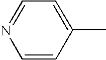 | —H | 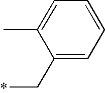 | B | ++ | +++ |
| 4-[1-Amino-2-(3-methyl- benzylcarbamoyl)-ethyl]-N- pyridin-4-yl-benzamide | Compound 232 | 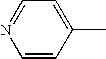 | —H | 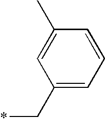 | B | ++ | +++ |
| 4-[1-Amino-2-(4-methyl- benzylcarbamoyl)-ethyl]-N- pyridin-4-yl-benzamide | Compound 233 | 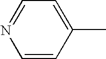 | —H | 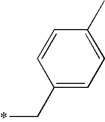 | B | ++ | +++ |
| 4-[1-Amino-2-(2,5-dimethyl- benzylcarbamoyl)-ethyl]-N- pyridin-4-yl-benzamide | Compound 234 | 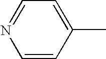 | —H | 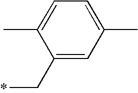 | B | ++ | +++ |
| 4-[1-Amino-2-(4-thiophen-2- yl-benzylcarbamoyl)-ethyl]- N-pyridin-4-yl-benzamide | Compound 235 | 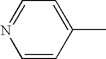 | —H | 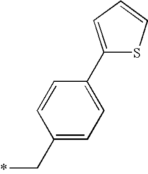 | B | + | +++ |
| 4-[1-Amino-2-(2-methoxy- benzylcarbamoyl)-ethyl]- N-pyridin-4-yl-benzamide | Compound 236 | 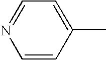 | —H | 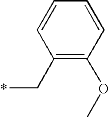 | B | ++ | +++ |
| 4-[1-Amino-2-(2-ethoxy- benzylcarbamoyl)-ethyl]- N-pyridin-4-yl-benzamide | Compound 237 | 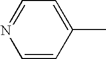 | —H | 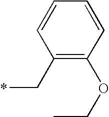 | B | ++ | +++ |
| 4-[1-Amino-2-(2,3- dimethoxy- benzylcarbamoyl)-ethyl]-N- pyridin-4-yl-benzamide | Compound 238 | 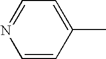 | —H | 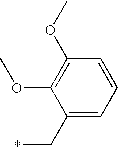 | B | + | +++ |
| 4-[1-Amino-2-(4-methoxy- benzylcarbamoyl)-ethyl]-N- pyridin-4-yl-benzamide | Compound 239 | 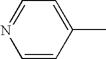 | —H | 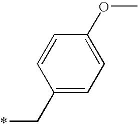 | B | ++ | +++ |
| 4-[1-Amino-2-(3,4- dimethoxy- benzylcarbamoyl)-ethyl]-N- pyridin-4-yl-benzamide | Compound 240 | 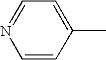 | —H | 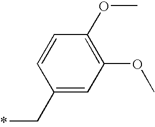 | B | + | +++ |
| 4-[1-Amino-2-(2,4- dimethoxy- benzylcarbamoyl)-ethyl]- N-pyridin-4-yl-benzamide | Compound 241 | 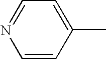 | —H | 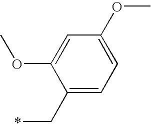 | B | ++ | +++ |
| 4-[1-Amino-2-(2,5- dimethoxy- benzylcarbamoyl)-ethyl]-N- pyridin-4-yl-benzamide | Compound 242 | 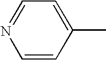 | —H | 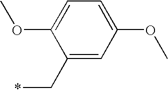 | B | ++ | +++ |
| 4-[1-Amino-2-(2,6- dimethoxy- benzylcarbamoyl)-ethy]-N- pyridin-4-yl-benzamide | Compound 243 | 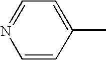 | —H | 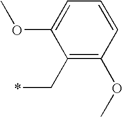 | B | ++ | +++ |
| 4-(1-Amino-2-{[bis-(4- methoxy-phenyl)-methyl]- carbamoyl}-ethyl)-N-pyridin- 4-yl-benzamide | Compound 244 | 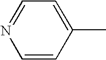 | —H | 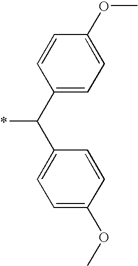 | B | ++ | +++ |
| 4-{1-Amino-2- [(benzo[1,3]dioxol-5- ylmethyl)-carbamoyl]-ethyl}- N-pyridin-4-yl-benzamide | Compound 245 | 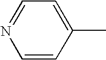 | —H | 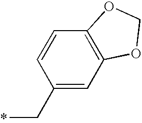 | B | ++ | +++ |
| 4-[1-Amino-2-(2-chloro- benzylcarbamoyl)-ethyl]-N- pyridin-4-yl-benzamide | Compound 246 | 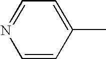 | —H | 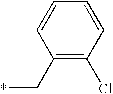 | B | ++ | +++ |
| 4-[1-Amino-2-(2-chloro-6- methyl-benzylcarbamoyl)- ethyl]-N-pyridin-4-yl- benzamide | Compound 247 | 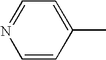 | —H | 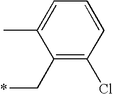 | B | ++ | +++ |
| 4-[1-Amino-2-(3-chloro- benzylcarbamoyl)-ethyl]-N- pyridin-4-yl-benzamide | Compound 248 | 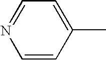 | —H | 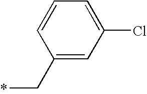 | B | +++ | +++ |
| 4-[1-Amino-2-(3-chloro-4- methyl-benzylcarbamoyl)- ethyl]-N-pyridin-4-yl- benzamide 7 | Compound 249 | 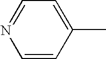 | —H | 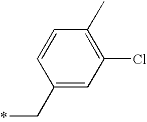 | B | ++ | +++ |
| 4-[1-Amino-2-(3,5-dichloro- benzylcarbamoyl)-ethyl]-N- pyridin-4-yl-benzamide | Compound 250 | 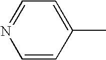 | —H | 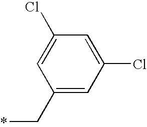 | B | ++ | +++ |
| 4-[1-Amino-2-(3,6-dichloro- benzylcarbamoyl)-ethyl]-N- pyridin-4-yl-benzamide | Compound 251 | 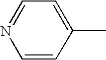 | —H | 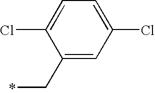 | B | ++ | +++ |
| 4-[1-Amino-2-(2,4-dichloro- benzylcarbamoyl)-ethyl]-N- pyridin-4-yl-benzamide | Compound 252 | 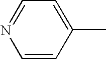 | —H | 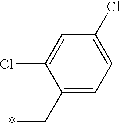 | B | ++ | +++ |
| 4-[1-Amino-2-(2,3-dichloro- benzylcarbamoyl)-ethyl]-N- pyridin-4-yl-benzamide | Compound 253 | 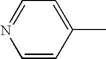 | —H | 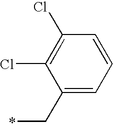 | B | +++ | +++ |
| 4-[1-Amino-2-(3,4-dichloro- 6-methyl-benzylcarbamoyl)- ethyl}-N-pyridin-4-yl- benzamide | Compound 254 | 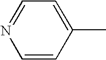 | —H | 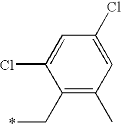 | B | ++ | +++ |
| 4-(1-Amino-2-{[(4-chloro- phenyl)-phenyl-methyl]- carbamoyl}-ethyl)-N-pyridin- 4-yl-benzamide | Compound 255 | 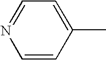 | —H | 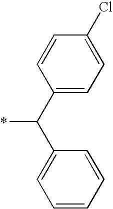 | B | ++ | +++ |
| 4-[1-Amino-2-(2-fluoro- benzylcarbamoyl)-ethyl]-N- pyridin-4-yl-benzamide | Compound 256 | 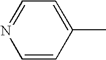 | —H | 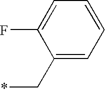 | B | ++ | +++ |
| 4-[1-Amino-2-(3-fluoro- benzylcarbamoyl)-ethyl]-N- pyridin-4-yl-benzamide | Compound 257 | 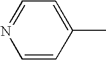 | —H | 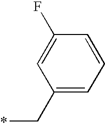 | B | ++ | +++ |
| 4-[1-Amino-2-(4-fluoro- benzylcarbamoyl)-ethyl]-N- pyridin-4-yl-benzamide | Compound 258 | 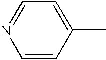 | —H | 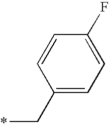 | B | +++ | +++ |
| 4-[1-Amino-2-(2-bromo- benzylcarbamoyl)-ethyl]-N- pyridin-4-yl-benzamide | Compound 259 | 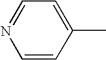 | —H | 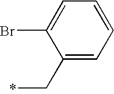 | B | ++ | +++ |
| 4-[1-Amino-2-(3-bromo- benzylcarbamoyl)-ethyl]-N- pyridin-4-yl-benzamide | Compound 260 | 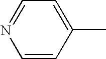 | —H | 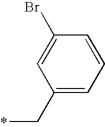 | B | ++ | +++ |
| 4-[1-Amino-2-(2-chloro-4- fluoro-benzylcarbamoyl)- ethyl]-N-pyridin-4-yl- benzamide | Compound 261 | 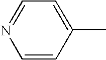 | —H | 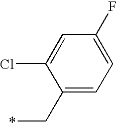 | B | ++ | +++ |
| 4-[1-Amino-2-(4-bromo-2- fluoro-benzylcarbamoyl)- ethyl]-N-pyridin-4-yl- benzamide | Compound 262 | 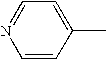 | —H | 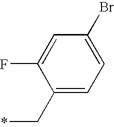 | B | ++ | +++ |
| 4-[1-Amino-2-(2- trifluoromethyl- benzylcarbamoyl)-ethyl]-N- pyridin-4-yl-benzamide | Compound 263 | 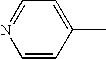 | —H | 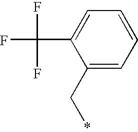 | B | ++ | +++ |
| 4-[1-Amino-2-(3- trifluoromethyl- benzylcarbamoyl)-ethyl]-N- pyridin-4-yl-benzamide | Compound 264 | 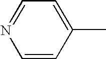 | —H | 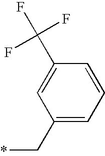 | B | ++ | +++ |
| 4-[1-Amino-2-(4- trifluoromethyl- benzylcarbamoyl)-ethyl]-N- pyridin-4-yl-benzamide | Compound 265 | 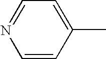 | —H | 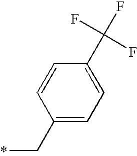 | B | ++ | +++ |
| 4-[1-Amino-2-(4-fluoro-3- trifluoromethyl- benzylcarbamoyl)-ethyl]-N- pyridin-4-yl-benzamide | Compound 266 | 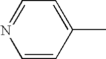 | —H | 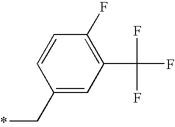 | B | ++ | +++ |
| 4-[1-Amino-2-(3-fluoro-4- trifluoromethyl- benzylcarbamoyl)-ethyl]-N- pyridin-4-yl-benzamide | Compound 267 | 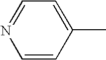 | —H | 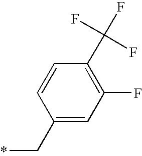 | B | ++ | +++ |
| 4-[1-Amino-2-(3,5-bis- trifluoromethyl- benzylcarbamoyl)-ethyl]-N- pyridin-4-yl-benzamide | Compound 268 | 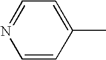 | —H | 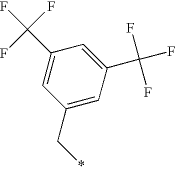 | B | ++ | +++ |
| 4-[1-Amino-2-(2- trifluoromethoxy- benzylcarbamoyl)-ethyl]-N- pyridin-4-yl-benzamide | Compound 269 | 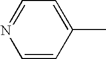 | —H | 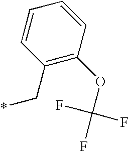 | B | ++ | +++ |
| 4-[1-Amino-2-(3- trifluoromethoxy- benzylcarbamoyl)-ethyl]-N- pyridin-4-yl-benzamide | Compound 270 | 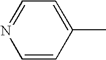 | —H | 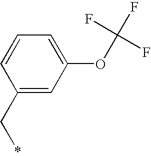 | B | ++ | +++ |
| 4-[1-Amino-2-(4- trifluoromethoxy- benzylcarbamoyl)-ethyl]-N- pyridin-4-yl-benzamide | Compound 271 | 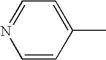 | —H | 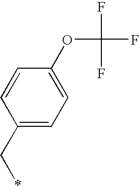 | B | ++ | +++ |
| 4-[1-Amino-2-(3-nitro- benzylcarbamoyl)-ethyl]-N- pyridin-4-yl-benzamide | Compound 272 | 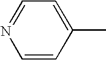 | —H | 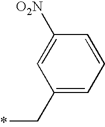 | B | ++ | +++ |
| 4-[1-Amino-2-(4-nitro- benzylcarbamoyl)-ethyl]-N- pyridin-4-yl-benzamide | Compound 273 | 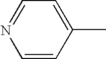 | —H | 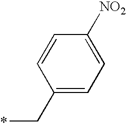 | B | ++ | +++ |
| 4-{1-Amino-2-[ethyl-(4-nitro- benzyl}-carbamoyl]-ethyl)-N- pyridin-4-yl-benzamide | Compound 274 | 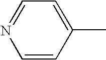 | 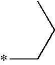 | 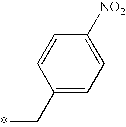 | B | ++ | +++ |
| 4-[1-Amino-2-(4- methanesulfonyl- benzylcarbamoyl)-ethyl]-N- pyridin-4-yl-benzamide | Compound 275 | 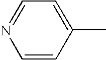 | —H | 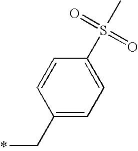 | B | ++ | +++ |
| 4-{1-Amino-2-[(pyridin-4- ylmethyl)-carbamoyl]-ethyl}- N-pyridin-4-yl-benzamide | Compound 276 | 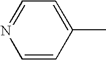 | —H | 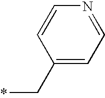 | B | ++ | +++ |
| 4-{1-Amino-2-[(pyridin-2- ylmethyl)-carbamoyl]-ethyl}- N-pyridin-4-yl-benzamide | Compound 277 | 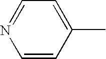 | —H | 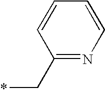 | B | ++ | +++ |
| 4-{1-Amino-2-[(furan-2- ylmethyl)-carbamoyl]-ethyl}- N-pyridin-4-yl-benzamide | Compound 278 | 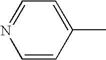 | —H | 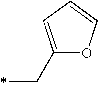 | B | ++ | +++ |
| 4-{1-Amino-2-[(thiophen-2- ylmethyl)-carbamoyl]-ethyl}- N-pyridin-4-yl-benzamide | Compound 279 | 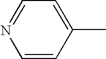 | —H | 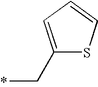 | B | ++ | +++ |
| 4-[1-amino-2-(indan-2- ylcarbamoyl)-ethyl]-N- pyridin-4-yl-benzamide | Compound 280 | 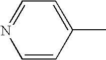 | —H | 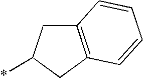 | B | ++ | +++ |
| 4-(1-Amino-2- phenethylcarbamoyl-ethyl)- N-pyridin-4-yl-benzamide | Compound 281 | 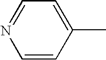 | —H | 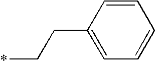 | B | ++ | +++ |
| 4-[1-Amino-2-(methyl- phenethyl-carbamoyl)-ethyl]- N-pyridin-4-yl-benzamide | Compound 282 | 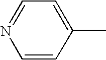 | —CH₃ | 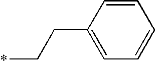 | B | +++ | +++ |
| 4-[1-Amino-2-(2-phenyl- propyl-carbamoyl)-ethyl]-N- pyridin-4-yl-benzamide | Compound 283 | 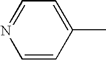 | —H | 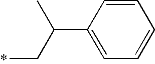 | B | ++ | +++ |
| 4-[1-Amino-2-(2-p-tolyl- ethyl-carbamoyl)-ethyl]-N- pyridin-4-yl-benzamide | Compound 284 | 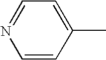 | —H | 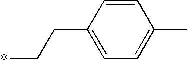 | B | + | +++ |
| 4-{1-Amino-2-[2-(4-tert- butyl-phenyl)- ethylcarbamoyl]-ethyl}-N- pyridin-4-yl-benzamide | Compound 285 | 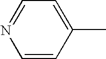 | —H | 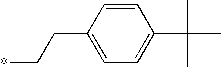 | B | + | +++ |
| 4-[1-Amino-2-(phenethyl- phenyl-carbamoyl)-ethyl]-N- pyridin-4-yl-benzamide | Compound 286 | 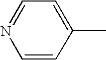 | —H | 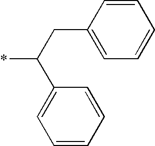 | B | ++ | +++ |
| 4-{1-Amino-2-[2-(2-ethoxy- phenyl)-ethylcarbamoyl]- ethyl}-N-pyridin-4-yl- benzamide | Compound 288 | 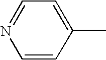 | —H | 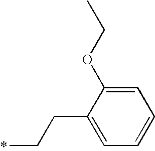 | B | ++ | +++ |
| 4-{1-Amino-2-[2-(3-methoxy- phenyl)-ethylcarbamoyl]- ethyl}-N-pyridin-4-yl- benzamide | Compound 289 | 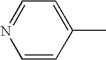 | —H | 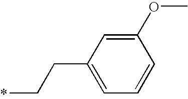 | B | ++ | +++ |
| 4-{1-Amino-2-[(2-(4-ethoxy- phenyl)-ethylcarbamoyl]- ethyl}-N-pyridin-4-yl- benzamide | Compound 290 | 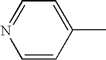 | —H | 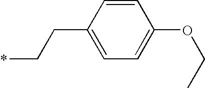 | B | ++ | +++ |
| 4-{1-Amino-2-[2-(4-methoxy- phenyl)-ethylcarbamoyl]- ethyl}-N-pyridin-4-yl- benzamide | Compound 291 | 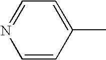 | —H | 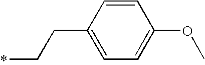 | B | ++ | +++ |
| 4-{1-Amino-2-[2-(4-phenoxy- phenyl)-ethylcarbamoyl]- ethyl}-N-pyridin-4-yl- benzamide | Compound 292 | 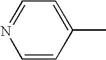 | —H | 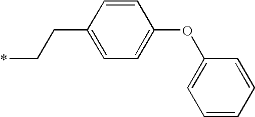 | B | ++ | +++ |
| 4-{1-Amino-2-[2-(3,4- dimethoxy-phenyl)- ethylcarbamoyl]-ethyl}-N- pyridin-4-yl-benzamide | Compound 293 | 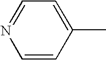 | —H | 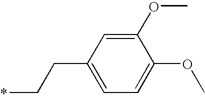 | B | + | ++ |
| 4-{1-Amino-2-[2-(3,5- dimethoxy-phenyl)- ethylcarbamoyl]-ethyl}-N- pyridin-4-yl-benzamide | Compound 294 | 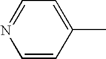 | —H | 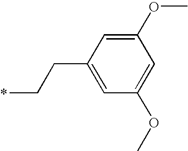 | B | + | +++ |
| 4-{1-Amino-2-[2-(3,6- dimethoxy-phenyl)- ethylcarbamoyl]-ethyl}-N- pyridin-4-yl-benzamide | Compound 295 | 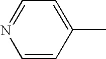 | —H | 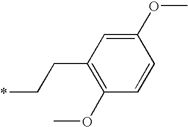 | B | + | +++ |
| 4-[1-Amino-2- (benzo[1,3]dioxol-5-yl- ethylcarbamoyl)-ethyl]-N- pyridin-4-yl-benzamide | Compound 296 | 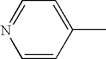 | —H | 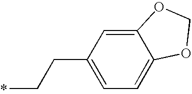 | B | + | +++ |
| 4-{1-Amino-2-[2-(2-chloro- phenyl)-ethylcarbamoyl]- ethyl}-N-pyridin-4-yl- benzamide | Compound 297 | 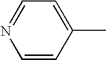 | —H | 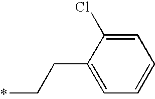 | B | ++ | +++ |
| 4-{1-Amino-2-[2-(3-chloro- phenyl)-ethylcarbamoyl]- ethyl}-N-pyridin-4-yl- benzamide | Compound 298 | 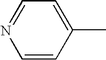 | —H | 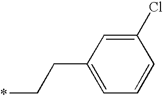 | B | ++ | +++ |
| 4-{1-Amino-2-[2-(4-chloro- phenyl)-ethylcarbamoyl]- ethyl}-N-pyridin-4-yl- benzamide | Compound 299 | 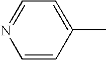 | —H | 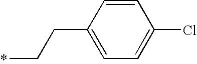 | B | ++ | +++ |
| 4-{1-Amino-2-(2-(4-chloro- phenyl)-1-methyl- ethylcarbamoyl]-ethyl}-N- pyridin-4-yl-benzamide | Compound 300 | 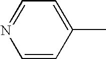 | —H | 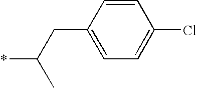 | B | ++ | +++ |
| 4-{1-Amino-2-[2-(4-chloro- phenyl)-propylcarbamoyl]- ethyl}-N-pyridin-4-yl- benzamide | Compound 301 | 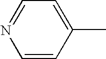 | —H | 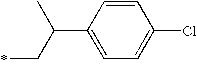 | B | +++ | +++ |
| 4-{1-Amino-2-[(2-(2,6- dichloro-phenyl)- ethylcarbamoyl]-ethyl}-N- pyridin-4-yl-benzamide | Compound 302 | 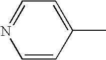 | —H | 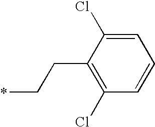 | B | ++ | +++ |
| 4-{1-Amino-2-[2-(2,4- dichloro-phenyl)- ethylcarbamoyl]-ethyl}-N- pyridin-4-yl-benzamide | Compound 303 | 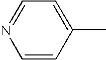 | —H | 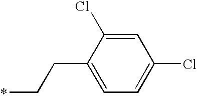 | B | ++ | +++ |
| 4-{1-Amino-2-[(2-(3,4- dichloro-phenyl)- ethylcarbamoyl]-ethyl}-N- pyridin-4-yl-benzamide | Compound 304 | 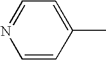 | —H | 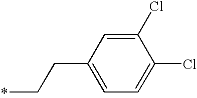 | B | ++ | +++ |
| 4-{1-Amino-2-[2-(2-fluoro- phenyl)-ethylcarbamoyl]- ethyl}-N-pyridin-4-yl- benzamide | Compound 305 | 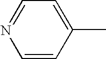 | —H | 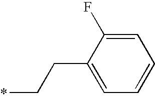 | B | ++ | +++ |
| 4-{1-Amino-2-[(2-(3-fluoro- phenyl)-ethylcarbamoyl]- ethyl}-N-pyridin-4-yl- benzamide | Compound 306 | 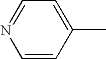 | —H | 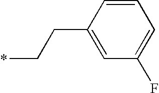 | B | + | ++ |
| 4-{1-amino-2-[2-(4-fluoro- phenyl)-ethylcarbamoyl]- ethyl}-N-pyridin-4-yl- benzamide | Compound 307 | 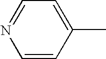 | —H | 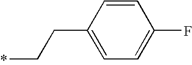 | B | +++ | +++ |
| 4-{1-amino-2-[2-(4-fluoro- phenyl)-1,1-dimethyl- ethylcarbamoyl]-ethyl}-N- pyridin-4-yl-benzamide | Compound 308 | 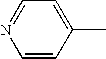 | —H | 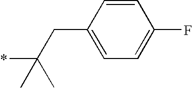 | B | +++ | +++ |
| 4-{1-amino-2-[2-(4- trifluoromethyl-phenyl)- ethylcarbamoyl]-ethyl}-N- pyridin-4-yl-benzamide | Compound 309 | 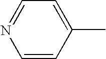 | —H | 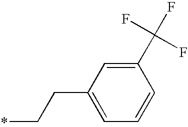 | B | ++ | +++ |
| 4-{1-amino-2-[2-(4-bromo-2- methoxy-phenyl)- ethylcarbamoyl]-ethyl}-N- pyridin-4-yl-benzamide | Compound 310 | 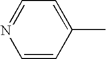 | —H | 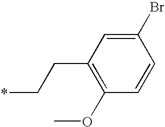 | B | ++ | +++ |
| 4-[1-amino-2-(2-thiophen-2- yl-ethylcarbamoyl)-ethyl]-N- pyridin-4-yl-benzamide | Compound 311 | 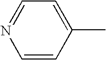 | —H | 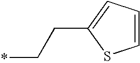 | B | ++ | +++ |
| 4-[1-amino-2-(2-pyrazol-1-yl- ethylcarbamoyl)-ethyl]-N- pyridin-4-yl-benzamide | Compound 312 | 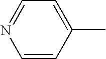 | —H | 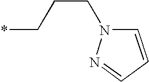 | B | ++ | +++ |
| 4-{1-Amino-2-[2-(1H-indol-3- yl)-ethylcarbamoyl]-ethyl}-N- pyridin-4-yl-benzamide | Compound 313 | 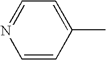 | —H | 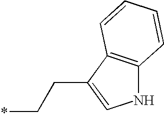 | B | ++ | +++ |
| 4-{1-Amino-2-[2-(5-methyl- 1H-indol-3-yl)- ethylcarbamoyl]-ethyl}-N- pyridin-4-yl-benzamide | Compound 314 | 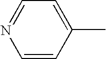 | —H | 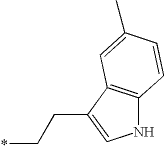 | B | ++ | +++ |
| 4-{1-Amino-2-[2-(1H-indol-3- yl)-1-methyl- ethylcarbamoyl]-ethyl}-N- pyridin-4-yl-benzamide | Compound 315 | 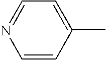 | —H | 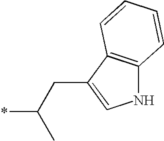 | B | ++ | +++ |
| 4-(1-Amino-2-phenyl- carbamoyl)-ethyl)-N-pyridin- 4-yl-benzamide | Compound 316 | 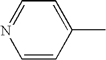 | —H | 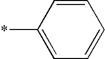 | B | ++ | +++ |
| 4-(1-Amino-2-p- tolylcarbamoyl-ethyl)-N- pyridin-4-yl-benzamide | Compound 317 | 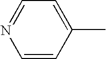 | —H | 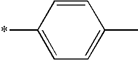 | B | nd | +++ |
| 4-{1-Amino-2-[2-(9H-fluoren- 2-yl)-ethylcarbamoyl]-ethyl}- N-pyridin-4-yl-benzamide | Compound 318 | 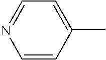 | —H | 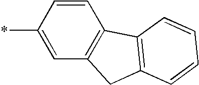 | B | + | +++ |
| 4-[1-Amino-2-(4-methoxy- phenyl-carbamoyl)-ethyl]-N- pyridin-4-yl-benzamide | Compound 319 | 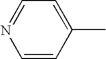 | —H | 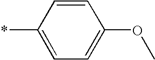 | B | ++ | +++ |
| 4-[1-Amino-2-(4- hydroxymethyl-phenyl- carbamoyl)-ethyl]-N-pyridin- 4-yl-benzamide | Compound 320 | 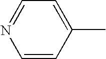 | —H | 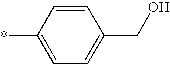 | B | ++ | +++ |
| 4-[1-Amino-2-(1H-indol-5- ylcarbamoyl)-ethyl]-N- pyridin-4-yl-benzamide | Compound 321 | 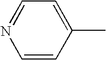 | —H | 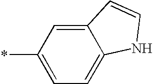 | B | ++ | +++ |
| 4-[1-Amino-2-(1H-indol-6- ylcarbamoyl)-ethyl]-N- pyridin-4-yl-benzamide | Compound 322 | 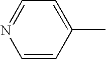 | —H | 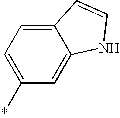 | B | +++ | +++ |
| 4-[1-Amino-2-(5-furan-2-yl- 1H-pyrazol-3-ylcarbamoyl)- ethyl]-N-pyridin-4-yl- benzamide | Compound 323 | 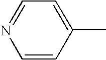 | —H | 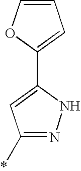 | B | ++ | +++ |
| 4-[1-amino-2-(N′-butyl- hydrazinocarbonyl)-ethyl]-N- pyridin-4-yl-benzamide | Compound 324 | 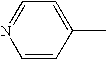 | —H | 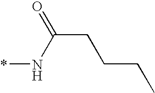 | B | ++ | +++ |
| 4-{1-amino-3[N′-(furan-2- carbonyl)-hydrazino]-3-oxo- propyl}-N-pyridin-4-yl- benzamide | Compound 325 | 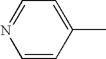 | —H | 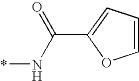 | B | ++ | +++ |
| 4-[1-Amino-2-((S)-indan-1- ylcarbamoyl)-ethyl]-N- pyridin-4-yl-benzamide | Compound 357 | 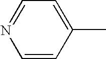 | —H | 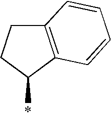 | C | ++ | +++ |
| 4-[1-Amino-2-((R)-indan-1- ylcarbamoyl)-ethyl]-N- pyridin-4-yl-benzamide | Compound 358 | 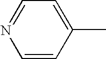 | —H | 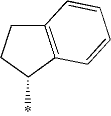 | C | +++ | +++ |
| 4-[1-Amino-2-(chroman-4- ylcarbamoyl)-ethyl]-N- pyridin-4-yl-benzamide | Compound 359 | 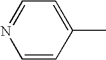 | —H | 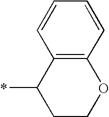 | C | +++ | nd |
| 4-[1-Amino-2-(thiochroman- 4-ylcarbamoyl)-ethyl]-N- pyridin-4-yl-benzamide | Compound 360 | 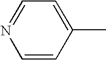 | —H | 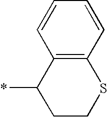 | C | +++ | nd |
| 4-[1-Amino-2-(5-fluoro- indan-1-ylcarbamoyl)-ethyl]- N-pyridin-4-yl-benzamide | Compound 361 | 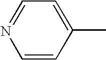 | —H | 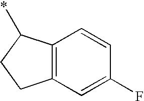 | C | +++ | nd |
| 4-[1-Amino-2-(6-fluoro- indan-1-ylcarbamoyl)-ethyl]- N-pyridin-4-yl-benzamide | Compound 362 | 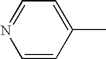 | —H | 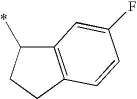 | C | +++ | nd |
| 4-[1-Amino-2-(5-chloro- indan-1-ylcarbamoyl)-ethyl]- N-pyridin-4-yl-benzamide | Compound 363 | 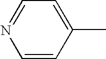 | —H | 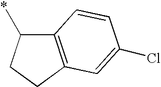 | C | nd | nd |
| 4-[1-Amino-2-(6,7,8,9- tetrahydro-5H- benzocyclohepten-5- ylcarbamoyl)-ethyl]-N- pyridin-4-yl-benzamide | Compound 364 | 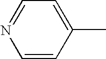 | —H | 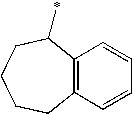 | C | nd | nd |
| 4-[1-Amino-2-(3-fluoro- 6,7,8,9-tetrahydro-5H- benzocyclohepten-5- ylcarbamoyl)-ethyl]-N- pyridin-4-yl-benzamide | Compound 365 | 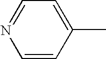 | —H | 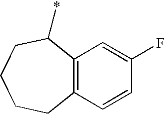 | C | nd | nd |
| 4-[1-Amino-2-(2,3-dihydro- benzofuran-3-ylcarbamoyl)- ethyl]-N-pyridin-4-yl- benzamide | Compound 366 | 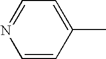 | —H | 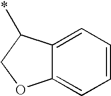 | C | +++ | nd |
| 4-[1-Amino-2-(4,5,6,7- tetrahydro- benzo[b]thiophen-4- ylcarbamoyl)-ethyl]-N- pyridin-4-yl-benzamide | Compound 367 | 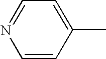 | —H | 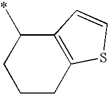 | C | +++ | nd |
| 4-(1-Amino-2-{[(4-chloro- phenyl)-cyclopropyl-methyl]- carbamoyl}-ethyl)-N-pyridin- 4-yl-benzamide | Compound 368 | 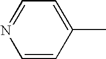 | —H | 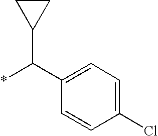 | C | ++ | nd |
| 4-(1-Amino-2-{[cyclobutyl-(4- fluoro-phenyl)-methyl]- carbamoyl}-ethyl)-N-pyridin- 4-yl-benzamide | Compound 369 | 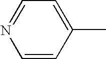 | —H | 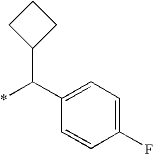 | C | +++ | nd |
| 4-{1-Amino-2-[1-(4-chloro- phenyl)-1-methyl- ethylcarbamoyl]-ethyl}-N- pyridin-4-yl-benzamide | Compound 622 | 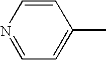 | —H | 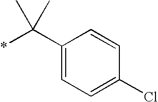 | C | +++ | +++ |
| 4-{1-Amino-2-[1-(4-chloro- phenyl)- cyclopropylcarbamoyl]- ethyl}-N-pyridin-4-yl- benzamide | Compound 623 | 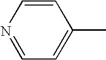 | —H | 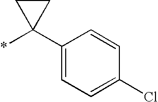 | C | nd | nd |
| 4-{1-Amino-2-[1-(4-fluoro- phenyl)-1-methyl- ethylcarbamoyl]-ethyl}-N- pyridin-4-yl-benzamide | Compound 624 | 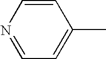 | —H | 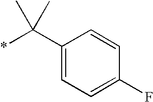 | C | nd | nd |
| 4-{1-Amino-2-[1-(4-fluoro- phenyl)- cyclopropylcarbamoyl]- ethyl}-N-pyridin-4-yl- benzamide | Compound 625 | 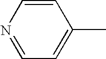 | —H | 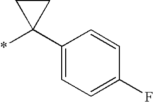 | C | nd | nd |
| 4-{1-Amino-2-[1-(4-fluoro- phenyl)- cyclobutylcarbamoyl]-ethyl}- N-pyridin-4-yl-benzamide | Compound 626 | 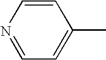 | —H | 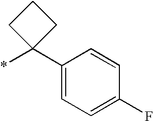 | C | nd | nd |
| 4-{1-Amino-2-[1-(4-fluoro- phenyl)- cyclopentylcarbamoyl]- ethyl}-N-pyridin-4-yl- benzamide | Compound 627 | 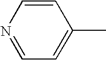 | —H | 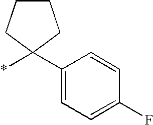 | C | nd | nd |
| 4-[1-Amino-2-(1-methyl-1- thiophen-2-yl- ethylcarbamoyl)-ethyl]-N- pyridin-4-yl-benzamide | Compound 628 | 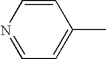 | —H | 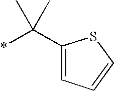 | C | nd | nd |
| 4-[1-Amino-2-(1-thiophen-2- yl-cyclopropylcarbamoyl)- ethyl]-N-pyridin-4-yl- benzamide | Compound 629 | 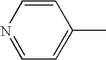 | —H | 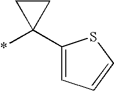 | C | nd | nd |
| 4-[1-Amino-2-(1-methyl-1- thiophen-3-yl- ethylcarbamoyl)-ethyl]-N- pyridin-4-yl-benzamide | Compound 630 | 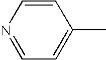 | —H | 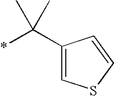 | C | nd | nd |
| 4-[1-Amino-2-(1-thiophen-3- yl-cyclopropylcarbamoyl)- ethyl]-N-pyridin-4-yl- benzamide | Compound 631 | 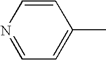 | —H | 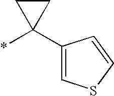 | C | nd | nd |
| 4-{1-Amino-2-[1-(3,4- difluoro-phenyl)-1-methyl- ethylcarbamoyl]-ethyl}-N- pyridin-4-yl-benzamide | Compound 632 | 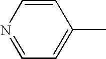 | —H | 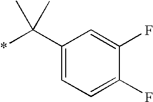 | C | nd | nd |
| 4-{1-Amino-2-[1-(3,4- difluoro-phenyl)- cyclopropylcarbamoyl]- ethyl}-N-pyridin-4-yl- benzamide | Compound 633 | 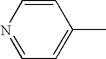 | —H | 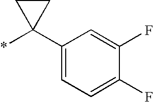 | C | nd | nd |
| 4-{1-Amino-2-[1-(3,4- difluoro-phenyl)- cyclobutylcarbamoyl]-ethyl}- N-pyridin-4-yl-benzamide | Compound 634 | 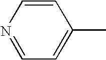 | —H | 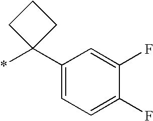 | C | nd | nd |
| 4-{1-Amino-2-[1-(3,4- difluoro-phenyl)- cyclopentylcarbamoyl]- ethyl}-N-pyridin-4-yl- benzamide | Compound 635 | 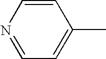 | —H | 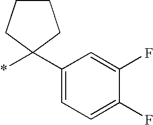 | C | nd | nd |
| 4-{1-Amino-2-[1-(3,4- dichloro-phenyl)-1-methyl- ethylcarbamoyl]-ethyl}-N- pyridin-4-yl-benzamide | Compound 636 | 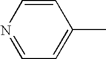 | —H | 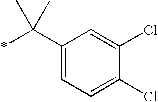 | C | nd | nd |
| 4-{1-Amino-2-[1-(3,4- dichloro-phenyl)- cyclopropylcarbamoyl]- ethyl}-N-pyridin-4-yl- benzamide | Compound 637 | 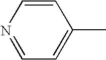 | —H | 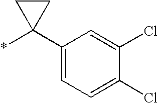 | C | nd | nd |
| 4-{1-Amino-2-[1-(2,4- difluoro-phenyl)-1-methyl- ethylcarbamoyl]-ethyl}-N- pyridin-4-yl-benzamide | Compound 638 | 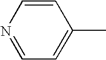 | —H | 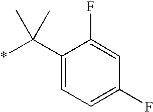 | C | nd | nd |
| 4-{1-Amino-2-[1-(2,4- difluoro-phenyl)- cyclopropylcarbamoyl]- ethyl}-N-pyridin-4-yl- benzamide | Compound 639 | 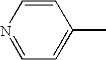 | —H | 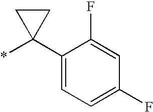 | C | nd | nd |
| 4-{1-Amino-2-[1-(3-chloro-4- fluoro-phenyl)-1-methyl- ethylcarbamoyl]-ethyl}-N- pyridin-4-yl-benzamide | Compound 640 | 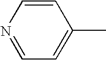 | —H | 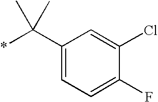 | C | nd | nd |
| 4-{1-Amino-2-[1-(3-chloro-4- fluoro-phenyl)- cyclopropylcarbamoyl]- ethyl}-N-pyridin-4-yl- benzamide | Compound 641 | 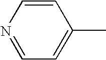 | —H | 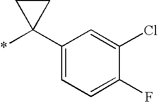 | C | nd | nd |
| 4-{1-Amino-2-[1-(2-chloro-4- fluoro-phenyl)-methyl- ethylcarbamoyl]-ethyl}-N- pyridin-4-yl-benzamide | Compound 642 | 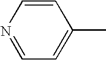 | —H | 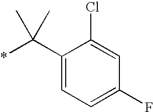 | C | nd | nd |
| 4-{1-Amino-2-[1-(2-chloro-4- fluoro-phenyl)- cyclopropylcarbamoyl]- ethyl}-N-pyridin-4-yl- benzamide | Compound 643 | 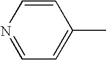 | —H | 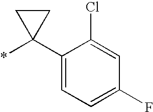 | C | nd | nd |
| 4-{1-Amino-2-[1-(4-chloro-2- fluoro-phenyl)-1-methyl- ethylcarbamoyl]-ethyl}-N- pyridin-4-yl-benzamide | Compound 644 | 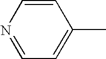 | —H | 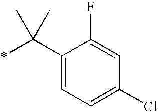 | C | nd | nd |
| 4-{1-Amino-2-[1-(4-chloro-2- fluoro-phenyl)- cyclopropylcarbamoyl]- ethyl}-N-pyridin-4-yl- benzamide | Compound 645 | 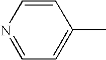 | —H | 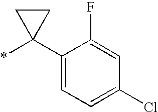 | C | nd | nd |

### Compound 370

### 4-(1-Amino-2-{[1-(4-chloro-phenyl)-cyclopropanecarbonyl]-amino}-ethyl)-N-(3-methyl-pyridin-4-yl)-benzamide dihydrochloric acid salt

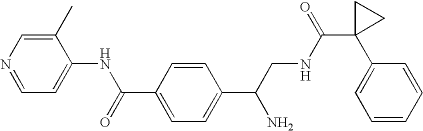

To a solution of Intermediate 48 (400 mg) in DCM (0-25 M) was added oxalyle chloride (2.5 eq) and a few drops of DMF. The reaction mixture was stirred at RT for 2 hours and then, was evaporated to give the corresponding acyl chloride as a yellow powder (100% yield).

To a solution of 3-methyl-pyridin-4-ylamine (24 mg) in acetonitrile, were added DIEA (1 eq) and the (1-(4-chlorocarbonyl-phenyl)-2-{[1-(4-chloro-phenyl)-cyclopropanecarbonyl]-amino}-ethyl)-carbamic acid tert-butyl ester (100 mg, 1 eq). The reaction mixture was stirred at RT, under a nitrogen atmosphere, for 2 hours. The solvent was removed under reduced pressure. The product was purified by flash chromatography (DCM/MeOH, 98/2 to 95/5), and then, by preparative HPLC, yielding the {2-{[1-(4-Chloro-phenyl)-cyclopropanecarbonyl]-amino}-1-[4-(3-methyl-pyridin-4-ylcarbamoyl)-phenyl]-ethyl}-carbamic acid tert-butyl ester (3% yield).

The compound from the previous step was dissolved in dry 1,4-dioxane. HCl gas was bubbled through the reaction mixture for 15 minutes. The solvent was evaporated to give the title compound (100% yield).

### Compound 371

### 4-(1-Amino-2-{[1-(4-chloro-phenyl)-cyclopropanecarbonyl]-amino}-ethyl)-N-(1H-pyrrolo[2,3-b]pyridin-4-yl)-benzamide dihydrochloric acid salt

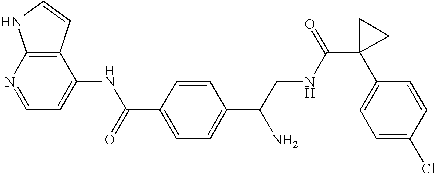

To a solution of Intermediate 48 (400 mg) in DCM (0.25 M) was added oxalyle chloride (2.5 eq) and a few drops of DMF. The reaction mixture was stirred at RT for 2 hours and then, was evaporated to give the corresponding acyl chloride as a yellow powder (100% yield).

To a solution of 4-amino-pyrrolo[2,3-b]pyridine-1-carboxylic acid tert-butyl ester (51 mg, prepared by known method) in acetonitrile, were added DIEA (1 eq) and the (1-(4-chlorocarbonyl-phenyl)-2-{[1-(4-chloro-phenyl)-cyclopropanecarbonyl]-amino}-ethyl)-carbamic acid tert-butyl ester (100 mg, 1 eq). The reaction mixture was stirred at RT, under a nitrogen atmosphere, for 2 hours. The solvent was removed under reduced pressure. The product was purified by flash chromatography (DCM /MeOH, 98/2 to 95/5), and then, by preparative HPLC, yielding the 4-[4-(1-tert-Butoxycarbonylamino-2-{[1-(4-chloro-phenyl)-cyclopropanecarbonyl]-amino}-ethyl)-benzoylamino]-pyrrolo[2,3-b]pyridine-1-carboxylic acid tert-butyl ester (3% yield).

The compound from the previous step was dissolved in dry 1,4-dioxane. HCl gas was bubbled through the reaction mixture for 15 minutes. The solvent was evaporated to give the title compound (100% yield).

### Further Compounds

The stereoisomers of compound 223 were prepared. The synthesis is given hereunder.

### Compound 372

### 4-{(R)-1-Amino-2-[(S)-(1,2,3,4-tetrahydro-naphthalen-1-yl)carbamoyl]-ethyl}-N-pyridin-4-yl-benzamide dihydrochloric acid salt

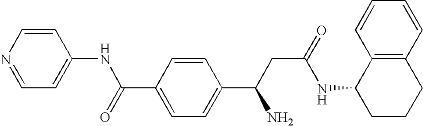

To a solution of Intermediate 4 (75 mg) in DMF (0.5 M) were added TBTU (1.3 eq.), HOBt (0.2 eq.) and DIEA (3 eq.). The reaction mixture was stirred at RT for 5 min, and the (R)-(1,2,3,4-tetrahydro-naphthalen-1-yl)amine (1 eq.) was added. DMF was evaporated, and water was added to the residue. The product was extracted with EtOAc. The organic layer was washed with 2N Na₂CO₃, water and then brine. The crude compound was dissolved in dry 1,4-dioxane. HCl gas was bubbled through the reaction mixture for 15 minutes. The title compound was purified by preparative HPLC (41% yield, white powder). ¹H NMR (300 MHz, DMSO-d6)-1.43-1.68 ppm (m, 4H); 2.63 ppm (m, 2H); 2.90 ppm (m, 2H); 4.84 ppm (m, 2H); 6.93 ppm (d, 1H, J=7.5 Hz); 7.07 ppm (m, 3H); 7.70 ppm (d, 2H, J=8.4 Hz); 8.15 ppm (d, 2H, J=8.4 Hz); 8.39 ppm (d, 2H, J=7.2 Hz); 8.75 ppm (d, 2H, J=7.2 Hz); 11.77 ppm (s, 1H).

### Compound 373

### 4-{(R)-1-Amino-2-[(R)-(1,2,3,4-tetrahydro-naphthalen-1-yl)carbamoyl]-ethyl}-N-pyridin-4-yl-benzamide dihydrochloric acid salt

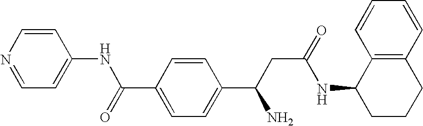

Compound 373 was prepared according to the protocol described for Compound 372 starting from Intermediate 4 and (S)-(1,2,3,4-tetrahydro-naphthalen-1-yl)amine (34% yield, white powder). ¹H NMR (300 MHz, DMSO-d6): 1.55-1.77 ppm (m, 4H); 2.67 ppm (m, 2H); 2.92 ppm (m, 2H); 4.79 ppm (m, 2H); 6.37 ppm (d, 1H, J=7.5 Hz); 6.84 ppm (m, 1H); 7.00 ppm (m, 2H); 7.74 ppm (d, 2H, J=8.4 Hz); 8.18 ppm (d, 2H, J=8.4 Hz); 8.44 ppm (d, 2H, J=7.2 Hz); 8.76 ppm (d, 2H, J=7.2 Hz); 11.87 ppm (s, 1H).

### Compound 374

### 4-{(S)-1-Amino-2-[(R)-(1,2,3,4-tetrahydro-naphthalen-1-yl)carbamoyl]-ethyl}-N-pyridin-4-yl-benzamide dihydrochloric acid salt

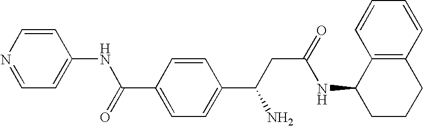

Compound 374 was prepared according to the protocol described for Compound 372 starting from Intermediate 5 and (R)-(1,2,3,4-tetrahydro-naphthalen-1-yl)amine (28% yield, white powder). ¹H NMR (300 MHz, DMSO-d6+D₂O): 1.39-1.62 ppm (m, 4H); 2.61 ppm (m, 2H); 2.86 ppm (m, 2H); 4.73 ppm (dd, 1H, J=7.1 & 6.7 Hz); 4.80 ppm (dd, 1H, J=6.7 & 6.3 Hz); 6.85 ppm (d, 1H, J=7.1 Hz); 6.99-7.11 (m, 3H); 7.64 ppm (d, 2H, J=8.5 Hz); 8.06 ppm (d, 2H, J=8.5 Hz); 8.27 ppm (d, 2H, J=7.1 Hz); 8.68 ppm (d, 2H, J=7.1 Hz).

### Compound 375

### 4{(S)-1-Amino-2-[(S)-(1,2,3,4-tetrahydro-naphthalen-1-yl)carbamoyl]-ethyl}-N-pyridin-4-yl-benzamide dihydrochloric acid salt

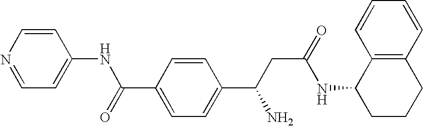

The title compound was prepared according to the protocol described for Compound 372 starting from Intermediate 5 and (S)-(1,2,3,4-tetrahydro-naphthalen-1-yl)amine (54% yield, white powder). ¹H NMR (300 MHz, DMSO-d6+D₂O): 1.60 ppm (m, 2H); 1.77 ppm (m, 2H); 2.61 ppm (m, 2H); 2.86 ppm (m, 2H); 4.74 ppm (m, 2H); 6.31 ppm (d, 1H, J=7.7 Hz); 6.81 (t, 1H, J=7.8 Hz); 7.00 ppm (m, 2H); 7.68 ppm (d, 2H, J=8.5 Hz); 8.08 ppm (d, 2H, J=8.5 Hz); 8.29 ppm (d, 2H, J=7.3 Hz); 8.68 ppm (d, 2H, J=7.3 Hz).

The inhibition results of the prepared compounds are shown under Table 4.

TABLE 4

| | | | IC₅₀ | IC₅₀ |
|---|---|---|---|---|
| | | | PKCε | ROCK |
| Structure | Compound | Configuration | (μM) | (μM) |
| 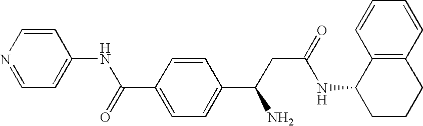 | Compound 372 | R, S | ++ | +++ |
| 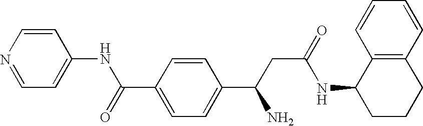 | Compound 373 | R, R | ++++ | ++++ |
| 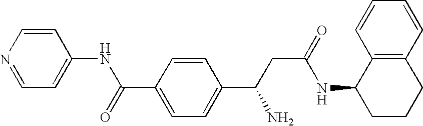 | Compound 374 | S, R | ++ | +++ |
| 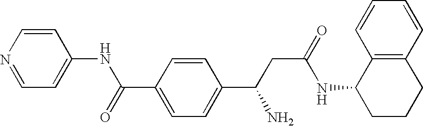 | Compound 375 | S, S | + | +++ |

### Compound 397

### 4-((S)-1-Amino-2-{[1-(4-chloro-phenyl)-cyclopropanecarbonyl]-amino}-ethyl)-N-pyridin-4-yl-benzamide dihydrochloric acid salt

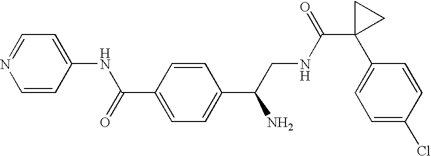

Compound 397 was obtained by preparative chiral HPLC separation of Compound 168 (Column: OD-H, 10×250 mm, 5 μm; Solvent: hexane/ethanol 93/7 with 0.1% DIPEA).

% ee=93% (chiral HPLC: column OD-H, 0.46×250 mm, hexane/ethanol 90/10 with 0.1% DIPEA, Tᵣₑₜ: 30 min).

### Compound 398

### 4-((R)-1-Amino-2-{[1-(4-chloro-phenyl)-cyclopropanecarbonyl]-amino}-ethyl)-N-pyridin-4-yl-benzamide dihydrochloric acid salt

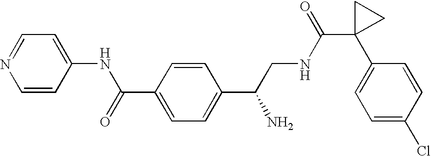

Compound 398 was obtained by preparative chiral HPLC separation of Compound 168 (Column: OD-H, 10×250 mm, 5 μm; Solvent: hexane/ethanol 93/7 with 0.1% DIPEA).

% ee=99% (chiral HPLC: column OD-H, 0.46×250 mm, hexane/ethanol 90 /10 with 0.1% DIPEA, Tᵣₑₜ: 36 min).

### Compound 399

### 4-{(S)-1-Amino-2-[2-(4-chloro-phenyl)-2-methyl-propionylamino]-ethyl}-N-pyridin-4-yl-benzamide dihydrochloric acid salt

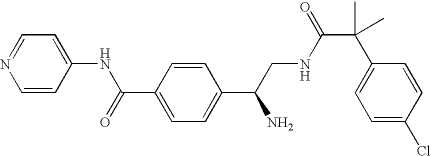

Compound 399 was obtained by preparative chiral HPLC separation of Compound 335 (Column: AD-H, 10×250 mm, 5 μm; Solvent: hexane/ethanol 85/15 with 0.1% DIPEA).

% ee=100% (chiral HPLC: column AD-H, 0.46×250 mm, hexane/ethanol 85115 with 0.1% DIPEA, Tᵣₑₜ: 36.5 min).

### Alternative Synthesis of Compound 399

A solution of dichloro(p-cymene)ruthenium (II) dimer (0.05 eq.) and (1S,2S)-(+)-N-p-tosyl-1,2-diphenylethylenediamine (0-1 eq.) in 2-propanol was heated at 80° C. for 1 hour to give the Noyori's catalyst (JACS, 1996, 118, 2521; JACS, 2005, 127, 4596).

To a solution of 4-{2-[2-(4-chloro-phenyl)-2-methyl-propionylamino]-acetyl}-N-pyridin-4-yl-benzamide (300 mg, 1 eq.) in DMF (2 ml) at 0° C. were added formic acid (15 eq.) and triethylamine (6 eq.) simultaneously. The solution was aged for 10 min and then warmed to RT. At RT Noyori catalyst was added and the mixture was aged for 24 h at 40° C. The reaction mixture was cooled down to RT, diluted with water, and extracted with ethyl acetate. The combined organic layers were washed with brine, dried over MgSO₄, filtered, and the filtrate was evaporated. The product was purified by filtration through a silica gel plug to give the 4-{(R)-2-[2-(4-chloro-phenyl)-2-methyl-propionylamino]-1-hydroxy-ethyl}-N-pyridin-4-yl-benzamide in 99% yield as a yellowish oil (% ee=94% determined by chiral HPLC: column OJ-H, 0.46×250 mm, hexane/isopropanol 90/10 with 0.1% DIPEA, Tᵣₑₜ: 68.4 min).

To a solution of 4-{(R)-2-[2-(4-chloro-phenyl)-2-methyl-propionylamino]-1-hydroxy-ethyl}-N-pyridin-4-yl-benzamide in toluene-DMF (9:1, 4 ml) were added DPPA (2 eq.) and DBU (2 eq.). The solution was stirred at RT for 1.5 hours and then at 50° C. for 16 hours. The reaction mixture was diluted with water and extracted with ethyl acetate (×3). The combined organic layers were washed with brine, dried over MgSO₄, filtered, and the solvent was evaporated. The 4-{(S)-1-Azido-2-[2-(4-chlorophenyl)-2-methyl-propionylamino]-ethyl}-N-pyridin-4-yl-benzamide (% ee>80%) was purified by flash chromatography on silica gel (DCM/MeOH 95/5 to 4/1, 77% yield).

To a solution of the previous azide in MeOH (60 ml) was added Pd/C (10%) and the reaction mixture was stirred in atmosphere of hydrogen (1 atm) for 17 hours. The reaction mixture was filtered through a layer of Celite and washed with MeOH. The solvent was evaporated. Purification by flash chromatography on silica gel (DCM/MeOH 95/5 to 4/1) provided 0.1089 of a mixture of the desired amine and diphenyl phosphate (byproduct of the previous step). This mixture was dissolved in ethylacetate and extracted with 1M HCl. The aqueous layer was basified with 2M NaOH, extracted with ethyl acetate (×3), washed with water, dried over MgSO₄, filtered off and then the solvent was evaporated. The enantiomeric excess could be increased by chiral HPLC or by crystallization.

### Compound 400

### 4-((R)-1-Amino-2-{[1-(4-chloro-phenyl)-cyclopropanecarbonyl]-amino}-ethyl)-N-pyridin-4-yl-benzamide dihydrochloric acid salt

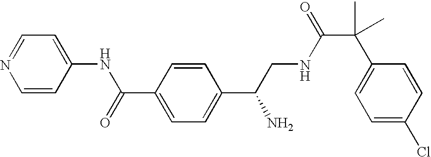

Compound 400 was obtained by preparative chiral HPLC separation of Compound 335 (Column: AD-H, 10×250 mm, 5 μm; Solvent: hexane/ethanol 85/15 with 0.1% DIPEA).

% ee=98.7% (chiral HPLC: column AD-H, 0.46×250 mm, hexane/ethanol 85/15 with 0.1% DIPEA, Tᵣₑₜ: 50.6 min).

### Compound 401

### 4-[(R)-1-Amino-2-(4-fluoro-benzylcarbamoyl)-ethyl]-N-pyridin-4-yl-benzamide dihydrochloric acid salt

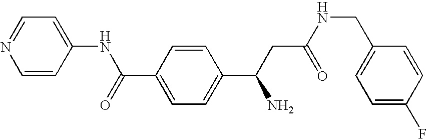

Compound 401 was prepared according to the protocol described for Compound 372 starting from Intermediate 4 and 4-fluorobenzylamine (35% yield, white powder). ¹H NMR (300 MHz, DMSO-d6+D₂O): 2.84-2.88 ppm (m, 2H); 4.11 ppm [AB system, 2H, J_{AB}=35.4 Hz, (A, 4.05 ppm (d, 2H, J=15.3 Hz)), (B, 4.17 ppm (d, 2H, J=15.3 Hz))]; 4.68 ppm (t, 1H, J=7.3 Hz); 6.94 ppm (d, 4H, J_{H—F}=7.5 Hz); 7.60 ppm (d, 2H, J=8.5 Hz); 7.99 ppm (d, 2H, J=8.4 Hz); 8.26 ppm (d, 2H, J=7.3 Hz); 8.65 ppm (d, 2H, J=7.3 Hz).

% ee=99% (chiral HPLC: column AD-H, 0.46×250 mm, hexane/ethanol 80/20 with 0.1% DIPEA, Tᵣₑₜ: 23 min).

### Compound 402

### 4-[(S)-1-Amino-2-(4-fluoro-benzylcarbamoyl)-ethyl]-N-pyridin-4-yl-benzamide dihydrochloric acid salt

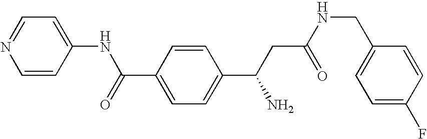

Compound 402 was prepared according to the protocol described for Compound 372 starting from Intermediate 5 and 4-fluorobenzylamine (21% yield, white powder). ¹H NMR (300 MHz, DMSO-d6+D₂O): 2.84-2.88 ppm (m, 2H); 4.11 ppm [AB system, 2H, J_{AB}=35.4 Hz, (A, 4.05 ppm (d, 2H, J=15.3 Hz)), (B, 4.17 ppm (d, 2H, J=15.3 Hz))]; 4.68 ppm (t, 1H, J=7.3 Hz); 6.94 ppm (d, 4H, J_{H—F}=7.5 Hz); 7.60 ppm (d, 2H, J=8.5 Hz); 7.99 ppm (d, 2H, J=8.4 Hz); 8.26 ppm (d, 2H, J=7.3 Hz); 8.65 ppm (d, 2H, J=7.3 Hz).

% ee=97% (chiral HPLC: column AD-H, 0.46×250 mm, hexane/ethanol 80/20 with 0.1% DIPEA, Tᵣₑₜ: 21 min).

### Compound 403

### 4-[(R)-1-Amino-2-(2,3-dihydro-benzofuran-3-ylcarbamoyl)-ethyl]-N-pyridin-4-yl-benzamide dihydrochloric acid salt

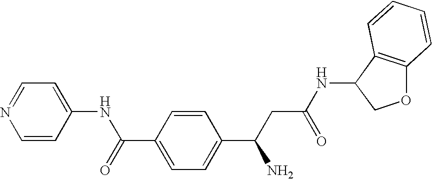

Compound 403 was prepared according to the protocol described for Compound 372 starting from Intermediate 4 and Intermediate 17 (% yield, beige powder). ¹H NMR (300 MHz, DMSO-d6): 2.80-2.95 ppm (m, 2H); 3.99 ppm (ddd, 2H, diastereoisomer 1, J₁=51.5 Hz, J₂=9.2 Hz, J₃=4.6 Hz); 4.54-4.62 ppm (m, 2H, diastereoisomer 2); 4.73-4.76 ppm (m, 1H); 5.35-5.40 ppm (m, 1H); 6.75-6.90 ppm (m, 2H); 7.13-7.21 ppm (m, 2H); 7.68 ppm (dd, 2H, J₁=8.3 Hz, J₂=4.0 Hz); 8.11 ppm (d, 2H, J=8.0 Hz); 8.35-8.40 ppm (m, 2H); 8.65-8.80 ppm (m, 2H); 8.74 ppm (d, 2H, J=6.4 Hz); 11.71 ppm (s, 1H).

### Compound 404

### 4-[(S)-1-Amino-2-(2,3-dihydro-benzofuran-3-ylcarbamoyl)-ethyl]-N-pyridin-4-yl-benzamide dihydrochloric acid salt

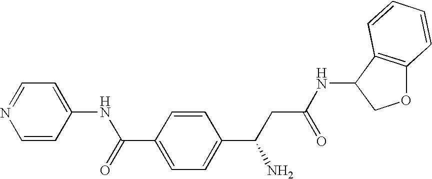

Compound 404 was prepared according to the protocol described for Compound 372 starting from Intermediate 5 and Intermediate 17 (% yield, beige powder). ¹H NMR (300 MHz, DMSO-d6): ¹H NMR (300 MHz, DMSO-d6): 2.80-2.95 ppm (m, 2H); 3.99 ppm (ddd, 2H, diastereoisomer 1, J₁=51.5 Hz, J₂=9.2 Hz, J₃=4.6 Hz); 4.54-4.62 ppm (m, 2H, diastereoisomer 2); 4.73-4.76 ppm (m, 1H); 5.35-5.40 ppm (m, 1H); 6.75-6.90 ppm (m, 2H); 7.13-7.21 ppm (m, 2H); 7.68 ppm (dd, 2H, J₁=8.3 Hz, J₂=4.0 Hz); 8.11 ppm (d, 2H, J=8.0 Hz); 8.35-8.40 ppm (m, 2H); 8.65-8.80 ppm (m, 2H); 8.74 ppm (d, 2H, J=6.4 Hz); 11.71 ppm (s, 1H).

### Compound 405

### 4-{(R)-1-Amino-2-[(S)-(2,3-dihydro-benzofuran-3-yl)carbamoyl]-ethyl}-N-pyridin-4-yl-benzamide dihydrochloric acid salt

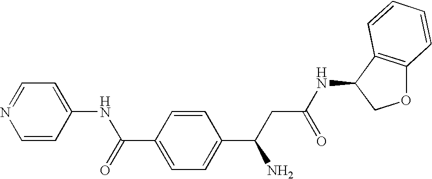

Compound 405 was obtained by preparative chiral HPLC separation of Compound 403 (Column: AD-H, 10×250 mm, 5 μm; Solvent: hexane/ethanol 87/13 with 0.1% DIPEA).

% de=98% (chiral HPLC: column AD-H, 0.46×250 mm, hexane/ethanol 85/15 with 0.1% DIPEA, Tᵣₑₜ: 45 min).

### Compound 406

### 4-{(R)-1-Amino-2-[(R)-(2,3-dihydro-benzofuran-3-yl)carbamoyl]-ethyl}-N-pyridin-4-yl-benzamide dihydrochloric acid salt

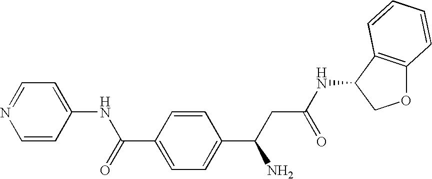

Compound 406 was obtained by preparative chiral HPLC separation of Compound 403 (Column: AD-H, 10×250 mm, 5 μm; Solvent: hexane/ethanol 87/13 with 0.1% DIPEA).

% de=95% (chiral HPLC: column AD-H, 0.46×250 mm, hexane/ethanol 85/15 with 0.1% DIPEA, Tᵣₑₜ: 53 min).

### Compound 407

### 4{(S)-1-Amino-2-[(S)-(2,3-dihydro-benzofuran-3-yl)carbamoyl]-ethyl}-N-pyridin-4-yl-benzamide dihydrochloric acid salt

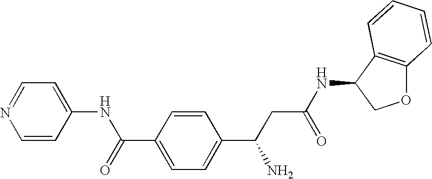

Compound 407 was obtained by preparative chiral HPLC separation of Compound 404 (Column: AD-H, 10×250 mm, 5 μm; Solvent: hexane/ethanol 85/15 with 0.1% DIPEA).

% de=99.5% (chiral HPLC: column AD-H, 0.46×250 mm, hexane/ethanol 85/15 with 0.1% DIPEA, Tᵣₑₜ: 62 min).

### Compound 408

### 4-{(S)-1-Amino-2-[(R)-(2,3-dihydro-benzofuran-3-yl)carbamoyl]-ethyl}-N pyridin-4-yl-benzamide dihydrochloric acid salt

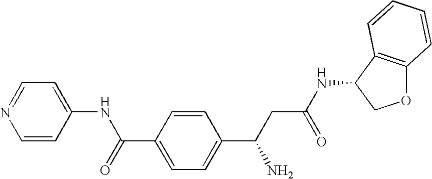

Compound 408 was obtained by preparative chiral HPLC separation of Compound 404 (Column: AD-H, 10×250 mm, 5 μm; Solvent: hexane/ethanol 85/15 with 0.1% DIPEA).

% de=98% (chiral HPLC: column AD-H, 0.46×250 mm, hexane/ethanol 85/15 with 0.1% DIPEA, Tᵣₑₜ: 39 min).

### Compound 409

### (R)-1,2,3,4-Tetrahydro-naphthalene-1-carboxylic acid {(S)-2-amino-2-[4-(pyridin-4-ylcarbamoyl)-phenyl]-ethyl}-amide dihydrochloric acid salt

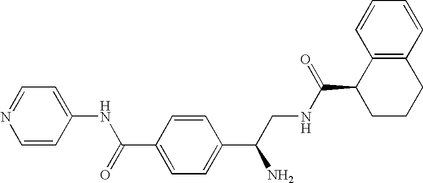

Compound 409 was obtained by preparative chiral HPLC separation of Compound 350.

### Compound 410

### (S)-1,2,3,4-Tetrahydro-naphthalene-1-carboxylic acid {(S)-2-amino-2-[4-(pyridin-4-ylcarbamoyl)-phenyl]-ethyl}-amide dihydrochloric acid salt

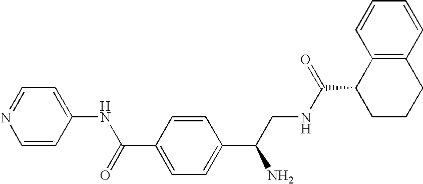

Compound 410 was obtained by preparative chiral HPLC separation of Compound 350.

### Compound 411

### (R)-1,2,3,4-Tetrahydro-naphthalene-1-carboxylic acid {(R)-2-amino-2-[4 (pyridin-4-ylcarbamoyl)-phenyl]-ethyl}-amide dihydrochloric acid salt

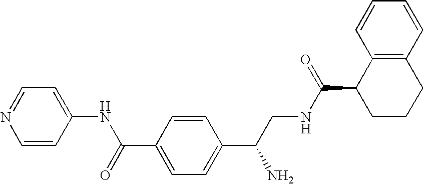

Compound 411 was obtained by preparative chiral HPLC separation of Compound 350.

### Compound 412

### (S)-1,2,3,4-Tetrahydro-naphthalene-1-carboxylic acid {(R)-2-amino-2-[4 (pyridin-4-ylcarbamoyl)-phenyl]-ethyl}-amide dihydrochloric acid salt

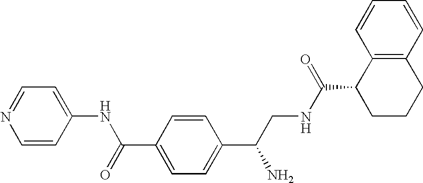

Compound 412 was obtained by preparative chiral HPLC separation of Compound 350.

### Compound 413

### (R)-Indan-1-carboxylic acid {(S)-2-amino-2-[4-(pyridin-4-ylcarbamoyl)phenyl]-ethyl}-amide dihydrochloric acid salt

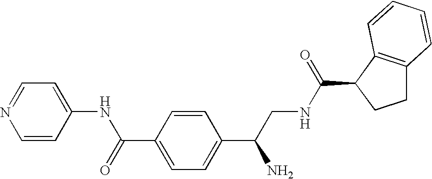

Compound 413 was obtained by preparative chiral HPLC separation of Compound 351.

### Compound 414

### (S)-Indan-1-carboxylic acid {(S)-2-amino-2-[4-(pyridin-4-ylcarbamoyl)phenyl]-ethyl}-amide dihydrochloric acid salt

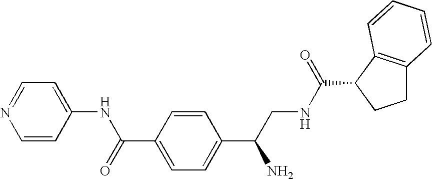

Compound 414 was obtained by preparative chiral HPLC separation of Compound 351.

### Compound 415

### (R)-Indan-1-carboxylic acid {(R)-2-amino-2-[(4-(pyridin-4-ylcarbamoyl)phenyl]-ethyl}-amide dihydrochloric acid salt

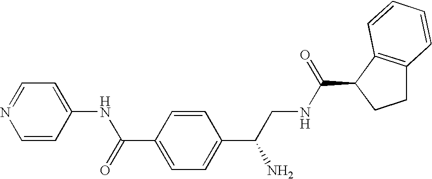

Compound 415 was obtained by preparative chiral HPLC separation of Compound 351.

### Compound 416

### (S)-Indan-1-carboxylic acid {(R)-2-amino-2-[4-(pyridin-4-ylcarbamoyl)phenyl]-ethyl}-amide dihydrochloric acid salt

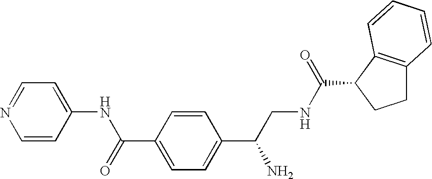

Compound 416 was obtained by preparative chiral HPLC separation of Compound 351.

### Compound 417

### 4-((S)-1-Amino-2-{[1-(4-fluoro-phenyl)-cyclopentanecarbonyl]-amino}-ethyl)-N-pyridin-4-yl-benzamide dihydrochloric acid salt

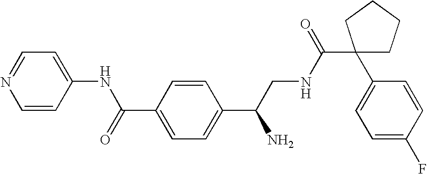

Compound 417 was obtained by preparative chiral HPLC separation of Compound 336 (Column: AD-H, 10×250 mm, 5 μm; Solvent: hexane/ethanol 85/15 with 0.1% DIPEA).

% ee=100% (chiral HPLC: column AD-H, 0.46×250 mm, hexane/ethanol 85/15 with 0.1% DIPEA, Tᵣₑₜ: 38 min).

### Compound 418

### 4-((R)-1-Amino-2-{[1-(4-fluoro-phenyl)-cyclopentanecarbonyl]-amino}-ethyl)-N-pyridin-4-yl-benzamide dihydrochloric acid salt

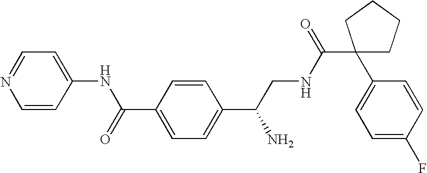

Compound 418 was obtained by preparative chiral HPLC separation of Compound 336 (Column: AD-H, 10×250 mm, 5 μm; Solvent: hexane/ethanol 85/15 with 0.1% DIPEA).

% ee=100% (chiral HPLC: column AD-H, 0.46×250 mm, hexane/ethanol 85/15 with 0.1% DIPEA, Tᵣₑₜ: 48 min).

### Compound 419

### 4-{1-Amino-2-[2-(4-fluoro-phenyl)-2-methyl-thiopropionylamino]-ethyl}-N pyridin-4-yl-benzamide dihydrochloric acid salt

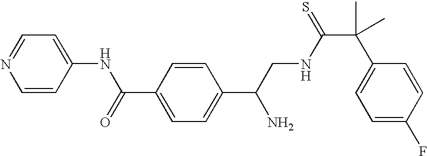

A solution of 2-(4-Fluoro-phenyl)-2-methyl-propionic acid (400 mg) and Davy's reagent (2,4-Bis-methylsulfanyl-[1,3,2,4]dithiadiphosphetane 2,4-disulfide, 1.5 eq.) in toluene (15 ml) was heated by microwave at 140° C. for 40 min. The reaction mixture was filtered off and the filtrate was evaporated. The crude product was purified by flash chromatography (Eluent: pentane, Rf=0.17) to afford the 2-(4-fluoro-phenyl)-2-methyl-dithiopropionic acid methyl ester as a yellow oil (46% yield).

A solution of 2-(4-fluoro-phenyl)-2-methyl-dithiopropionic acid methyl ester (220 mg), intermediate 4 (1 eq.) and triethylamine was stirred at 65° C. for 10 days. The reaction mixture was evaporated and the residue was purified by flash chromatography (eluent: DCM to DCM/MeOH 96/4) to give the {2-[2-(4-Fluoro-phenyl)-2-methyl-thiopropionylamino]-1-[4-(pyridin-4-ylcarbamoyl)-phenyl]-ethyl}-carbamic acid tert-butyl ester. The title compound was obtained after tert-butyloxy group deprotection as a pale yellow powder (15% yield).

The inhibition results of compounds 397 to 419 are shown under Table 5.

TABLE 5

| | | IC₅₀ | IC₅₀ |
|---|---|---|---|
| | | μM | μM |
| Name | Compound | PKCε | ROCK |
| 4-((S)-1-Amino-2-{[1-(4-chloro-phenyl)-cyclopropanecarbonyl]- | Compound | ++++ | +++ |
| amino}-ethyl)-N-pyridin-4-yl-benzamide | 397 | | |
| 4-((R)-1-Amino-2-{[1-(4-chloro-phenyl)-cyclopropanecarbonyl]- | Compound | ++ | ++ |
| amino}-ethyl)-N-pyridin-4-yl-benzamide | 398 | | |
| 4-{(S)-1-Amino-2-[2-(4-chloro-phenyl)-2-methyl- | Compound | ++++ | +++ |
| propionylamino]-ethyl}-N-pyridin-4-yl-benzamide | 399 | | |
| 4-{(R)-1-Amino-2-[2-(4-chloro-phenyl)-2-methyl- | Compound | ++ | ++ |
| propionylamino]-ethyl}-N-pyridin-4-yl-benzamide | 400 | | |
| 4-[(R)-1-Amino-2-(4-fluoro-benzylcarbamoyl)-ethyl]-N-pyridin-4 | Compound | ++++ | +++ |
| yl-benzamide | 401 | | |
| 4-[(S)-1-Amino-2-(4-fluoro-benzylcarbamoyl)-ethyl]-N-pyridin-4- | Compound | +++ | +++ |
| yl-benzamide | 402 | | |
| 4-[(R)-1-Amino-2-(2,3-dihydro-benzofuran-3-ylcarbamoyl)- | Compound | ++++ | ++++ |
| ethyl]-N-pyridin-4-yl-benzamide | 403 | | |
| 4-[(S)-1-Amino-2-(2,3-dihydro-benzofuran-3-ylcarbamoyl)- | Compound | ++ | +++ |
| ethyl]-N-pyridin-4-yl-benzamide | 404 | | |
| 4-{(R)-1-Amino-2-[(S)-(2,3-dihydro-benzofuran-3-yl)carbamoyl]- | Compound | ++++ | ++++ |
| ethyl}-N-pyridin-4-yl-benzamide | 405 | | |
| 4-{(R)-1-Amino-2-[(R)-(2,3-dihydro-benzofuran-3-yl)carbamoyl]- | Compound | +++ | +++ |
| ethyl}-N-pyridin-4-yl-benzamide | 406 | | |
| 4-{(S)-1-Amino-2-[(S)-(2,3-dihydro-benzofuran-3-yl)carbamoyl]- | Compound | nd | nd |
| ethyl}-N-pyridin-4-yl-benzamide | 407 | | |
| 4-{(S)-1-Amino-2-[(R)-(2,3-dihydro-benzofuran-3-yl)carbamoyl]- | Compound | nd | nd |
| ethyl}-N-pyridin-4-yl-benzamide | 408 | | |
| (R)-1,2,3,4-Tetrahydro-naphthalene-1-carboxylic acid {(S)-2- | Compound | nd | nd |
| amino-2-[4-(pyridin-4-ylcarbamoyl)-phenyl]-ethyl}-amide | 409 | | |
| (S)-1,2,3,4-Tetrahydro-naphthalene-1-carboxylic acid {(S)-2- | Compound | nd | nd |
| amino-2-[4-(pyridin-4-ylcarbamoyl)-phenyl]-ethyl}-amide | 410 | | |
| (R)-1,2,3,4-Tetrahydro-naphthalene-1-carboxylic acid {(R)-2- | Compound | nd | nd |
| amino-2-[4-(pyridin-4-ylcarbamoyl)-phenyl]-ethyl}-amide | 411 | | |
| (S)-1,2,3,4-Tetrahydro-naphthalene-1-carboxylic acid {(R)-2- | Compound | nd | nd |
| amino-2-[4-(pyridin-4-ylcarbamoyl)-phenyl]-ethyl}-amide | 412 | | |
| (R)-Indan-1-carboxylic acid {(S)-2-amino-2-[4-(pyridin-4- | Compound | nd | nd |
| ylcarbamoyl)-phenyl]-ethyl}-amide | 413 | | |
| (S)-Indan-1-carboxylic acid {(S)-2-amino-2-[4-(pyridin-4- | Compound | nd | nd |
| ylcarbamoyl)-phenyl]-ethyl}-amide | 414 | | |
| (R)-Indan-1-carboxylic acid {(R)-2-amino-2-[4-(pyridin-4- | Compound | nd | nd |
| ylcarbamoyl)-phenyl]-ethyl}-amide | 415 | | |
| (S)-Indan-1-carboxylic acid {(R)-2-amino-2-[4-(pyridin-4- | Compound | nd | nd |
| ylcarbamoyl)-phenyl]-ethyl}-amide | 416 | | |
| 4-((S)-1-Amino-2-{[1-(4-fluoro-phenyl)-cyclopentanecarbonyl]- | Compound | ++++ | +++ |
| amino}-ethyl)-N-pyridin-4-yl-benzamide | 417 | | |
| 4-((R)-1-Amino-2-{[1-(4-fluoro-phenyl)-cyclopentanecarbonyl]- | Compound | +++ | ++ |
| amino}-ethyl)-N-pyridin-4-yl-benzamide | 418 | | |
| 4-{1-Amino-2-[2-(4-fluoro-phenyl)-2-methyl-thiopropionylamino]- | Compound | ++++ | +++ |
| ethyl}-N-pyridin-4-yl-benzamide | 419 | | |

Table 6 shows the results for compounds of Formula XV. As used herein the term “nd” means “not determined yet” and “Pr” is “Protocol”.

TABLE 6

| | |
|---|---|
| | XV |
| 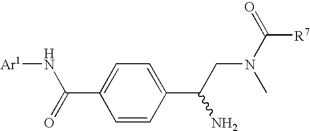 | |

| | | | | | IC₅₀ | IC₅₀ |
|---|---|---|---|---|---|---|
| | | | | | μM | μM |
| Name | Compound | Ar¹— | —R⁷ | Pr | PKCε | ROCK |
| 4-[1-Amino-2-(methyl- phenylacetyl-amino)-ethyl]- N-pyridin-4-yl-benzamide | Compound 420 | 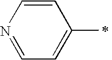 | 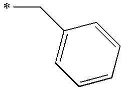 | B | + | ++ |
| 4-{1-Amino-2-[(2-ethyl-2- phenyl-butyryl)-methyl- amino]-ethyl}-N-pyridin-4-yl- benzamide | Compound 421 | 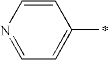 | 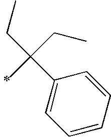 | B | nd | nd |
| 4-{1-Amino-2-[methyl-(2-o- tolyl-acetyl)-amino]-ethyl}-N- pyridin-4-yl-benzamide | Compound 422 | 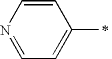 | 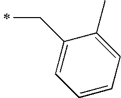 | B | + | ++ |
| 4-{1-Amino-2-[methyl-(2-m- tolyl-acetyl)-amino]-ethyl}-N- pyridin-4-yl-benzamide | Compound 423 | 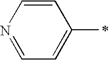 | 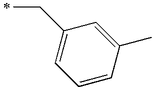 | B | + | ++ |
| 4-{1-Amino-2-[methyl-(2-p- tolyl-acetyl)-amino]-ethyl}-N- pyridin-4-yl-benzamide | Compound 424 | 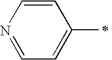 | 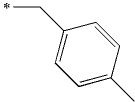 | B | + | ++ |
| 4-(1-Amino-2-{[2-(2- methoxy-phenyl)-acetyl]- methyl-amino}-ethyl)-N- pyridin-4-yl-benzamide | Compound 425 | 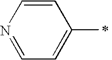 | 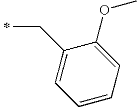 | B | + | ++ |
| 4-(1-Amino-2-{[2-(3- methoxy-phenyl)-acetyl}- methyl-amino}-ethyl)-N- pyridin-4-yl-benzamide | Compound 426 | 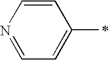 | 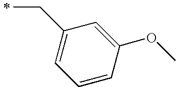 | B | + | +++ |
| 4-(1-Amino-2-{[2-(4- methoxy-phenyl)-acetyl]- methyl-amino}-ethyl)-N- pyridin-4-yl-benzamide | Compound 427 | 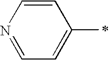 | 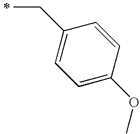 | B | + | +++ |
| 4-(1-Amino-2-{[1-(4- methoxy-phenyl)- cyclopropanecarbonyl]- methyl-amino}-ethyl)-N- pyridin-4-yl-benzamide | Compound 428 | 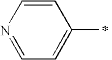 | 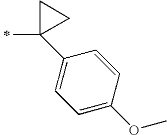 | B | + | ++ |
| 4-(1-Amino-2-{[1-(4- methoxy-phenyl)- cyclopentanecarbonyl]- methyl-amino}-ethyl)-N- pyridin-4-yl-benzamide | Compound 429 | 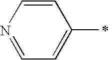 | 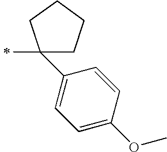 | B | nd | nd |
| 4-(1-Amino-2-{[2-(2,4- dimethoxy-phenyl)-acetyl]- methyl-amino}-ethyl)-N- pyridin-4-yl-benzamide | Compound 430 | 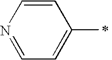 | 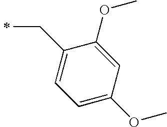 | B | + | +++ |
| 4-(1-Amino-2-{[2-(2,3,4- trimethoxy-phenyl)-acetyl]- methyl-amino}-ethyl)-N- pyridin-4-yl-benzamide | Compound 431 | 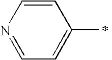 | 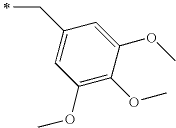 | B | nd | + |
| 4-{1-Amino-2-[(2- benzo[1,3]dioxol-5-yl- acetyl)-methyl-amino]-ethyl}- N-pyridin-4-yl-benzamide | Compound 432 | 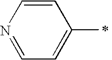 | 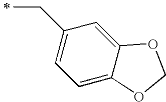 | B | + | ++ |
| 4-{1-Amino-2-[methyl-(2-9H- xanthen-9-yl-acetyl)- amino]-ethyl}- N-pyridin-4-yl-benzamide | Compound 433 | 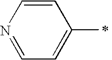 | 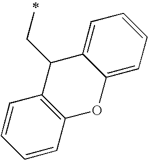 | B | nd | nd |
| 4-{1-Amino-2-[methyl-(2- naphthalen-2-yl-acetyl)- amino]-ethyl}- N-pyridin-4-yl-benzamide | Compound 434 | 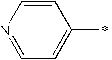 | 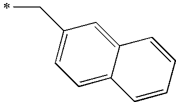 | B | ++ | +++ |
| 4-{1-Amino-2-[methyl-(2- naphthalen-1-yl-acetyl)- amino]-ethyl}- N-pyridin-4-yl-benzamide | Compound 435 | 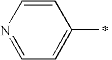 | 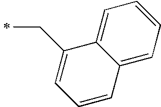 | B | + | ++ |
| 4-{1-Amino-2-[(2-biphenyl-4- yl-acetyl)-methyl- amino]-ethyl}- N-pyridin-4-yl-benzamide | Compound 436 | 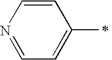 | 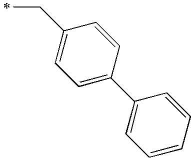 | B | + | ++ |
| 4-{1-Amino-2-[(2,2-diphenyl- propionyl)-methyl- amino]-ethyl}- N-pyridin-4-yl-benzamide | Compound 437 | 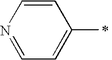 | 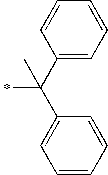 | B | nd | nd |
| 4-(1-Amino-2-{[2-(4-nitro- phenyl)-acetyl]-methyl- amino}-ethyl)- N-pyridin-4-yl-benzamide | Compound 438 | 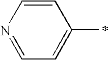 | 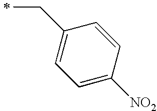 | B | nd | nd |
| 4-(1-Amino-2-{[2-(4- hydroxy-3-nitro-phenyl)- acetyl]-methyl- amino]-ethyl)- N-pyridin-4-yl-benzamide | Compound 439 | 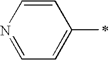 | 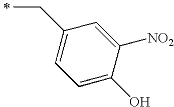 | B | + | +++ |
| 4-{1-Amino-2-[methyl-(2- pyridin-2-yl-acetyl)- amino]-ethyl}- N-pyridin-4-yl-benzamide | Compound 440 | 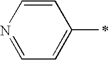 | 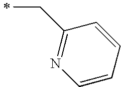 | B | nd | nd |
| 4-{1-Amino-2-[methyl-(2- pyridin-4-yl-acetyl)- amino]-ethyl}- N-pyridin-4-yl-benzamide | Compound 441 | 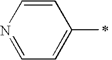 | 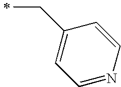 | B | nd | nd |
| 4-(1-Amino-2-{[2-(5-bromo- pyridin-3-yl)-acetyl]-methyl- amino}-ethyl)- N-pyridin-4-yl-benzamide | Compound 442 | 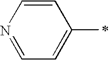 | 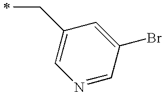 | B | + | ++ |
| 4-{1-Amino-2-[(2-1H-indol-3- yl-acetyl)-methyl- amino]-ethyl}- N-pyridin-4-yl-benzamide | Compound 443 | 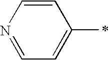 | 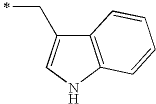 | B | + | ++ |
| 4-{1-Amino-2-[(2- benzo[b]thiophen-3-yl- acetyl)-methyl- amino]-ethyl}- N-pyridin-4-yl-benzamide | Compound 444 | 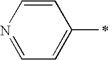 | 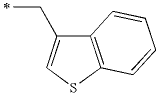 | B | + | ++ |
| 4-(1-Amino-2-{methyl-[2-(2- phenyl-thiazol-4-yl)-acetyl]- amino}-ethyl)- N-pyridin-4-yl-benzamide | Compound 445 | 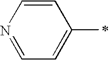 | 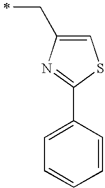 | B | + | ++ |
| 4-{1-Amino-2-[methyl-(2- thiophen-2-yl-acetyl)- amino]-ethyl}- N-pyridin-4-yl-benzamide | Compound 446 | 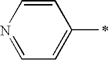 | 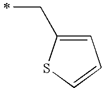 | B | nd | nd |
| 4-{1-Amino-2-[methyl-(2- methyl-2-thiophen-2-yl- propionyl)- amino]-ethyl}- N-pyridin-4-yl-benzamide | Compound 447 | 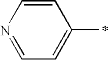 | 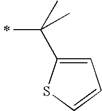 | B | ++ | ++ |
| 4-{1-Amino-2-[methyl-(1- thiophen-2-yl- cyclopropanecarbonyl)- amino]-ethyl}- N-pyridin-4-yl-benzamide | Compound 448 | 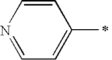 | 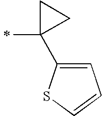 | B | + | ++ |
| 4-{1-Amino-2-[methyl-(2- methyl-2-thiophen-3-yl- propionyl)- amino]-ethyl}- N-pyridin-4-yl-benzamide | Compound 449 | 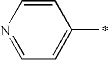 | 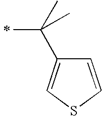 | B | + | ++ |
| 4-{1-Amino-2-[methyl-(1- thiophen-3-yl- cyclopropanecarbonyl)- amino]-ethyl}- N-pyridin-4-yl-benzamide | Compound 450 | 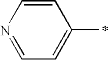 | 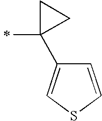 | B | + | ++ |
| Bicyclo[4.2.0]octa-1(6),2,4- triene-7-carboxylic acid {2- amino-2-[4-(pyridin-4- ylcarbamoyl)-phenyl]-ethyl}- methyl-amide | Compound 451 | 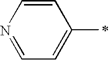 | 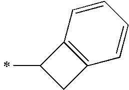 | B | + | +++ |
| 4-{1-Amino-2-[methyl-(1- phenyl- cyclopropanecarbonyl)- amino]-ethyl}- N-pyridin-4-yl-benzamide | Compound 452 | 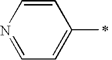 | 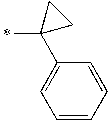 | B | + | ++ |
| 4-{1-Amino-2-[methyl-(1- phenyl- cyclohexanecarbonyl)- amino]-ethyl}- N-pyridin-4-yl-benzamide | Compound 453 | 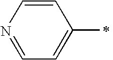 | 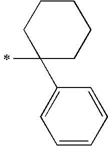 | B | nd | nd |
| 4-{1-Amino-2-[(2-cyclohexyl- 2-phenyl-acetyl)-methyl- amino]-ethyl}- N-pyridin-4-yl-benzamide | Compound 454 | 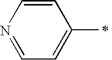 | 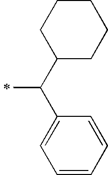 | B | nd | nd |
| 4-{1-Amino-2-[(2- cyclopentyl-2-phenyl- acetyl)-methyl- amino]-ethyl}- N-pyridin-4-yl-benzamide | Compound 455 | 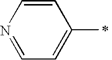 | 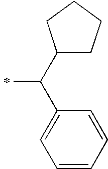 | B | + | + |
| 4-(1-Amino-2-{[2-(3-bromo- phenyl)-acetyl]-methyl- amino}-ethyl)- N-pyridin-4-yl-benzamide | Compound 456 | 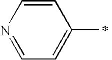 | 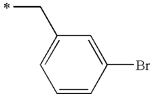 | B | + | ++ |
| 4-(1-Amino-2-{[2-(4-bromo- phenyl)-acetyl]-methyl- amino}-ethyl)- N-pyridin-4-yl-benzamide | Compound 457 | 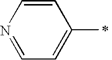 | 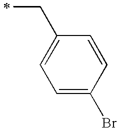 | B | + | ++ |
| 4-(1-Amino-2-{[2-(4- trifluoromethyl-phenyl)- acetyl]-methyl- amino}-ethyl)- N-pyridin-4-yl-benzamide | Compound 458 | 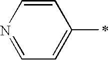 | 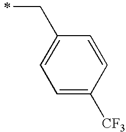 | B | + | ++ |
| 4-(1-Amino-2-{[2-(3-chloro- phenyl)-acetyl]-methyl- amino}-ethyl)- N-pyridin-4-yl-benzamide | Compound 459 | 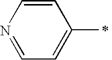 | 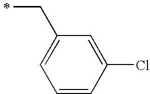 | B | + | ++ |
| 4-(1-Amino-2-{[2-(4-chloro- phenyl)-acetyl]-methyl- amino}-ethyl)- N-pyridin-4-yl-benzamide | Compound 460 | 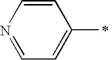 | 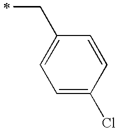 | B | + | ++ |
| 4-(1-Amino-2-{[2-(4-chloro- phenyl)-3-methyl-butyryl]- methyl- amino}-ethyl)- N-pyridin-4-yl-benzamide | Compound 461 | 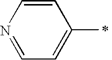 | 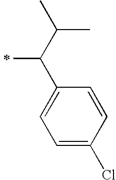 | B | + | ++ |
| 4-(1-Amino-2-{[2-(4-chloro- phenyl)-2-methyl-propionyl]- methyl- amino}-ethyl)- N-pyridin-4-yl-benzamide | Compound 462 | 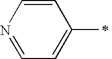 | 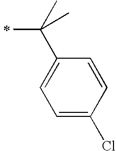 | C | ++ | ++ |
| 4-(1-Amino-2-{[1-(4-chloro- phenyl)- cyclopropanecarbonyl]- methyl- amino}-ethyl)- N-pyridin-4-yl-benzamide | Compound 463 | 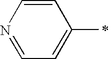 | 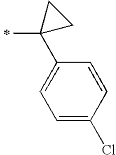 | B | ++ | ++ |
| 4-(1-Amino-2-{[1-(4-chloro- phenyl)- cyclobutanecarbonyl]- methyl- amino}-ethyl)- N-pyridin-4-yl-benzamide | Compound 464 | 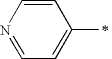 | 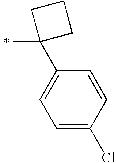 | B | ++ | ++ |
| 4-(1-Amino-2-{[1-(4-chloro- phenyl)- cyclopentanecarbonyl]- methyl- amino}-ethyl)- N-pyridin-4-yl-benzamide | Compound 465 | 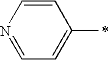 | 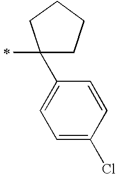 | B | ++ | ++ |
| 4-(1-Amino-2-{[2-(2-fluoro- phenyl)-acetyl]-methyl- amino}-ethyl)- N-pyridin-4-yl-benzamide | Compound 466 | 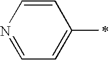 | 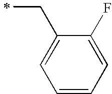 | B | + | ++ |
| 4-(1-Amino-2-{[1-(2-fluoro- phenyl)- cyclopentanecarbonyl]- methyl- amino}-ethyl)- N-pyridin-4-yl-benzamide | Compound 467 | 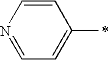 | 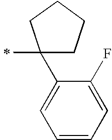 | B | nd | nd |
| 4-(1-Amino-2-{[2-(3-fluoro- phenyl)-acetyl]-methyl- amino}-ethyl)- N-pyridin-4-yl-benzamide | Compound 468 | 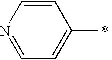 | 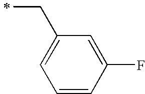 | B | + | ++ |
| 4-(1-Amino-2-{[1-(3-fluoro- phenyl)- cyclopentanecarbonyl]- methyl- amino}-ethyl)- N-pyridin-4-yl-benzamide | Compound 469 | 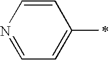 | 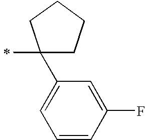 | B | nd | nd |
| 4-(1-Amino-2-{[2-(3-fluoro-4- methoxy-phenyl)-acetyl]- methyl- amino}-ethyl)- N-pyridin-4-yl-benzamide | Compound 470 | 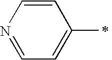 | 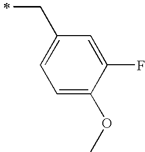 | B | + | ++ |
| 4-(1-Amino-2-{[2-(4-fluoro- phenyl)-acetyl]-methyl- amino}-ethyl)- N-pyridin-4-yl-benzamide | Compound 471 | 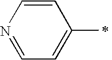 | 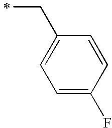 | B | + | ++ |
| 4-(1-Amino-2-{[2-(4-fluoro- phenyl)-2-methyl-propionyl]- methyl- amino}-ethyl)- N-pyridin-4-yl-benzamide | Compound 472 | 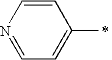 | 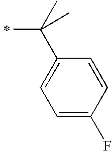 | C | ++ | ++ |
| 4-(1-Amino-2-{[1-(4-fluoro- phenyl)- cyclopropanecarbonyl]- methyl- amino}-ethyl)- N-pyridin-4-yl-benzamide | Compound 473 | 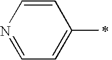 | 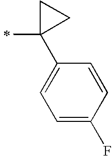 | B | ++ | ++ |
| 4-(1-Amino-2-{[1-(4-fluoro- phenyl)- cyclobutanecarbonyl]- methyl-amino}-ethyl)-N- pyridin-4-yl-benzamide | Compound 474 | 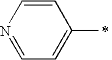 | 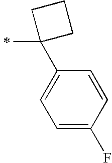 | B | ++ | ++ |
| 4-(1-Amino-2-{[1-(4-fluoro- phenyl)- cyclopentanecarbonyl]- methyl-amino}-ethyl)-N- pyridin-4-yl-benzamide | Compound 475 | 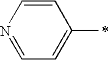 | 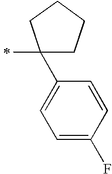 | C | ++ | ++ |
| 4-(1-Amino-2-{[1-(4-fluoro- phenyl)- cyclohexanecarbonyl]- methyl-amino}-ethyl)-N- pyridin-4-yl-benzamide | Compound 476 | 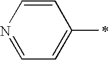 | 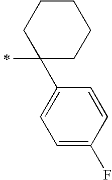 | B | nd | nd |
| 4-(1-Amino-2-{[2-(2,3- difluoro-phenyl)-acetyl]- methyl-amino}-ethyl)-N- pyridin-4-yl-benzamide | Compound 477 | 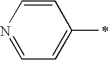 | 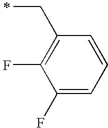 | B | + | ++ |
| 4-(1-Amino-2-{[2-(2,5- difluoro-phenyl)-acetyl]- methyl-amino}-ethyl)-N- pyridin-4-yl-benzamide | Compound 478 | 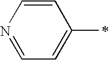 | 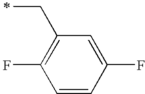 | B | + | ++ |
| 4-(1-Amino-2-{[2-(2,4- difluoro-phenyl)-acetyl]- methyl-amino}-ethyl)-N- pyridin-4-yl-benzamide | Compound 479 | 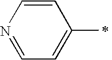 | 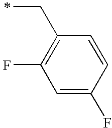 | B | + | ++ |
| 4-(1-Amino-2-{[2-(2,4- difluoro-phenyl)-2-methyl- propionyl]-methyl-amino}-ethyl)-N- pyridin-4-yl-benzamide | Compound 480 | 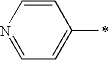 | 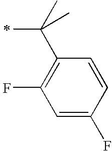 | B | nd | nd |
| 4-(1-Amino-2-{[1-(2,4- difluoro-phenyl)- cyclopropanecarbonyl]- methyl-amino}-ethyl)-N- pyridin-4-yl-benzamide | Compound 481 | 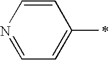 | 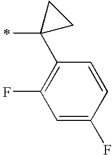 | B | + | ++ |
| 4-(1-Amino-2-{[2-(3,5- difluoro-phenyl)-acetyl]- methyl-amino}-ethyl)-N- pyridin-4-yl-benzamide | Compound 482 | 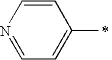 | 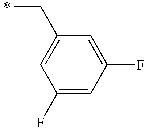 | B | nd | nd |
| 4-(1-Amino-2-{[2-(2,6- difluoro-phenyl)-acetyl]- methyl-amino}-ethyl)-N- pyridin-4-yl-benzamide | Compound 483 | 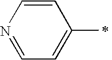 | 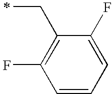 | B | nd | nd |
| 4-(1-Amino-2-{[2-(3,4- difluoro-phenyl)-acetyl]- methyl-amino}-ethyl)-N- pyridin-4-yl-benzamide | Compound 484 | 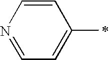 | 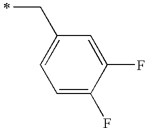 | C | nd | ++ |
| 4-(1-Amino-2-{[2-(3,4- difluoro-phenyl)-2-methyl- propionyl]-methyl-amino}-ethyl)-N- pyridin-4-yl-benzamide | Compound 485 | 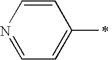 | 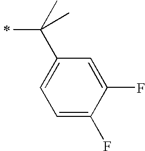 | C | ++ | ++ |
| 4-(1-Amino-2-{[1-(3,4- difluoro-phenyl)- cyclopropanecarbonyl]- methyl-amino}-ethyl)-N- pyridin-4-yl-benzamide | Compound 486 | 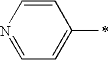 | 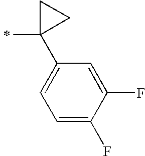 | B | ++ | ++ |
| 4-(1-Amino-2-{[1-(3,4- difluoro-phenyl)- cyclobutanecarbonyl]- methyl-amino}-ethyl)-N- pyridin-4-yl-benzamide | Compound 487 | 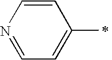 | 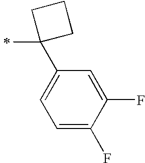 | B | ++ | ++ |
| 4-(1-Amino-2-{[1-(3,4- difluoro-phenyl)- cyclopentanecarbonyl]- methyl-amino}-ethyl)-N- pyridin-4-yl-benzamide | Compound 488 | 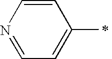 | 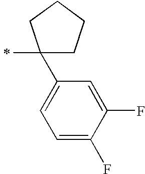 | B | ++ | ++ |
| 4-(1-Amino-2-{[2-(2,6- dichloro-phenyl)-acetyl]- methyl-amino}-ethyl)-N- pyridin-4-yl-benzamide | Compound 489 | 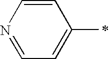 | 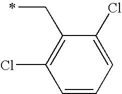 | B | nd | nd |
| 4-(1-Amino-2-{[2-(2,4- dichloro-phenyl)-acetyl]- methyl-amino}-ethyl)-N- pyridin-4-yl-benzamide | Compound 490 | 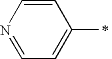 | 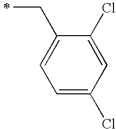 | B | + | ++ |
| 4-(1-Amino-2-{[2-(3,4- dichloro-phenyl)-acetyl]- methyl-amino}-ethyl)-N- pyridin-4-yl-benzamide | Compound 491 | 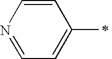 | 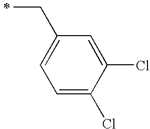 | B | + | ++ |
| 4-(1-Amino-2-{[2-(3,4- dichloro-phenyl)-2-methyl- propionyl]-methyl-amino}-ethyl)-N- pyridin-4-yl-benzamide | Compound 492 | 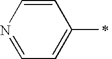 | 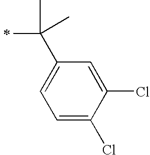 | B | nd | nd |
| 4-(1-Amino-2-{[1-(3,4- dichloro-phenyl)- cyclopropanecarbonyl]- methyl-amino}-ethyl)-N- pyridin-4-yl-benzamide | Compound 493 | 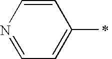 | 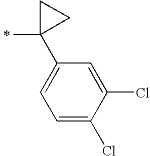 | B | + | ++ |
| 4-(1-Amino-2-{[1-(3,4- dichloro-phenyl)- cyclobutanecarbonyl]- methyl-amino}-ethyl)-N- pyridin-4-yl-benzamide | Compound 494 | 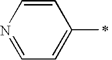 | 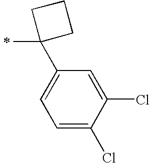 | B | + | ++ |
| 4-(1-Amino-2-{[1-(2,4- dichloro-phenyl)- cyclopropanecarbonyl]- methyl-amino}-ethyl)-N- pyridin-4-yl-benzamide | Compound 495 | 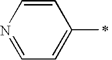 | 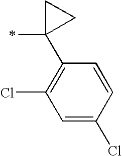 | B | + | ++ |
| 4-(1-Amino-2-{[2-(2-chloro- 6-fluoro-phenyl)-acetyl]- methyl-amino}-ethyl)-N- pyridin-4-yl-benzamide | Compound 496 | 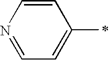 | 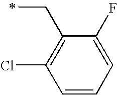 | B | + | ++ |
| 4-(1-Amino-2-{[2-(4-chloro- 2-fluoro-phenyl)-acetyl]- methyl-amino}-ethyl)-N- pyridin-4-yl-benzamide | Compound 497 | 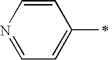 | 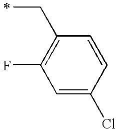 | B | nd | nd |
| 4-(1-Amino-2-{[2-(2-chloro- 4-fluoro-phenyl)-acetyl]- methyl-amino}-ethyl)-N- pyridin-4-yl-benzamide | Compound 498 | 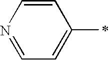 | 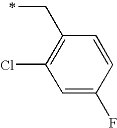 | B | + | ++ |
| 4-(1-Amino-2-{[1-(2-chloro- 4-fluoro-phenyl)- cyclopropanecarbonyl]- methyl-amino}-ethyl)-N- pyridin-4-yl-benzamide | Compound 499 | 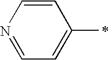 | 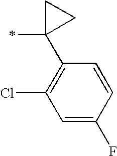 | B | nd | nd |
| 4-(1-Amino-2-{[1-(2-chloro- 4-fluoro-phenyl)- cyclopentanecarbonyl]- methyl-amino}-ethyl)-N- pyridin-4-yl-benzamide | Compound 500 | 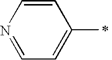 | 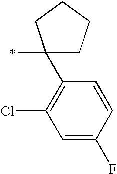 | B | nd | nd |
| 4-(1-Amino-2-{[2-(3-chloro- 2-fluoro-phenyl)-acetyl]- methyl-amino}-ethyl)-N- pyridin-4-yl-benzamide | Compound 501 | 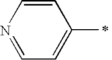 | 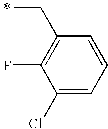 | B | + | ++ |
| 4-(1-Amino-2-{[2-(4-chloro- 2-fluoro-phenyl)-acetyl]- methyl-amino}-ethyl)-N- pyridin-4-yl-benzamide | Compound 502 | 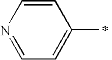 | 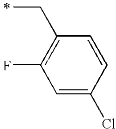 | B | + | ++ |
| 4-(1-Amino-2-{[2-(4-chloro- 2-fluoro-phenyl)-2-methyl- propionyl]-methyl-amino}-ethyl)-N- pyridin-4-yl-benzamide | Compound 503 | 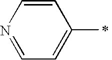 | 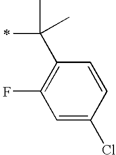 | B | nd | nd |
| 4-(1-Amino-2-{[1-(4-chloro- 2-fluoro-phenyl)- cyclopropanecarbonyl]- methyl-amino}-ethyl)-N- pyridin-4-yl-benzamide | Compound 504 | 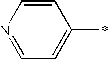 | 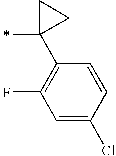 | B | ++ | ++ |
| 4-(1-Amino-2-{[2-(3-chloro- 4-fluoro-phenyl)-2-methyl- propionyl]-methyl-amino}-ethyl)-N- pyridin-4-yl-benzamide | Compound 505 | 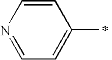 | 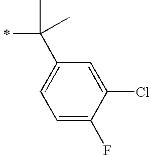 | B | + | ++ |
| 4-(1-Amino-2-{[1-(3-chloro- 4-fluoro-phenyl)- cyclopropanecarbonyl]- methyl-amino}-ethyl)-N- pyridin-4-yl-benzamide | Compound 506 | 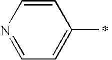 | 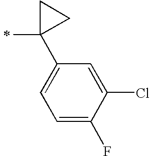 | B | + | ++ |
| Indan-1-carboxylic acid {2- amino-2-[4-(pyridin-4- ylcarbamoyl)-phenyl]-ethyl}- methyl-amide | Compound 507 | 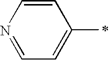 | 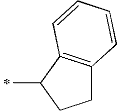 | B | + | ++ |
| 3-Oxo-indan-1-carboxylic acid {2-amino-2-[4-(pyridin- 4-ylcarbamoyl)-phenyl]- ethyl}-methyl-amide | Compound 508 | 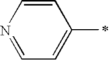 | 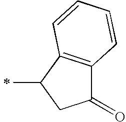 | B | nd | ++ |
| 5-Fluoro-indan-1-carboxylic acid {2-amino-2-[4-(pyridin- 4-ylcarbamoyl)-phenyl]- ethyl}-methyl-amide | Compound 509 | 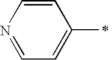 | 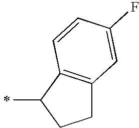 | B | nd | nd |
| 6-Fluoro-indan-1-carboxylic acid {2-amino-2-[4-(pyridin- 4-ylcarbamoyl)-phenyl]- ethyl}-methyl-amide | Compound 510 | 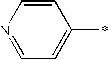 | 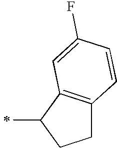 | B | nd | nd |
| 5-Chloro-indan-1-carboxylic acid {2-amino-2-[4-(pyridin- 4-ylcarbamoyl)-phenyl]- ethyl}-methyl-amide | Compound 511 | 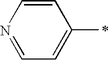 | 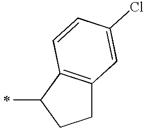 | B | nd | nd |
| 1,2,3,4-Tetrahydro- naphthalene-1-carboxylic acid {2-amino-2-[4-(pyridin- 4-ylcarbamoyl)-phenyl]- ethyl}-methyl-amide | Compound 512 | 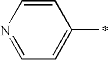 | 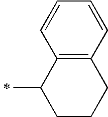 | B | + | ++ |
| 1,2,3,4-Tetrahydro- phenanthrene-4-carboxylic acid {2-amino-2-[4-(pyridin- 4-ylcarbamoyl)-phenyl]- ethyl}-methyl-amide | Compound 513 | 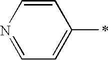 | 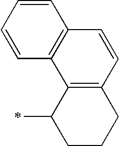 | B | nd | nd |
| Chroman-4-carboxylic acid {2-amino-2-[4-(pyridin-4- ylcarbamoyl)-phenyl]-ethyl}- methyl-amide | Compound 514 | 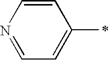 | 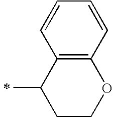 | B | + | ++ |
| 6-Chloro-chroman-4- carboxylic acid {2-amino-2- [4-(pyridin-4-ylcarbamoyl)- phenyl]-ethyl}-methyl-amide | Compound 515 | 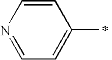 | 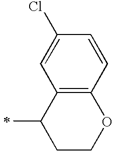 | B | + | ++ |
| 6-Fluoro-chroman-4- carboxylic acid {2-amino-2- [4-(pyridin-4-ylcarbamoyl)- phenyl]-ethyl}-methyl-amide | Compound 516 | 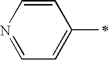 | 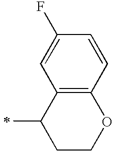 | B | + | ++ |
| Thiochroman-4-carboxylic acid {2-amino-2- {4-(pyridin-4-ylcarbamoyl)- phenyl]-ethyl}-methyl-amide | Compound 517 | 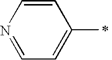 | 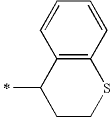 | B | + | + |
| 6-Fluoro-thiochroman-4- carboxylic acid {2-amino-2- [4-(pyridin-4-ylcarbamoyl)- phenyl]-ethyl}-methyl-amide | Compound 518 | 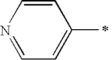 | 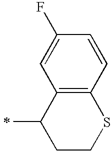 | B | + | + |
| 6,7,8,9-Tetrahydro-5H- benzocycloheptane-5- carboxylic acid {2-amino-2- [4-(pyridin-4-ylcarbamoyl)- phenyl]-ethyl}-methyl-amide | Compound 519 | 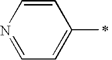 | 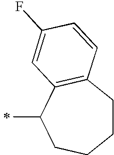 | B | nd | nd |
| 4,5,6,7-Tetrahydro- benzo[b]thiophene-4- carboxylic acid {2-amino-2- [4-(pyridin-4-ylcarbamoyl)- phenyl]-ethyl}-methyl-amide | Compound 520 | 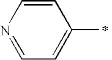 | 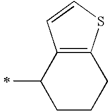 | B | + | ++ |

Table 7 shows the results for compounds of Formula XV. As used herein the term “nd” means “not determined yet” and “Pr” is “Protocol”.

TABLE 7

| | |
|---|---|
| | XVI |
| 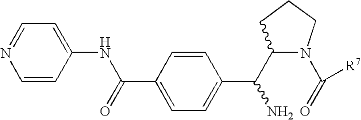 | |

| | | | | IC₅₀ | IC₅₀ |
|---|---|---|---|---|---|
| | | | | μM | μM |
| Name | Compound | —R⁷ | Pr | PKCε | ROCK |
| 4-[Amino-(1-phenylacetyl- pyrrolidin-2-yl)-methyl]-N-pyridin- 4-yl-benzamide | Compound 521 | 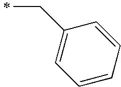 | B | ++ | +++ |
| 4-{Amino-[1-(2-ethyl-2-phenyl- butyryl)-pyrrolidin-2-yl]-methyl}-N- pyridin-4-yl-benzamide | Compound 522 | 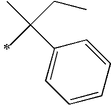 | B | nd | nd |
| 4-{Amino-[1-(2-o-tolyl-acetyl)- pyrrolidin-2-yl]-methyl}-N-pyridin- 4-yl-benzamide | Compound 523 | 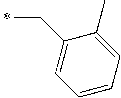 | B | nd | nd |
| 4-{Amino-[1-(2-m-tolyl-acetyl)- pyrrolidin-2-yl]-methyl}-N-pyridin- 4-yl-benzamide | Compound 524 | 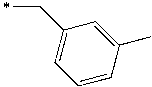 | B | + | ++ |
| 4-{Amino-[1-(2-p-tolyl-acetyl)- pyrrolidin-2-yl]-methyl}-N-pyridin- 4-yl-benzamide | Compound 525 | 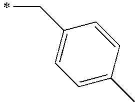 | B | ++ | ++ |
| 4-(Amino-{1-[2-(2-methoxy- phenyl)-acetyl]-pyrrolidin-2-yl]- methyl}-N-pyridin- 4-yl-benzamide | Compound 526 | 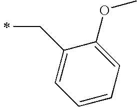 | B | + | ++ |
| 4-(Amino-{1-[2-(3-methoxy- phenyl)-acetyl]-pyrrolidin-2-yl}- methyl)-N-pyridin-4-yl-benzamide | Compound 527 | 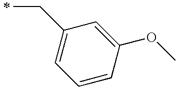 | B | ++ | ++ |
| 4-(Amino-{1-[2-(4-methoxy- phenyl)-acetyl]-pyrrolidin-2-yl}- methyl)-N-pyridin-4-yl-benzamide | Compound 528 | 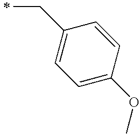 | B | ++ | +++ |
| 4-(Amino-{1-[1-(4-methoxy- phenyl)-cyclopropanecarbonyl]- pyrrolidin-2-yl}-methyl)-N-pyridin- 4-yl-benzamide | Compound 529 | 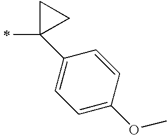 | B | ++ | ++ |
| 4-(Amino-{1-[1-(4-methoxy- phenyl)-cyclopentanecarbonyl]- pyrrolidin-2-yl}-methyl)-N-pyridin- 4-yl-benzamide | Compound 530 | 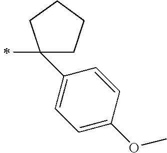 | B | ++ | ++ |
| 4-(Amino-{1-[2-(2,4-dimethoxy- phenyl)-acetyl]-pyrrolidin-2-yl}- methyl)-N-pyridin-4-yl-benzamide | Compound 531 | 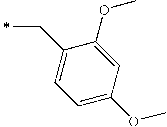 | B | ++ | ++ |
| 4-(Amino-{1-[2-(3,4,5-trimethoxy- phenyl)-acetyl]-pyrrolidin-2-yl}- methyl)-N-pyridin-4-yl-benzamide | Compound 532 | 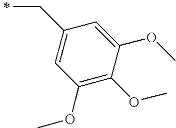 | B | + | ++ |
| 4-{Amino-[1-(2-benzo[1,3]dioxol-5- yl-acetyl)-pyrrolidin-2-yl]-methyl}- N-pyridin-4-yl-benzamide | Compound 533 | 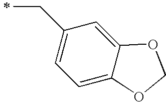 | B | nd | nd |
| 4-{Amino-[1-(9H-xanthene-9- carbonyl)-pyrrolidin-2-yl]-methyl}- N-pyridin-4-yl-benzamide | Compound 534 | 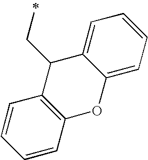 | B | nd | nd |
| 4-{Amino-[1-(2-naphthalen-2-yl- acetyl)-pyrrolidin-2-yl]-methyl}-N- pyridin-4-yl-benzamide | Compound 535 | 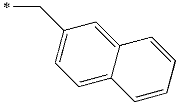 | B | ++ | ++ |
| 4-{Amino-[1-(2-naphthalen-1-yl- acetyl)-pyrrolidin-2-yl]-methyl}-N- pyridin-4-yl-benzamide | Compound 536 | 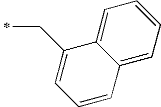 | B | ++ | ++ |
| 4-{Amino-[1-(2-biphenyl-4-yl- acetyl)-pyrrolidin-2-yl]-methyl}-N- pyridin-4-yl-benzamide | Compound 537 | 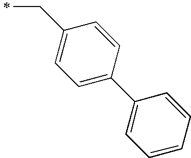 | B | ++ | ++ |
| 4-{Amino-[1-(2,2-diphenyl- propionyl)-pyrrolidin-2-yl]-methyl}- N-pyridin-4-yl-benzamide | Compound 538 | 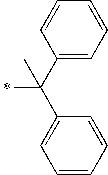 | B | nd | nd |
| 4-(Amino-{1-[2-(4-nitro-phenyl)- acetyl]-pyrrolidin-2-yl}-methyl)-N- pyridin-4-yl-benzamide | Compound 539 | 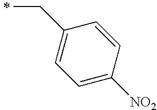 | B | nd | nd |
| 4-(Amino-{1-[2-(4-hydroxy-3-nitro- phenyl)-acetyl]-pyrrolidin-2-yl}- methyl)-N-pyridin-4-yl-benzamide | Compound 540 | 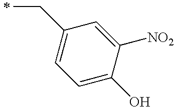 | B | + | ++ |
| 4-{Amino-[1-(2-pyridin-2-yl-acetyl)- pyrrolidin-2-yl]-methyl}-N-pyridin- 4-yl-benzamide | Compound 541 | 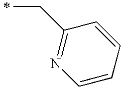 | B | nd | Nd |
| 4-{Amino-[1-(2-pyridin-4-yl-acetyl)- pyrrolidin-2-yl]-methyl}-N-pyridin- 4-yl-benzamide | Compound 542 | 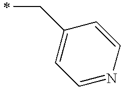 | B | nd | nd |
| 4-(Amino-{1-[2-(5-bromo-pyridin-3- yl)-acetyl]-pyrrolidin-2-yl}-methyl)- N-pyridin-4-yl-benzamide | Compound 543 | 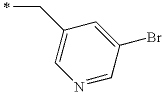 | B | + | ++ |
| 4-{Amino-[1-(2-1H-indol-3-yl- acetyl)-pyrrolidin-2-yl]-methyl}-N- pyridin-4-yl-benzamide | Compound 544 | 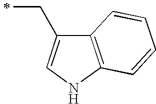 | B | ++ | ++ |
| 4-{Amino-[1-(2-1H-indol-3-yl- acetyl)-pyrrolidin-2-yl]-methyl}-N- pyridin-4-yl-benzamide | Compound 545 | 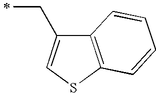 | B | ++ | +++ |
| 4-(Amino-{1-[2-(2-phenyl-thiazol-4- yl)-acetyl]-pyrrolidin-2-yl}-methyl)- N-pyridin-4-yl-benzamide | Compound 546 | 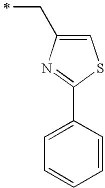 | B | ++ | ++ |
| 4-{Amino-[1-(2-thiophen-2-yl- acetyl)-pyrrolidin-2-yl]-methyl}-N- pyridin-4-yl-benzamide | Compound 547 | 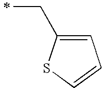 | B | + | +++ |
| 4-{Amino-[1-(2-methyl-2-thiophen- 2-yl-propionyl)-pyrrolidin-2-yl]- methyl}-N-pyridin-4-yl-benzamide | Compound 548 | 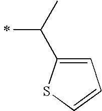 | B | ++ | ++ |
| 4-{Amino-[1-(1-thiophen-2-yl- cyclopropanecarbonyl)-pyrrolidin- 2-yl]-methyl}-N-pyridin-4-yl- benzamide | Compound 549 | 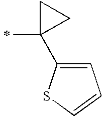 | B | + | ++ |
| 4-{Amino-[1-(2-methyl-2-thiophen- 3-yl-propionyl)-pyrrolidin-2-yl]- methyl}-N-pyridin-4-yl-benzamide | Compound 550 | 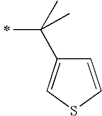 | B | nd | ++ |
| 4-{Amino-[1-(1-thiophen-3-yl- cyclopropanecarbonyl)-pyrrolidin- 2-yl]-methyl}-N-pyridin-4-yl- benzamide | Compound 551 | 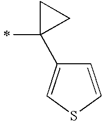 | B | + | ++ |
| 4-{Amino-[1-(bicyclo[4.2.0]octa- 1,3,5-triene-7-carbonyl)-pyrrolidin- 2-yl]-methyl}-N-pyridin-4-yl- benzamide | Compound 552 | 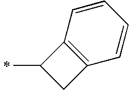 | B | + | +++ |
| 4-{Amino-[1-(1-phenyl- cyclopropanecarbonyl)-pyrrolidin- 2-yl]-methyl}-N-pyridin-4-yl- benzamide | Compound 553 | 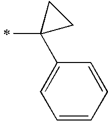 | B | nd | nd |
| 4-{Amino-[1-(1-phenyl- cyclohexanecarbonyl)-pyrrolidin-2- yl]-methyl}-N-pyridin-4-yl- benzamide | Compound 554 | 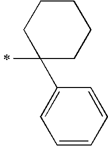 | B | nd | nd |
| 4-{Amino-[1-(2-cyclohexyl-2- phenyl-acetyl)-pyrrolidin-2-yl]- methyl}-N-pyridin-4-yl-benzamide | Compound 555 | 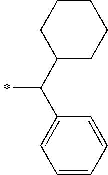 | B | ++ | ++ |
| 4-{Amino-[1-(2-cyclopentyl-2- phenyl-acetyl)-pyrrolidin-2-yl]- methyl}-N-pyridin-4-yl-benzamide | Compound 556 | 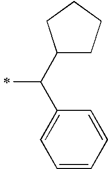 | B | + | ++ |
| 4-(Amino-{1-[2-(3-bromo-phenyl)- acetyl]-pyrrolidin-2-yl}-methyl)-N- pyridin-4-yl-benzamide | Compound 557 | 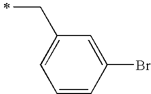 | B | ++ | +++ |
| 4-(Amino-{1-[2-(4-bromo-phenyl)- acetyl]-pyrrolidin-2-yl}-methyl)-N- pyridin-4-yl-benzamide | Compound 558 | 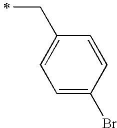 | B | ++ | +++ |
| 4-(Amino-{1-[2-(4-trifluoromethyl- phenyl)-acetyl]-pyrrolidin-2-yl}- methyl)-N-pyridin-4-yl-benzamide | Compound 559 | 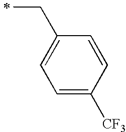 | B | + | ++ |
| 4-(Amino-{1-[2-(3-chloro-phenyl)- acetyl]-pyrrolidin-2-yl}-methyl)-N- pyridin-4-yl-benzamide | Compound 560 | 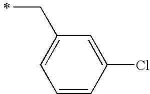 | B | + | ++ |
| 4-(Amino-{1-[2-(4-chloro-phenyl)- acetyl]-pyrrolidin-2-yl}-methyl)-N- pyridin-4-yl-benzamide | Compound 561 | 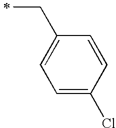 | B | ++ | ++ |
| 4-(Amino-{1-[2-(4-chloro-phenyl)- 3-methyl-butyryl]-pyrrolidin-2-yl}- methyl)-N-pyridin-4-yl-benzamide | Compound 562 | 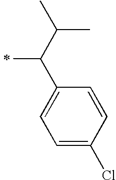 | B | ++ | ++ |
| 4-(Amino-{1-[2-(4-chloro-phenyl)- 2-methyl-propionyl]-pyrrolidin-2- yl}-methyl)-N-pyridin-4-yl- benzamide | Compound 563 | 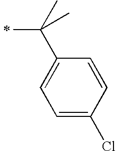 | C | ++ | ++ |
| 4-(Amino-{1-[1-(4-chloro-phenyl)- cyclopropanecarbonyl]-pyrrolidin- 2-yl}-methyl)-N-pyridin-4-yl- benzamide | Compound 564 | 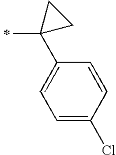 | B | ++ | ++ |
| 4-(Amino-{1-[1-(4-chloro-phenyl)- cyclobutanecarbonyl]-pyrrolidin-2- yl}-methyl)-N-pyridin-4-yl- benzamide | Compound 565 | 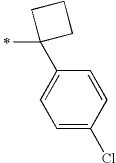 | B | ++ | ++ |
| 4-(Amino-{1-[1-(4-chloro-phenyl)- cyclopentanecarbonyl]-pyrrolidin- 2-yl}-methyl)-N-pyridin-4-yl- benzamide | Compound 566 | 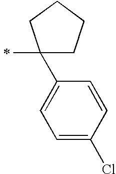 | B | ++ | ++ |
| 4-(Amino-{1-[2-(2-fluoro-phenyl)- acetyl]-pyrrolidin-2-yl}-methyl)-N- pyridin-4-yl-benzamide | Compound 567 | 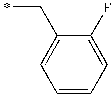 | B | + | +++ |
| 4-(Amino-{1-[1-(2-fluoro-phenyl)- cyclopentanecarbonyl]-pyrrolidin- 2-yl}-methyl)-N-pyridin-4-yl- benzamide | Compound 568 | 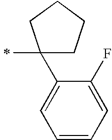 | B | nd | nd |
| 4-(Amino-{1-[2-(3-fluoro-phenyl)- acetyl-pyrrolidin-2-yl}-methyl-N- pyridin-4-yl-benzamide | Compound 569 | 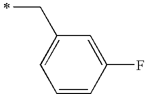 | B | + | ++ |
| 4-(Amino-{1-[1-(3-fluoro-phenyl)- cyclopentanecarbonyl]-pyrrolidin- 2-yl}-methyl)-N-pyridin-4-yl- benzamide | Compound 570 | 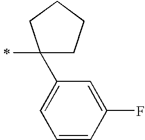 | B | ++ | ++ |
| 4-(Amino-{1-[2-(3-fluoro-4- methoxy-phenyl)-acetyl]-pyrrolidin- 2-yl}-methyl)-N-pyridin-4-yl- benzamide | Compound 571 | 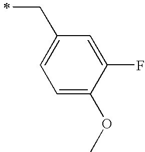 | B | + | ++ |
| 4-(Amino-{1-[2-(4-fluoro-phenyl)- acetyl]-pyrrolidin-2-yl}-methyl)-N- pyridin-4-yl-benzamide | Compound 572 | 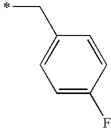 | C | + | ++ |
| 4-(Amino-{1-[2-(4-fluoro-phenyl)-2- methyl-propionyl]-pyrrolidin-2-yl}- methyl)-N-pyridin-4-yl-benzamide | Compound 573 | 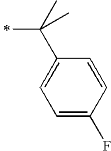 | C | ++ | ++ |
| 4-(Amino-{1-[1-(4-fluoro-phenyl)- cyclopropanecarbonyl]-pyrrolidin- 2-yl}-methyl)-N-pyridin-4-yl- benzamide | Compound 574 | 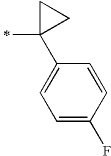 | B | ++ | ++ |
| 4-(Amino-{1-[1-(4-fluoro-phenyl)- cyclobutanecarbonyl]-pyrrolidin- 2-yl}-methyl)-N-pyridin-4-yl- benzamide | Compound 575 | 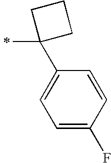 | B | ++ | ++ |
| 4-(Amino-{1-[1-(4-fluoro-phenyl)- cyclopentanecarbonyl]-pyrrolidin- 2-yl}-methyl)-N-pyridin-4-yl- benzamide | Compound 576 | 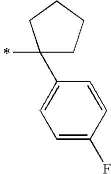 | C | ++ | ++ |
| 4-(Amino-{1-[1-(4-fluoro-phenyl)- cyclohexanecarbonyl]-pyrrolidin-2- yl}-methyl)-N-pyridin-4-yl- benzamide | Compound 577 | 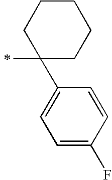 | B | nd | nd |
| 4-(Amino-{1-[2-(2,3-difluoro- phenyl)-acetyl]-pyrrolidin-2-yl}- methyl)-N-pyridin-4-yl-benzamide | Compound 578 | 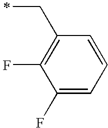 | B | ++ | ++ |
| 4-(Amino-{1-[2-(2,5-difluoro- phenyl)-acetyl]-pyrrolidin-2-yl}- methyl)-N-pyridin-4-yl-benzamide | Compound 579 | 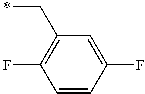 | B | + | ++ |
| 4-(Amino-{1-[2-(2,4-difluoro- phenyl)-acetyl]-pyrrolidin-2-yl}- methyl)-N-pyridin-4-yl-benzamide | Compound 580 | 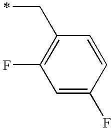 | B | ++ | +++ |
| 4-(Amino-{1-[2-(2,4-difluoro- phenyl)-2-methyl-propionyl]- pyrrolidin-2-yl}-methyl)-N-pyridin- 4-yl-benzamide | Compound 581 | 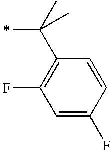 | B | nd | nd |
| 4-(Amino-{1-[1-(2,4-difluoro- phenyl)-cyclopropanecarbonyl]- pyrrolidin-2-yl}-methyl)-N-pyridin- 4-yl-benzamide | Compound 582 | 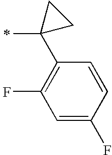 | B | ++ | ++ |
| 4-(Amino-{1-[2-(3,5-difluoro- phenyl)-acetyl]-pyrrolidin-2-yl}- methyl)-N-pyridin-4-yl-benzamide | Compound 583 | 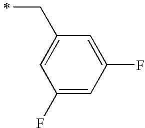 | B | + | ++ |
| 4-(Amino-{1-[2-(2,6-difluoro- phenyl)-acetyl]-pyrrolidin-2-yl}- methyl)-N-pyridin-4-yl-benzamide | Compound 584 | 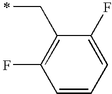 | B | + | ++ |
| 4-(Amino-{1-[2-(3,4-difluoro- phenyl)-acetyl]-pyrrolidin-2-yl}- methyl)-N-pyridin-4-yl-benzamide | Compound 585 | 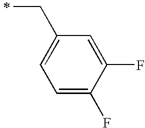 | B | nd | nd |
| 4-(Amino-{1-[2-(4-fluoro-phenyl)-2- methyl-propionyl]-pyrrolidin-2-yl}- methyl)-N-pyridin-4-yl-benzamide | Compound 586 | 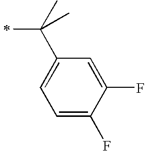 | C | ++ | ++ |
| 4-(Amino-{1-[1-(3,4-difluoro- phenyl)-cyclopropanecarbonyl]- pyrrolidin-2-yl}-methyl)-N-pyridin- 4-yl-benzamide | Compound 587 | 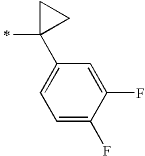 | B | ++ | ++ |
| 4-(Amino-{1-[1-(3,4-difluoro- phenyl)-cyclobutanecarbonyl]- pyrrolidin-2-yl}-methyl)-N-pyridin- 4-yl-benzamide | Compound 588 | 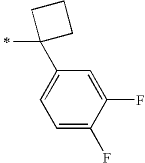 | B | ++ | ++ |
| 4-(Amino-{1-[1-(3,4-difluoro- phenyl)-cyclopentanecarbonyl]- pyrrolidin-2-yl}-methyl)-N-pyridin- 4-yl-benzamide | Compound 589 | 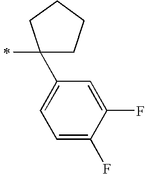 | B | ++ | ++ |
| 4-(Amino-{1-[2-(2,6-dichloro- phenyl)-acetyl]-pyrrolidin-2-yl}- methyl)-N-pyridin-4-yl-benzamide | Compound 590 | 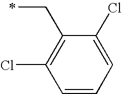 | B | + | ++ |
| 4-(Amino-{1-[2-(2,4-dichloro- phenyl)-acetyl]-pyrrolidin-2-yl}- methyl)-N-pyridin-4-yl-benzamide | Compound 591 | 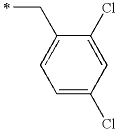 | B | + | ++ |
| 4-(Amino-{1-[2-(3,4-dichloro- phenyl)-acetyl]-pyrrolidin-2-yl}- methyl)-N-pyridin-4-yl-benzamide | Compound 592 | 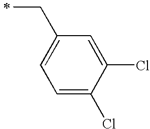 | B | ++ | ++ |
| 4-(Amino-{1-[2-(3,4-dichloro- phenyl)-2-methyl-propionyl]- pyrrolidin-2-yl}- methyl)-N-pyridin-4-yl-benzamide | Compound 593 | 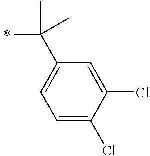 | B | + | ++ |
| 4-(Amino-{1-[1-(3,4-dichloro- phenyl)-cyclopropanecarbonyl]- pyrrolidin-2-yl}-methyl)-N-pyridin- 4-yl-benzamide | Compound 594 | 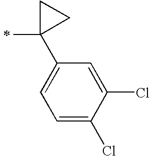 | B | ++ | ++ |
| 4-(Amino-{1-[1-(3,4-dichloro- phenyl)-cyclobutanecarbonyl]- pyrrolidin-2-yl}-methyl)-N-pyridin- 4-yl-benzamide | Compound 595 | 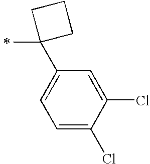 | B | ++ | ++ |
| 4-(Amino-{1-[1-(2,4-dichloro- phenyl)-cyclopropanecarbonyl]- pyrrolidin-2-yl}-methyl)-N-pyridin- 4-yl-benzamide | Compound 596 | 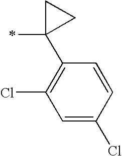 | B | + | ++ |
| 4-(Amino-{1-[2-(2-chloro-6-fluoro- phenyl)-acetyl]-pyrrolidin-2-yl}- methyl)-N-pyridin-4-yl-benzamide | Compound 597 | 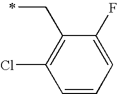 | B | + | ++ |
| 4-(Amino-{1-[2-(4-chloro-2-fluoro- phenyl)-acetyl]-pyrrolidin-2-yl}- methyl)-N-pyridin-4-yl-benzamide | Compound 598 | 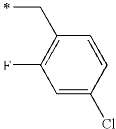 | B | + | ++ |
| 4-(Amino-{1-[2-(2-chloro-4-fluoro- phenyl)-acetyl]-pyrrolidin-2-yl}- methyl)-N-pyridin-4-yl-benzamide | Compound 599 | 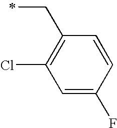 | B | ++ | ++ |
| 4-(Amino-{1-[1-(2-chloro-4-fluoro- phenyl)-cyclopropanecarbonyl]- pyrrolidin-2-yl}- methyl)-N-pyridin-4-yl-benzamide | Compound 600 | 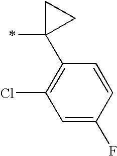 | B | ++ | ++ |
| 4-(Amino-{1-[1-(2-chloro-4-fluoro- phenyl)-cyclopentanecarbonyl]- pyrrolidin-2-yl}- methyl)-N-pyridin-4-yl-benzamide | Compound 601 | 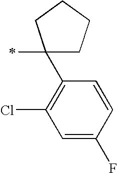 | B | nd | nd |
| 4-(Amino-{1-[2-(3-chloro-2-fluoro- phenyl)-acetyl]-pyrrolidin-2-yl}- methyl)-N-pyridin-4-yl-benzamide | Compound 602 | 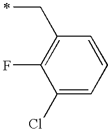 | B | + | ++ |
| 4-(Amino-{1-[2-(3-chloro-4-fluoro- phenyl)-acetyl]-pyrrolidin-2-yl}- methyl)-N-pyridin-4-yl-benzamide | Compound 603 | 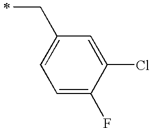 | B | ++ | +++ |
| 4-(Amino-{1-[2-(4-chloro-2-fluoro- phenyl)-2-methyl-propionyl]- pyrrolidin-2-yl}-methyl)-N-pyridin- 4-yl-benzamide | Compound 604 | 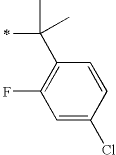 | B | nd | nd |
| 4-(Amino-{1-[1-(4-chloro-2-fluoro- phenyl)-cyclopropanecarbonyl]- pyrrolidin-2-yl}-methyl)-N-pyridin- 4-yl-benzamide | Compound 605 | 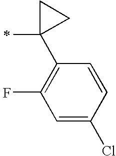 | B | ++ | ++ |
| 4-(Amino-{1-[2-(3-chloro-4-fluoro- phenyl)-2-methyl-propionyl]- pyrrolidin-2-yl}-methyl)-N-pyridin- 4-yl-benzamide | Compound 606 | 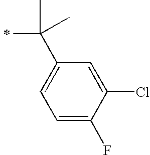 | B | ++ | ++ |
| 4-(Amino-{1-[1-(3-chloro-4-fluoro- phenyl)-cyclopropanecarbonyl]- pyrrolidin-2-yl}-methyl)-N-pyridin- 4-yl-benzamide | Compound 607 | 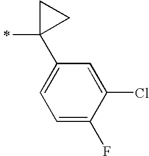 | B | ++ | ++ |
| 4-{Amino-[1-(indane-1-carbonyl)- pyrrolidin-2-yl]-methyl}-N-pyridin- 4-yl-benzamide | Compound 608 | 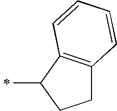 | B | ++ | ++ |
| 4-{Amino-[1-(3-oxo-indane-1- carbonyl)-pyrrolidin-2-yl]-methyl}- N-pyridin-4-yl-benzamide | Compound 609 | 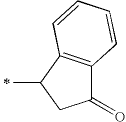 | B | ++ | ++ |
| 4-{Amino-[1-(5-fluoro-indane-1- carbonyl)-pyrrolidin-2-yl]-methyl}- N-pyridin-4-yl-benzamide | Compound 610 | 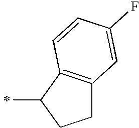 | B | + | ++ |
| 4-{Amino-[1-(6-fluoro-indane-1- carbonyl)-pyrrolidin-2-yl]-methyl}- N-pyridin-4-yl-benzamide | Compound 611 | 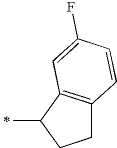 | B | nd | nd |
| 4-{Amino-[1-(5-chloro-indane-1- carbonyl)-pyrrolidin-2-yl]-methyl}- N-pyridin-4-yl-benzamide | Compound 612 | 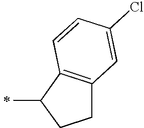 | B | nd | nd |
| 4-{Amino-[1-(1,2,3,4-tetrahydro- naphthalene-1-carbonyl)- pyrrolidin-2-yl]-methyl}-N-pyridin- 4-yl-benzamide | Compound 613 | 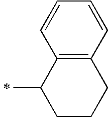 | B | ++ | ++ |
| 4-{Amino-[1-(1,2,3,4-tetrahydro- phenanthrene-4-carbonyl)- pyrrolidin-2-yl]-methyl}-N-pyridin- 4-yl-benzamide | Compound 614 | 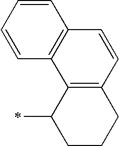 | B | nd | nd |
| 4-{Amino-[1-(chroman-4- carbonyl)-pyrrolidin-2-yl]-methyl}- N-pyridin-4-yl-benzamide | Compound 615 | 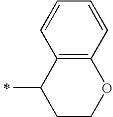 | B | + | ++ |
| 4-{Amino[1-(6-chloro-chroman-4- carbonyl)-pyrrolidin-2-yl]-methyl}- N-pyridin-4-benzamide | Compound 616 | 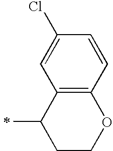 | B | ++ | ++ |
| 4-{Amino[1-(6-fluoro-chroman-4- carbonyl)-pyrrolidin-2-yl]-methyl}- N-pyridin-4-benzamide | Compound 617 | 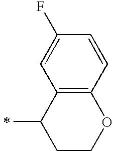 | B | ++ | ++ |
| 4-{Amino[1-(thiochroman-4- carbonyl)-pyrrolidin-2-yl]-methyl}- N-pyridin-4-yl-benzamide | Compound 618 | 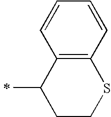 | B | ++ | ++ |
| 4-{Amino[1-(6-fluoro- thiochroman-4- carbonyl)-pyrrolidin-2-yl]-methyl}- N-pyridin-4-yl-benzamide | Compound 619 | 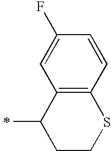 | B | ++ | ++ |
| 4-{Amino-[1-(3-fluoro-6,7,8,9- tetrahydro-5H-benzocycloheptene- 5-carbonyl)-pyrrolidin-2-yl]- methyl}-N-pyridin-4-yl-benzamide | Compound 620 | 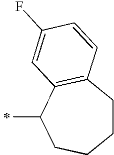 | B | + | + |
| 4-{Amino-[1-(4,5,6,7-tetrahydro- benzo[b]thiophene-4-carbonyl)- pyrrolidin-2-yl]-methyl}-N-pyridin- 4-yl-benzamide | Compound 621 | 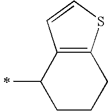 | B | ++ | +++ |

Table 8 shows the results for compounds of Formula XVI. As used herein the term “nd” means “not determined yet”.

TABLE 8

| | |
|---|---|
| | XVI |
| 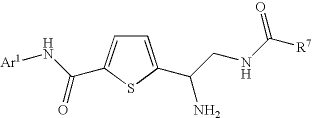 | |

| | | | | IC₅₀ | IC₅₀ |
|---|---|---|---|---|---|
| | | | | μM | μM |
| Name | Compound | Ar¹— | —R⁷ | PKCε | ROCK |
| 5-{1-Amino-2-[(1-phenyl- cyclopropanecarbonyl)-amino]-ethyl}- thiophene-2-carboxylic acid pyridin-4-ylamide | Compound 656 | 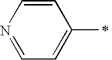 | 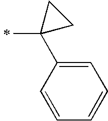 | nd | nd |
| 5-(1-Amino-2-{[1-(4-chloro-phenyl)- cyclopropanecarbonyl]-amino}-ethyl)- thiophene-2-carboxylic acid pyridin-4-ylamide | Compound 657 | 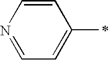 | 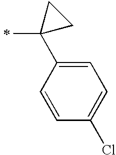 | nd | nd |
| 5-{1-Amino-2-[2-(4-chloro-phenyl)-2-methyl- propionylamino]-ethyl}-thiophene-2-carboxylic acid pyridin-4-ylamide | Compound 658 | 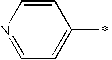 | 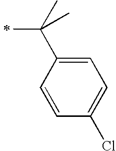 | nd | nd |
| 5-{1-Amino-2-[2-(4-fluoro-phenyl)-2-methyl- propionylamino]-ethyl}-thiophene-2-carboxylic acid pyridin-4-ylamide | Compound 659 | 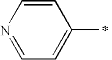 | 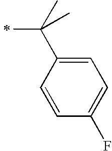 | nd | nd |
| 5-{1-Amino-2-[2-(3,4-dichloro-phenyl)-2- methyl-propionylamino]-ethyl}-thiophene-2- carboxylic acid pyridin-4-ylamide | Compound 660 | 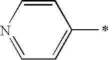 | 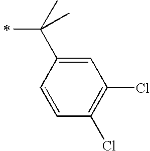 | nd | nd |

The inhibition results of some compounds of the invention on the activity on PKC theta are shown under Table 9.

TABLE 9

| | | IC₅₀ |
|---|---|---|
| | | PKCθ |
| Compound | Name | (μM) |
| Compound | 4-(1-Amino-2-{[1-(4-chloro-phenyl)- | |
| 168 | cyclopropanecarbonyl]-amino}-ethyl)-N-pyridin- | +++ |
| | 4-yl-benzamide dihydrochloride | |
| Compound | 4-{1-Amino-2-[2-(4-chloro-phenyl)-2-methyl- | +++ |
| 335 | propionylamino]-ethyl}-N-pyridin-4-yl- | |
| | benzamide | |
| Compound | 4-{(S)-1-Amino-2-[2-(4-chloro-phenyl)-2-methyl- | ++++ |
| 399 | propionylamino]-ethyl}-N-pyridin-4-yl- | |
| | benzamide dihydrochloric acid salt | |
| Compound | 4-{1-Amino-2-[2-(4-fluoro-phenyl)-2-methyl- | ++++ |
| 339 | propionylamino]-ethyl}-N-pyridin-4-yl- | |
| | benzamide | |
| Compound | 4-(1-Amino-2-{[1-(4-chloro-phenyl)- | ++++ |
| 371 | cyclopropanecarbonyl]-amino}-ethyl)-N-(1H- | |
| | pyrrolo[2,3-b]pyridin-4-yl)-benzamide | |
| | dihydrochloric acid salt | |

The present invention encompasses compounds 1 to 662 and stereoisomers, tautomers, racemics or a pharmaceutically acceptable salt and/or solvate thereof.

### Biological Activity on PKC Epsilon and PKC Theta

The compounds according to the present invention are inhibitors of the novel PKCs, epsilon and theta, and are particularly suitable to treat a variety of inflammatory and auto immune diseases. PKCε plays a role in Toll-like receptors (TLR) 4 mediated cytokine expression in macrophages and dendritic cells. Inhibitors of PKCε impair production of inflammatory cytokines including TNFα from macrophages and IL-12 secretion from dendritic cells.

The compounds of the invention are particularly potent and orally bio-available inhibitors of both PKCθ and PKCε.

The compounds are orally active and have been evaluated in vivo in models of inflammation and autoimmune disease.

The present compounds provide therefore an oral approach to treat chronic inflammatory diseases.

### Potency and Selectivity

Potency and selectivity of compounds of the invention to closely related kinases was evaluated (data not shown).

Compounds of the invention are particularly selective, are particularly potent (nanomolar range) and are particularly selective to PKCθ and PKCε and ROCK versus closely related protein kinases of the AGC-family such as PKA and PKB.

### Cell Models for Inhibitors of PKCε

Cellular models were used to evaluate potential for in vivo potency of PKCε inhibitors.

Whereas the reporter assay is focused on PKCε specific inhibition in a recombinant system, the monocytes assay is a biomarker assay that can be performed in isolated monocytes, full blood and in vivo to follow the pharmacological reduction of cytokine production:

### Reporter Assay

The reporter assay exploits the effect of over-expressed PKCε on kinase activation and phosphorylation of a transcription factor to evaluate the potency and selectivity of PKCε inhibitors in a cellular context.

It has been shown by jae-Won Soh et al (MCB, February 1999 p 1313-1324) that over-expression of PKC epsilon results in the activation of MAPK-pathway (c-raf/MEK/ERK) leading to the activation of Elk transcription factor. This event can be monitored using the PathDetect trans reporter assay system (Stratagene): pFA2-ELK codes for a fusion protein between the Gal4 DNA binding domain and the transactivation domain of Elk which drives the expression of a secreted alkaline phosphatase (SEAP) reporter gene.

Compounds are incubated with reporter cell-lines either over-expressing PKCε, leading to continued activation of the kinase, or mock transfected cells. Inhibition of the PKCε induced signal in the absence of an effect on the basal level (mock transfected cells) is indicative for selective PKCε inhibition.

Compounds of the invention showed potency and selectivity in this reporter assay. To position these data, the results obtained were compared with the results obtained with bisindolylmaleimide (BIM I) in this assay. BIM I is the most potent, yet unselective, PKCε inhibitor known in literature.

The results demonstrate that the compound of the present invention is at least as potent or 10-fold more potent than BIM I. For example, compounds 335, 339, 340, 356, 371, 373 and 399 have IC₅₀<5 μM.

### LPS Induced TNF Release in Human Whole Blood

This assay is based on the observation that lipopolysaccharide (LPS) induced TNF-alpha release in human monocytes/macrophages is in part dependent on PKCε (Antonio Castrillo et al J Exp Med November 2001 p 1231-1242) and ROCK (Jean-pierre Segain et al, Gastroenterology 2003 124 p 1180-1187).

Monocytes and macrophages from different sources can be used: two examples are given below.
Human blood is collected in heparin tubes and transferred into tissue culture plates. Blood samples are pre-incubated with compound for 1 h before stimulation with LPS. 18 h later the cells are spun down and the supernatant analyzed for TNF-alpha using standard ELISA (R&D systems)
Mouse thioglycolate elicited macrophages are transferred into tissue culture plates. Macrophages are pre-incubated with compound for 1 h before stimulation with LPS. 3 h later the tissue culture medium can be analyzed for TNF-alpha

Data (IC₅₀) (not shown) corroborate the fact that the compound of the invention is capable of inhibiting LPS induced TNF release in human whole blood.

### LPS Induced TNFα Release In Vitro (Bio-Marker Assay).

In addition to the reporter assay, which relies on over-expression of PKCε, a cellular assay measuring PKCε activity in a physiological context was used.

This assay was based on the observation that LPS induced TNFα release in monocytes/macrophages is dependent on PKCε.

The assay can be performed in vitro as well as in vivo. As an example for the in vitro application, the concentration-response curve of compounds of the invention and BIM I on TNFα release was measured 24 h after LPS stimulation in whole blood.

The results (not shown) corroborate the fact that the compound of the invention is capable of suppressing the TNFα release efficiently when compared to BIM I.

Similarly, a cellular assay was performed, based on the observation that LPS induced

TNFα release in monocytes/macrophages is dependent on ROCK. The assay can be performed in vitro as well as in vivo.

As an example for the in vitro application, the concentration-response curve of compounds of the invention and the Y compound on TNFα release was measured 24 h after LPS stimulation in whole blood.

The results (not shown) corroborate the fact that the compound of the invention is capable of suppressing the TNFα release efficiently when compared to the Y-27632 compound.

### In Vivo Results

### LPS Induced TNFα Release In Vivo (Bio-Marker Assay).

A typical in vivo experiment was also performed.

The concept is the same as the in vitro assay, but this time the animals receive compound (or vehicle) via oral IP injection xh (for example 2 and 4 in the examples) before they are challenged with IP injection of LPS. 1 h after the LPS challenge a terminal blood sample is taken and the amount of TNFalpha in the serum determined using standard ELISA (R&D systems). This assay not only gives information on the appropriate route and dose to obtain efficacy in vivo but it also gives an idea on the duration of action (how long is the compound around at sufficiently high levels to exert an effect on the target) by varying the time between dosing of compound and the LPS challenge.

The data (not shown) corroborate the fact that the compound of the invention is capable of inhibiting the PKCε and/or ROCK dependent TNFα release (i.p. and/or p.o. dosing) in vivo.

For example, 30 mg/kg of a compound of the invention inhibited the LPS induced TNFα release by more than 80% in vivo whereas the best known but unselective PKC inhibitor reduced TNFα release only by 50%.

### Carrageenin Induced Paw Edema

This is an acute model of inflammation useful in the initial in vivo evaluation of anti-inflammatory compounds developed for the treatment of diseases such as rheumatoid arthritis and multiple sclerosis. Injection of carrageenin into the subplantar region of the hindlimb results in joint inflammation within hours after induction. The response is in part due to TNFalpha production.

The basic measurement in this model is comparison of paw volume (swelling) between the right, carrageenin treated, and left, untreated, hindlimb over a period of 6 hours after the injection with carrageenin.

The efficiency of compounds of the invention in acute inflammation was evaluated using the Carrageenin model: 5-6 weeks old Swiss Webster mice (Harlan) were weighed and the right paw volume measured by water displacement at the start of the experiment. The animals (n=10) were administered either vehicle or different doses of compound of the invention by the oral route. Two hours after oral administration animals were anesthetized and injected with 50 μl (10 mg/ml) carrageenin in the sub plantar region of the paw. Two, four and six hours following the injection the paw volumes were measured.

Mice were dosed with: vehicle alone, 10 mg/kg and 30 mg/kg of a compound of the invention orally, 2 h before Carrageenin injection into the paw. Paw volume was investigated for groups of 10 treated animals (n=10). The two hour value has a significance of >99% according to the T test.

The results (not shown) corroborate the fact that the compound of the invention is capable of suppressing the inflammatory response in the Carrageenin model.

### Selectivity and Combi-PK Studies

The kinase profiles obtained with compounds of the invention revealed that these inhibitors are highly selective versus kinases belonging to non-AGC kinase families and non-closely related kinases within the AGC family. No significant activities on unrelated biological targets are present, indicating a low risk for liabilities related to off target side-effects at therapeutic concentrations.

All compounds were highly stable as solids and in solution. All representatives tested from this large series of PKCε inhibitors exhibited extremely high aqueous solubility.

The oral bioavailability of the compounds of the invention is above 10%.

The clearance measured in rodents, combined with the metabolic stabilities and plasma protein binding observed when using rodent and human material, suggests that these compounds are suitable for once or twice daily oral dosing.

In vitro testing for cytochrome P450 inhibition, mutagenicity or hERG activity found no liabilities for the present compounds. Results obtained with a standard panel of five CYPs did not indicate any liabilities.

The compound of the invention was inactive in the Ames test using the TA98 and TA100 strains, with and without S9 mix.

So far, no overt signs of toxicity have been observed. The present inhibitors are tolerated in mice up to 60 mg/kg/day.

Several of the compounds have been evaluated in a five-day toxicity study. At doses of 30 mg/kg/day p.o. and i.p., compounds of the invention are tolerated.

The profiles obtained with the most advanced leads are very comparable to the profile of successful known PKCβ inhibitors.

All patents, patent applications, and published references cited herein are hereby incorporated by reference in their entirety. While this invention has been particularly shown and described with references to preferred embodiments, it will be understood by those skilled in the art that various changes in form and details may be made without departing from the scope of the invention encompassed by the claims.

1. A compound of Formula I or II or a stereoisomer, tautomer, racemic, salt, hydrate, or solvate thereof, 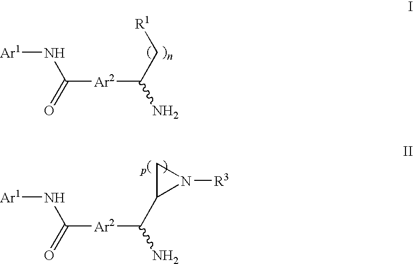
wherein:
Ar¹ is an aromatic 6-membered first ring containing carbon atoms and at least one nitrogen atom, said first ring being optionally fused to a saturated, unsaturated or aromatic 4-, 5-, 6-, or 7-membered second ring containing carbon atoms and optionally at least one nitrogen atom, said first or said second rings being independently substituted with one or more substituents independently selected from the group comprising hydrogen, halogen, alkyl, cycloalkyl, alkenyl, alkynyl, aralkyl, heteroarylalkyl, cycloalkylalkyl, acyl, aryl or heteroaryl wherein said substituents are optionally substituted by one or more further substituents selected from the group comprising halo, hydroxyl, oxo, nitro, amido, carboxy, amino, cyano, haloalkoxy, and haloalkyl;
Ar² is an aromatic 5- or 6-membered third ring containing carbon atoms and optionally one or two heteroatoms, said third ring optionally fused to an aromatic 6-membered fourth ring containing carbon atoms and optionally at least one heteroatom atom, wherein said third ring is optionally substituted with one or more substituents selected from the group comprising halogen, alkenyl, alkyl, alkynyl, acylamino, alkoxy, arylamino, nitro, haloalkoxy, aryl or heteroaryl, wherein said substituents are optionally substituted by one or more further substituents selected from the group comprising halo, hydroxyl, oxo, nitro, amido, carboxy, amino, cyano, haloalkoxy, and haloalkyl;
n is an integer selected from 1, 2 or 3; and
p is an integer selected from 2, 3, 4 or 5; and
R¹ is selected from the Formula: 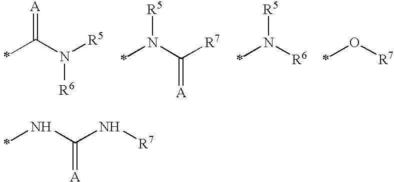
wherein
R³ is selected from the Formula: 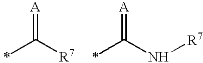
A is an oxygen or sulfur atom;
R⁵, R⁶ and R⁷ are each independently selected from the group comprising
hydrogen, a group selected from alkoxy, alkyl, alkylamino, alkylaminoalkyl, alkylcarbonyl, alkylcarbonylamino, amino, aralkyl, aryl, carbonylamino, cycloalkyl, formylamino, heteroaryl, heteroarylalkyl, heterocyclyl, or fused to the cycloalkyl, aryl, heterocyclyl or heteroaryl group may be one or more cycloalkyl, aryl, heterocyclyl or heteroaryl,
each group being optionally substituted by one or more substituent selected from halo, alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylalkenylaminooxy, heteroarylalkyl, heteroarylcarbonylamino, heterocyclyl, hydroxyalkyl, nitro, oxo, sulfonyl, or fused to the cycloalkyl, aryl, heterocyclyl substituent or heteroaryl may be one or more cycloalkyl, aryl, heterocyclyl or heteroaryl,
each of said substituent being optionally substituted by one or more further substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl.

2. The compound according to claim 1 wherein n is 1.

3. The compound according to claim 1 wherein n is 2.

4. The compound according to claim 1 wherein R¹ is selected from the Formula: 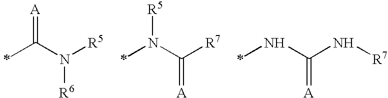
wherein A, R⁵, R⁶ and R⁷ have the same meaning as in any of the previous claims.

5. The compound according to claim 1 having one of the structural Formula 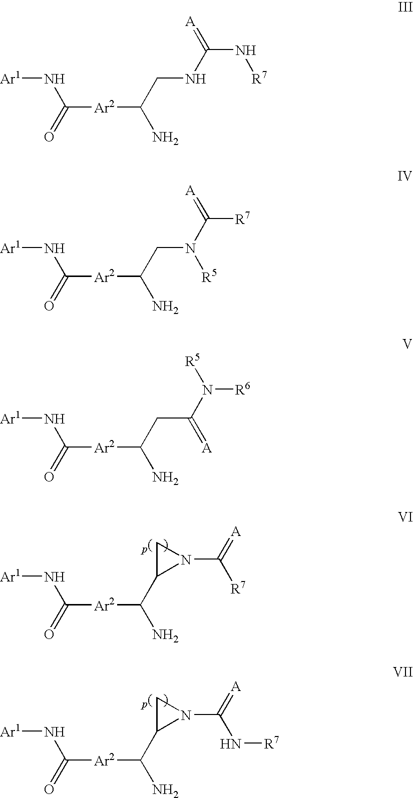
wherein Ar¹, Ar², A, R¹, R⁶ and R⁷ have the same meaning as in any of the previous claims.

6. The compound of claim 5 wherein R⁵ is hydrogen, alkyl or cycloalkyl and A is an oxygen or a sulfur atom.

7. The compound according to claim 1 wherein Ar¹— is of any of the Formula: 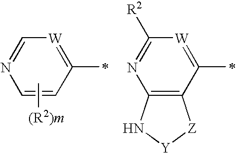
wherein
m is an integer selected from 0, 1, 2 or 3, preferably 0;
W is C(R²) or N, preferably C(R²), more preferably CH;
Y and Z are independently selected from the group comprising N and CR²
R² is selected from hydrogen, halogen, or a group selected from alkyl, cycloalkyl, alkenyl, alkynyl, aryl or heteroaryl wherein each of said group is optionally substituted by one or more substituents selected from the group comprising halo, hydroxyl, amido, carboxy, amino, cyano, haloalkoxy, and haloalkyl.

8. The compound according to claim 7 wherein W is N or C(R²), wherein R² has the same meaning as in claim 7.

9. The compound according to claim 8 wherein W is N or CH.

10. The compound according to claim 7 wherein m is 0.

11. The compound according to claim 1 wherein p is 3 or 4.

12. The compound according to claim 1
wherein —Ar²— is of any of the Formula: 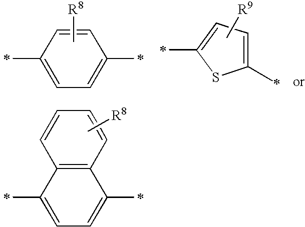
wherein
R⁸ is selected from hydrogen or halogen, or a group selected from alkenyl, alkyl, alkynyl, acylamino, alkoxy, arylamino, nitro, cyano, haloalkoxy, aryl or heteroaryl, each group being optionally substituted by one or more substituents selected from the group comprising halogen, alkyl, haloalkyl, nitro, haloalkoxy, aryl and heteroaryl; and
R⁹ is selected from the group comprising hydrogen, halogen and alkyl.

13. The compound according to claim 12, wherein R⁸ is hydrogen.

14. The compound according to claim 1, wherein R⁶ and R⁷ are each independently selected from 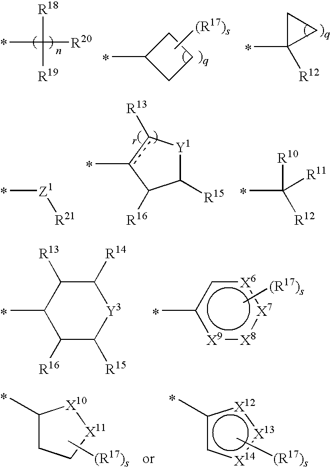
wherein
Y¹ is selected from —CH₂—, —CH(R¹⁴)—, —NH—, —O—, —S— or —C(═O)—,
Y³ is selected from —CH₂—, —CH₂—CH₂—, —O—, —S— or —NH—,
X⁶ is selected from N or CH,
X⁷ is selected from N, C(═O) or CH,
X⁸ is selected from N, NH or CH,
X⁹ is selected from N or CH,
X¹⁰ is selected from S, O or NH,
X¹¹ is selected from O, CH₂, C(═O), S or NH,
X¹² is selected from N, NH, O, S or CH,
X¹³ is selected from NH, O, S or CH,
X¹⁴ is selected from S, N, NH or CH,
Z¹ is selected from O or NH,
q is an integer selected from 1, 2, 3 or 4,
n is an integer selected from 1, 2, 3, 4, 5, 6 or 7,
wherein R¹⁰ and R¹¹ are each independently a selected from hydrogen, alkyl, cycloalkyl, aryl, or aralkyl,
wherein R¹² is selected from aryl, cycloalkyl, heteroaryl or heterocyclyl, each being optionally substituted by one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
r is an integer selected from 0, 1, 2 or 3,
wherein R¹³ and R¹⁴ are each independently selected from hydrogen or alkyl,
or R¹³ and R¹⁴ form together with the carbon atoms to which they are attached form an aryl, an heteroaryl, a cycloalkyl or a heterocyclyl,
or r is 2 and two R¹³ form together with the carbon atoms to which they are attached form an aryl, an heteroaryl, a cycloalkyl or a heterocyclyl,
wherein R¹⁵ and R¹⁶ together with the carbon atom to which they are attached form an aryl, a cycloalkyl, a heteroaryl a heterocyclyl, each being optionally substituted with one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
s is an integer selected from 0, 1, 2, 3 or 4,
wherein R¹⁷ is selected from halo, or a group selected from alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylalkenylaminooxy, heteroarylalkyl, heteroarylcarbonylamino, heterocyclyl, hydroxyalkyl, nitro, oxo, sulfonyl, or two R¹⁷ together with the atoms to which they are attached form an aryl, heteroaryl, cycloalkyl, or heterocyclyl, each group being optionally substituted with one or more substituents selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
wherein R¹⁸ and R¹⁹ are each independently selected from hydrogen, alkyl, cycloalkyl, cycloalkylalkyl, aryl, aralkyl, heteroaryl or heteroarylalkyl,
wherein R²⁰ is selected from hydrogen, or a group selected from alkyl, cycloalkyl, alkylaminoalkyl, alkylamino, alkylcarbonylamino, alkylcarbonylaminoalkyl, alkylsulfonyl, alkylsulfonylamino, alkylsulfonylaminoalkyl, amino, aminoalkyl, heterocyclyl, heterocyclylalkyl, cyano, cyanoalkyl, hydroxyl, hydroxyalkyl, alkoxy, alkoxyalkyl, alkoxycarbonyl, carboxy, alkoxycarbonylalkyl, aryl, aralkyl, heteroaryl, heteroarylalkyl, each group being optionally substituted by one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
wherein R²¹ is selected from alkyl, aryl, alkylcarbonyl, heteroaryl or heteroarylcarbonyl.

15. The compound according to claim 1 having one of the structural Formula 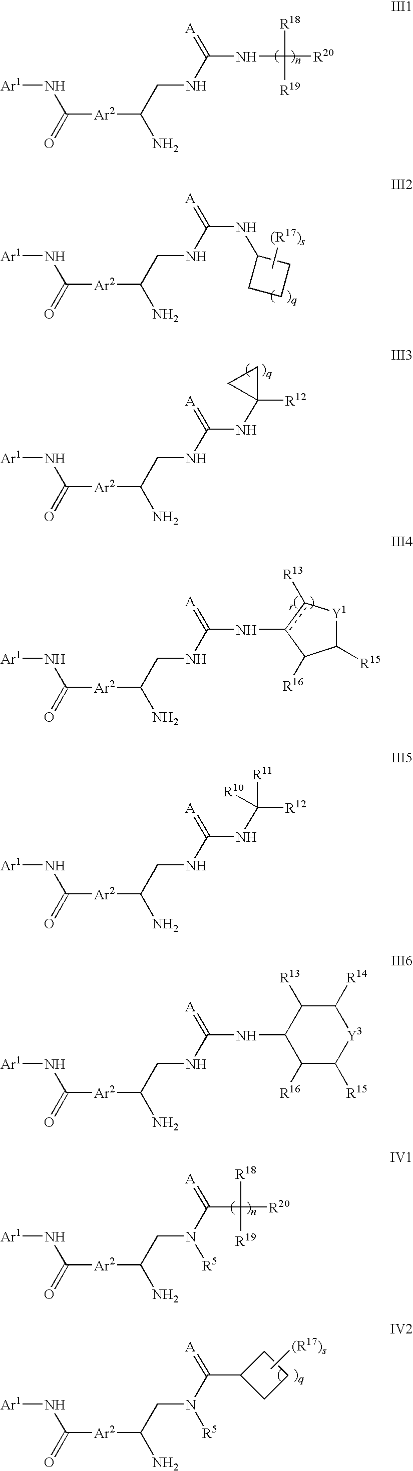 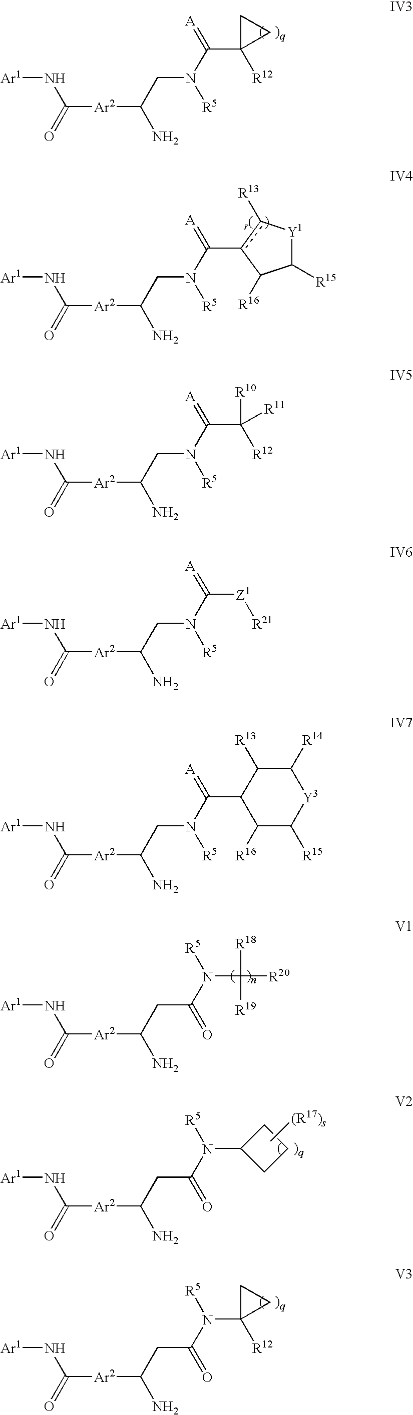 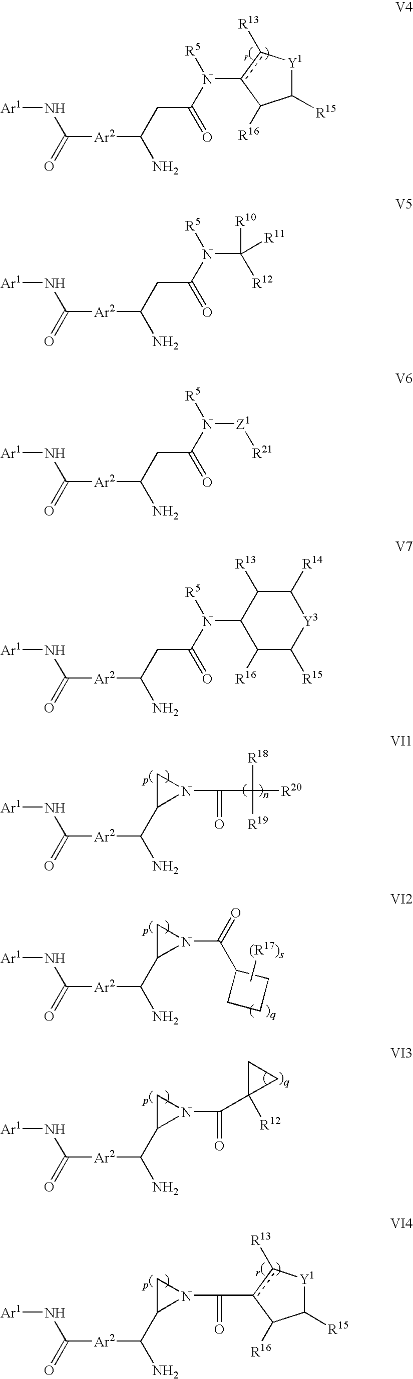 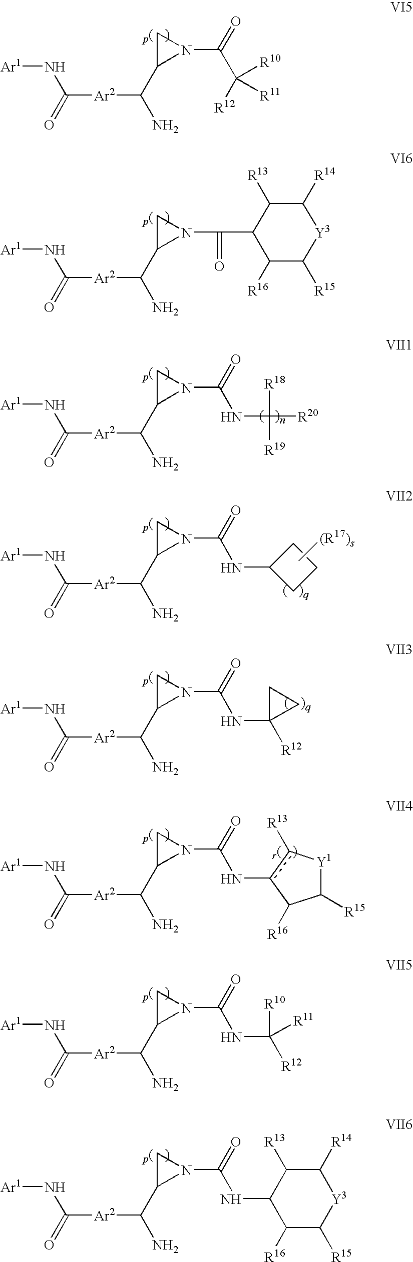
wherein Ar¹, Ar², A, s, p, q, r, n, Y¹, R⁵, R¹⁰, R¹¹, R¹², R¹³, R¹⁵, R¹⁶, and R¹⁷ same meaning as in any of the previous claims.

16. The compound according to claim 1 having one of the structural Formula 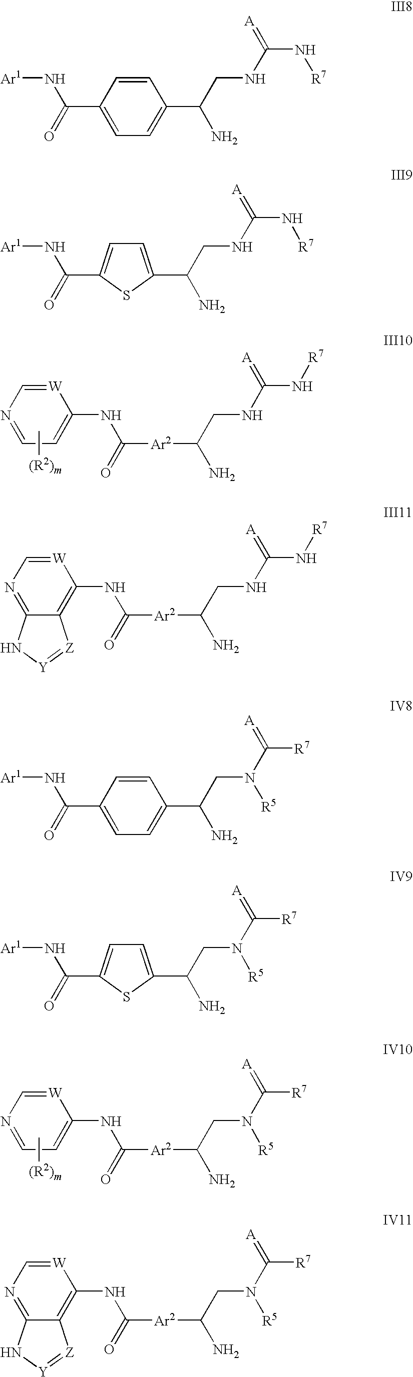 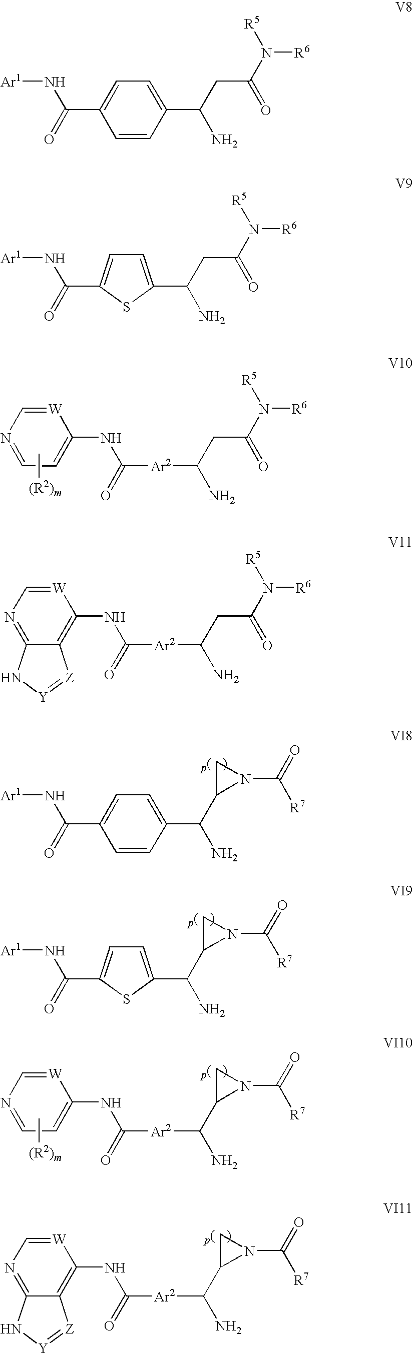 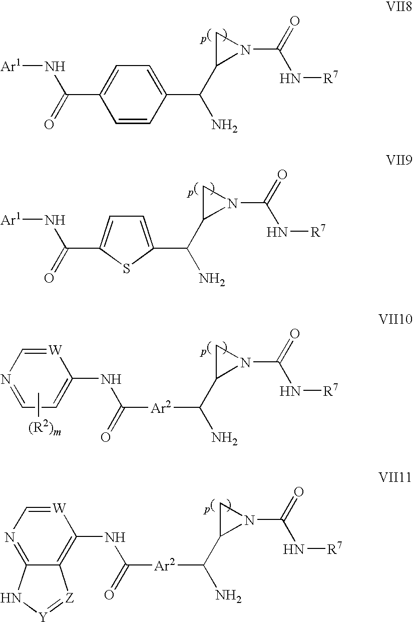
wherein Ar¹, Ar², A, s, p, q, r, n, W, Y, Y¹, Z, R², R⁵, R⁶, R⁷, R¹⁰, R¹¹, R¹², R¹³, R¹⁵, R¹⁶, and R¹⁷ have the same meaning as in any of the previous claims.

17. The compound according to claim 1 having one of the structural Formula 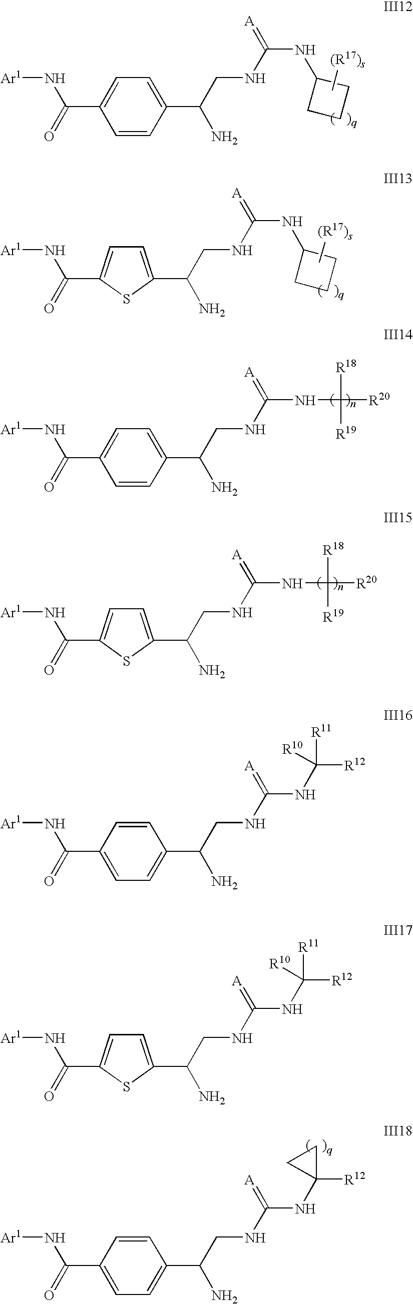 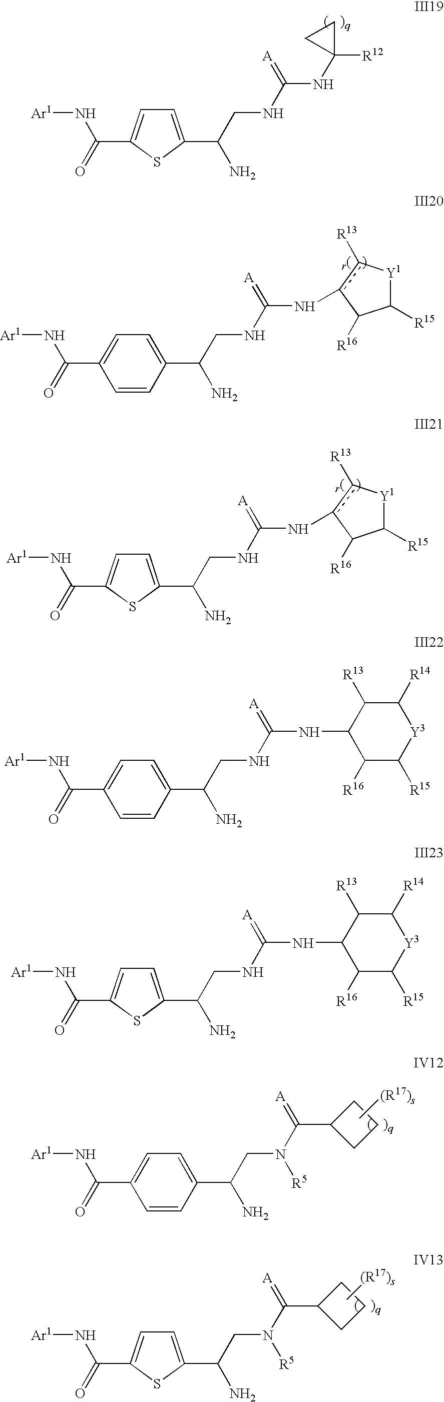 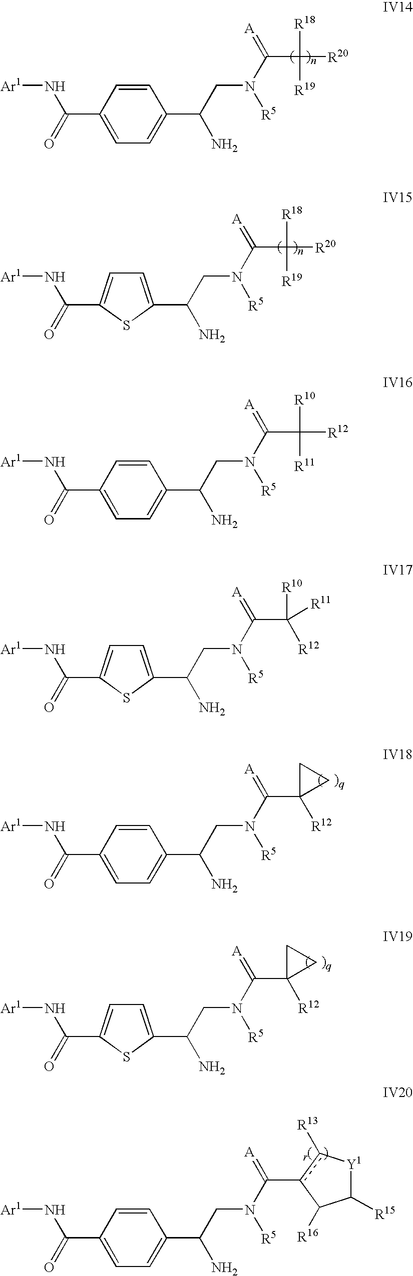 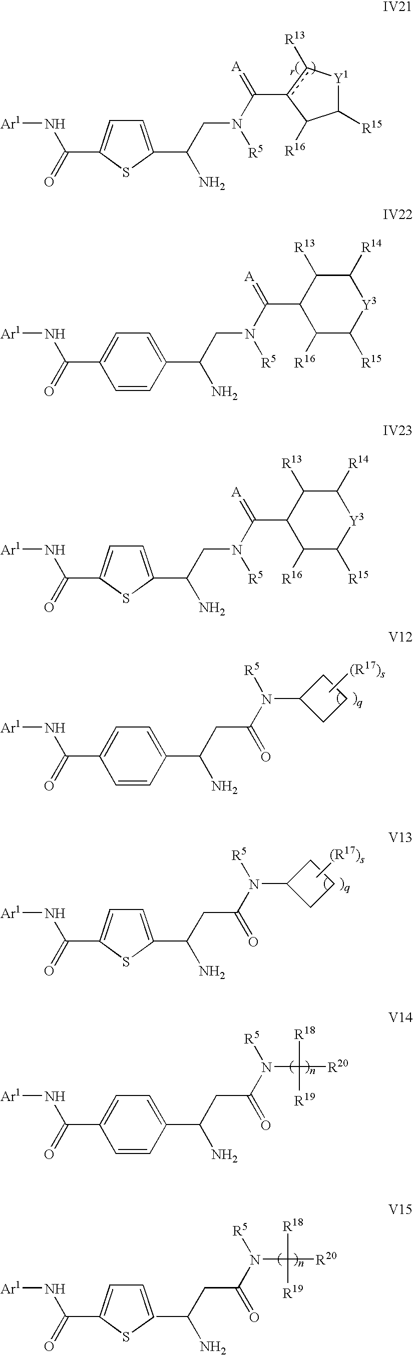 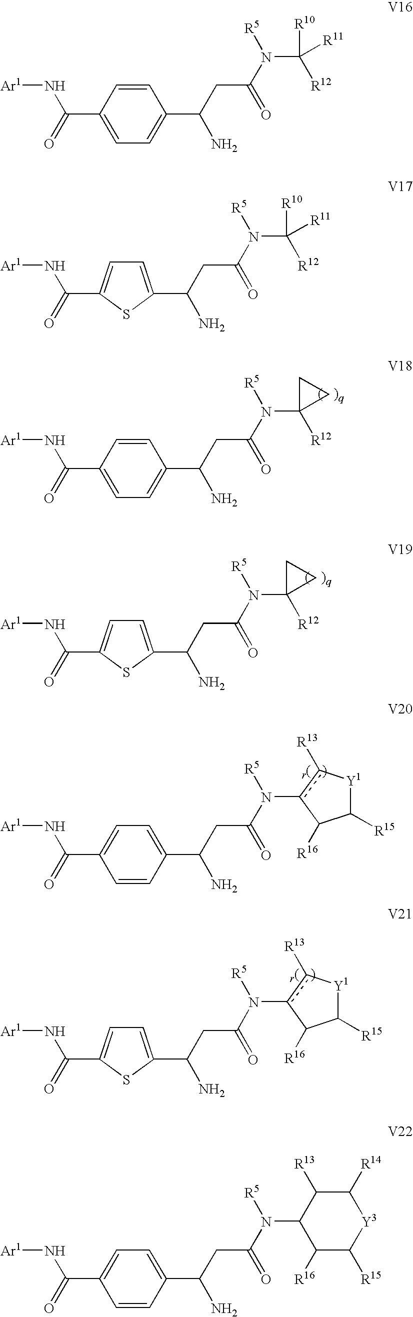 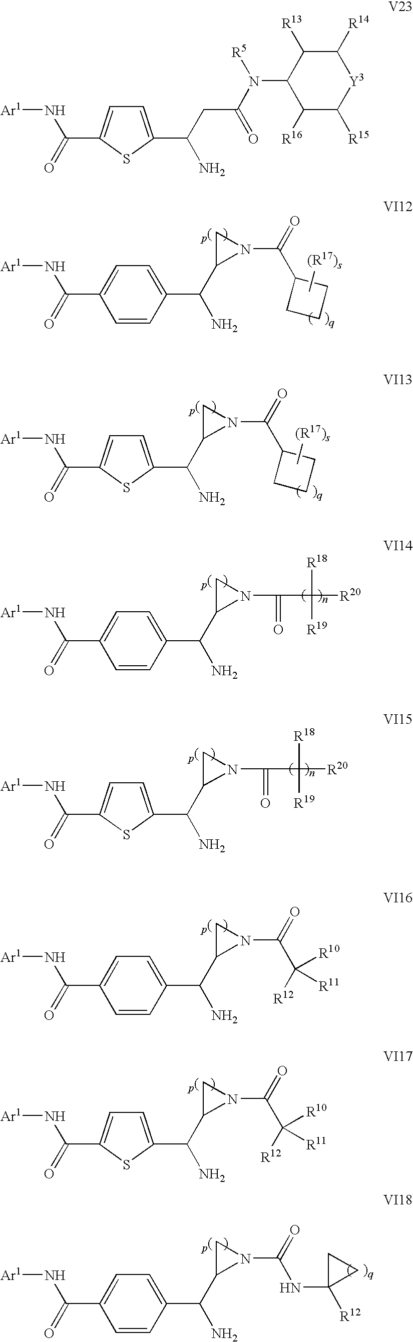 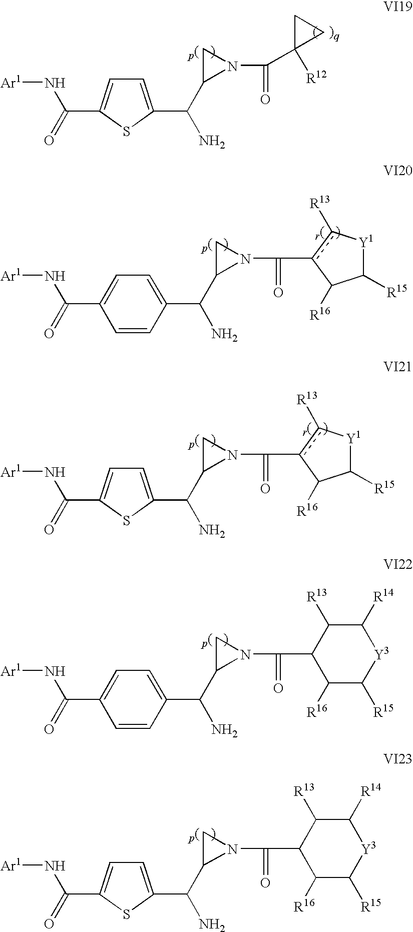
wherein Ar¹, A, s, p, q, r, n, Y¹, R⁵, R¹⁰, R¹¹, R¹², R¹³, R¹⁵, R¹⁶, R¹⁷, R¹⁸, R¹⁹ and R²⁰ have the same meaning as in any of the previous claims.

18. The compound according to claim 1 having one of the structural Formula 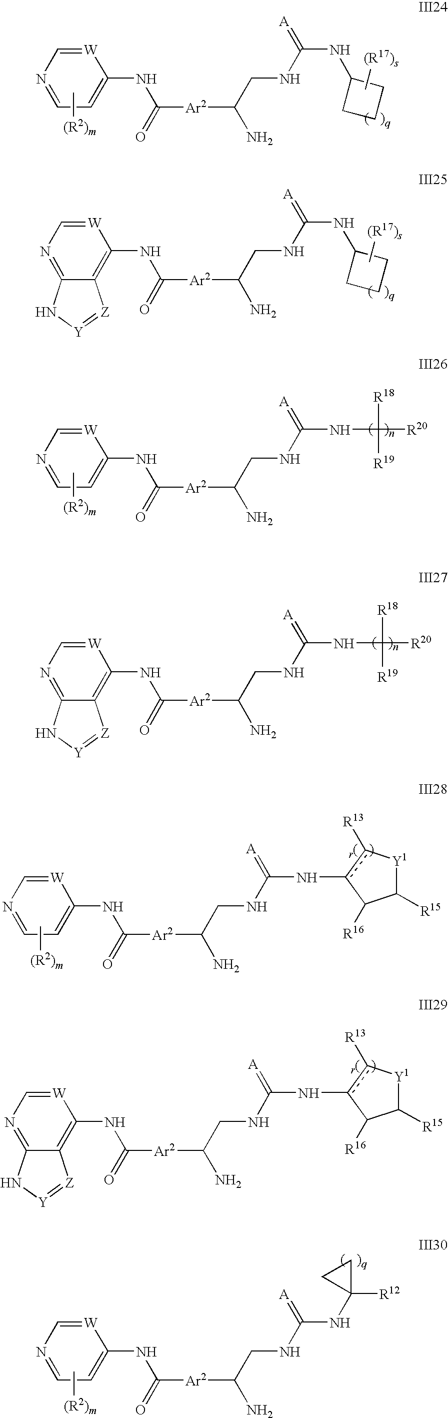 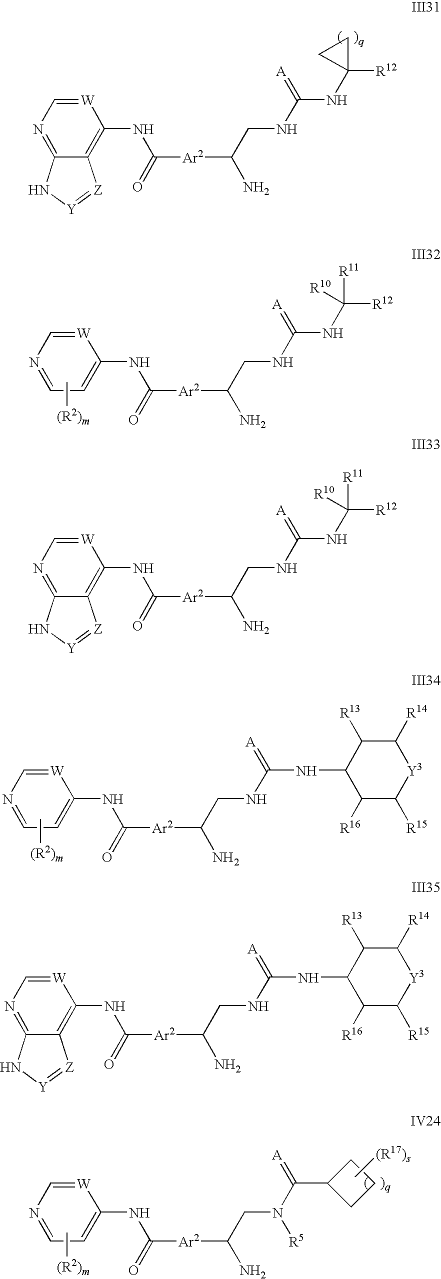 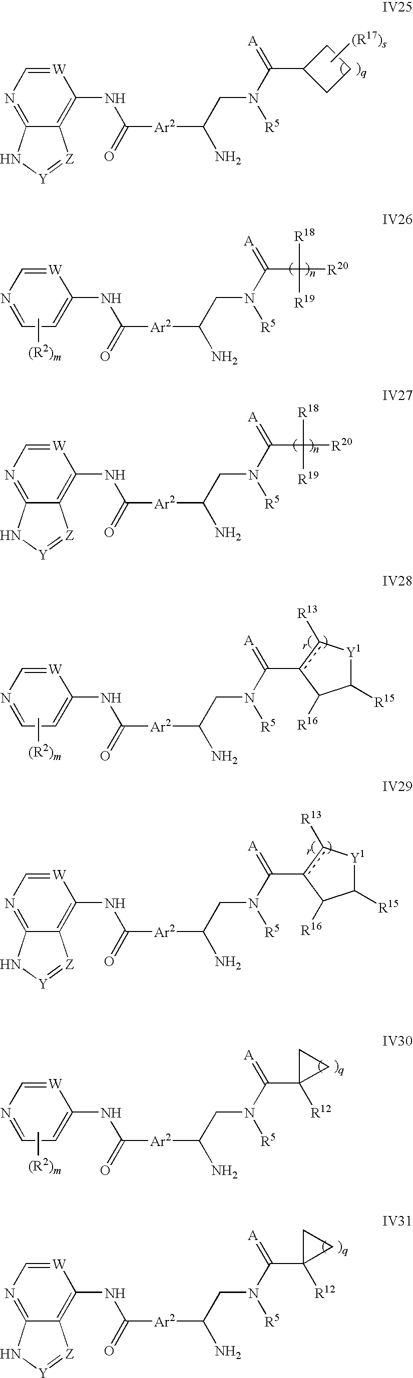 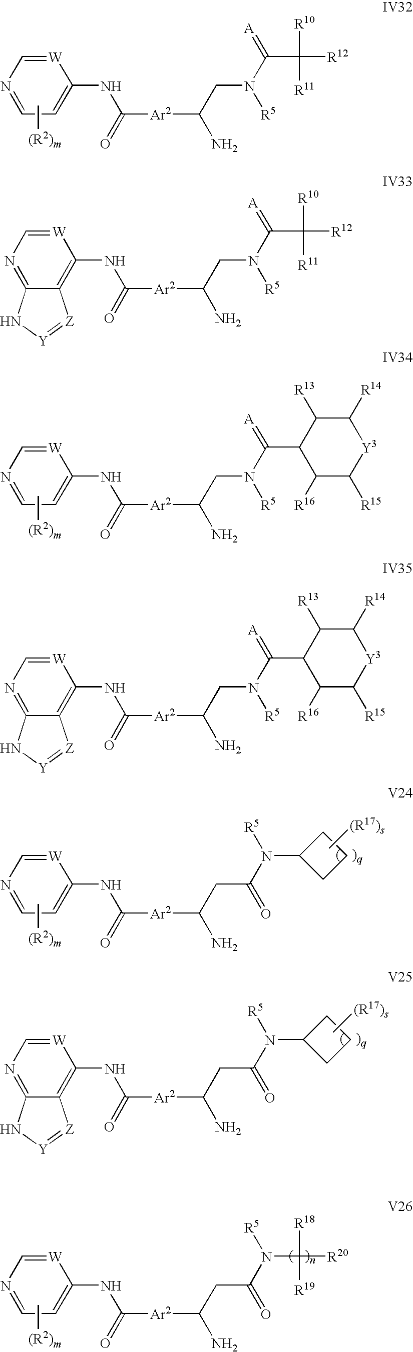 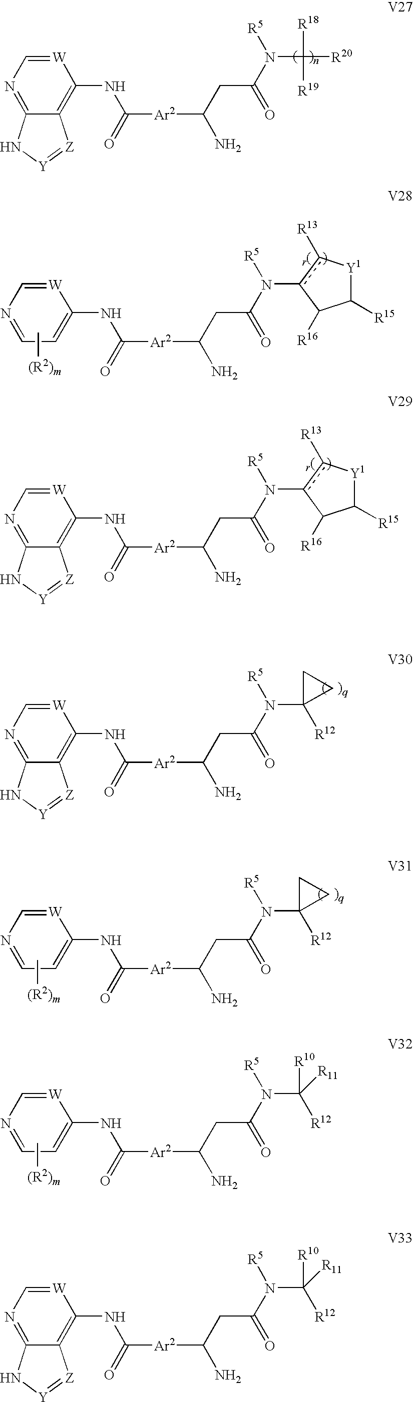 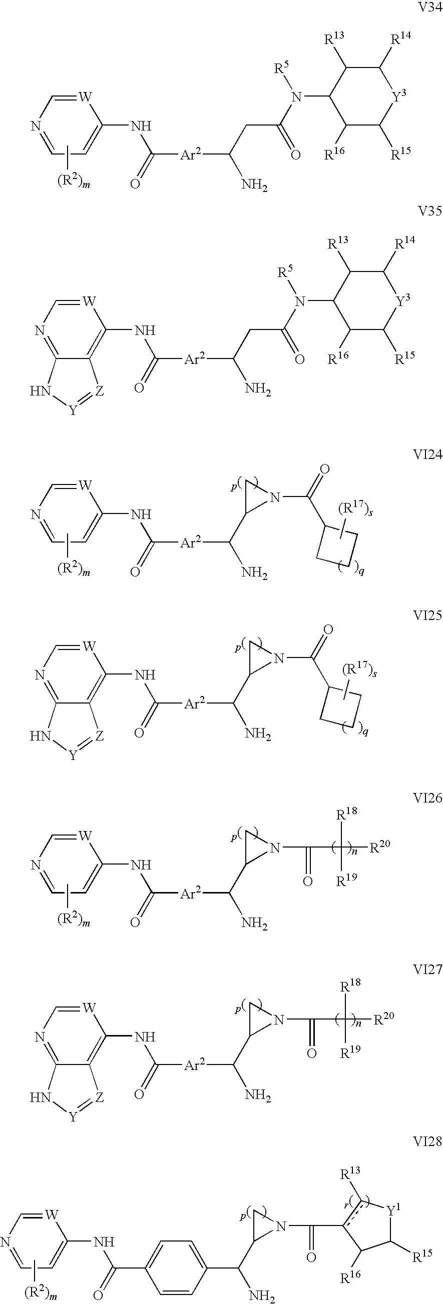 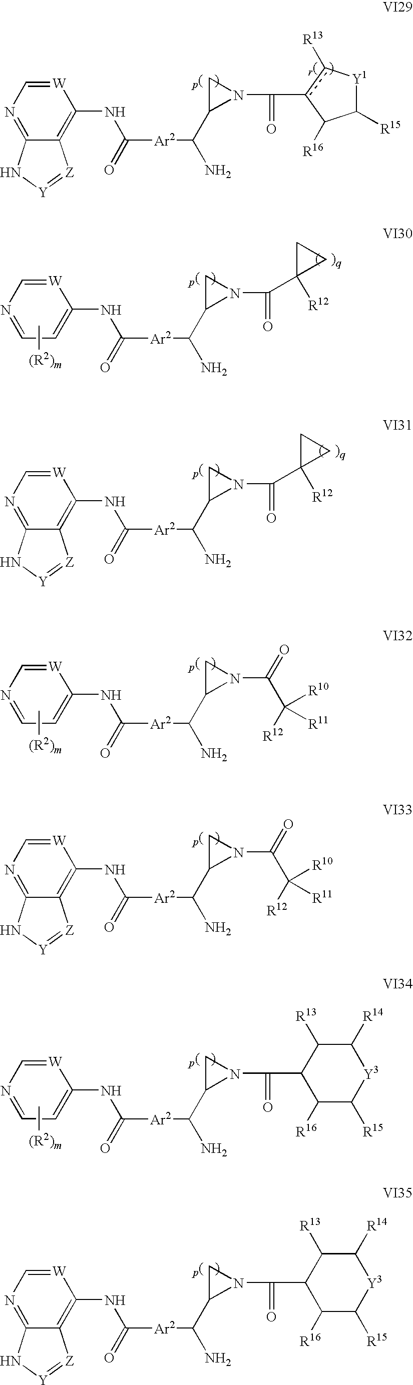
wherein Ar², A, s, p, q, r, m, n, W, Y, Y¹, Z, R², R⁵, R¹⁰, R¹¹, R¹², R¹³, R¹⁵, R¹⁶, R¹⁷, R¹⁸, R¹⁹ and R²⁰ have the same meaning as in any of the previous claims.

19. The compound according to claim 1 having one of the structural Formula 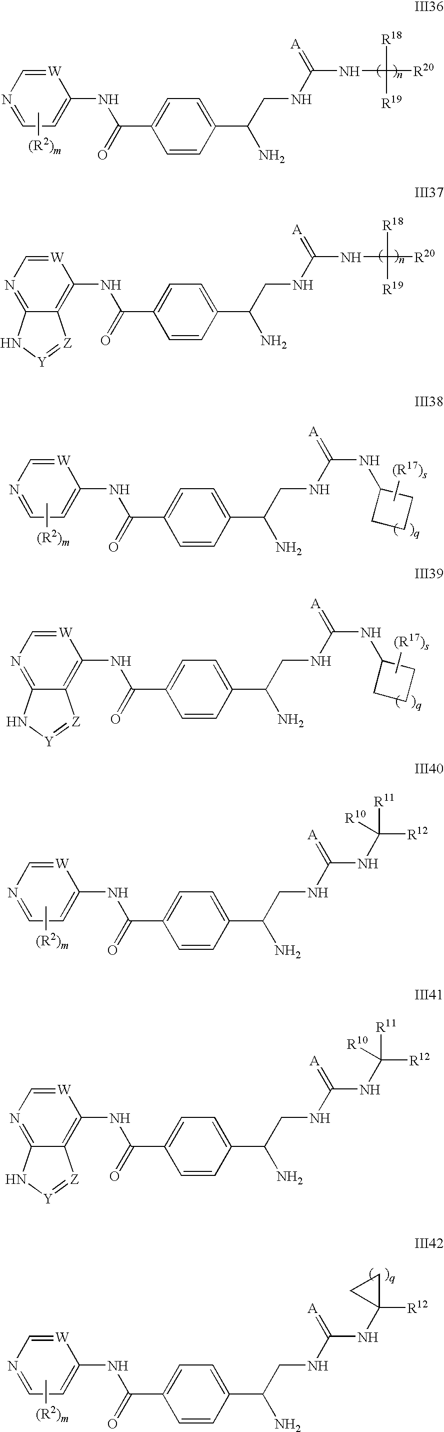 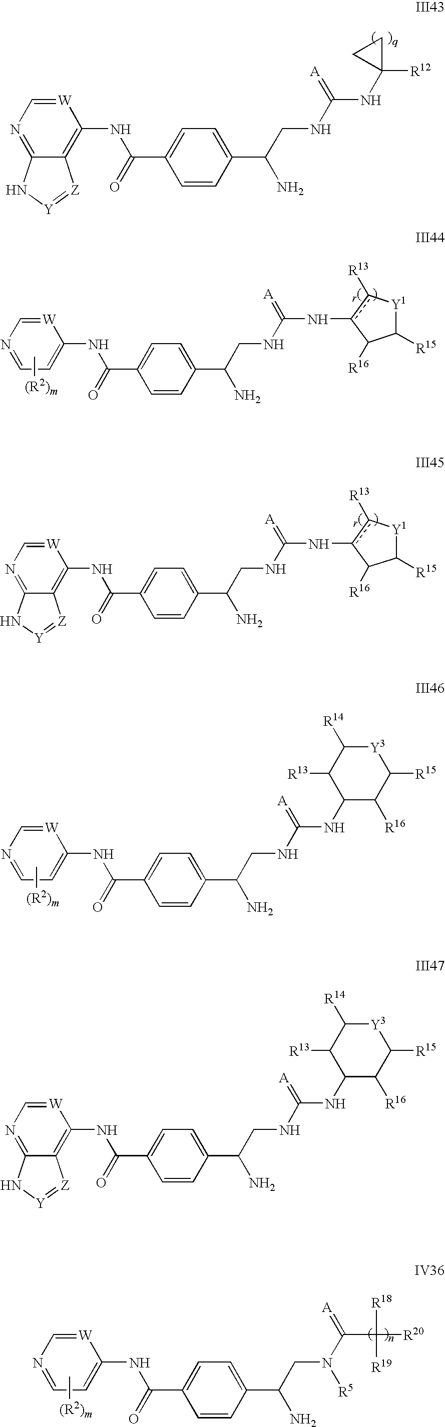 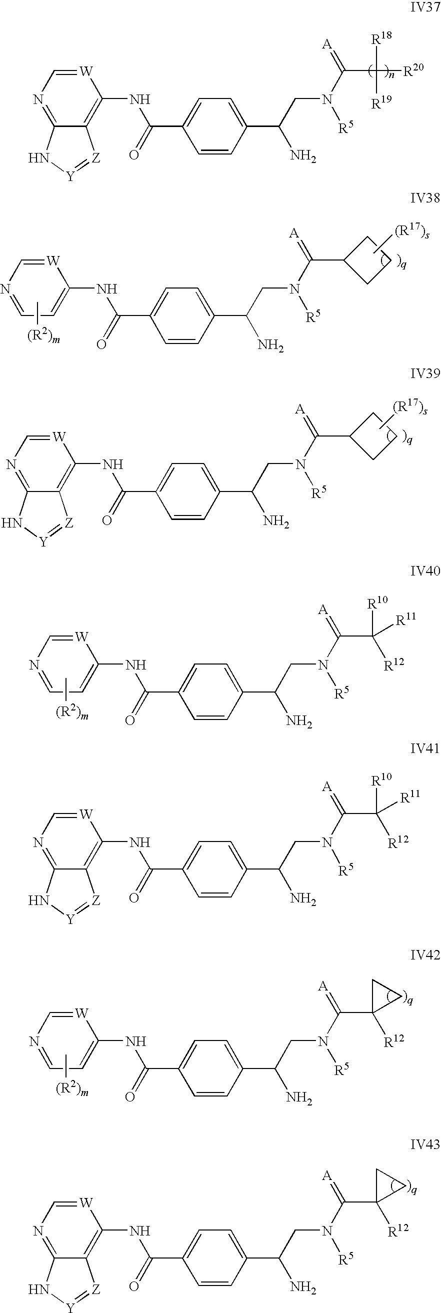 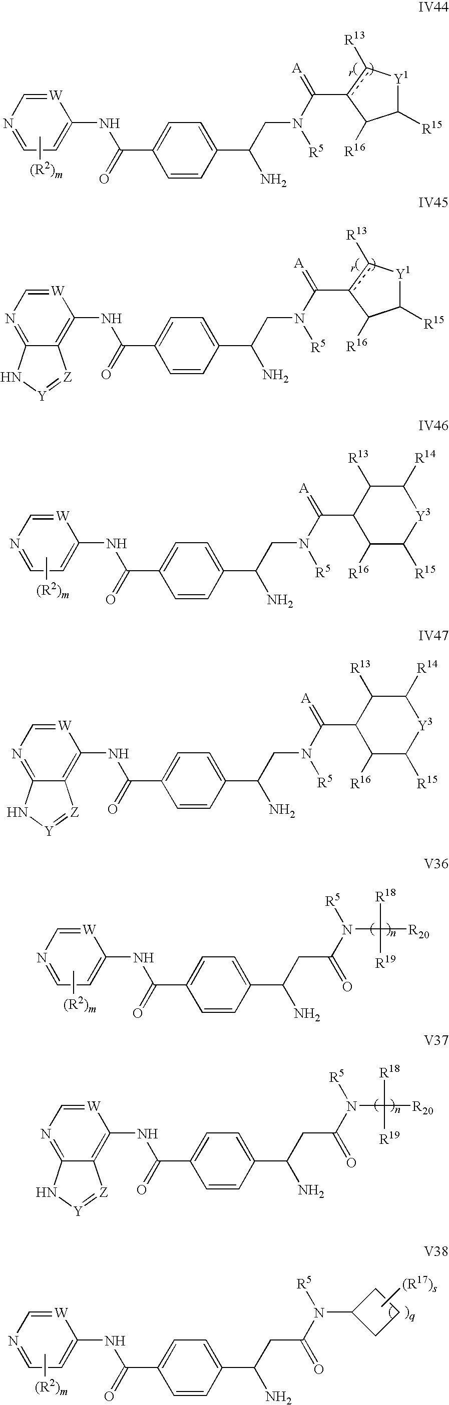 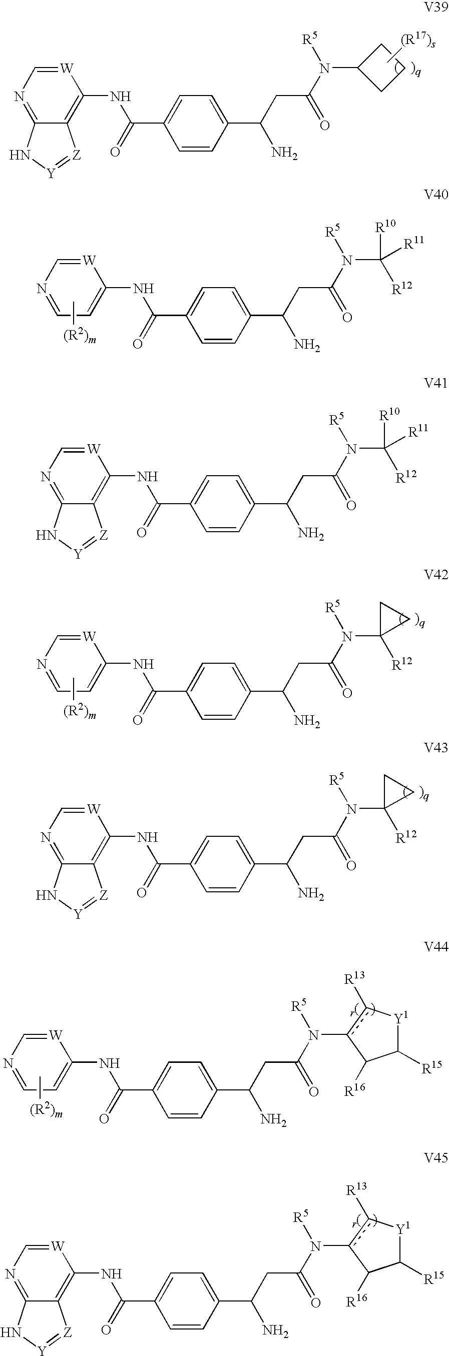 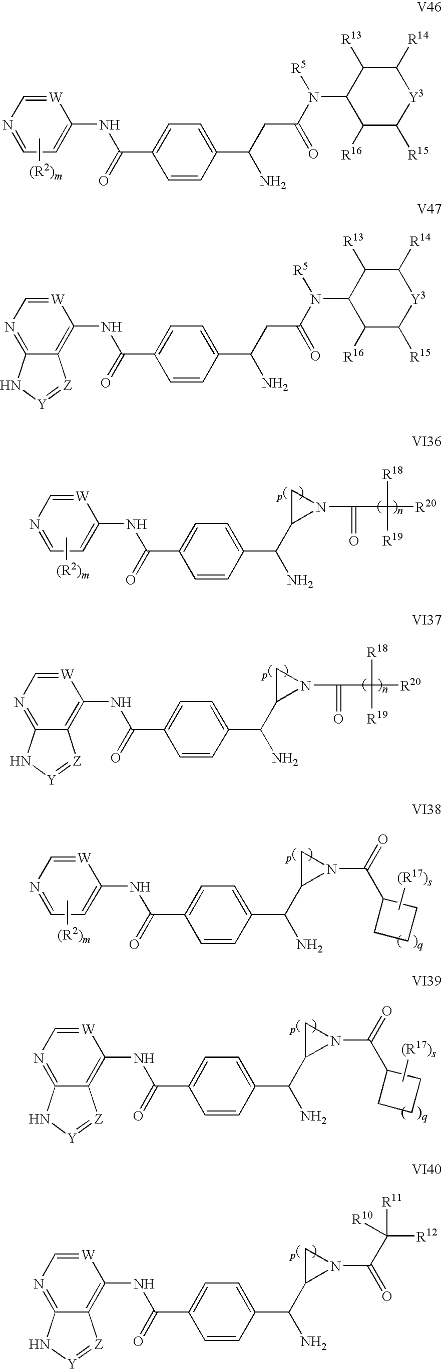 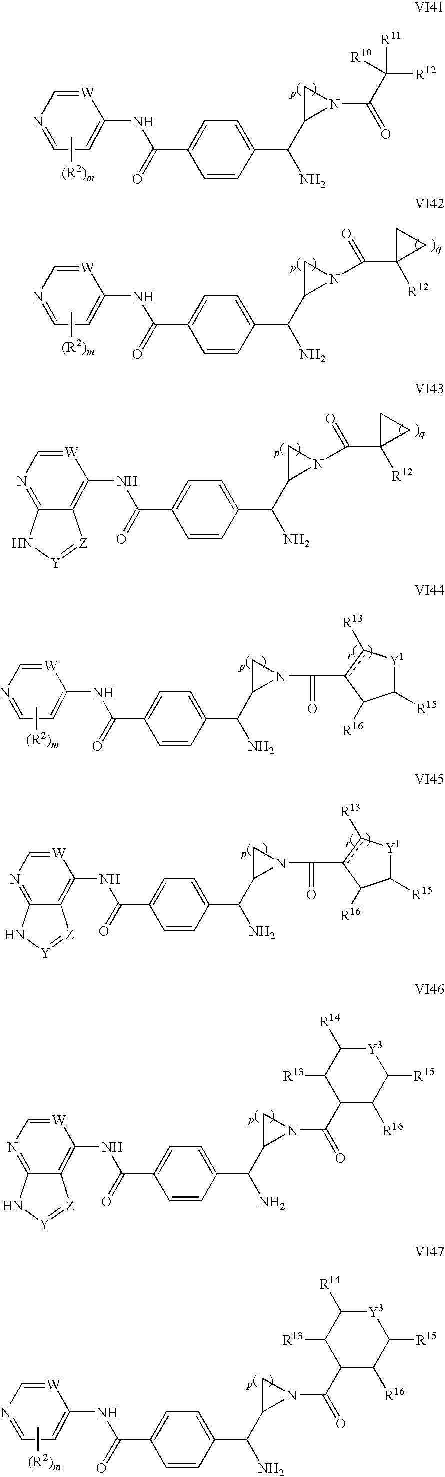
wherein A, s, p, q, r, n, m, W, Y, Y¹, Z, R², R⁵, R¹⁰, R¹¹, R¹², R¹³, R¹⁵, R¹⁶, R¹⁷, R¹⁸, R¹⁹ and R²⁰ have the same meaning as in any of the previous claims.

20. The compound according to claim 1 wherein:
Y¹ is selected from —CH₂—, —CH(R¹⁴)—, —NH—, —O—, —S— or —C(═O)—,
Y³ is selected from —CH₂—, —CH₂—CH₂—, —O—, —S— or —NH—,
A is O or S, W is N or CR², Y is N or CR², Z is N or CR², wherein R² is hydrogen or alkyl,
R⁵ is hydrogen, alkyl or cycloalkyl,
p is 3 or 4,
q is an integer selected from 1, 2, 3 or 4,
n is an integer selected from 1, 2, 3, 4, 5, 6 or 7,
wherein R¹⁰ and R¹¹ are each independently a selected from hydrogen, alkyl, cycloalkyl, aryl, or aralkyl,
wherein R¹² is selected from aryl, cycloalkyl, heteroaryl or heterocyclyl, each optionally substituted by one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
r is an integer selected from 0, 1, 2 or 3,
wherein R¹³ and R¹⁴ are each independently selected from hydrogen or alkyl,
or R¹³ and R¹⁴ form together with the carbon atoms to which they are attached form an aryl, an heteroaryl, a cycloalkyl or a heterocyclyl,
or r is 2 and two R¹³ form together with the carbon atoms to which they are attached form an aryl, an heteroaryl, a cycloalkyl or a heterocyclyl,
wherein R¹⁵ and R¹⁶ together with the carbon atom to which they are attached form an aryl, a cycloalkyl, a heteroaryl a heterocyclyl, each optionally substituted with one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
s is an integer selected from 0, 1, 2, 3 or 4,
wherein R¹⁷ is selected from halo, or a group selected from alkenylaminooxy, alkoxy, alkyl, alkylamino, alkylaminosulfonyl, alkylcarbonyl, alkylcarbonylamino, alkyloxyaminoalkenyl, alkyloxycarbonyl, alkylsulfonyl, alkylsulfonylamino, alkylthio, amino, aralkyl, aryl, arylalkenylaminooxy, arylamino, arylaminosulfonyl, arylcarbonyl, arylcarbonylamino, aryloxy, cyano, cycloalkyl, haloalkoxy, haloalkyl, haloaryl, heteroaryl, heteroarylalkenylaminooxy, heteroarylalkyl, heteroarylcarbonylamino, heterocyclyl, hydroxyalkyl, nitro, oxo, sulfonyl, or two R¹⁷ together with the atoms to which they are attached form an aryl, heteroaryl, cycloalkyl, or heterocyclyl, each group being optionally substituted with one or more substituents selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
wherein R¹⁸ and R¹⁹ are each independently selected from hydrogen, alkyl, cycloalkyl, cycloalkylalkyl, aryl, aralkyl, heteroaryl or heteroarylalkyl,
wherein R²⁰ is selected from hydrogen, or a group selected from alkyl, cycloalkyl, alkylaminoalkyl, alkylamino, alkylcarbonylamino, alkylcarbonylaminoalkyl, alkylsulfonyl, alkylsulfonylamino, alkylsulfonylaminoalkyl, amino, aminoalkyl, heterocyclyl, heterocyclylalkyl, cyano, cyanoalkyl, hydroxyl, hydroxyalkyl, alkoxy, alkoxyalkyl, alkoxycarbonyl, carboxy, alkoxycarbonylalkyl, aryl, aralkyl, heteroaryl, heteroarylalkyl, each group being optionally substituted by one or more substituent selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
wherein R²¹ is selected from alkyl, aryl, alkylcarbonyl, heteroaryl or heteroarylcarbonyl.

21. The compound according to claim 1 wherein:
A is O or S,
W is N or CR², Y is N or CR², Z is CH or N, wherein R² is hydrogen or methyl,
p is 3 or 4, m is 0, s is selected from 0, 1, 2 or 3, r is 1 or 2, n is selected from 0, 1, 2, 3, 4, 5, 6, 7 or 8,
wherein R⁵ is selected from hydrogen, alkyl or cycloalkyl,
q is selected from 1, 2, 3 or 4,
R¹⁰ and R¹¹ are each independently a selected from hydrogen, alkyl, cycloalkyl, aryl, or aralkyl,
R¹⁷ is selected from halo, alkoxy, alkyl, alkylamino, alkylcarbonyl, alkylheteroaryl, alkylsuphonyl, aralkyl, aryl, arylamino, aryloxy, cyano, haloalkoxy, haloalkyl, heteroaryl, heteroarylalkyl, heteroarylcarbonyl, heterocyclyl, hydroxy, nitro, oxo, or sulfonyl,
Y² is selected from —CH(R¹⁴)—, —S—, —NH—, —O—, —C(═O)—,
R¹³ and R¹⁴ are each independently selected from hydrogen or alkyl or together with the carbon atoms to which they are attached form an aryl ring,
Y³ is selected from —CH₂—, —S—, —CH₂—CH₂—, —NH—, —O—, —C(═O)—,
wherein when X¹, X² or X³ are each independently selected from CH or N,
X⁴ is selected from N, S or CH,
X⁵ is selected from CH or S.

22. The compound according to claim 1, selected from a compound as listed in any of Tables 1 to 8.

23. A method for synthesizing a compound of Formula XXIII comprising the steps of reacting a compound of Formula XX: 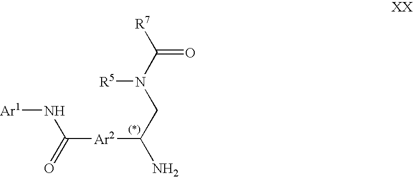
with Noyori's catalyst thereby obtaining a compound of Formula XXI, 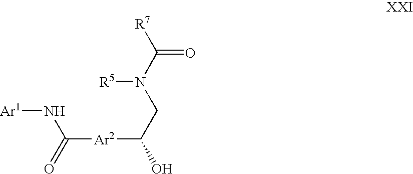
reacting compound of Formula XXI with diphenylphosphoryl azide (DPPA) and 1,8-diazabicyclo[5.4.0]undec-7-ene (DBU) thereby obtaining azide of Formula XXII, and 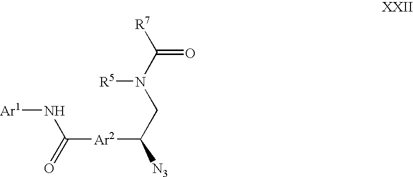
reacting compound of Formula XXII with Pd/C thereby obtaining the compound of Formula XXIII, 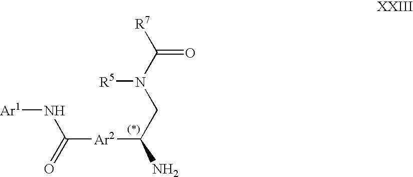
wherein Ar² is phenylene and Ar¹, R⁵ and R⁷ have the same meaning as defined in any of the preceding claims.

24. A compound obtainable by the method of claim 23.

25. A pharmaceutical and/or veterinary composition comprising a compound as defined in claim 1.

26. A pharmaceutical and/or veterinary composition according to claim 25 comprising at least one carrier, excipient or diluent acceptable for pharmaceutical and/or veterinary purposes.

27. (canceled)

28. A method for the prevention and/or treatment of at least one disease and/or disorder selected from the group consisting of metabolic diseases; diabetes; anxiety; addiction; withdrawal symptoms; muscle spasms; convulsive seizures; epilepsy; pain; cardiovascular disease; vascular diseases; inflammatory diseases and/or for regulating the immune system and/or an immune response and/or inflammatory response in a mammal comprising administering to an individual in need of such treatment an effective amount of a compound according to claim 1.

29. A method according to claim 28 wherein said metabolic disease or disorder is at least one of the following:
hyperglycemic conditions and/or other conditions and/or diseases that are associated with insulin, selected from the group consisting of Type I and Type II diabetes, severe insulin resistance, hyperinsulinemia, hyperlipidemia, and insulin-resistant diabetes comprising Mendenhall's Syndrome, Werner Syndrome, leprechaunism and lipoatrophic diabetes, and other lipoatrophies;
obesity;
conditions caused or usually associated with hyperglycemic conditions and/or obesity, including hypertension, osteoporosis and/or lipodystrophy; or
metabolic syndrome;
and inherited metabolic diseases; and/or for preventing, treating and/or alleviating complications and/or symptoms associated with these metabolic diseases.

30. A method for the prevention and/or treatment of type II diabetes, and/or for preventing, treating and/or alleviating complications and/or symptoms associated therewith comprising administering to an individual in need of such treatment an effective amount of a compound according to claim 1.

31. A method for the prevention and/or treatment of obesity, and/or for preventing, treating and/or alleviating complications and/or symptoms associated therewith comprising administering to an individual in need of such treatment an effective amount of a compound according to claim 1.

32. A method for the prevention, treatment and/or management of pain, and/or for preventing, treating and/or alleviating complications and/or symptoms associated therewith comprising administering to an individual in need of such treatment an effective amount of a compound according to claim 1.

33. A method for the prevention and/or treatment of inflammatory diseases selected from the group consisting of contact dermatitis, psoriasis, rheumatoid arthritis, inflammatory bowel disease, Crohn's disease, ulcerative colitis, and/or for preventing, treating and/or alleviating complications and/or symptoms and/or inflammatory responses associated therewith comprising administering to an individual in need of such treatment an effective amount of a compound according to claim 1.

34. A method for the prevention and/or treatment of cardiovascular and vascular diseases selected from the group consisting of acute stroke, congestive heart failure, cardiovascular ischemia, heart disease, cardiac remodeling, angina, coronary vasospasm, cerebral vasospasm, pulmonary vasoconstriction, restenosis, hypertension, pulmonary hypertension, arteriosclerosis, thrombosis and platelet related diseases, and/or for preventing, treating and/or alleviating complications and/or symptoms associated therewith comprising administering to an individual in need of such treatment an effective amount of a compound according to claim 1.

35. A method for the prevention and/or treatment of at least one disease and/or disorder selected from the group consisting of eye diseases; erectile dysfunction; cardiovascular diseases; vascular diseases; proliferative diseases; inflammatory diseases; neurological diseases and disease of the central nervous system (CNS); bronchial asthma; osteoporosis; renal diseases and AIDS comprising administering to an individual in need of such treatment an effective amount of a compound according to claim 1.

36. A method for the prevention and/or treatment of at least one disease and/or disorder selected from the group consisting of erectile dysfunction, bronchial asthma, osteoporosis, inflammatory diseases, renal diseases or AIDS, and/or for preventing, treating and/or alleviating complications and/or symptoms associated therewith comprising administering to an individual in need of such treatment an effective amount of a compound according to claim 1.

37. A method for the prevention and/or treatment of eyes diseases selected from the group consisting of retinopathy, macular degeneration and glaucoma, and/or for preventing, treating and/or alleviating complications and/or symptoms associated therewith comprising administering to an individual in need of such treatment an effective amount of a compound according to claim 1.

38. A method for the prevention, treatment and/or management of neurological and CNS disorders selected from the group consisting of stroke, multiple sclerosis, brain or spinal cord injury, inflammatory diseases and demyelinating diseases comprising Alzheimer's disease, MS and neuropathic pain, and/or for preventing, treating and/or alleviating complications and/or symptoms associated therewith comprising administering to an individual in need of such treatment an effective amount of a compound according to claim 1.

39. A method for the prevention and/or treatment of cancer selected from the group consisting of cancer of the brain (gliomas), breast, colon, intestine, skin, head and neck, kidney, lung, liver, ovarian, pancreatic, prostate, or thyroid; leukemia; lymphoma; sarcoma; melanoma; and/or for preventing, treating and/or alleviating complications and/or symptoms and/or inflammatory responses associated therewith comprising administering to an individual in need of such treatment an effective amount of a compound according to claim 1.

40. A method for inhibiting the activity of at least one kinase, in vitro or in vivo using a compound according to claim 1, or a composition comprising such a compound.

41. The method of claim 40 wherein said use is in vitro.

42. The method of claim 40 wherein the at least one kinase is PKC epsilon.

43. The method of claim 40 for inhibiting the activity of PKC epsilon and PKC theta.

44. The method of claim 40 wherein the at least one kinase is ROCK.

45. The method according to claim 44 in which the at least one kinase is chosen from the alpha and/or beta isoforms of ROCK.

46. The method according to claim 44 in which the at least one kinase is chosen from the alpha isoform of ROCK.
